(19) United States
(12) Patent Application Publication
(10) Pub. No.: US 2023/0320993 A1
(43) Pub. Date: Oct. 12, 2023

(54) METHODS FOR ENCAPSULATING POLYNUCLEOTIDES INTO REDUCED SIZES OF LIPID NANOPARTICLES AND NOVEL FORMULATION THEREOF

(71) Applicant: Generation Bio Co., Cambridge, MA (US)

(72) Inventors: Nolan Gallagher, Cambridge, MA (US); Matthew G. Stanton, Cambridge, MA (US); Gregory Feinstein, Cambridge, MA (US)

(21) Appl. No.: 18/015,575

(22) PCT Filed: Jul. 16, 2021
(86) PCT No.: PCT/US2021/042033

§ 371 Date: Jan. 11, 2023

Related U.S. Application Data
(60) Provisional application No. 63/194,620, filed on May 28, 2021.
(60) Provisional application No. 63/053,274, filed on Jul. 17, 2020.

(51) Int. Cl. A61K 9/51; C12N 15/88; A61K 31/713; A61K 47/28; A61K 47/54

(52) U.S. Cl. CPC A61K 9/5123; A61K 31/713; A61K 47/28; A61K 47/543; C12N 15/88

(57) ABSTRACT

Provided herein are lipid formulations of reduced size, comprising a lipid and a capsid free, non-viral vector (e.g., ceDNA), and methods of producing said lipid formulations. Lipid particles (e.g., lipid nanoparticles) of the disclosure include a lipid formulation that can be used to deliver a capsid-free, non-viral DNA vector to a target site of interest (e.g., cell, tissue, organ, and the like).

### RELATED APPLICATIONS

This application claims priority to U.S. Provisional Application No. 63/053,274, filed on Jul. 17, 2020, and U.S. Provisional Application No. 63/194,620, filed on May 28, 2021, the contents of each of which are hereby incorporated by reference in their entireties.

### BACKGROUND

Lipid nanoparticles (LNPs) are a clinically validated strategy for delivering small interfering RNA (siRNA) cargos to liver hepatocytes. Despite these advances, LNP-mediated delivery of larger, rigid polynucleotide cargos (e.g., double stranded linear DNA, plasmid DNA, closed-ended double stranded DNA (ceDNA)) presents additional challenges relative to the smaller and/or flexible cargos (e.g., siRNA). One such challenge involves the size of the resulting LNP when large, rigid cargo is encapsulated. For example, LNPs encapsulating closed-ended linear DNA (ceDNA) of length > 3000 bp (base pairs) to possess diameters of 80-120 nm have been routinely observed, with a diameter mean of 92 nm (n = 28) using a ‘state of the art’ process involving high pressure microfluidic mixing of aqueous ceDNA in H₂O or in aqueous buffer) from one stream with ethanolic lipids (100% EtOH from another stream (see, e.g., International Application No. PCT/US2020/021328) in an acidic buffer (pH 3-4)).

The relatively large size of these LNPs reduces the therapeutic index for liver indications by several mechanisms: (1) larger LNPs are unable to efficiently bypass the fenestrae of the endothelial cells that line liver sinusoids, preventing access to target cells (hepatocytes); (2) larger LNPs are unable to be efficiently internalized by hepatocytes via clathrin-mediated endocytosis with several different receptors (e.g. asialoglycoprotein receptor (ASGPR), low-density lipoprotein (LDL) receptor); and (3) LNPs above a certain threshold size are prone to preferential uptake by cells of the reticuloendothelial system, which can provoke dose-limiting immune responses. Therefore, manufacturing processes that can encapsulate large, rigid therapeutic nucleic acid molecules into relatively smaller size LNPs (<75 nm diameter) are urgently needed.

### SUMMARY

Provided herein are new formulation processes and methods that are used to produce LNPs that are considerably smaller in diameter than those previously described. The new formulation process described herein comprises reversible compaction of TNA in 80% to 100% low molecular weight alcohol (e.g., ethanol, propanol, isopropanol, butanol, or methanol) prior to the microfluidic nanoparticle assembly with alcoholic (e.g., enthnolic) lipids, which results in LNPs of a mean diameter of 75 nm (± 3 nm) or less.

According to some embodiments, the LNPs described by the present disclosure range in mean diameter from about 20 nm to about 75 nm, about 20 nm to about 70 nm, from about 20 nm to about 60 nm, from about 30 nm to about 75 nm, about 30 nm to about 70 nm, from about 30 nm to about 60 nm, from about 40 nm to 75 nm, or from about 40 nm to 70 nm. Smaller size LNPs provide more efficient tissue diffusion, and more efficient uptake and/or targeting. Particularly in the liver, LNPs of a smaller size are needed to pass liver sinusoidal endothelial cells (LSEC) fenestrae (< 100 nm) and to undergo ASGPR-mediated endocytosis (≤ 70 nm). Such smaller size is also advantageous is in targeting and circumventing unwanted immune responses as they can readily evade immune cells. The formulation process and methods described by the present disclosure can encapsulate considerably more therapeutic nucleic acid (e.g., rigid double stranded DNA including ceDNA) than has been previously reported. The LNPs described herein can encapsulate greater than about 60% to about 90% of rigid double stranded DNA, like ceDNA. According to some embodiments, the LNPs described herein can encapsulate greater than about 60% of rigid double stranded DNA, like ceDNA, greater than about 65% of rigid double stranded DNA, like ceDNA, greater than about 70% of rigid double stranded DNA, like ceDNA, greater than about 75% of rigid double stranded DNA, like ceDNA, greater than about 80% of rigid double stranded DNA, like ceDNA, greater than about 85% of rigid double stranded DNA, like ceDNA, or greater than about 90% of rigid double stranded DNA, like ceDNA.

The formulation process described herein takes advantage of the finding that ceDNA compaction occurs in solvents with 80% to 100% of low molecular weight (LMW) alcohol. The LMW alcohol that can be used for compaction includes, but is not limited to, methanol, ethanol, propanol, isopropanol, butanol or other organic solvent like acetone. Preferably, the compaction of a rigid DNA like ceDNA can be prepared using an ethanolic solution or ethanol-methanol mixture (e.g., EtOH—MeOH 1:1 mixture) at the final concentration of about 80% to about 98%. According to some embodiments, the final concentration of the low molecular weight alcohol in the solution is between about 80% to about 98%, about 80% to about 95%, about 80% to about 92%, about 80% to about 90%, about 80% to about 85%, about 85% to about 98%, about 85% to about 95%, about 85% to about 92%, about 85% to about 90%, about 90% to about 98%, about 87% to about 97%, about, about 87% to about 95%, about 87% to about 92%, about 87% to about 90%, about 90% to about 95%, about 90% to about 92%, about 95% to about 98%, or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98%. For example, when ceDNA in aqueous 90% EtOH is added to or mixed with another ethanolic solution (e.g., 90% EtOH) of lipids in a ratio such that the resulting solution is, for example, 90-92% ethanol and 8-10% water or aqueous buffer, the ceDNA is observed to exist in a highly compacted or denatured state by dynamic light scattering. In such a solvent (e.g., 90-92% ethanol, 8-10% water), both the lipids and ceDNA are solubilized with no detectable precipitation of either component, leading to successful and more efficient encapsulation of a rigid double stranded DNA like ceDNA into smaller sizes of LNP.

Accordingly, the formulation process described herein reduces the LNP diameter, while maintaining similar or better encapsulation efficiency for rigid TNA like ceDNA relative to the standard process. Without wishing to be bound by theory, this change may likely be attributed to compaction of rigid TNA like ceDNA preferably in 90-92% or up to 95% in an LMW alcohol solution such as ethanol solvent prior to formation of LNPs. When LNP formation is then initiated by mixing with the acidic aqueous buffer solution, the lipids are able to nucleate around a smaller and compact DNA (e.g., ceDNA) core as opposed to the standard aqueous process, resulting in significantly smaller particles. Using the process described herein, a rigid TNA like ceDNA can be efficiently encapsulated at a higher number, resulting in TNA-LNPs with much smaller diameters that are beneficial attributes of LNPs to target various tissues that pose size constraints.

In some embodiments, a formulation comprises TNA (e.g., ceDNA) encapsulated in LNPs having a mean diameter of about 75 nm (± 3 nm). In some embodiments, a formulation comprises TNA (e.g., ceDNA) encapsulated in LNPs having a mean diameter of about 72 nm (±3 nm). In some embodiments, a formulation comprises TNA (e.g., ceDNA) encapsulated in LNPs having a mean diameter of about 70 nm (±4 nm). In some embodiments, a formulation comprises TNA (e.g., ceDNA) encapsulated in LNPs having a mean diameter of about 68 nm (±4 nm). In some embodiments, a formulation comprises TNA (e.g., ceDNA) encapsulated in LNPs having a mean diameter of about 65 nm (±4 nm). In some embodiments, a formulation comprises TNA (e.g., ceDNA) encapsulated in LNPs having a mean diameter of about 60 nm (±4 nm). In some embodiments, a formulation comprises TNA (e.g., ceDNA) encapsulated in LNPs having a mean diameter of about 55 nm (±4 nm). In some embodiments, a formulation comprises TNA (e.g., ceDNA) encapsulated in LNPs having a mean diameter of about 50 nm (±4 nm).

According to a first aspect, the disclosure provides a pharmaceutical composition comprising lipid nanoparticle (LNP), wherein the LNP comprises a lipid and a rigid nucleic acid therapeutic (rTNA), wherein the mean diameter of the LNP is between about 20 nm and about 75 nm.

According to some embodiments, the rigid nucleic acid therapeutic is a double stranded nucleic acid. According to some embodiments, the rigid nucleic acid therapeutic is a closed ended DNA.

According to some embodiments, the lipid is selected from an ionizable lipid, a non-cationic lipid, a sterol or a derivative thereof, a conjugated lipid, or any combination thereof. According to some embodiments, the ionizable lipid is a cationic lipid. According to some embodiments, the cationic lipid is an SS-cleavable lipid.

According to some embodiments of the aspects and embodiments disclosed herein, the ionizable lipid is represented by Formula (I):

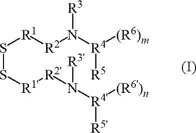

or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R³′ are each independently optionally substituted linear or branched C₁₋₆ alkyl;
or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or —[C(R^{a})₂]₂CR^{a};
R^{a}, for each occurrence, is independently H or C₁₋₃ alkyl;
or alternatively, when R⁴ is —C(R^{a})₂CR^{a}, or —[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or —[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{5′} are each independently C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

According to some embodiments of the aspects and embodiments disclosed herein, the ionizable lipid is represented by Formula (II):

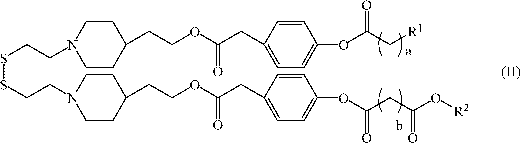

or a pharmaceutically acceptable salt thereof, wherein:
a is an integer ranging from 1 to 20;
b is an integer ranging from 2 to 10;
R¹ is absent or is selected from (C₂-C₂₀)alkenyl, -C(O)O(C₂-C₂₀)alkyl, and cyclopropyl substituted with (C₂-C₂₀)alkyl; and
R² is (C₂-C₂₀)alkyl.

According to some embodiments of the aspects and embodiments disclosed herein, the ionizable lipid is represented by the Formula (V):

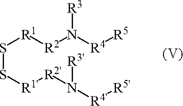

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

According to some embodiments, the ionizable lipid is represented by Formula (XV):

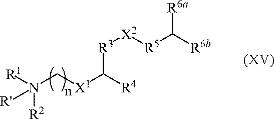

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₆ unbranched alkyl, C₂-C₁₆ unbranched alkenyl, or

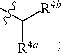

; wherein:
   _{R}^{4a} and R^{4b} are each independently C₁-C₁₆ unbranched alkyl or C₂-C₁₆ unbranched alkenyl;
R⁵ is absent, C₁-C₈ alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and n is an integer selected from 1, 2, 3, 4, 5, and 6.

According to some embodiments, the ionizable lipid is represented by Formula (XX):

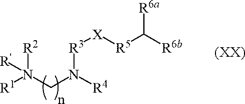

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆ unbranched alkenyl, or

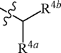

; wherein:
   R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})2O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and n is an integer selected from 1, 2, 3, 4, 5, and 6.

According to some embodiments, the ionizable lipid is selected from any lipid in Table 2, Table 5, Table 6, Table 7, or Table 8.

According to some embodiments, the ionizable lipid is a lipid having the structure:

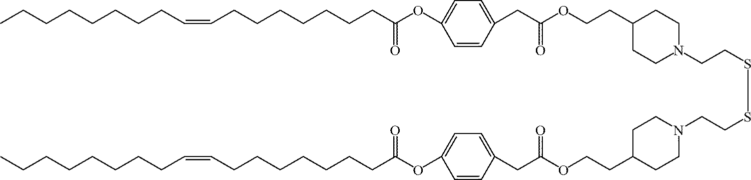 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

or a pharmaceutically acceptable salt thereof.

According to some embodiments, the cationic lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

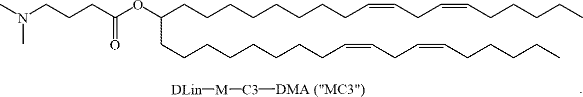

According to some embodiments of the aspects and embodiments disclosed herein, the LNP further comprises a sterol. According to some embodiments, the sterol is a cholesterol.

According to some embodiments of the aspects and embodiments disclosed herein, the LNP further comprises a polyethylene glycol (PEG). According to some embodiments, the PEG is 1-(monomethoxy-polyethyleneglycol)-2,3-dimyristoylglycerol (PEG-DMG).

According to some embodiments of the aspects and embodiments disclosed herein, the LNP further comprises a non-cationic lipid. According to some embodiments, the non-cationic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3 -pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof.

According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE).

According to some embodiments, the PEG or PEG-lipid conjugate is present at about 1.5% to about 3%.

According to some embodiments, the cholesterol is present at a molar percentage of about 20% to about 40%, and wherein the lipid is present at a molar percentage of about 80% to about 60%.

According to some embodiments, the cholesterol is present at a molar percentage of about 40%, and wherein the lipid is present at a molar percentage of about 50%.

According to some embodiments, the composition further comprises a cholesterol, a PEG or PEG-lipid conjugate, and a non-cationic lipid.

According to some embodiments, the PEG or PEG-lipid conjugate is present at about 1.5% to about 3%, about 1.5% to about 2.75%, about 1.5% to about 2.5%, about 1.5% to about 2%, about 2% to about 3%, about 2% to about 2.75%, about 2% to about 2.5%, about 2.5% to about 3% about 2.5% to about 2.75%, or about 2.5% to about 3%.

According to some embodiments, the cholesterol is present at a molar percentage of about 30% to about 50%, about 30% to about 45%, about 30% to about 40%, about 30% to about 35%, about 35% to about 40%, about 35% to about 45%, about 35% to about 50%, about 40% to about 45%, about 40% to about 50%, or about 45% to about 50%.

According to some embodiments, the lipid is present at a molar percentage of about 42.5% to about 62.5%, about 42.5% to about 57.5%, about 42.5% to about 52.5%, about 42.5% to about 47.5%, about 47.5% to about 62.5%, about 47.5% to about 57.5%, about 47.5% to about 52.5%, about 52.5% to about 62.5%, about 52.5% to about 57.5%, or about 57.5% to about 62.5%.

According to some embodiments, the non-cationic lipid is present at a molar percentage of about 2.5% to about 12.5%, about 2.5% to about 10.5%, about 2.5% to about 8.5%, about 2.5% to about 6.5%, about 2.5% to about 4.5%, about 4.5% to about 12.5%, about 4.5% to about 10.5%, about 4.5% to about 8.5%, about 4.5% to about 6.5%, about 6.5% to about 12.5%, about 6.5% to about 10.5%, about 6.5% to about 8.5%, about 8.5% to about 12.5%, about 8.5% to about 10.5%, or about 10.5% to about 12.5%.

According to some embodiments of the aspects and embodiments herein, the cholesterol is present at a molar percentage of about 40%, the lipid is present at a molar percentage of about 52.5%, the non-cationic lipid is present at a molar percentage of about 7.5%, and wherein the PEG is present at about 3%.

According to some embodiments of the aspects and embodiments disclosed herein, the composition further comprises dexamethasone palmitate.

According to some embodiments of the aspects and embodiments disclosed herein, the LNP is less than about 75 nm in size. According to some embodiments of the aspects and embodiments disclosed herein, the LNP is less than about 70 nm in size, for example less than about 65 nm, less than about 60 nm, less than about 55 nm, less than about 50 nm, less than about 45 nm, less than about 40 nm, less than about 35 nm, less than about 30 nm, less than about 25 nm, less than about 20 nm, less than about 15 nm, or less than about 10 nm in size. According to some embodiments of the aspects and embodiments disclosed herein, the LNP is less than about 70 nm, 69 nm, 68 nm, 67 nm, 66 nm, 65 nm, 64 nm, 63 nm, 62 nm, 61 nm, 60 nm, 59 nm, 58 nm, 57 nm, 56 nm, 55 nm, 54 nm, 53 nm, 52 nm, 51 nm, or 50 nm in size.

According to some embodiments of the aspects and embodiments disclosed herein, the composition has a total lipid to rigid therapeutic nucleic acid (rTNA) ratio of about 15:1.

According to some embodiments of the aspects and embodiments disclosed herein, the composition has a total lipid to rigid therapeutic nucleic acid (rTNA) ratio of about 30:1.

According to some embodiments of the aspects and embodiments disclosed herein, the composition has a total lipid to rigid therapeutic nucleic acid (rTNA) ratio of about 40:1.

According to some embodiments of the aspects and embodiments disclosed herein, the composition has a total lipid to rigid therapeutic nucleic acid (rTNA) ratio of about 50:1. According to some embodiments of the aspects and embodiments disclosed herein, the composition has a total lipid to rigid therapeutic nucleic acid (rTNA) ratio of between about 15:1 to about 30: 1. According to some embodiments of the aspects and embodiments disclosed herein, the composition has a total lipid to rigid therapeutic nucleic acid (rTNA) ratio of between about 15:1 to about 40: 1. According to some embodiments of the aspects and embodiments disclosed herein, the composition has a total lipid to rigid therapeutic nucleic acid (rTNA) ratio of between about 15:1 to about 50: 1. According to some embodiments of the aspects and embodiments disclosed herein, the composition has a total lipid to rigid therapeutic nucleic acid (rTNA) ratio of between about 30:1 to about 40: 1. According to some embodiments of the aspects and embodiments disclosed herein, the composition has a total lipid to rigid therapeutic nucleic acid (rTNA) ratio of between about 30:1 to about 50: 1. According to some embodiments of the aspects and embodiments disclosed herein, the composition has a total lipid to rigid therapeutic nucleic acid (rTNA) ratio of between about 40:1 to about 50: 1. According to some embodiments of the aspects and embodiments disclosed herein, the composition further comprises N-Acetylgalactosamine (GalNAc). According to some embodiments, the GalNAc is present in the LNP at a molar percentage of 0.5% of the total lipid. According to some embodiments, the GalNAc is present in the LNP at a molar percentage of between about 0.3% to about 0.9%, between about 0.4% to about 0.8%, between about 0.5% to about 0.6% of the total lipid.

According to some embodiments of the aspects and embodiments disclosed herein, the rigid therapeutic nucleic acid (rTNA)is closed-ended DNA (ceDNA).

According to some embodiments of the aspects and embodiments disclosed herein, the rigid therapeutic nucleic acid (rTNA)comprises an expression cassette comprising a promoter sequence and a transgene.

According to some embodiments, the rigid therapeutic nucleic acid (rTNA)comprises an expression cassette comprising a polyadenylation sequence.

According to some embodiments, the rigid therapeutic nucleic acid (rTNA) comprises at least one inverted terminal repeat (ITR) flanking either the 5′ or the 3′ end of said expression cassette.

According to some embodiments, the expression cassette is flanked by two ITRs, wherein the two ITRs comprise one 5′ ITR and one 3′ ITR.

According to some embodiments, the expression cassette is connected to an ITR at a 3′ end (3′ ITR). According to some embodiments, the expression cassette is connected to an ITR at a 5′ end (5′ ITR).

According to some embodiments, at least one of the 5′ ITR or the 3′ ITR is a wild-type AAV ITR. According to some embodiments, at least one of a 5′ ITR and a 3′ ITR is a modified ITR.

According to some embodiments, the rigid therapeutic nucleic acid (rTNA) further comprises a spacer sequence between a 5′ ITR and the expression cassette.

According to some embodiments, the rigid therapeutic nucleic acid (rTNA) further comprises a spacer sequence between a 3′ ITR and the expression cassette.

According to some embodiments, the spacer sequence is at least 5 base pairs long. According to some embodiments, the spacer sequence is 5 to 100 base pairs long. According to some embodiments, the spacer sequence is 5, 10, 15, 20, 25, 30, 35, 40, 45, 50, 55, 60, 65, 70, 75, 80, 85, 90, 95 or 100 base pairs long. According to some embodiments, the spacer sequence is 5 to 500 base pairs long. According to some embodiments, the spacer sequence is 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 base pairs long.

According to some embodiments, the rigid therapeutic nucleic acid (rTNA) has a nick or a gap.

According to some embodiments, the ITR is an ITR selected from an ITR derived from an AAV serotype, an ITR derived from an ITR of goose virus, an ITR derived from a B19 virus ITR, or a wild-type ITR from a parvovirus.

According to some embodiments, said AAV serotype is selected from the group consisting of: AAV1, AAV2, AAV3, AAV4, AAV5, AAV6, AAV7, AAV8, AAV9, AAV10, AAV11 and AAV12.

According to some embodiments, the ITR is a mutant ITR, and the ceDNA optionally comprises an additional ITR which differs from the first ITR.

According to some embodiments, the ceDNA comprises two mutant ITRs in both 5′ and 3′ ends of the expression cassette, optionally wherein the two mutant ITRs are symmetric mutants.

According to some embodiments of the aspects and embodiments disclosed herein, the rigid therapeutic nucleic acid (rTNA) is selected from the group consisting of minigenes, plasmids, minicircles, small interfering RNA (siRNA), microRNA (miRNA), antisense oligonucleotides (ASO), ribozymes, ceDNA, ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof.

According to some embodiments of the aspects and embodiments disclosed herein, the rigid therapeutic nucleic acid is a plasmid.

According to some embodiments of the aspects and embodiments disclosed herein, the pharmaceutical composition further comprises a pharmaceutically acceptable excipient.

According to another aspect, the disclosure provides a method of producing a lipid nanoparticle (LNP) formulation, wherein the LNP comprises an ionizable lipid and a closed-ended DNA (ceDNA), the method comprising adding aqueous ceDNA to one or more low molecular weight alcohols (e.g., ethanol, methanol, propanol, or isopropanol) solution comprising cationic or ionizable lipids, wherein the final concentration of alcohol in the solution is between about 80% to about 98% form a ceDNA/lipid solution; mixing the ceDNA/lipid solution with an acidic aqueous buffer; and buffer exchanging with a neutral-pH aqueous buffer, thereby producing an LNP formulation. According to some embodiments, the final concentration of the low molecular weight alcohol in the solution is between about 80% to about 98%, about 80% to about 95%, about 80% to about 92%, about 80% to about 90%, about 80% to about 85%, about 85% to about 98%, about 85% to about 95%, about 85% to about 92%, about 85% to about 90%, about 90% to about 98%, about 87% to about 97%, about, about 87% to about 95%, about 87% to about 92%, about 87% to about 90%, about 90% to about 95%, about 90% to about 92%, about 95% to about 98%, or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98%.

According to another aspect, the disclosure provides a method of producing a lipid nanoparticle (LNP) formulation comprising an ionizable lipid and a closed-ended DNA (ceDNA), the method comprising adding ceDNA to one or more low molecular weight alcohols (e.g., ethanol, methanol, propanol, or isopropanol) solution, wherein the alcohol content of the resulting solution is greater than 80%, adding said ceDNA in >80% alcohol content to cationic or ionizable lipids in 80% alcohol, wherein the concentration of the low molecular weight alcohol in the ceDNA-lipid solution is between about 80% to about 95% (e.g., 80%, 81%, 82%, 83%, 84%, 85%, 86%, 87%, 88%, 89%, 90%, 91%, 92%, 93%, 94%, or 95%) to form a ceDNA/lipid solution; mixing the ceDNA/lipid solution with an acidic aqueous buffer; and buffer exchanging with a neutral-pH aqueous buffer, thereby producing an LNP formulation. According to some embodiments, the final concentration of the low molecular weight alcohol in the solution is between about 80% to about 98%, about 80% to about 95%, about 80% to about 92%, about 80% to about 90%, about 80% to about 85%, about 85% to about 98%, about 85% to about 95%, about 85% to about 92%, about 85% to about 90%, about 90% to about 98%, about 87% to about 97%, about, about 87% to about 95%, about 87% to about 92%, about 87% to about 90%, about 90% to about 95%, about 90% to about 92%, about 95% to about 98%, or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98%.

According to some embodiments, the method further comprises a step of diluting the mixed ceDNA/lipid solution with an acidic aqueous buffer.

According to some embodiments, the one or more low molecular weight alcohol is selected from the group consisting of methanol, ethanol, propanol and isopropanol. According to some embodiments, the one or more low molecular weight alcohol is ethanol. According to some embodiments, the one or more low molecular weight alcohol is propanol. According to some embodiments, the one or more low molecular weight alcohol is methanol. According to some embodiments, the one or more low molecular weight alcohol is a mixture of ethanol and methanol.

According to some embodiments, the acid aqueous buffer is selected from malic acid/sodium malate or acetic acid/sodium acetate. According to some embodiments, the acidic aqueous buffer is at a concentration of between about 10 to 40 millimolar (mM), for example about about 10 mM to about 20 mM, about 10 mM to about 30 mM, about 20 mM to about 30 mM, about 20 mM to about 40 mM, about 30 mM to about 40 mM, or about 10 mM to about 15 mM. According to some embodiments, the acidic aqueous buffer is at a pH of between about 3 to 5.

According to some embodiments, the neutral-pH aqueous buffer is Dulbecco’s phosphate buffered saline, pH 7.4.

According to some embodiments, the ceDNA/lipid solution is mixed with the acidic aqueous buffer using microfluidic mixing.

According to some embodiments, the final alcohol content following the diluting step is between about 4% to about 15% (e.g., about 4%, 5%, 6%, 7%, 8%, 9%, 10%, 11%, 12%, 13%, 14%, or 15%).

According to some embodiments, the flow rate ratio between the acidic aqueous buffer and the ceDNA/lipid solution is 2:1, 3:2, 3:1, 5:1, 6:1, 7:1, 8:1, 9:1, 10:1, 15:1 or 20:1.

According to some embodiments, the LNP has a mean diameter of between about 20 nm and about 70 nm, for example about 20 nm, about 25 nm, about 30 nm, about 35 nm, about 40 nm, about 45 nm, about 50 nm, about 55 nm, about 60 nm, about 65 nm or about 70 nm.

According to some embodiments, the cationic lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

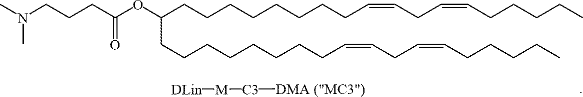

According to some embodiments, the ionizable lipid is a SS-cleavable lipid comprising a disulfide bond and a tertiary amine.

According to some embodiments, the SS-cleavable lipid comprises an ss-OP lipid of the formula:

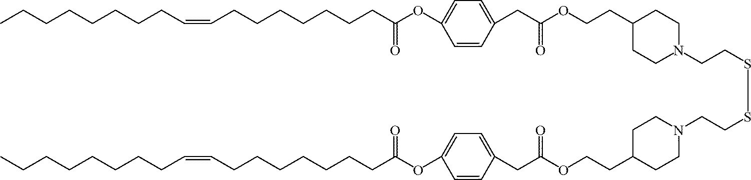 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

or a pharmaceutically acceptable salt thereof.

According to some embodiments, the disclosure provides an LNP formulation produced by the methods described in the aspects and embodiments herein.

According to another aspect, the disclosure provides a method of treating a genetic disorder in a subject, the method comprising administering to the subject an effective amount of the pharmaceutical composition according to any of the previous claims.

According to some embodiments, the subject is a human.

According to some embodiments, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson’s disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi’s anemia, retinitis pigmentosa, ataxia telangiectasia, Bloom’s syndrome, retinoblastoma, mucopolysaccharide storage diseases (e.g., Hurler syndrome (MPS Type I), Scheie syndrome (MPS Type IS), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson’s disease, Alzheimer’s disease, Huntington’s disease, spinocerebellar ataxia, spinal muscular atrophy, Friedreich’s ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency. According to some embodiments, the genetic disorder is Leber congenital amaurosis (LCA).

According to some embodiments, the LCA is LCA10.

According to some embodiments, the genetic disorder is Niemann-Pick disease. According to some embodiments, the genetic disorder is Stargardt macular dystrophy. According to some embodiments, the genetic disorder is glucose-6-phosphatase (G6Pase) deficiency (glycogen storage disease type I) or Pompe disease (glycogen storage disease type II). According to some embodiments, the genetic disorder is hemophilia A (Factor VIII deficiency). According to some embodiments, the genetic disorder is hemophilia B (Factor IX deficiency). According to some embodiments, the genetic disorder is hunter syndrome (Mucopolysaccharidosis II). According to some embodiments, the genetic disorder is cystic fibrosis. According to some embodiments, the genetic disorder is dystrophic epidermolysis bullosa (DEB). According to some embodiments, the genetic disorder is phenylketonuria (PKU). According to some embodiments, wherein the genetic disorder is hyaluronidase deficiency.

According to some embodiments of the aspects and embodiments disclosed herein, the method further comprises administering an immunosuppressant.

According to some embodiments, the immunosuppressant is dexamethasone.

According to some embodiments of the aspects and embodiments disclosed herein, the subject exhibits a diminished immune response level against the pharmaceutical composition, as compared to an immune response level observed with an LNP comprising MC3 as a main cationic lipid, wherein the immune response level against the pharmaceutical composition is at least 50% lower than the level observed with the LNP comprising MC3.

According to some embodiments, the immune response is measured by detecting the levels of a pro-inflammatory cytokine or chemokine.

According to some embodiments, the pro-inflammatory cytokine or chemokine is selected from the group consisting of IL-6, IFNα, IFNγ, IL-18, TNFα, IP-10, MCP-1, MIP1α, MIP1β, and RANTES.

According to some embodiments, at least one of the pro-inflammatory cytokines is under a detectable level in serum of the subject at 6 hours after the administration of the pharmaceutical composition.

According to some embodiments, the LNP comprising the SS-cleavable lipid and the closed-ended DNA (ceDNA) is not phagocytosed; or exhibits diminished phagocytic levels by at least 50% as compared to phagocytic levels of LNPs comprising MC3 as a main cationic lipid administered at a similar condition.

According to some embodiments, the SS-cleavable lipid comprises an ssOP lipid of the formula:

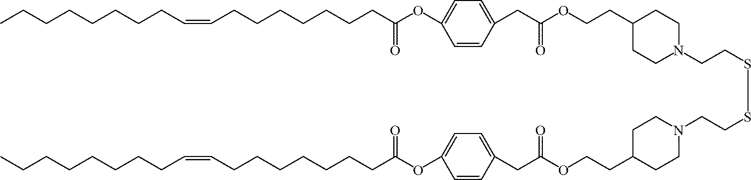 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

or a pharmaceutically acceptable salt thereof.

According to some embodiments, the LNP further comprises cholesterol and a PEG-lipid conjugate.

According to some embodiments, the LNP further comprises a noncationic lipid.

According to some embodiments, the noncationic lipid is selected from the group consisting of dioleoylphosphatidylcholine (DOPC), distearoylphosphatidylcholine (DSPC), and dioleoyl-phosphatidylethanolamine (DOPE).

According to some embodiments, the LNP further comprises N-Acetylgalactosamine (GalNAc).

According to some embodiments, the GalNAc is present in the LNP at a molar percentage of 0.5% of the total lipid.

According to another aspect, the disclosure provides a method of increasing therapeutic nucleic acid targeting to the liver of a subject in need of treatment, the method comprising administering to the subject an effective amount of the pharmaceutical composition according to any of the previous claims, wherein the LNP comprises a therapeutic nucleic acid, ss-cleavable lipid, sterol, and polyethylene glycol (PEG) and N-Acetylgalactosamine (GalNAc).

According to some embodiments, the PEG is 1-(monomethoxy-polyethyleneglycol)-2,3-dimyristoylglycerol (PEG-DMG).

According to some embodiments, the LNP further comprises a non-cationic lipid.

According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE).

According to some embodiments, the GalNAc is present in the LNP at a molar percentage of 0.5% of the total lipid.

According to some embodiments, the subject is suffering from a genetic disorder.

According to some embodiments, the genetic disorder is hemophilia A (Factor VIII deficiency). According to some embodiments, the genetic disorder is hemophilia B (Factor IX deficiency). According to some embodiments, the genetic disorder is phenylketonuria (PKU).

According to some embodiments, the therapeutic nucleic acid is selected from the group consisting of minigenes, plasmids, minicircles, small interfering RNA (siRNA), microRNA (miRNA), antisense oligonucleotides (ASO), ribozymes, ceDNA, ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof.

According to some embodiments, the therapeutic nucleic acid is ceDNA.

According to some embodiments, the ceDNA comprises an expression cassette comprising a promoter sequence and a transgene.

According to some embodiments, the ceDNA comprises at least one inverted terminal repeat (ITR) flanking either 5′ or 3′ end of said expression cassette.

According to some embodiments, the ceDNA is selected from the group consisting of a CELiD, a MIDGE, a ministering DNA, a dumbbell shaped linear duplex closed-ended DNA comprising two hairpin structures of ITRs in the 5′ and 3′ ends of an expression cassette, or a doggybone™ DNA, wherein the ceDNA is capsid free and linear duplex DNA.

According to some aspects, the disclosure provides a method of mitigating a complement response in a subject in need of treatment with a therapeutic nucleic acid (TNA), the method comprising administering to the subject an effective amount of the pharmaceutical composition according to any of the previous claims, wherein the LNP comprises the TNA, a ss-cleavable lipid, a sterol, polyethylene glycol (PEG), and N-Acetylgalactosamine (GalNAc).

According to some embodiments, the subject is suffering from a genetic disorder.

According to some embodiments, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson’s disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi’s anemia, retinitis pigmentosa, ataxia telangiectasia, Bloom’s syndrome, retinoblastoma, mucopolysaccharide storage diseases (e.g., Hurler syndrome (MPS Type I), Scheie syndrome (MPS Type IS), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson’s disease, Alzheimer’s disease, Huntington’s disease, spinocerebellar ataxia, spinal muscular atrophy, Friedreich’s ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency.

According to some embodiments, the rigid therapeutic nucleic acid is selected from the group consisting of minigenes, plasmids, minicircles, small interfering RNA (siRNA), microRNA (miRNA), antisense oligonucleotides (ASO), ribozymes, ceDNA, ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral vector, non-viral vector and any combination thereof.

According to some embodiments, the ceDNA is selected from the group consisting of a CELiD, a MIDGE, a ministering DNA, a dumbbell shaped linear duplex closed-ended DNA comprising two hairpin structures of ITRs in the 5′ and 3′ ends of an expression cassette, or a doggybone™ DNA, wherein the ceDNA is capsid free and linear duplex DNA.

According to some embodiments, the PEG is 1-(monomethoxy-polyethyleneglycol)-2,3-dimyristoylglycerol (PEG-DMG).

According to some embodiments, the PEG is present in the LNP at a molecular percentage of about 2 to 4%. According to some embodiments, the PEG is present in the LNP at a molecular percentage of about 3%.

According to some embodiments, the LNP further comprises a non-cationic lipid. According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE).

According to some embodiments, the GalNAc is present in the LNP at a molar percentage of about 0.3 to 1% of the total lipid. According to some embodiments, the GalNAc is present in the LNP at a molar percentage of about 0.5% of the total lipid.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A is a graph that shows condensation of ceDNA as determined by dynamic light scattering. Dynamic light scattering correlation functions show condensation of ceDNA as ethanol content increases. FIG. 1B is a graph that shows compaction is reversible upon rehydration.

FIG. 2 is a graph that shows a comparison of diameter of ceDNA LNPs produced by the standard formulation process and the new formulation process described herein.

FIGS. 3A and 3B are transmission electron microscopy (TEM) images of a ceDNA sample and a plasmid DNA (pDNA) sample, respectively, stored in deionized (DI) water. FIG. 3A depics a TEM image of ceDNA stored in deionized water. FIG. 3B depics a TEM image of a plasimid stored in deionized water.

FIGS. 4A and 4B are TEM images of a ceDNA sample and a pDNA sample, respectively, stored in a low molecular weight alcohol/water solution of 90.9% 1:1 ethanol:methanol in deionized water. FIG. 4A depics a TEM image of ceDNA stored in 90.9% 1:1 ethanol:methanol in deionized water. FIG. 4B depics a TEM image of a plasimid stored in 90.9% 1:1 ethanol:methanol in deionized water.

FIG. 5 is a TEM image of a ceDNA sample stored in 100% low molecular weight alcohol (i.e., 1:1 ethanol:methanol with no water).

FIGS. 6A and 6B are TEM images of ceDNA and pDNA, respectively, stored in a basic denaturing condition of 100 mM sodium hydroxide (NaOH) aqueous solution.

### DETAILED DESCRIPTION

The immunogenicity associated with viral vector-based gene therapies has limited the number of patients who could be treated due to pre-existing background immunity, as well as prevented the re-dosing of patients either to titrate to effective levels in each patient, or to maintain effects over the longer term. Because of the lack of pre-existing immunity, the presently described therapeutic nucleic acid lipid particles (e.g., lipid nanoparticles) allow for additional doses of the therapeutic nucleic acid as necessary, and further expands patient access, including into pediatric populations who may require a subsequent dose upon tissue growth. The therapeutic nucleic acid lipid particles (e.g., lipid nanoparticles) produced by the process described herein, and comprising in particular cationic or ionizable lipid compositions comprising one or more tertiary amino groups to provide more efficient delivery of the therapeutic nucleic acid, better tolerability and an improved safety profile due to their smaller size as compared to that of LNP produced from conventional LNP making processes. Because the presently described therapeutic nucleic acid lipid particles (e.g., lipid nanoparticles) have no packaging constraints imposed by the space within the viral capsid, in theory, the only size limitation of the therapeutic nucleic acid lipid particles (e.g., lipid nanoparticles) resides in the DNA replication efficiency of the host cell. As described and exemplified herein, according to some embodiments, the therapeutic nucleic acid is a therapeutic nucleic acid (TNA) like double stranded DNA (e.g., ceDNA). Described and exemplified herein, according to some embodiments, the therapeutic nucleic acid is a ceDNA. As also described herein, according to some embodiments, the therapeutic nucleic acid is a mRNA.

One of the most difficult hurdles in the development of therapeutics, particularly in rare diseases, is the large number of individual conditions. Around 350 million people on earth are living with rare disorders, defined by the National Institutes of Health as a disorder or condition with fewer than 200,000 people diagnosed. About 80 percent of these rare disorders are genetic in origin, and about 95 percent of them do not have treatment approved by the FDA (rarediseases.info.nih.gov/diseases/pages/311faqs-about-rare-diseases). Among the advantages of the ceDNA lipid particles (e.g., lipid nanoparticles) described herein is in providing an approach that can be rapidly adapted to multiple diseases, and particularly to rare monogenic diseases that can meaningfully change the current state of treatments for many of the genetic disorder or diseases.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6th Edition, published by Lippincott Williams & Wilkins, Philadelphia, PA, USA (2013), Knipe, D.M. and Howley, P.M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), Taylor & Francis Limited, 2014 (ISBN 0815345305, 9780815345305); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al. Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan), ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used in this specification and the appended claims, the singular forms “a”, “an” and “the” include plural references unless the content clearly dictates otherwise.

The abbreviation, “e.g.” is derived from the Latin exempli gratia and is used herein to indicate a non-limiting example. Thus, the abbreviation “e.g.” is synonymous with the term “for example.”

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, preferred materials and methods are described herein.

As used herein the terms, “administration,” “administering” and variants thereof refers to introducing a composition or agent (e.g., nucleic acids, in particular ceDNA) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the phrase “anti-therapeutic nucleic acid immune response”, “anti-transfer vector immune response”, “immune response against a therapeutic nucleic acid”, “immune response against a transfer vector”, or the like is meant to refer to any undesired immune response against a therapeutic nucleic acid, viral or non-viral in its origin. In some embodiments, the undesired immune response is an antigen-specific immune response against the viral transfer vector itself. In some embodiments, the immune response is specific to the transfer vector which can be double stranded DNA, single stranded RNA, or double stranded RNA. In other embodiments, the immune response is specific to a sequence of the transfer vector. In other embodiments, the immune response is specific to the CpG content of the transfer vector.

As used herein, the term “aqueous solution” is meant to refer to a composition comprising in whole, or in part, water.

As used herein, the term “bases” includes purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the term “carrier” is meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “ceDNA” is meant to refer to capsid-free closed-ended linear double stranded (ds) duplex DNA for non-viral gene transfer, synthetic or otherwise. According to some embodiments, the ceDNA is a closed-ended linear duplex (CELiD) CELiD DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministering DNA. According to some embodiments, the ceDNA is a dumbbell shaped linear duplex closed-ended DNA comprising two hairpin structures of ITRs in the 5′ and 3′ ends of an expression cassette. According to some embodiments, the ceDNA is a doggybone™ DNA. Detailed description of ceDNA is described in International Patent Application No. PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International Patent Application Nos. PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “ceDNA-bacmid” is meant to refer to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” is meant to refer to a baculovirus that comprises a ceDNA genome as an intermolecular duplex within the baculovirus genome.

As used herein, the terms “ceDNA-baculovirus infected insect cell” and “ceDNA-BIIC” are used interchangeably, and are meant to refer to an invertebrate host cell (including, but not limited to an insect cell (e.g., an Sf9 cell)) infected with a ceDNA-baculovirus.

As used herein, the term “ceDNA genome” is meant to refer to an expression cassette that further incorporates at least one inverted terminal repeat (ITR) region. A ceDNA genome may further comprise one or more spacer regions. In some embodiments the ceDNA genome is incorporated as an intermolecular duplex polynucleotide of DNA into a plasmid or viral genome.

As used herein, the terms “DNA regulatory sequences,” “control elements,” and “regulatory elements,” are used interchangeably herein, and are meant to refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

As used herein, the term “rigid therapeutic nucleic acid”, “rigid TNA” or “rTNA” refers to a therapeutic nucleic acid as defined herein that has a compact structure or is in a compact state, for example, as a result of a process during the preparation of an LNP composition comprising the rTNA as described herein. In one embodiment, the preparation includes an LMW alcohol-based process whereby the rTNA and lipids are mixed in an LMW alcohol solution and the LMW alcohol mixture containing the rTNA and lipids is introduced to a microfluidic synthesis system (e.g., NanoAssemblr) through one channel and an aqueous buffer is introduced to the system via a separate channel to produce LNP compositions encapsulating the rTNA. As used herein, the term “terminal repeat” or “TR” includes any viral or non-viral terminal repeat or synthetic sequence that comprises at least one minimal required origin of replication and a region comprising a palindromic hairpin structure. A Rep-binding sequence (“RBS” or also referred to as Rep-binding element (RBE)) and a terminal resolution site (“TRS”) together constitute a “minimal required origin of replication” for an AAV and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs plays a critical role in mediating replication, viral particle and DNA packaging, DNA integration and genome and provirus rescue. TRs that are not inverse complement (palindromic) across their full length can still perform the traditional functions of ITRs, and thus, the term ITR is used to refer to a TR in an viral or non-viral AAV vector that is capable of mediating replication of in the host cell. It will be understood by one of ordinary skill in the art that in a complex AAV vector configurations more than two ITRs or asymmetric ITR pairs may be present.

The “ITR” can be artificially synthesized using a set of oligonucleotides comprising one or more desirable functional sequences (e.g., palindromic sequence, RBS). The ITR sequence can be an AAV ITR, an artificial non-AAV ITR, or an ITR physically derived from a viral AAV ITR (e.g., ITR fragments removed from a viral genome). For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, ITR can be present in only one of end of the linear vector. For example, the ITR can be present on the 5′ end only. Some other cases, the ITR can be present on the 3′ end only in synthetic AAV vector. For convenience herein, an ITR located 5′ to (“upstream of”) an expression cassette in a synthetic AAV vector is referred to as a “5′ ITR” or a “left ITR”, and an ITR located 3′ to (“downstream of”) an expression cassette in a vector or synthetic AAV is referred to as a “3′ ITR” or a “right ITR”.

As used herein, a “wild-type ITR” or “WT-ITR” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a synthetic AAV vector that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C—C′ and B—B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably and refer to an ITR with a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single synthetic AAV genome that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C—C′ and B—B′ loops in 3D space (e.g., one ITR may have a short C—C′ arm and/or short B—B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C—C′ arm and the other ITR can have a different modification (e.g., a single arm, or a short B—B′ arm etc.) such that they have different three-dimensional spatial organization as compared to the cognate asymmetric mod-ITR.

As used herein, the term “symmetric ITRs” refers to a pair of ITRs within a single stranded AAV genome that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a synthetic AAV vector is referred to as a “5′ ITR” or a “left ITR”, and an ITR located 3′ to (downstream of) an expression cassette in a synthetic AAV vector is referred to as a “3′ ITR” or a “right ITR”.

As used herein, the terms “substantially symmetrical modified-ITRs” or a “substantially symmetrical mod-ITR pair” refers to a pair of modified-ITRs within a synthetic AAV that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C—C′ and B—B′ loops organized in 3D space. In some embodiments, the ITRs from a mod-ITR pair may have different reverse complement nucleotide sequences but still have the same symmetrical three-dimensional spatial organization - that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C—C′ and B-B′ loops in 3D space, e.g., if a modified ITR in a substantially symmetrical mod-ITR pair has a deletion of a C—C′ arm, then the cognate mod-ITR has the corresponding deletion of the C—C′ loop and also has a similar 3D structure of the remaining A and B—B′ loops in the same shape in geometric space of its cognate mod-ITR.

As used herein, the phrase an “effective amount” or “therapeutically effective amount” of an active agent or therapeutic agent, such as a therapeutic nucleic acid, is an amount sufficient to produce the desired effect, e.g., inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

As used herein, the term “expression” is meant to refer to the cellular processes involved in producing RNA and proteins and as appropriate, secreting proteins, including where applicable, but not limited to, for example, transcription, transcript processing, translation and protein folding, modification and processing. As used herein, the phrase “expression products” include RNA transcribed from a gene (e.g., transgene), and polypeptides obtained by translation of mRNA transcribed from a gene.

As used herein, the term “expression vector” is meant to refer to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. The expression vector may be a recombinant vector.

As used herein, the term “flanking” is meant to refer to a relative position of one nucleic acid sequence with respect to another nucleic acid sequence. Generally, in the sequence ABC, B is flanked by A and C. The same is true for the arrangement AxBxC. Thus, a flanking sequence precedes or follows a flanked sequence but need not be contiguous with, or immediately adjacent to the flanked sequence.

As used herein, the term “spacer region” is meant to refer to an intervening sequence that separates functional elements in a vector or genome. In some embodiments, spacer regions keep two functional elements at a desired distance for optimal functionality. In some embodiments, the spacer regions provide or add to the genetic stability of the vector or genome. In some embodiments, spacer regions facilitate ready genetic manipulation of the genome by providing a convenient location for cloning sites and a gap of design number of base pair.

As used herein, the terms “expression cassette” and “expression unit” are used interchangeably, and meant to refer to a heterologous DNA sequence that is operably linked to a promoter or other DNA regulatory sequence sufficient to direct transcription of a transgene of a DNA vector, e.g., synthetic AAV vector. Suitable promoters include, for example, tissue specific promoters. Promoters can also be of AAV origin.

As used herein, the phrase “genetic disease” or “genetic disorder” is meant to refer to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence.

As used herein, the term “lipid” is meant to refer to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

In one embodiment, the lipid compositions comprise one or more tertiary amino groups, one or more phenyl ester bonds, and a disulfide bond.

As used herein, the term “lipid conjugate” is meant to refer to a conjugated lipid that inhibits aggregation of lipid particles (e.g., lipid nanoparticles). Such lipid conjugates include, but are not limited to, PEGylated lipids such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in International Patent Application Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

As used herein, the term “lipid encapsulated” is meant to refer to a lipid particle that provides an active agent or therapeutic agent, such as a nucleic acid (e.g., a ceDNA), with full encapsulation, partial encapsulation, or both. In a preferred embodiment, the nucleic acid is fully encapsulated in the lipid particle (e.g., to form a nucleic acid containing lipid particle).

As used herein, the terms “lipid particle” or “lipid nanoparticle” is meant to refer to a lipid formulation that can be used to deliver a therapeutic agent such as nucleic acid therapeutics to a target site of interest (e.g., cell, tissue, organ, and the like). In one embodiment, the lipid particle of the disclosure is a nucleic acid containing lipid particle, which is typically formed from a cationic lipid, a non-cationic lipid, and optionally a conjugated lipid that prevents aggregation of the particle. In other preferred embodiments, a therapeutic agent such as a therapeutic nucleic acid may be encapsulated in the lipid portion of the particle, thereby protecting it from enzymatic degradation. In one embodiment, the lipid particle comprises a nucleic acid (e.g., ceDNA) and a lipid comprising one or more a tertiary amino groups, one or more phenyl ester bonds and a disulfide bond.

According to some embodiments, the lipid particles of the disclosure typically have a mean diameter of from about 20 nm to about 75 nm, about 20 nm to about 70 nm, about 25 nm to about 75 nm, about 25 nm to about 70 nm, from about 30 nm to about 75 nm, from about 30 nm to about 70 nm, from about 35 nm to about 75 nm, from about 35 nm to about 70 nm, from about 40 nm to about 75 nm, from about 40 nm to about 70 nm, from about 45 nm to about 75 nm, from about 50 nm to about 75 nm, from about 50 nm to about 70 nm, from about 60 nm to about 75 nm, from about 60 nm to about 70 nm, from about 65 nm to about 75 nm, from about 65 nm to about 70 nm, or about 20 nm, about 25 nm, about 30 nm, about 35 nm, about 40 nm, about 45 nm, about 50 nm, about 51 nm, about 52 nm, about 53 nm, about 54 nm, about 55 nm, about 56 nm, about 57 nm, about 58 nm, about 59 nm about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, or about 75 nm (± 3 nm) in size.

Generally, the lipid particles (e.g., lipid nanoparticles) of the disclosure have a mean diameter selected to provide an intended therapeutic effect.

According to some embodiments, the lipid particles of the disclosure typically have a mean diameter of less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm in size.

As used herein, the term “cationic lipid” refers to any lipid that is positively charged at physiological pH. The cationic lipid in the lipid particles may comprise, e.g., one or more cationic lipids such as 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), “SS-cleavable lipid”, or a mixture thereof. In some embodiments, a cationic lipid is also an ionizable lipid, i.e., an ionizable cationic lipid. Corresponding quaternary lipids of all cationic lipids described herein (i.e., where the nitrogen atom in the cationic moiety is protonated and has four substituents) are contemplated within the scope of this disclosure. Any cationic lipid described herein may be converted to corresponding quaternary lipids, for example, by treatment with chloromethane (CH₃Cl) in acetonitrile (CH₃CN) and chloroform (CHCl₃).

As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

As used herein, the term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

As used herein, the term “ionizable lipid” is meant to refer to a lipid, e.g., cationic lipid, having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. In some embodiments, ionizable lipid may include “cleavable lipid” or “SS-cleavable lipid”.

As used herein, the term “neutral lipid” is meant to refer to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “non-cationic lipid” is meant to refer to any amphipathic lipid as well as any other neutral lipid or anionic lipid.

As used herein, the term “cleavable lipid” or “SS-cleavable lipid” refers to a lipid comprising a disulfide bond cleavable unit. Cleavable lipids may include cleavable disulfide bond (“ss”) containing lipid-like materials that comprise a pH-sensitive tertiary amine and self-degradable phenyl ester. For example, a SS-cleavable lipid can be an ss-OP lipid (COATSOME^{®} SS-OP), an ss-M lipid (COATSOME^{®} SS-M), an ss-E lipid (COATSOME^{®} SS-E), an ss-EC lipid (COATSOME^{®} SS-EC), an ss-LC lipid (COATSOME^{®} SS-LC), an ss-OC lipid (COATSOME^{®} SS-OC), and an ss-PalmE lipid (see, for example, Formulae I-IV), or a lipid described by Togashi et al., (2018) Journal of Controlled Release “A hepatic pDNA delivery system based on an intracellular environment sensitive vitamin E -scaffold lipid-like material with the aid of an anti-inflammatory drug” 279:262-270. Additional examples of cleavable lipids are described in U.S. Pat. 9,708,628, and U.S. Pat. No. 10,385,030, the entire contents of which are incorporated herein by reference. In one embodiment, cleavable lipids comprise a tertiary amine, which responds to an acidic compartment, e.g., an endosome or lysosome for membrane destabilization and a disulfide bond that can be cleaved in a reducing environment, such as the cytoplasm. In one embodiment, a cleavable lipid is a cationic lipid. In one embodiment, a cleavable lipid is an ionizable cationic lipid. Cleavable lipids are described in more detail herein.

As used herein, the term “organic lipid solution” is meant to refer to a composition comprising in whole, or in part, an organic solvent having a lipid.

As used herein, the term “liposome” is meant to refer to lipid molecules assembled in a spherical configuration encapsulating an interior aqueous volume that is segregated from an aqueous exterior. Liposomes are vesicles that possess at least one lipid bilayer. Liposomes are typical used as carriers for drug/ therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “local delivery” is meant to refer to delivery of an active agent such as an interfering RNA (e.g., siRNA) directly to a target site within an organism. For example, an agent can be locally delivered by direct injection into a disease site such as a tumor or other target site such as a site of inflammation or a target organ such as the liver, heart, pancreas, kidney, and the like.

As used herein, the term “nucleic acid,” is meant to refer to a polymer containing at least two nucleotides (i.e., deoxyribonucleotides or ribonucleotides) in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone™ DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA / CELiD), plasmids, bacmids, DOGGYBONE™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, “nucleotides” contain a sugar deoxyribose (DNA) or ribose (RNA), a base, and a phosphate group. Nucleotides are linked together through the phosphate groups.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the term “gap” is meant to refer to a discontinued portion of synthetic DNA vector of the present disclosure, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As used herein, the term “nick” refers to a discontinuity in a double stranded DNA molecule where there is no phosphodiester bond between adjacent nucleotides of one strand typically through damage or enzyme action. It is understood that one or more nicks allow for the release of torsion in the strand during DNA replication and that nicks are also thought to play a role in facilitating binding of transcriptional machinery.

As used herein, the term “subject” is meant to refer to a human or animal, to whom treatment, including prophylactic treatment, with the therapeutic nucleic acid according to the present disclosure, is provided. Usually, the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the phrase “subject in need” refers to a subject that (i) will be administered a ceDNA lipid particle (or pharmaceutical composition comprising a ceDNA lipid particle) according to the described disclosure, (ii) is receiving a ceDNA lipid particle (or pharmaceutical composition comprising aceDNA lipid particle) according to the described disclosure; or (iii) has received a ceDNA lipid particle (or pharmaceutical composition comprising a ceDNA lipid particle) according to the described disclosure, unless the context and usage of the phrase indicates otherwise.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the term “systemic delivery” is meant to refer to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles (e.g., lipid nanoparticles) can be by any means known in the art including, for example, intravenous, subcutaneous, and intraperitoneal. In a preferred embodiment, systemic delivery of lipid particles (e.g., lipid nanoparticles) is by intravenous delivery.

As used herein, the terms “therapeutic amount”, “therapeutically effective amount”, an “amount effective”, or “pharmaceutically effective amount” of an active agent (e.g., a ceDNA lipid particle as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described disclosure. In prophylactic or preventative applications of the described disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman’s The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug’s plasma concentration can be measured and related to therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “alkyl” refers to a saturated monovalent hydrocarbon radical of 1 to 20 carbon atoms (i.e., C₁₋₂₀ alkyl). “Monovalent” means that alkyl has one point of attachment to the remainder of the molecule. In one embodiment, the alkyl has 1 to 12 carbon atoms (i.e., C₁₋₁₂ alkyl) or 1 to 10 carbon atoms (i.e., C₁₋₁₀ alkyl). In one embodiment, the alkyl has 1 to 8 carbon atoms (i.e., C₁₋₈ alkyl), 1 to 7 carbon atoms (i.e., C₁₋₇ alkyl), 1 to 6 carbon atoms (i.e., C₁₋₆ alkyl), 1 to 4 carbon atoms (i.e., C₁₄ alkyl), or 1 to 3 carbon atoms (i.e., C₁₋₃ alkyl). Examples include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. A linear or branched alkyl, such as a “linear or branched C₁₋₆ alkyl,” “linear or branched C_{I-4} alkyl,” or “linear or branched C₁₋₃ alkyl” means that the saturated monovalent hydrocarbon radical is a linear or branched chain.

The term “alkylene” as used herein refers to a saturated divalent hydrocarbon radical of 1 to 20 carbon atoms (i.e., C₁₋₂₀ alkylene), examples of which include, but are not limited to, those having the same core structures of the alkyl groups as exemplified above. “Divalent” means that the alkylene has two points of attachment to the remainder of the molecule. In one embodiment, the alkylene has 1 to 12 carbon atoms (i.e., C₁₋₁₂ alkylene) or 1 to 10 carbon atoms (i.e., C₁₋₁₀ alkylene). In one embodiment, the alkylene has 1 to 8 carbon atoms (i.e., C₁₋₈ alkylene), 1 to 7 carbon atoms (i.e., C₁₋₇ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), ethylene, or methylene. A linear or branched alkylene, such as a “linear or branched C₁₋₆ alkylene,” “linear or branched C₁₋₄ alkylene,” or “linear or branched C₁₋₃ alkylene” means that the saturated divalent hydrocarbon radical is a linear or branched chain.

The term “alkenyl” refers to straight or branched aliphatic hydrocarbon radical with one or more (e.g., one or two) carbon-carbon double bonds, wherein the alkenyl radical includes radicals having “cis” and “trans” orientations, or by an alternative nomenclature, “E” and “Z” orientations.

“Alkenylene” as used herein refers to aliphatic divalent hydrocarbon radical of 2 to 20 carbon atoms (i.e., C₂₋₂₀ alkenylene) with one or two carbon-carbon double bonds, wherein the alkenylene radical includes radicals having “cis” and “trans” orientations, or by an alternative nomenclature, “E” and “Z” orientations. “Divalent” means that alkenylene has two points of attachment to the remainder of the molecule. In one embodiment, the alkenylene has 2 to 12 carbon atoms (i.e., C₂₋₁₆ alkenylene), 2 to 10 carbon atoms (i.e., C₂₋₁₀ alkenylene). In one embodiment, the alkenylene has 2 to four carbon atoms (C₂₋₄). Examples include, but are not limited to, ethylenylene or vinylene (—CH═CH—), allyl (—CH₂CH═CH—), and the like. A linear or branched alkenylene, such as a “linear or branched C₂₋₆ alkenylene,” “linear or branched C₂₋₄ alkenylene,” or “linear or branched C₂₋₃ alkenylene” means that the unsaturated divalent hydrocarbon radical is a linear or branched chain.

“Cycloalkylene” as used herein refers to a divalent saturated carbocyclic ring radical having 3 to 12 carbon atoms as a monocyclic ring, or 7 to 12 carbon atoms as a bicyclic ring. “Divalent” means that the cycloalkylene has two points of attachment to the remainder of the molecule. In one embodiment, the cycloalkylene is a 3- to 7-membered monocyclic or 3- to 6-membered monocyclic. Examples of monocyclic cycloalkyl groups include, but are not limited to, cyclopropylene, cyclobutylene, cyclopentylene, cyclohexylene, cycloheptylene, cyclooctylene, cyclononylene, cyclodecylene, cycloundecylene, cyclododecylene, and the like. In one embodiment, the cycloalkylene is cyclopropylene.

The terms “heterocycle,” “heterocyclyl,” heterocyclic and “heterocyclic ring” are used interchangeably herein and refer to a cyclic group which contains at least one N atom has a heteroatom and optionally 1-3 additional heteroatoms selected from N and S, and are non-aromatic (i.e., partially or fully saturated). It can be monocyclic or bicyclic (bridged or fused). Examples of heterocyclic rings include, but are not limited to, aziridinyl, diaziridinyl, thiaziridinyl, azetidinyl, diazetidinyl, triazetidinyl, thiadiazetidinyl, thiazetidinyl, pyrrolidinyl, pyrazolidinyl, imidazolinyl, isothiazolidinyl, thiazolidinyl, piperidinyl, piperazinyl, hexahydropyrimidinyl, azepanyl, azocanyl, and the like. The heterocycle contains 1 to 4 heteroatoms, which may be the same or different, selected from N and S. In one embodiment, the heterocycle contains 1 to 3 N atoms. In another embodiment, the heterocycle contains 1 or 2 N atoms. In another embodiment, the heterocycle contains 1 N atom. A “4- to 8-membered heterocyclyl” means a radical having from 4 to 8 atoms (including 1 to 4 heteroatoms selected from N and S, or 1 to 3 N atoms, or 1 or 2 N atoms, or 1 N atom) arranged in a monocyclic ring. A “5- or 6-membered heterocyclyl” means a radical having from 5 or 6 atoms (including 1 to 4 heteroatoms selected from N and S, or 1 to 3 N atoms, or 1 or 2 N atoms, or 1 N atom) arranged in a monocyclic ring. The term “heterocycle” is intended to include all the possible isomeric forms. Heterocycles are described in Paquette, Leo A., Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs (John Wiley & Sons, New York, 1950 to present), in particular Volumes 13, 14, 16, 19, and 28; and J. Am. Chem. Soc. (1960) 82:5566. The heterocyclyl groups may be carbon (carbon-linked) or nitrogen (nitrogen-linked) attached to the rest of the molecule where such is possible.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent.

Suitable substituents for an alkyl, alkylene, alkenylene, cycloalkylene, and heterocyclyl, are those which do not significantly adversely affect the biological activity of the bifunctional compound. Unless otherwise specified, exemplary substituents for these groups include linear, branched or cyclic alkyl, alkenyl or alkynyl having from 1 to 10 carbon atoms, aryl, heteroaryl, heterocyclyl, halogen, guanidinium [—NH(C═NH)NH₂], -OR₁₀₀, NR₁₀₁R₁₀₂, —NO₂, -NR₁₀₁COR₁₀₂, -SR₁₀₀, a sulfoxide represented by -SOR₁₀₁, a sulfone represented by -SO₂R₁₀₁, a sulfonate -SO₃M, a sulfate -OSO₃M, a sulfonamide represented by -SO₂NR₁₀₁R₁₀₂, cyano, an azido, -COR₁₀₁, -OCOR₁₀₁, -OCONR₁₀₁R₁₀₂ and a polyethylene glycol unit (-OCH₂CH₂)ₙR₁₀₁ wherein M is H or a cation (such as Na⁺ or K⁺); R₁₀₁, R₁₀₂ and R₁₀₃ are each independently selected from H, linear, branched or cyclic alkyl, alkenyl or alkynyl having from 1 to 10 carbon atoms, a polyethylene glycol unit (-OCH₂CH₂)ₙ-R₁₀₄, wherein n is an integer from 1 to 24, an aryl having from 6 to 10 carbon atoms, a heterocyclic ring having from 3 to 10 carbon atoms and a heteroaryl having 5 to 10 carbon atoms; and R₁₀₄ is H or a linear or branched alkyl having 1 to 4 carbon atoms, wherein the alkyl, alkenyl, alkynyl, aryl, heteroaryl and heterocyclcyl in the groups represented by R₁₀₀, R₁₀₁, R₁₀₂, R₁₀₃ and R₁₀₄ are optionally substituted with one or more (e.g., 2, 3, 4, 5, 6 or more) substituents independently selected from halogen, —OH, —CN, —NO₂, and unsubstituted linear or branched alkyl having 1 to 4 carbon atoms. Preferably, the substituent for the optionally substituted alkyl, alkylene, alkenylene, cycloalkylene, and heterocyclyl described above is selected from the group consisting of halogen, —CN, -NR₁₀₁R₁₀₂, —CF₃, -OR₁₀₀, aryl, heteroaryl, heterocyclyl, -SR₁₀₁, -SOR₁₀₁, -SO₂R₁₀₁, and -SO₃M. Alternatively, the suitable substituent is selected from the group consisting of halogen, —OH, —NO₂, —CN, C₁₋₄ alkyl, -OR₁₀₀, NR₁₀₁R₁₀₂, -NR₁₀₁COR₁₀₂, -SR₁₀₀, -SO₂R₁₀₁, -SO₂NR₁₀₁R₁₀₂, -COR₁₀₁, -OCOR₁₀₁, and -OCONR₁₀₁R₁₀₂, wherein R₁₀₀, R₁₀₁, and R₁₀₂ are each independently —H or C₁₋₄ alkyl.

“Halogen” as used herein refers to F, Cl, Br or I. “Cyano” is —CN.

“Amine” or “amino” as used herein interchangeably refers to a functional group that contains a basic nitrogen atom with a lone pair.

The term “pharmaceutically acceptable salt” as used herein refers to pharmaceutically acceptable organic or inorganic salts of an ionizable lipid of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

### II. Lipid Nanoparticle Compositions

Provided herein are pharmaceutical compositions comprising lipid nanoparticles (LNPs), wherein the LNPs comprises a lipid and a rigid therapeutic nucleic acid (rTNA), wherein the mean diameter of the LNP is between about 20 nm and about 70 nm. The LNPs described herein provides numerous therapeutic advantages, including a smaller size that can encapsulate large, rigid therapeutic nucleic acid molecules. According to some embodiments, the lipid is a cationic lipid. According to some embodiment, the rigid therapeutic nucleic acid is closed-ended DNA (ceDNA). According to some embodiments, the LNP further comprises a non-cationic lipid. According to some embodiments, the LNP further comprises a sterol or a derivative thereof. According to some embodiments, the LIP further comprises a PEG conjugated to a lipid.

### Cationic Lipids

In some embodiments, the lipid nanoparticle having mean diameter of 20-74 nm comprises a cationic lipid. In some embodiments, the cationic lipid is, e.g., a non-fusogenic cationic lipid. By a “non-fusogenic cationic lipid” is meant a cationic lipid that can condense and/or encapsulate the nucleic acid cargo, such as ceDNA, but does not have, or has very little, fusogenic activity.

In some embodiments, the cationic lipid is described in the international and U.S. patent application publications listed below in Table 1, and determined to be non-fusogenic, as measured, for example, by a membrane-impermeable fluorescent dye exclusion assay, e.g., the assay described in the Examples section herein. Contents of all of these patent documents international and U.S. Pat. application publications listed below in Table 1 are incorporated herein by reference in their entireties.

TABLE 1

Exemplary patent documents describing cationic or ionizable lipids

| International Patent Application Publication No. | U.S. Pat. Application Publication No. |
|---|---|
| WO2015/095340 | US2016/0311759 |
| WO2015/199952 | US2015/0376115 |
| WO2018/011633 | US2016/0151284 |
| WO2017/049245 | US2017/0210697 |
| WO2015/061467 | US2015/0140070 |
| WO2012/040184 | US2013/0178541 |
| WO2012/000104 | US2013/0303587 |
| WO2015/074085 | US2015/0141678 |
| WO2016/081029 | US2015/0239926 |
| WO2017/004143 | US2016/0376224 |
| WO2017/075531 | US2017/0119904 |
| WO2017/117528 | |
| WO2011/022460 | US2012/0149894 |
| WO2013/148541 | US2015/0057373 |
| WO2013/116126 | |
| WO2011/153120 | US2013/0090372 |
| WO2012/044638 | US2013/0274523 |
| WO2012/054365 | US2013/0274504 |
| WO2011/090965 | US2013/0274504 |
| WO2013/016058 | |
| WO2012/162210 | |
| WO2008/042973 | US2009/0023673 |
| WO2010/129709 | US2012/0128760 |
| WO2010/144740 | US201/003241240 |
| WO2012/099755 | US2014/0200257 |
| WO2013/049328 | US2015/0203446 |
| WO2013/086322 | US2018/0005363 |
| WO2013/086373 | US2014/0308304 |
| WO2011/071860 | US2013/0338210 |
| WO2009/132131 | |
| WO2010/048536 | |
| WO2010/088537 | US2012/0101148 |
| WO2010/054401 | US2012/0027796 |
| WO2010/054401 | |
| WO2010/054405 | |
| WO2010/054384 | US2012/0058144 |
| WO2012/016184 | US2013/0323269 |
| WO2009/086558 | US2011/0117125 |
| WO2010/042877 | US2011/0256175 |
| WO2011/000106 | US2012/0202871 |
| WO2011/000107 | US2011/0076335 |
| WO2005/120152 | US2006/0083780 |
| WO2011/141705 | US2013/0123338 |
| WO2013/126803 | US2015/0064242 |
| WO2006/07712 | US2006/0051405 |
| WO2011/038160 | US2013/0065939 |
| WO2005/121348 | US2006/0008910 |
| WO2011/066651 | US2003/0022649 |
| WO2009/127060 | US2010/0130588 |
| WO2011/141704 | US2013/0116307 |
| WO2006/069782 | US2010/0062967 |
| WO2012/031043 | US2013/0202684 |
| WO2013/006825 | US2014/0141070 |
| WO2013/033563 | US2014/0255472 |
| WO2013/089151 | US2014/0039032 |
| WO2017/099823 | US2018/0028664 |
| WO2015/095346 | US2016/0317458 |
| WO2013/086354 | US2013/0195920 |

In some embodiments, the cationic lipid is selected from the group consisting of N-[1-(2,3-dioleyloxy)propyll-N,N,N-trimethylammonium chloride (DOTMA); N-[1-(2,3-dioleoyloxy)propyll-N,N,N-trimethylammonium chloride (DOTAP); 1,2-dioleoyl-sn-glycero -3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl- sn-glycero-3-ethylphosphocholine (14:1), N1-[2-((1S)-1-[(3-aminopropyl)amino]-4-[di(3-amino-propyl) aminolbutylc arboxamidoiethy11-3, 4 -di[oleyloxy]-benzamide(MVL5); Dioctadecylamido-glycylspermine (DOGS); 3b-[N-(N′,N′-dimethylaminoethyl)carb amoyl] cholesterol (DC-Chol); Dioctadecyldimethylammonium Bromide (DDAB); a Saint lipid (e.g., SAINT-2, N-methyl-4-(dioleyl)methylpyridinium); 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); Di-alkylated Amino Acid (DILA2) (e.g., C18 :1 -norArg -C16); Dioleyldimethylammonium chloride (DODAC); 1-palmitoyl-2-oleoyl-sn-glycero-3 -ethylpho sphocholine (POEPC); and 1,2 -dimyristoleoyl-sn-glycero-3-ethylphosphocholine (MOEPC). In some variations, the condensing agent, e.g. a cationic lipid, is a lipid such as, e.g., Dioctadecyldimethylammonium bromide (DDAB), 1,2-dilinoleyloxy-3-dimethylaminopropane (DLinDMA), 2,2-dilinoleyl-4-(2dimethylaminoethyl)-[1,31-dioxolane (DLin-KC2-DMA), heptatriaconta-6,9,28,31-tetraen-19- yl-4-(dimethylamino)butanoate (DLin-MC3-DMA), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diyl dioleate hydrochloride (DODAPen-C1), (R)-5-guanidinopentane-1,2-diy1 dioleate hydrochloride (DOPen-G), (R)-N,N,N-trimethyl-4,5-bis(oleoyloxy)pentan-1-aminium chloride(DOTAPen). In some embodiments, the condensing lipid is DOTAP.

### Ionizable Lipids

According to some embodiments, also provided herein are pharmaceutical compositions containing LNPs having mean diameter of 20-70 nm, the LNPs comprising an ionizable lipid and a rigid therapeutic nucleic acid like non-viral vector (e.g., ceDNA). Such LNPs can be used to deliver, e.g., the capsid-free, non-viral DNA vector to a target site of interest (e.g., cell, tissue, organ, and the like).

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and U.S. Pat. publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

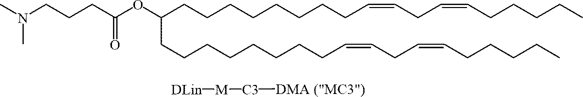

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, the contents of which is incorporated herein by reference in its entirety.

In some embodiments, the ionizable lipid is (13Z,16Z)-N,N-dimethyl-3-nonyldocosa-13,16-dien-1-amine (Compound 32), as described in WO2012/040184, the contents of which is incorporated herein by reference in its entirety.

In some embodiments, the ionizable lipid is Compound 6 or Compound 22 as described in WO2015/199952, the contents of which is incorporated herein by reference in its entirety.

### Formula (I) and Formula (I′)

According to some embodiments, the ionizable lipids are represented by Formula (I):

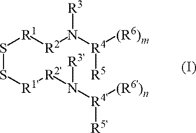

or a pharmaceutically acceptable salt thereof, wherein:
R¹ and R^{1′} are each independently C₁₋₃ alkylene;
R² and R^{2′} are each independently linear or branched C₁₋₆ alkylene, or C₃₋₆ cycloalkylene;
R³ and R^{3′} are each independently optionally substituted C₁₋₆ alkyl or optionally substituted C₃₋₆ cycloalkyl;
or alternatively, when R² is branched C₁₋₆ alkylene and when R³ is C₁₋₆ alkyl, R² and R³, taken together with their intervening N atom, form a 4- to 8-membered heterocyclyl;
or alternatively, when R^{2′} is branched C₁₋₆ alkylene and when R³′ is C₁₋₆ alkyl, R^{2′} and R^{3′}, taken together with their intervening N atom, form a 4- to 8-membered heterocyclyl;
R⁴ and R^{4′} are each independently —CH, —CH₂CH, or —(CH₂)₂CH;
R⁵ and R^{5′} are each independently C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

Alternatively, according to some embodiments, the ionizable lipids are represented by Formula (I′):

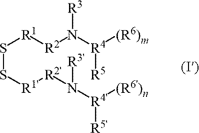

or a pharmaceutically acceptable salt thereof, wherein:
R¹ and R^{1′} are each independently C₁₋₃ alkylene;
R² and R^{2′} are each independently linear or branched C₁₋₆ alkylene, or C₃₋₆ cycloalkylene;
R³ and R^{3′} are each independently optionally substituted C₁₋₆ alkyl or optionally substituted C₃₋₆ cycloalkyl;
or alternatively, when R² is branched C₁₋₆ alkylene and when R³ is C₁₋₆ alkyl, R² and R³, taken together with their intervening N atom, form a 4- to 8-membered heterocyclyl;
or alternatively, when R^{2′} is branched C₁₋₆ alkylene and when R^{3′} is C₁₋₆ alkyl, R^{2′} and R^{3′}, taken together with their intervening N atom, form a 4- to 8-membered heterocyclyl;
R⁴ and R^{4′} are each independently —CH, —CH₂CH, or —(CH₂)₂CH;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

According to some embodiments of any of the aspects or embodiments herein, R² and R^{2′} are each independently C₁₋₃ alkylene.

According to some embodiments of any of the aspects or embodiments herein, the linear or branched C₁₋₃ alkylene represented by R¹ or R^{1′}, the linear or branched C₁₋₆ alkylene represented by R² or R^{2′}, and the optionally substituted linear or branched C₁₋₆ alkyl are each optionally substituted with one or more halo and cyano groups.

According to some embodiments of any of the aspects or embodiments herein, R¹ and R² taken together are C₁₋₃ alkylene and R^{1′} and R^{2′} taken together are C₁₋₃ alkylene, e.g., ethylene.

According to some embodiments of any of the aspects or embodiments herein, R³ and R^{3′} are each independently optionally substituted C₁₋₃ alkyl, e.g., methyl.

According to some embodiments of any of the aspects or embodiments herein, R⁴ and R^{4′} are each —CH.

According to some embodiments of any of the aspects or embodiments herein, R² is optionally substituted branched C₁₋₆ alkylene; and R² and R³, taken together with their intervening N atom, form a 5- or 6-membered heterocyclyl. According to some embodiments of any of the aspects or embodiments herein, R^{2′} is optionally substituted branched C₁₋₆ alkylene; and R^{2′} and R^{3′}, taken together with their intervening N atom, form a 5- or 6-membered heterocyclyl, such as pyrrolidinyl or piperidinyl.

According to some embodiments of any of the aspects or embodiments herein, R⁴ is — C(R^{a})₂CR^{a}, or —[C(R^{a})₂]₂CR^{a} and R^{a} is C₁₋₃ alkyl; and R³ and R⁴, taken together with their intervening N atom, form a 5- or 6-membered heterocyclyl. According to some embodiments of any of the aspects or embodiments herein, R^{4′} is —C(R^{a})₂CR^{a}, or —[C(R^{a})₂]₂CR^{a} and R^{a} is C₁₋₃ alkyl; and R^{3′} and R^{4′}, taken together with their intervening N atom, form a 5- or 6-membered heterocyclyl, such as pyrrolidinyl or piperidinyl.

According to some embodiments of any of the aspects or embodiments herein, R³ and R^{5′} are each independently C₁₋₁₀ alkylene or C₂₋₁₀ alkenylene. In one embodiment, R⁵ and R^{5′} are each independently C₁₋₈ alkylene or C₁₋₆ alkylene.

According to some embodiments of any of the aspects or embodiments herein, R⁶ and R^{6′}, for each occurrence, are independently C₁₋₁₀ alkylene, C₃₋₁₀ cycloalkylene, or C₂₋₁₀ alkenylene. In one embodiment, C₁₋₆ alkylene, C₃₋₆ cycloalkylene, or C₂₋₆ alkenylene. In one embodiment the C₃₋₁₀ cycloalkylene or the C₃₋₆ cycloalkylene is cyclopropylene. According to some embodiments of any of the aspects or embodiments herein, m and n are each 3.

According to some embodiments of any of the aspects or embodiments herein, the ionizable lipid is selected from any one of the lipids in Table 2 or a pharmaceutically acceptable salt thereof.

TABLE 2

Exemplary ionizable lipids of Formula (I) or (I′)

| Lipid No. | Structure and Name |
|---|---|
| 1 | 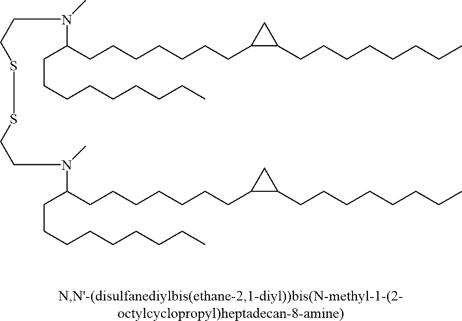 |
| 2 | 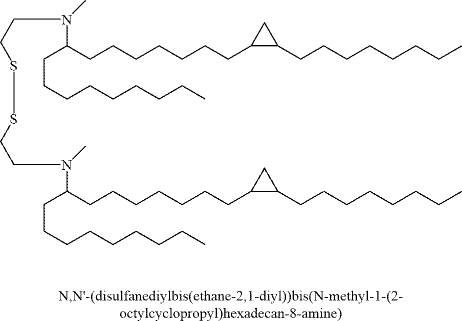 |
| 3 | 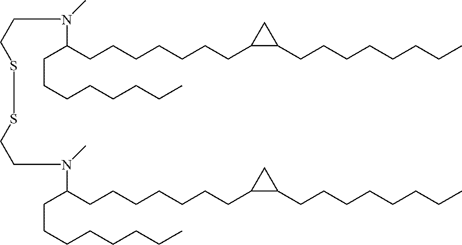 [SMILES: CCCCCCCCC1CC1CCCCCCCC(CCCCCCCC)N(C)CCCSSCCN(C)C(CCCCCCCC)CCCCCCCC1CC1CCCCCCCC] |
| | 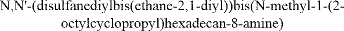 |
| 4 | 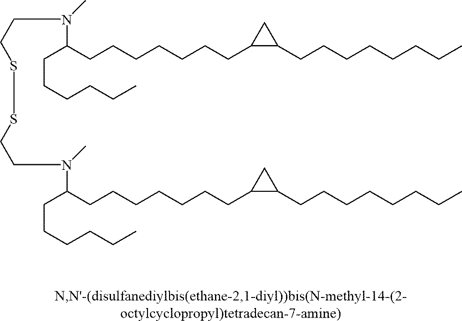 |
| 5 | 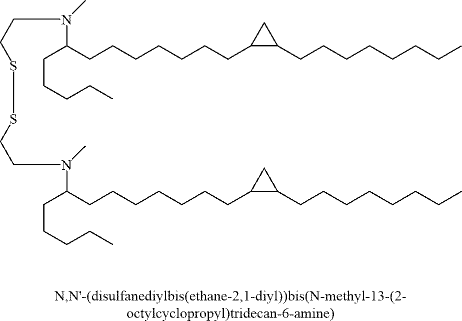 |
| 6 | 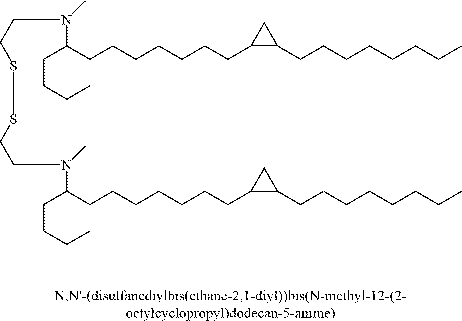 |
| 7 | 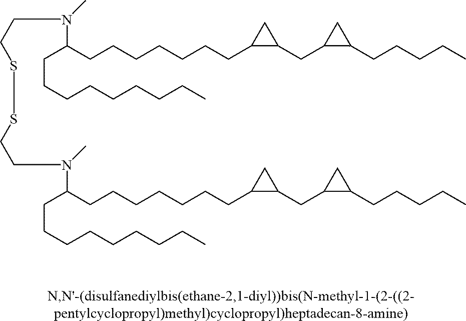 |
| 8 | 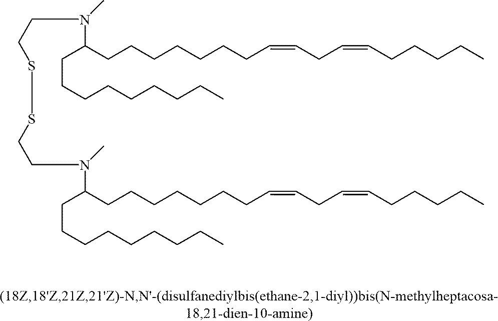 |
| 9 | 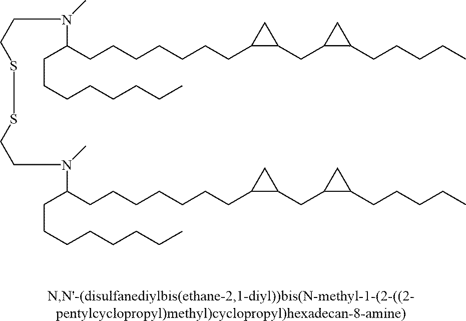 |
| 10 | 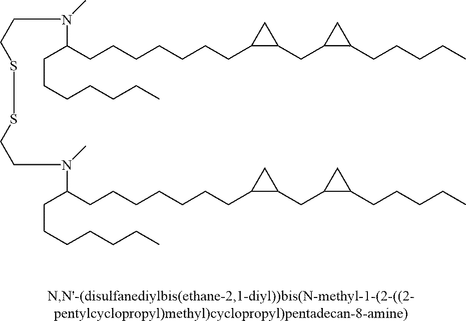 |
| 11 | 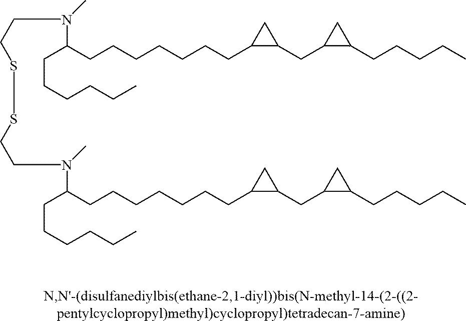 |
| 12 | 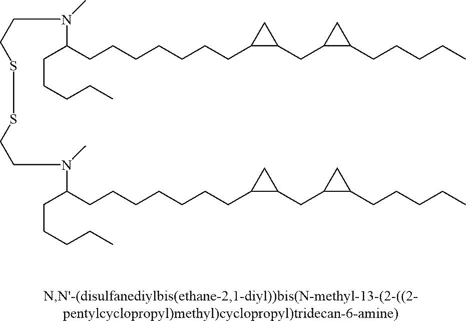 |
| 13 | 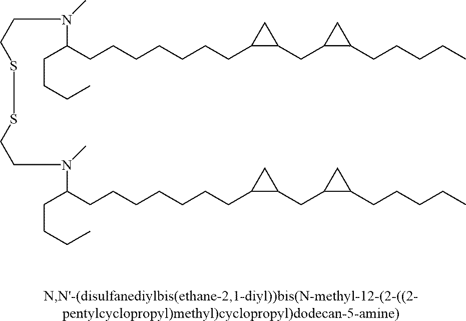 |
| 14 | 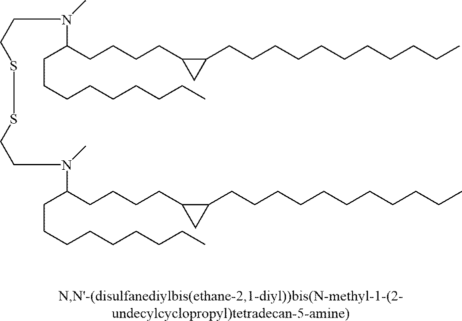 |
| 15 | 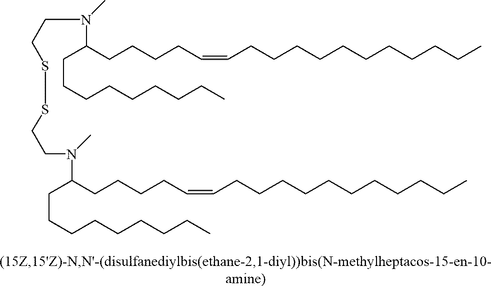 |
| 16 | 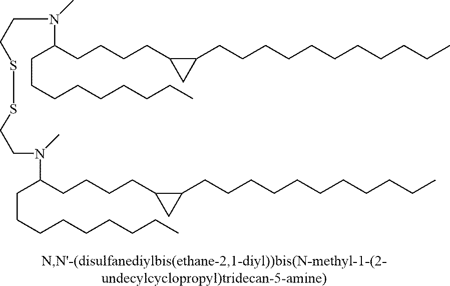 |
| 17 | 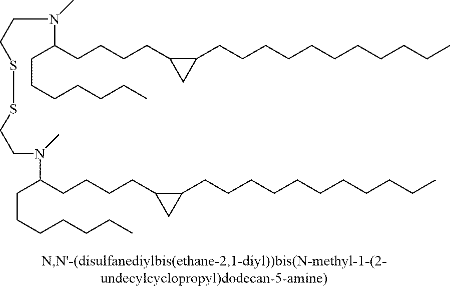 |
| 18 | 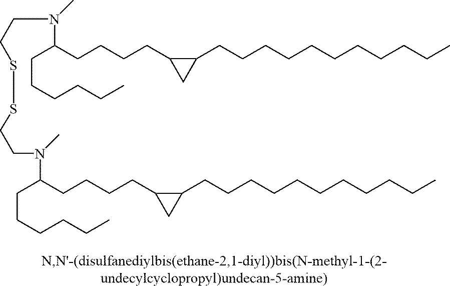 |
| 19 | 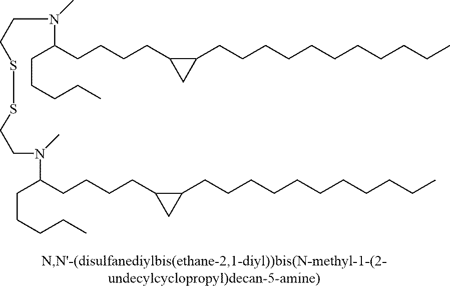 |
| 20 | 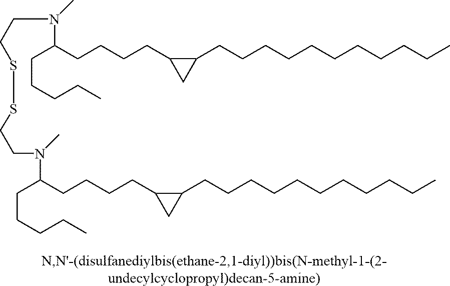 |
| 21 | 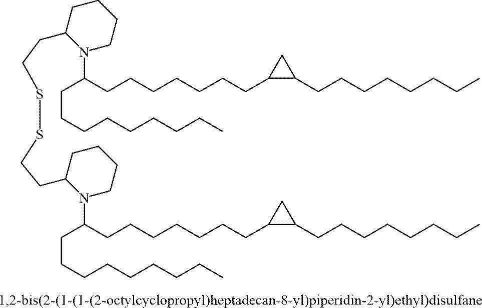 |
| 22 | 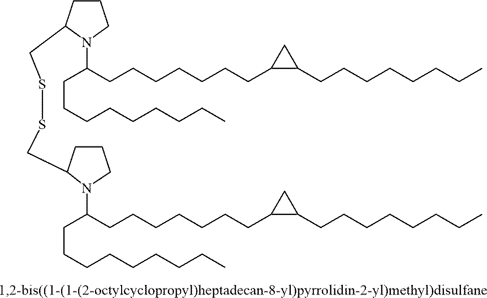 |
| 23 | 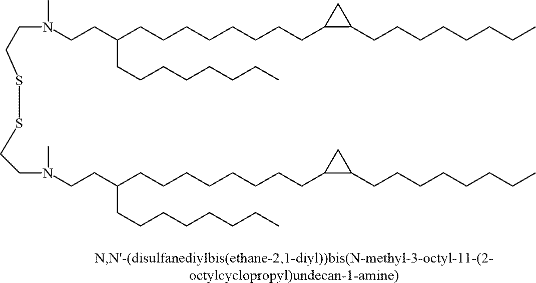 |
| 24 | 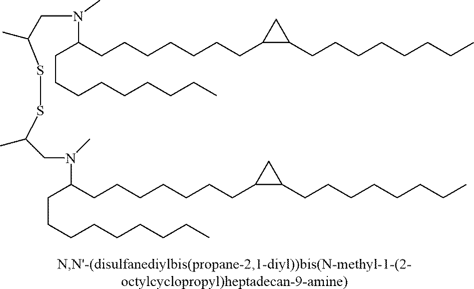 |
| 25 | 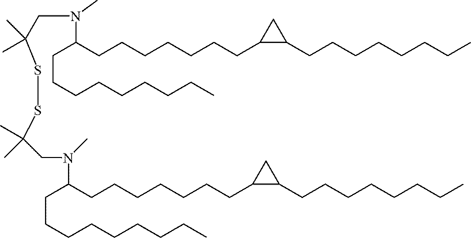 [SMILES: CCCCCCCCCC(CCCCCCCC1CC1CCCCCCCC)N(C)CC(C)(C)SSC(C)(C)CN(C)C(CCCCCCCCC)CCCCCCCC1CC1CCCCCCCC] |
| | 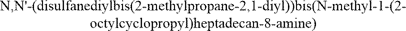 |
| 26 | 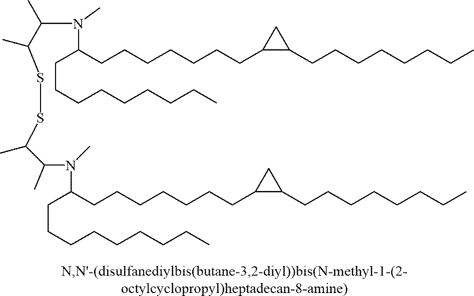 |
| 27 | 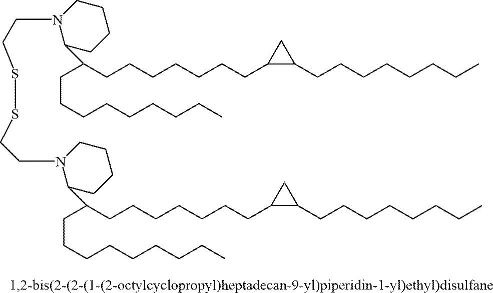 |
| 28 | 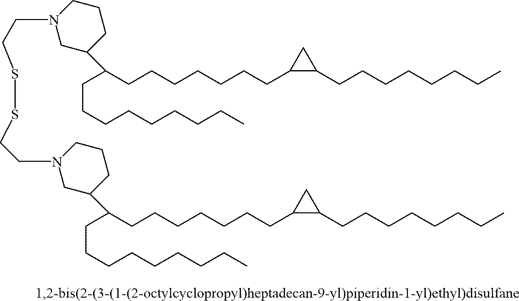 |
| 29 | 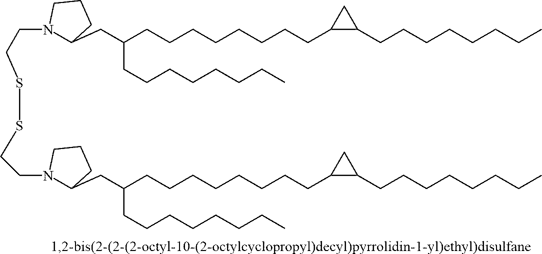 |
| 30 | 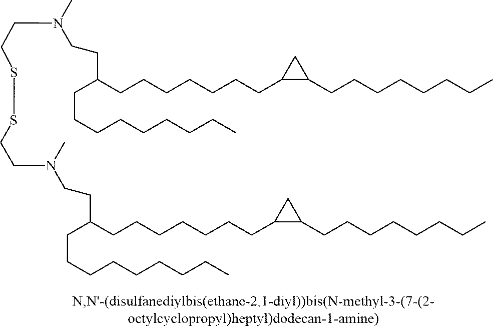 |
| 31 | 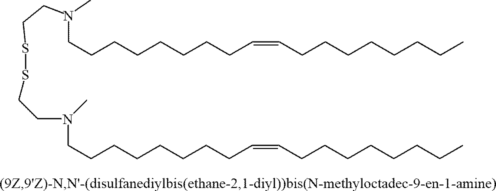 |
| 32 | 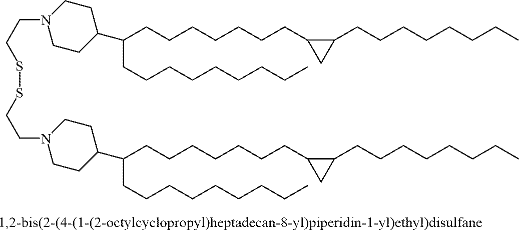 |
| 33 | 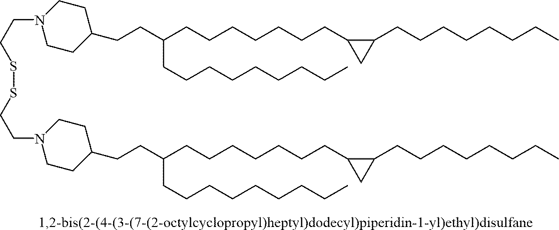 |
| 34 | 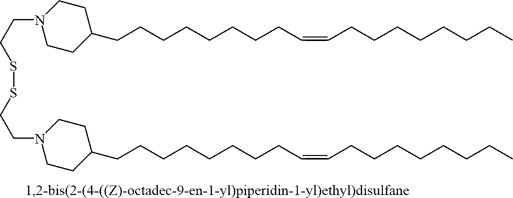 |
| 35 | 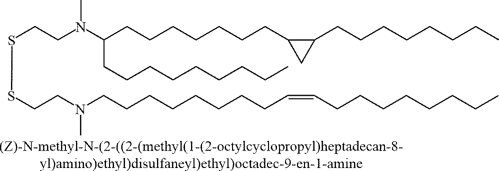 |
| 36 | 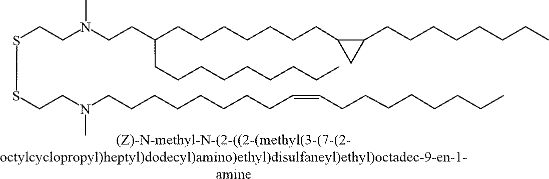 |
| 37 | 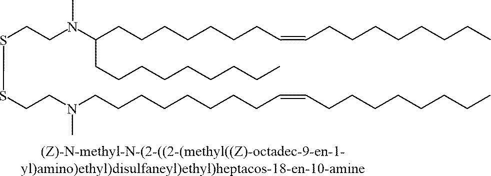 |
| 38 | 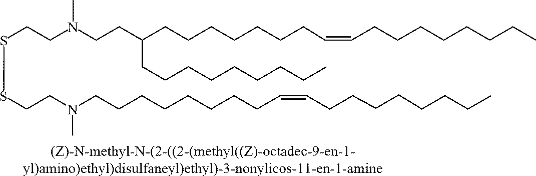 |
| 39 | 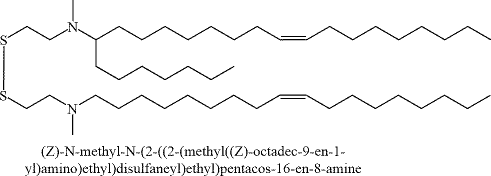 |
| 40 | 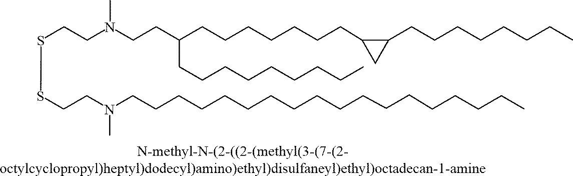 |
| 41 | 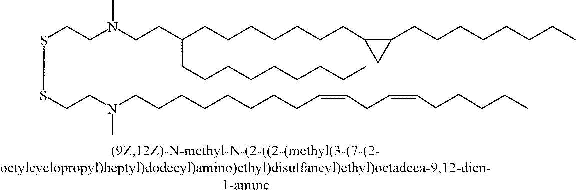 |
| 42 | 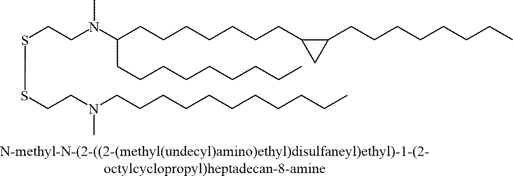 |
| 43 | 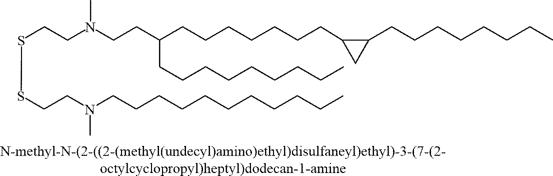 |
| 44 | 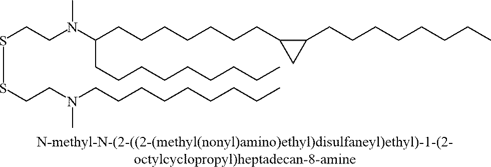 |
| 45 | 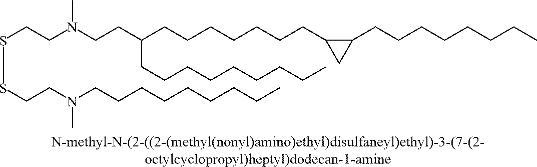 |
| 46 | 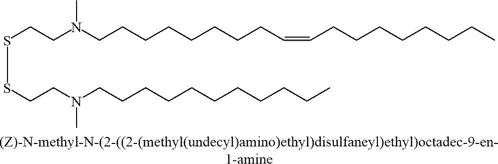 |
| 47 | 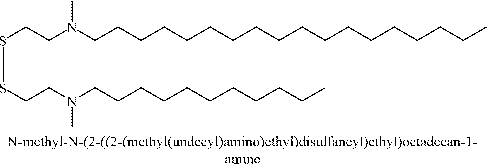 |
| 48 | 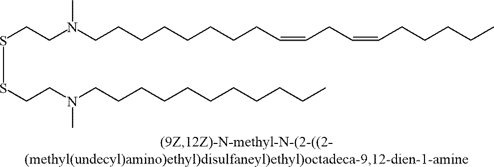 |
| 49 | 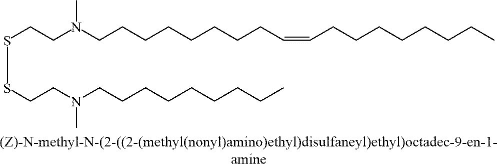 |
| 50 | 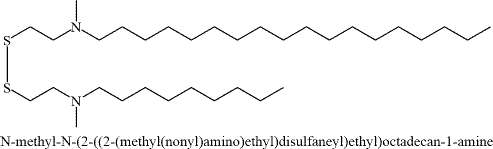 |
| 51 | 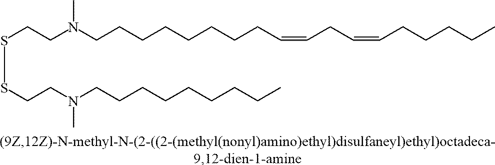 |

### Formula (II)

In some aspects, the ionizable lipids are of the Formula (II):

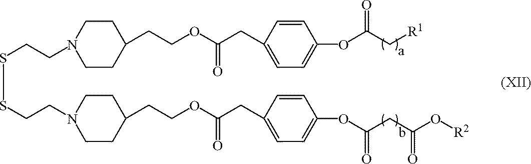

or a pharmaceutically acceptable salt thereof, wherein:
a is an integer ranging from 1 to 20 (e.g., a is 1, 2, 3, 4, 5, 6, 7, 8, 9, 10, 11, 12, 13, 14, 15, 16, 17, 18, 19, or 20);
b is an integer ranging from 2 to 10 (e.g., b is 2, 3, 4, 5, 6, 7, 8, 9, or 10);
R¹ is absent or is selected from (C₂-C₂₀)alkenyl, -C(O)O(C₂-C₂₀)alkyl, and cyclopropyl substituted with (C₂-C₂₀)alkyl; and
R² is (C₂-C₂₀)alkyl.

In a second chemical embodiment, the ionizable lipid of the Formula (II) is of the Formula (XIII):

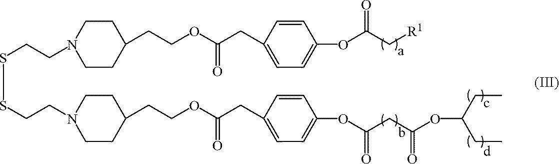

or a pharmaceutically acceptable salt thereof, wherein c and d are each independently integers ranging from 1 to 8 (e.g., 1, 2, 3, 4, 5, 6, 7, or 8), and wherein the remaining variables are as described for Formula (XII).

In a third chemical embodiment, c and d in the ionizable lipid of Formula (II) or (III) are each independently integers ranging from 2 to 8, 3 to 8, 3 to 7, 3 to 6, 3 to 5, 4 to 8, 4 to 7, 4 to 6, 5 to 8, 5 to 7, or 6 to 8, wherein the remaining variables are as described for Formula (XII).

In a fourth chemical embodiment, c in the ionizable lipid of Formula (II) or (III) is 2, 3, 4, 5, 6, 7, or 8, wherein the remaining variables are as described for Formula (XII) or the second or third chemical embodiment. Alternatively, as part of a fourth chemical embodiment, c and d in the ionizable lipid of Formula (XII) or (XIII) or a pharmaceutically acceptable salt thereof are each independently 1, 3, 5, or 7, wherein the remaining variables are as described for Formula (XII) or the second or third chemical embodiment.

In a fifth chemical embodiment, d in the ionizable lipid of Formula (II) or (III) is 2, 3, 4, 5, 6, 7, or 8, wherein the remaining variables are as described for Formula (II) or the second or third or fourth chemical embodiment. Alternatively, as part of a fourth chemical embodiment, at least one of c and d in the ionizable lipid of Formula (II) or (III) or a pharmaceutically acceptable salt thereof is 7, wherein the remaining variables are as described for Formula (II) or the second or third or fourth chemical embodiment.

In a sixth chemical embodiment, the ionizable lipid of Formula (II) or (III) is of the Formula (IV):

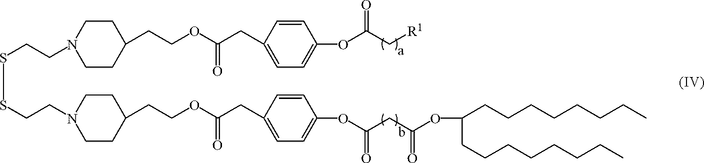

or a pharmaceutically acceptable salt thereof, wherein the remaining variables are as described for Formula (I).

In a seventh chemical embodiment, b in the ionizable lipid of Formula (II), (III), or (IV) is an integer ranging from 3 to 9, wherein the remaining variables are as described for Formula (II), or the second, third, fourth or fifth chemical embodiment. Alternatively, as part of a seventh chemical embodiment, b in the ionizable lipid of Formula (II), (III), or (IV) is an integer ranging from 3 to 8, 3 to 7, 3 to 6, 3 to 5, 4 to 9, 4 to 8, 4 to 7, 4 to 6, 5 to 9, 5 to 8, 5 to 7, 6 to 9, 6 to 8, or 7 to 9, wherein the remaining variables are as described for Formula (II), or the second, third, fourth or fifth chemical embodiment. In another alternative, as part of a seventh chemical embodiment, b in the ionizable lipid of Formula (II), (III), or (IV) is 3, 4, 5, 6, 7, 8, or 9, wherein the remaining variables are as described for Formula (XII), or the second, third, fourth or fifth chemical embodiment.

In an eighth chemical embodiment, a in the ionizable lipid of Formula (II), (III), or (IV) is an integer ranging from 2 to 18, wherein the remaining variables are as described for Formula (II), or the second, third, fourth, fifth, or seventh chemical embodiment. Alternatively, as part of an eighth embodiment, a in the ionizable lipid of Formula (II), (III), or (IV) is an integer ranging from 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 5 to 18, 5 to 17, 5 to 16, 5 to 15, 5 to 14, 5 to 13, 5 to 12, 5 to 11, 5 to 10, 5 to 9, 25 to 8, 5 to 7, 6 to 18, 6 to 17, 6 to 16, 6 to 15, 6 to 14, 6 to 13, 6 to 12, 6 to 11, 6 to 10, 6 to 9, 6 to 8, 7 to 18, 7 to 17, 7 to 16, 7 to 15, 7 to 14, 7 to 13, 7 to 12, 7 to 11, 7 to 10, 7 to 9, 8 to 18, 8 to 17, 8 to 16, 8 to 15, 8 to 14, 8 to 13, 8 to 12, 8 to 11, 8 to 10, 9 to 18, 9 to 17, 9 to 16, 9 to 15, 9 to 14, 9 to 13, 9 to 12, 9 to 11, 10 to 18, 10 to 17, 10 to 16, 10 to 15, 10 to 14, 10 to 13, 11 to 18, 11 to 17, 11 to 16, 11 to 15, 11 to 14, 11 to 13, 12 to 18, 12 to 17, 12 to 16, 12 to 15, 12 to 14, 13 to 18, 13 to 17, 13 to 16, 13 to 15, 14 to 18, 14 to 17, 14 to 16, 15 to 18, 15 to 17, or 16 to 18, wherein the remaining variables are as described for Formula (II), or the second, third, fourth, fifth, or seventh chemical embodiment. In another alternative, as part of an eighth embodiment, a in the ionizable lipid of Formula (II), (III), or (IV) is 2, 3, 4, 5, 6, 7, 8, 9, 10, 11, 12, 13, 14, 15, 16, 17, or 18, wherein the remaining variables are as described for Formula (II), or the second, third, fourth, fifth, or seventh chemical embodiment.

In a ninth chemical embodiment, R¹ in the ionizable lipid of Formula (II), (III), or (IV) or a pharmaceutically acceptable salt thereof is absent or is selected from (C₅-C₁₅)alkenyl, -C(O)O(C₄-C₁₈)alkyl, and cyclopropyl substituted with (C₄-C₁₆)alkyl, wherein the remaining variables are as described for Formula (II), (III), or (IV) or the second, third, fourth, fifth, seventh, or eighth chemical embodiment. Alternatively, as part of a ninth chemical embodiment, R¹ in the ionizable lipid of Formula (II), (III), or (IV) or a pharmaceutically acceptable salt thereof is absent or is selected from (C₅-C₁₅)alkenyl, -C(O)O(C₄-C₁₆)alkyl, and cyclopropyl substituted with (C₄-C₁₆)alkyl, wherein the remaining variables are as described for Formula (II), (III), or (IV) or the second, third, fourth, fifth, seventh, or eighth chemical embodiment. Alternatively, as part of a ninth chemical embodiment, R¹ in the ionizable lipid of Formula (II), (III), or (IV) or a pharmaceutically acceptable salt thereof is absent or is selected from (C₅-C₁₂)alkenyl, -C(O)O(C₄-C₁₂)alkyl, and cyclopropyl substituted with (C₄-C₁₂)alkyl, wherein the remaining variables are as described for Formula (II), (III), or (IV) or the second, third, fourth, fifth, seventh, or eighth chemical embodiment. In another alternative, as part of a ninth chemical embodiment, R¹ in the ionizable lipid of Formula (II), (III), or (IV) or a pharmaceutically acceptable salt thereof is absent or is selected from (C₅-C₁₀)alkenyl, -C(O)O(C₄-C₁₀)alkyl, and cyclopropyl substituted with (C₄-C₁₀)alkyl, wherein the remaining variables are as described for Formula (II), (III), or (IV) or the second, third, fourth, fifth, seventh, or eighth chemical embodiment.

In a tenth chemical embodiment, R¹ is C₁₀ alkenyl, wherein the remaining variables are as described in any one of the foregoing embodiments.

In an eleventh chemical embodiment, the alkyl in C(O)O(C₂-C₂₀)alkyl, -C(O)O(C₄-C₁₈)alkyl, -C(O)O(C₄-C₁₂)alkyl, or -C(O)O(C₄-C₁₀)alkyl of R¹ in the ionizable lipid of Formula (II), (III), or (IV) or a pharmaceutically acceptable salt thereof is an unbranched alkyl, wherein the remaining variables are as described in any one of the foregoing embodiments. In one chemical embodiment, R¹ is —C(O)O(C₉ alkyl). Alternatively, in an eleventh chemical embodiment, the alkyl in -C(O)O(C₄-C₁₈)alkyl, -C(O)O(C₄-C₁₂)alkyl, or -C(O)O(C₄-C₁₀)alkyl of R¹ in the ionizable lipid of Formula (II), (III), or (IV) or a pharmaceutically acceptable salt thereof is a branched alkyl, wherein the remaining variables are as described in any one of the foregoing chemical embodiments. In one chemical embodiment, R¹ is -C(O)O(C₁₇ alkyl), wherein the remaining variables are as described in any one of the foregoing chemical embodiments.

In a twelfth chemical embodiment, R¹ in the ionizable lipid of Formula (II), (III), or (IV) or a pharmaceutically acceptable salt thereof is selected from any group listed in Table 3 below, wherein the wavy bond in each of the groups indicates the point of attachment of the group to the rest of the lipid molecule, and wherein the remaining variables are as described for Formula (II), (III), or (IV) or the second, third, fourth, fifth, seventh, or eighth chemical embodiment. The present disclosure further contemplates the combination of any one of the R¹ groups in Table 4 with any one of the R² groups in Table 5, wherein the remaining variables are as described for Formula (II), (III), or (IV) or the second, third, fourth, fifth, seventh, or eighth chemical embodiment.

TABLE 3

Exemplary R¹ groups in Formula (II), (III), or (IV)

| | |
|---|---|
| 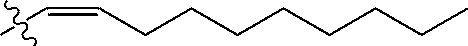 | 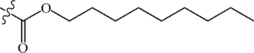 |
| 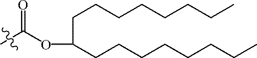 | 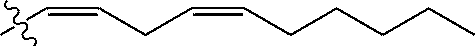 |
| 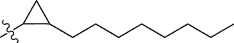 | |

In a thirteenth chemical embodiment, R² in the ionizable lipid of Formula (II) or a pharmaceutically acceptable salt thereof is selected from any group listed in Table 4 below, wherein the wavy bond in each of the groups indicates the point of attachment of the group to the rest of the lipid molecule, and wherein the remaining variables are as described for Formula (II), or the seventh, eighth, ninth, tenth, or eleventh chemical embodiment.

TABLE 4

Exemplary R² groups in Formula (II)

| | |
|---|---|
| 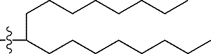 | 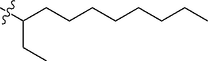 |
| 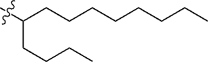 | 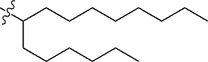 |
| 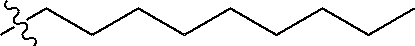 | |

Specific examples are provided in Table 5 the exemplification section below and are included as part of a fourteenth chemical embodiment herein of ionizable lipids of Formula (II). Pharmaceutically acceptable salts as well as ionized and neutral forms are also included.

TABLE 5

Exemplary ionizable lipids of Formula (II), (III), or (IV)

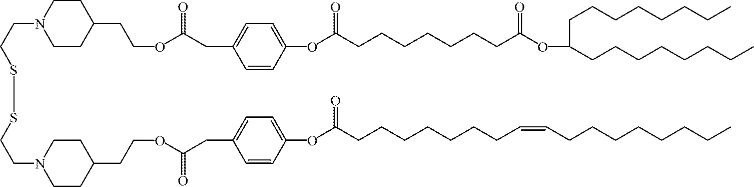 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 52 1-(heptadecan-9-yl) 9-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) nonanedioate

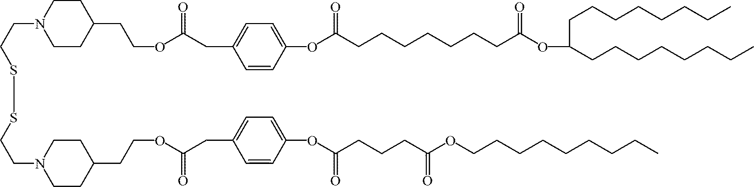 [SMILES: CCCCCCCCCOC(=O)CCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 53 1-(heptadecan-9-yl) 9-(4-(2-(2-(1-(2-((2-(4-(2-(2-(4-((5-(nonyloxy)-5-oxopentanoyl)oxy)phenyl)acetoxy)ethyl)piperidin-1-yl)ethyl)disulfaneyl)ethyl) piperidin-4-yl)ethoxy)-2-oxoethyl)phenyl) nonanedioate

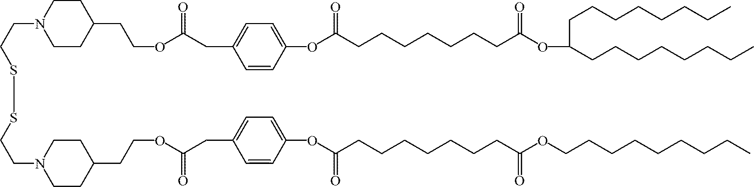 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 54 1-(heptadecan-9-yl) 9-(4-(2-(2-(1-(2-((2-(4-(2-(2-(4-((9-(nonyloxy)-9-oxononanoyl)oxy)phenyl)acetoxy)ethyl)piperidin-1-yl)ethyl)disulfaneyl)ethyl)piperidin-4-yl)ethoxy)-2-oxoethyl)phenyl) nonanedioate

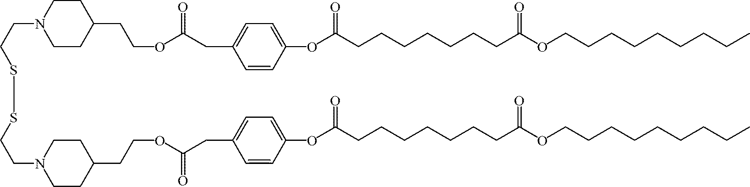 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCCCCCCCC)cc4)CC3)CC2)cc1]

Lipid 55 1-(heptadecan-9-yl) 9-(4-(2-(2-(1-(2-((2-(4-(2-(2-(4-((5-(nonyloxy)-5-oxopentanoyl)oxy)phenyl)acetoxy)ethyl) piperidin-1-yl)ethyl)disulfaneyl)ethyl)piperidin-4-yl)ethoxy)-2-oxoethyl)phenyl) nonanedioate

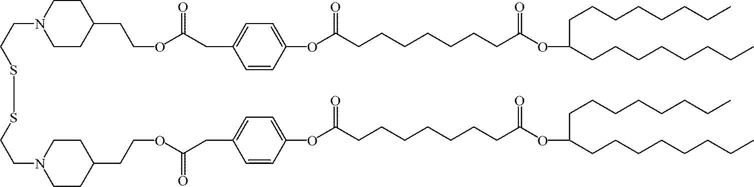 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 56 O′1,O1-((((((disulfanediylbis(ethane-2,1-diyl))bis(piperidine-1,4-diyl))bis(ethane-2,1-diyl))bis(oxy))bis(2-oxoethane-2,1-diyl))bis(4,1-phenylene)) 9,9′-di(heptadecan-9-yl) di(nonanedioate)

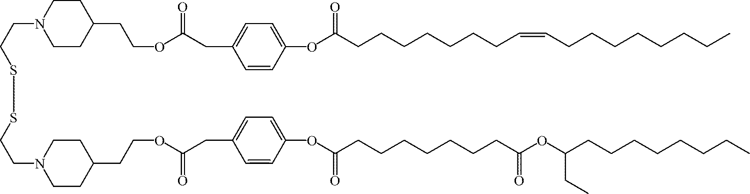 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

Lipid 57 1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(undecan-3-yl) nonanedioate

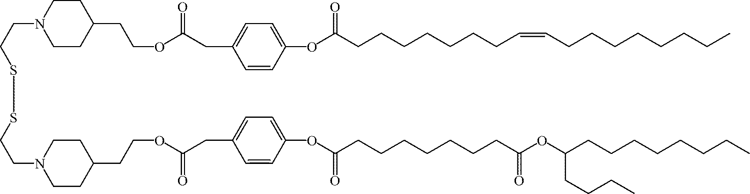 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

Lipid 58 1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(tridecan-5-yl) nonanedioate

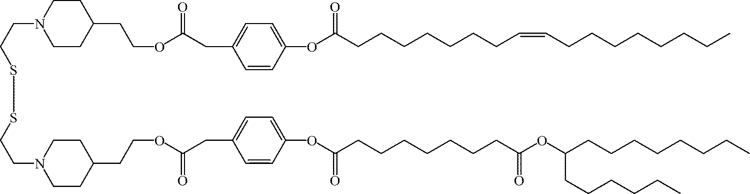 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

Lipid 59
1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(pentadecan-7-yl) nonanedioate

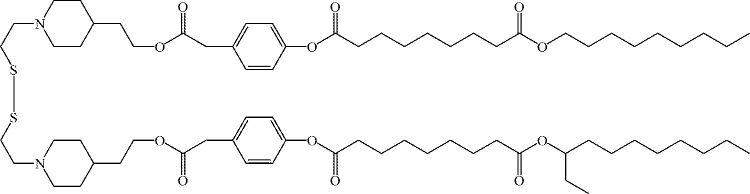 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

Lipid 60 1-nonyl 9-(4-(2-oxo-2-(2-(1-(2-((2-(4-(2-(2-(4-((9-oxo-9-(undecan-3-yloxy)nonanoyl)oxy)phenyl)acetoxy)ethyl)piperidin-1-yl)ethyl)disulfaneyl)ethyl)piperidin-4-yl)ethoxy)ethyl)phenyl) nonanedioate

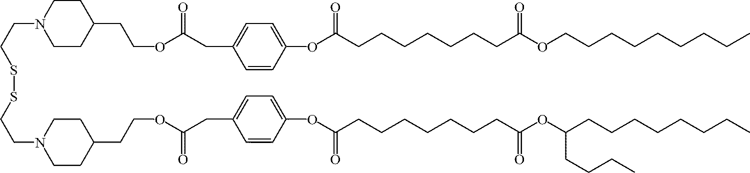 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

Lipid 61 1-nonyl 9-(4-(2-oxo-2-(2-(1-(2-((2-(4-(2-(2-(4-((9-oxo-9-(tridecan-5-yloxy)nonanoyl)oxy)phenyl)acetoxy)ethyl)piperidin-1-yl)ethyl)disulfaneyl)ethyl)piperidin-4-yl)ethoxy)ethyl)phenyl) nonanedioate

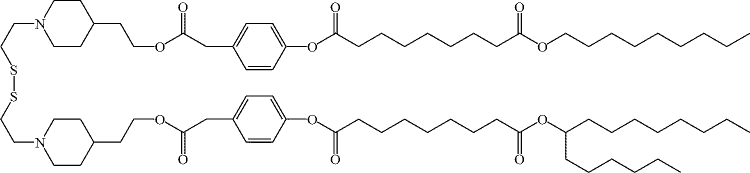 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

Lipid 62 1-nonyl 9-(4-(2-oxo-2-(2-(1-(2-((2-(4-(2-(2-(4-((9-oxo-9-(pentadecan-7-yloxy)nonanoyl)oxy)phenyl)acetoxy)ethyl)piperidin-1-yl)ethyl)disulfaneyl)ethyl)piperidin-4-yl)ethoxy)ethyl)phenyl) nonanedioate

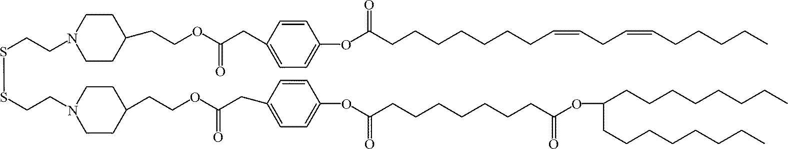 [SMILES: CCCCC/C=C\C/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 63 1-(heptadecan-9-yl) 9-(4-(2-(2-(1-(2-((2-(4-(2-(2-(4-(((9Z,12Z)-octadeca-9,12-dienoyl)oxy)phenyl)acetoxy)ethyl)piperidin-1-yl)ethyl)disulfaneyl)ethyl)piperidin-4-yl)ethoxy)-2-oxoethyl)phenyl) nonanedioate

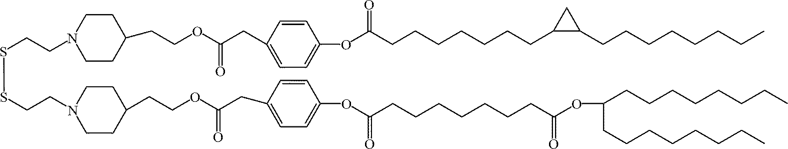 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC5CC5CCCCCCCC)cc4)CC3)CC2)cc1]

Lipid 64 1-(heptadecan-9-yl) 9-(4-(2-(2-(1-(2-((2-(4-(2-(2-(4-((8-(2-octylcyclopropyl)octanoyl)oxy)phenyl)acetoxy)ethyl)piperidin-1-yl)ethyl)disulfaneyl)ethyl)piperidin-4-yl)ethoxy)-2-oxoethyl)phenyl) nonanedioate

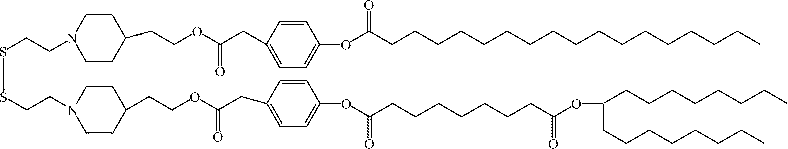 [SMILES: CCCCCCCCCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 65 1-(heptadecan-9-yl) 9-(4-(2-oxo-2-(2-(1-(2-((2-(4-(2-(2-(4-(stearoyloxy)phenyl)acetoxy)ethyl)piperidin-1-yl)ethyl)disulfaneyl)ethyl)piperidin-4-yl)ethoxy)ethyl)phenyl) nonanedioate

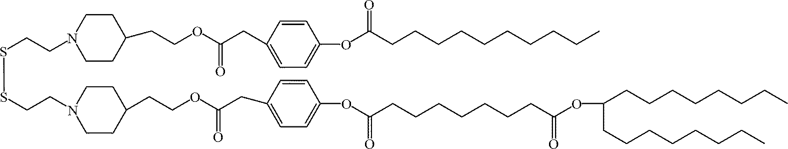 [SMILES: CCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 661-(heptadecan-9-yl) 9-(4-(2-oxo-2-(2-(1-(2-((2-(4-(2-(2-(4-(undecanoyloxy)phenyl)acetoxy)ethyl)piperidin-1-yl)ethyl)disulfaneyl)ethyl)piperidin-4-yl)ethoxy)ethyl)phenyl) nonanedioate 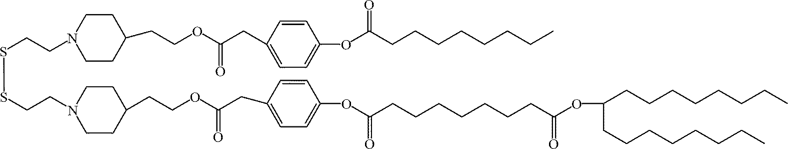 [SMILES: CCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 67 1-(heptadecan-9-yl) 9-(4-(2-(2-(1-(2-((2-(4-(2-(2-(4-(nonanoyloxy)phenyl)acetoxy)ethyl)piperidin-1-yl)ethyl)disulfaneyl)ethyl)piperidin-4-yl)ethoxy)-2-oxoethyl)phenyl) nonanedioate 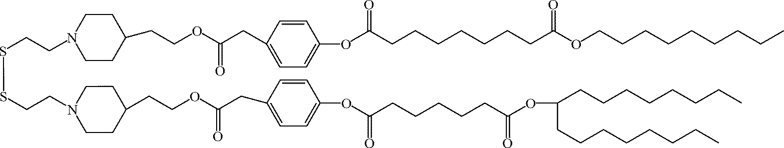 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]
Lipid 68 1-nonyl 9-(4-(2-(2-(1-(2-((2-(4-(2-(2-(4-((9-((3-octylundecyl)oxy)-9-oxononanoyl)oxy)phenyl)acetoxy)ethyl)piperidin-1-yl)ethyl)disulfaneyl)ethyl)piperidin-4-yl)ethoxy)-2-oxoethyl)phenyl) nonanedioate 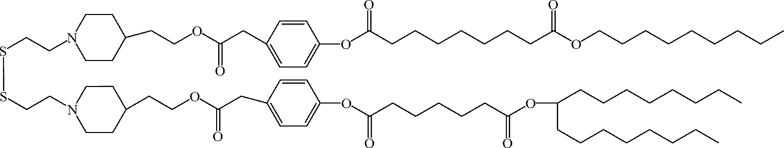 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]
Lipid 69 1-(4-(2-(2-(1-(2-((2-(4-(2-(2-(4-((7-(heptadecan-9-yloxy)-7-oxoheptanoyl)oxy)phenyl)acetoxy)ethyl)piperidin-1-yl)ethyl)disulfaneyl)ethyl)piperidin-4-yl)ethoxy)-2-oxoethyl)phenyl) 9-nonyl nonanedioate

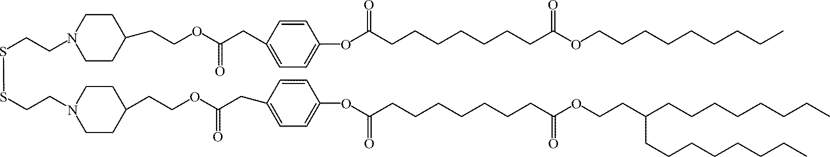 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCC(CCCCCCCC)CCCCCCCC)cc4)CC3)CC2)cc1]

Lipid 70
1-nonyl 9-(4-(2-(2-(1-(2-((2-(4-(2-(2-(4-((9-((3-octylundecyl)oxy)-9-oxononanoyl)oxy)phenyl)acetoxy)ethyl)piperidin-1-yl)ethyl)disulfaneyl)ethyl)piperidin-4-yl)ethoxy)-2-oxoethyl)phenyl) nonanedioate

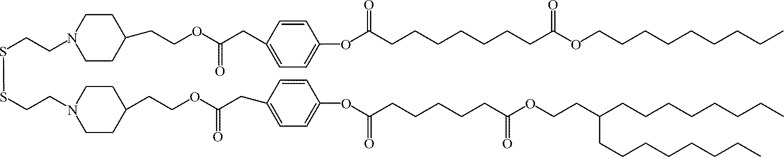 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OCCC(CCCCCCCC)CCCCCCCC)cc4)CC3)CC2)cc1]

Lipid 71 1-nonyl 9-(4-(2-(2-(1-(2-((2-(4-(2-(2-(4-((7-((3-octylundecyl)oxy)-7-oxoheptanoyl)oxy)phenyl)acetoxy)ethyl)piperidin-1-yl)ethyl)disulfaneyl)ethyl)piperidin-4-yl)ethoxy)-2-oxoethyl)phenyl) nonanedioate

### Formula (V)

In some aspects, the ionizable lipids are of the Formula (V):

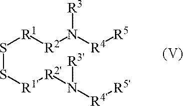

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

In a second chemical aspect, R¹ and R^{1′} in the ionizable lipids of the Formula (V) each independently (C₁-C₆)alkylene, wherein the remaining variables are as described above for Formula (V). Alternatively, as part of a second chemical aspect, R¹ and R^{1′} in the ionizable lipids of the Formula (V) each independently (C₁-C₃)alkylene, wherein the remaining variables are as described above for Formula (V).

In a third chemical aspect, the ionizable lipids of the Formula (V) are of the Formula (VI):

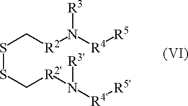

or a pharmaceutically acceptable salt thereof, wherein the remaining variables are as described above for Formula (V).

In a fourth chemical aspect, the ionizable lipids of the Formula (V) are of the Formula (VII) or (VIII):

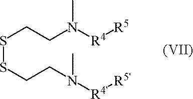

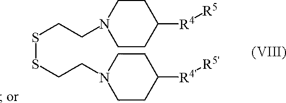

or a pharmaceutically acceptable salt thereof, wherein the remaining variables are as described above for Formula (V).

In a fifth chemical aspect, the ionizable lipids of the Formula (V) are of the Formula (IX) or (VI):

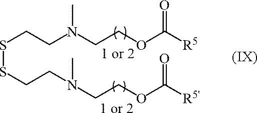

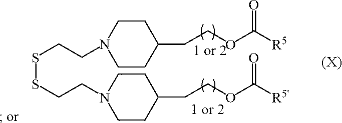

or a pharmaceutically acceptable salt thereof, wherein the remaining variables are as described above for Formula (V).

In a sixth chemical aspect, the ionizable lipids of the Formula (V) are of the Formula (XI), (XII), (XIII), or (XIV):

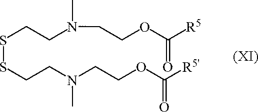

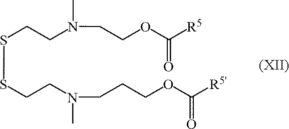

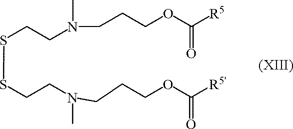

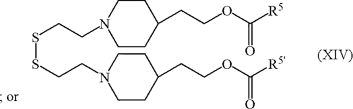

or a pharmaceutically acceptable salt thereof, wherein the remaining variables are as described above for Formula (XV).

In a seventh chemical aspect, at least one of R⁵ and R^{5′} in the ionizable lipid of Formula (V), (VI), (VII), (VIII), (IX), (X), (XI), (XII), (XIII), or (XIV) is a branched alkyl or branched alkenyl (number of carbon atoms as describeved above for Formula (V), (VI), (VII), (VIII), (IX), (X), (XI), (XII), (XIII), or (XIV)). In another alternative, as part of a seventh chemical aspect, one of R⁵ and R^{5′} in the ionizable lipid of Formula (V), (VI), (VII), (VIII), (IX), (X), (XI), (XII), (XIII), or (XIV) is a branched alkyl or branched alkenyl. In another alternative, as part of a seventh chemical aspect, R⁵ in the ionizable lipid of Formula (V), (VI), (VII), (VIII), (IX), (X), (XI), (XII), (XIII), or (XIV) is a branched alkyl or branched alkenyl. In another alternative, as part of a seventh chemical aspect, R^{5′} in the ionizable lipid of Formula (V), (VI), (VII), (VIII), (IX), (X), (XI), (XII), (XIII), or (XIV) is a branched alkyl or branched alkenyl.

In an eighth chemical aspect, R⁵ in the ionizable lipid of Formula (V), (VI), (VII), (VIII), (IX), (X), (XI), (XII), (XIII), or (XIV) is a (C₆-C₂₆)alkyl or (C₆-C₂₆)alkenyl, each of which are optionally interrupted with —C(O)O— or (C₃-C₆)cycloalkyl, wherein the remaining variables are as described above for Formula (I). Alternatively, as part of a seventh chemical aspect, R⁵ in the ionizable lipid of Formula (V), (VI), (VII), (VIII), (IX), (X), (XI), (XII), (XIII), or (XIV) is a (C₆-C₂₆)alkyl or (C₆-C₂₆)alkenyl, each of which are optionally interrupted with —C(O)O— or (C₃-C₅)cycloalkyl, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₇-C₂₆)alkyl or (C₇-C₂₆)alkenyl, each of which are optionally interrupted with —C(O)O— or (C₃-C₅)cycloalkyl, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₈-C₂₆)alkyl or (C₈-C₂₆)alkenyl, each of which are optionally interrupted with —C(O)O— or (C₃-C₅)cycloalkyl, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₆-C₂₄)alkyl or (C₆-C₂₄)alkenyl, each of which are optionally interrupted with —C(O)O— or cyclopropyl, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₈-C₂₄)alkyl or (C₈-C₂₄)alkenyl, wherein said (C₈-C₂₄)alkyl is optionally interrupted with —C(O)O— or cyclopropyl, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₈-C₁₀)alkyl, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₁₄-C₁₆)alkyl interrupted with cyclopropyl, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₁₀-C₂₄)alkyl interrupted with —C(O)O—, wherein the remaining variables are as described above for Formula (V). In another alternative, as part of an eighthchemical aspect, R⁵ in the ionizable lipid of Formula (V), (VI), (VII), (VIII), (IX), (X), (XI), (XII), (XIII), or (XIV) is a (C₁₆-C₁₈)alkenyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is — (CH₂)₃C(O)O(CH₂)₈CH₃, —(CH₂)₅C(O)O(CH₂)₈CH₃, —(CH₂)₇C(O)O(CH₂)₈CH₃, —(CH₂)₇C(O)OCH[(CH₂)₇CH₃]₂, —(CH₂)₇—C₃H₆—(CH₂)₇CH₃, —(CH₂)₇CH₃, —(CH₂)₉CH₃, —(CH₂)₁₆CH₃, —(CH₂)₇CH═CH(CH₂)₇CH₃, or —(CH₂)₇CH═CHCH₂CH═CH(CH₂)₄CH₃, wherein the remaining variables are as described above for Formula (XV).

In a ninth chemical aspect, R^{5′} in the ionizable lipid of Formula (V), (VI), (VII), (VIII), (IX), (X), (XI), (XII), (XIII), or (XIV) is a (C₁₅-C₂₈)alkyl interrupted with —C(O)O—, wherein the remaining variables are as described above for Formula (V) or the eighth chemical aspect. Alternatively, as part of a ninth chemical aspect, R^{5′} in the ionizable lipid of Formula (V), (VI), (VII), (VIII), (IX), (X), (XI), (XII), (XIII), or (XIV) is a (C₁₇-C₂₈)alkyl interrupted with —C(O)O—, wherein the remaining variables are as described above for Formula (V) or the eighth chemical aspect. In another alternative, as part of a ninth embodiment, R^{5′} in the ionizable lipid of Formula (V), (VI), (VII), (VIII), (IX), (X), (XI), (XII), (XIII), or (XIV) is a (C₁₉-C₂₈)alkyl interrupted with —C(O)O—, wherein the remaining variables are as described above for Formula (V) or the eighth chemical aspect. In another alternative, as part of a ninth chemical aspect, R^{5′} in the ionizable lipid of Formula (V), (VI), (VII), (VIII), (IX), (X), (XI), (XII), (XIII), or (XIV) is a (C₁₇-C₂₆)alkyl interrupted with —C(O)O—, wherein the remaining variables are as described above for Formula (V) or the eighth chemical aspect. In another alternative, as part of a ninth embodiment, R^{5′} in the ionizable lipid of Formula (V), (VI), (VII), (VIII), (IX), (X), (XI), (XII), (XIII), or (XIV) is a (C₁₉-C₂₆)alkyl interrupted with —C(O)O—, wherein the remaining variables are as described above for Formula (V) or the eighth chemical aspect. In another alternative, as part of a ninth chemical aspect, R^{5′} in the ionizable lipid of Formula (V), (VI), (VII), (VIII), (IX), (X), (XI), (XII), (XIII), or (XIV) is a (C₂₀-C₂₆)alkyl interrupted with —C(O)O—, wherein the remaining variables are as described above for Formula (V) or the eighth chemical aspect. In another alternative, as part of a ninth embodiment, R^{5′} is a (C₂₂-C₂₄)alkyl interrupted with —C(O)O—, wherein the remaining variables are as described above for Formula (V) or the eighth chemical aspect. In another alternative, as part of a ninth embodiment, R^{5′} is —(CH₂)₅C(O)OCH[(CH₂)₇CH₃]₂, — (CH₂)₇C(O)OCH[(CH₂)₇CH₃]₂, —(CH₂)₅C(O)OCH(CH₂)₂[(CH₂)₇CH₃]₂, or — (CH₂)₇C(O)OCH(CH₂)₂[(CH₂)₇CH₃]₂, wherein the remaining variables are as described above for Formula (V) or the eighth chemical aspect.

In another aspect, the ionizable lipid of Formula (V), (VI), (VIII), (VIII), (IX), (X), (XII), (XIII), or (XIV) may be selected from any of the following lipids in Table 6 or a pharmaceutically acceptable salt thereof.

TABLE 6

Exemplary ionizable lipids of Formula (V), (VI), (VIII), (VIII), (IX), (X), (XII), (XIII), or (XIV)

| Lipid No. | Lipid Structure and Name |
|---|---|
| 72 | 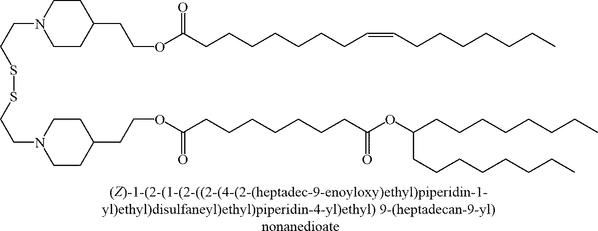 |
| 73 | 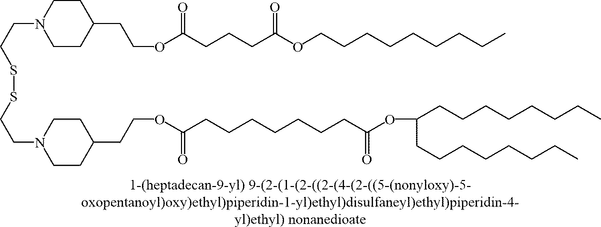 |
| 74 | 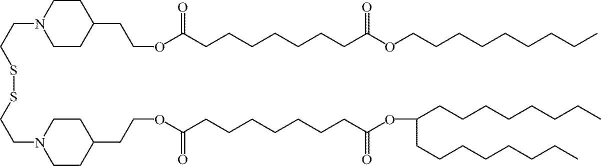 [SMILES: CCCCCCCCCOC(=O)CCCCCCCC(=O)OCCC1CCN(CCSSCCN2CCC(CCOC(=O)CCCCCCCC(=O)OC(CCCCCCCC)CCCCCCCC)CC2)CC1] |
| | 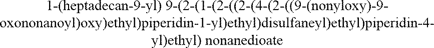 |
| 75 | 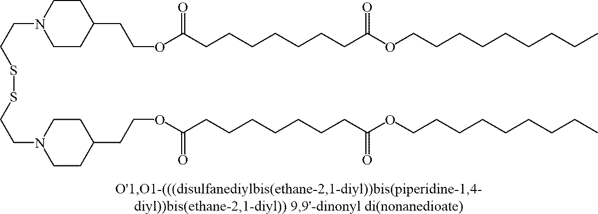 |
| 76 | 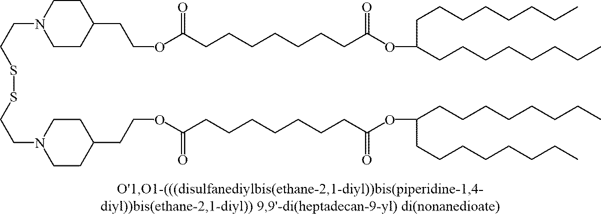 |

### Formula (XV)

In some aspects, the ionizable lipids are of the Formula (XV):

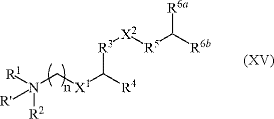

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₆ unbranched alkyl, C₂-C₁₆ unbranched alkenyl, or

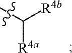

; wherein:
   R^{4a} and R^{4b} are each independently C₁-C₁₆ unbranched alkyl or C₂-C₁₆ unbranched alkenyl;
R⁵ is absent, C₁-C₆ alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CRª₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment, in the ionizable lipid according to the first embodiment, or a pharmaceutically acceptable salt thereof, X¹ and X² are the same; and all other remaining variables are as described for Formula (V) or the first embodiment.

In a third embodiment, in the ionizable lipid according to the first or second embodiment, or a pharmaceutically acceptable salt thereof, X¹ and X² are each independently —OC(═O)—, —SC(═O)—, —OC(═S)—, —C(═O)O—, —C(═O)S—, or —S—S—; or X¹ and X² are each independently —C(═O)O—, —C(═O)S—, or —S—S—; or X¹ and X² are each independently —C(═O)O— or —S—S—; and all other remaining variables are as described for Formula V or any one of the preceding embodiments.

In a fourth embodiment, the ionizable lipid of the present disclosure is represented by Formula (XVI):

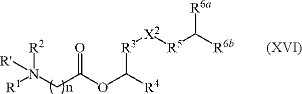

or a pharmaceutically acceptable salt thereof, wherein n is an integer selected from 1, 2, 3, and 4; and all other remaining variables are as described for Formula (XV) or any one of the preceding embodiments.

In a fifth embodiment, the ionizable lipid of the present disclosure is represented by Formula (XVII):

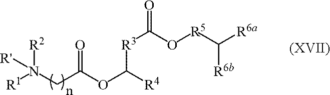

or a pharmaceutically acceptable salt thereof, wherein n is an integer selected from 1, 2, and 3; and all other remaining variables are as described for Formula (XV), Formula (XVI) or any one of the preceding embodiments.

In a sixth embodiment, the ionizable lipid of the present disclosure is represented by Formula (XVIII):

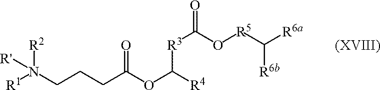

or a pharmaceutically acceptable salt thereof; and all other remaining variables are as described for Formula (XV), Formula (XVI), Formula (XVII) or any one of the preceding embodiments.

In a seventh embodiment, in the ionizable lipid according to Formula (XV), Formula (XVI), Formula (XVII), Formula (XVIII) or any one of the preceding embodiments, or a pharmaceutically acceptable salt thereof, R¹ and R² are each independently hydrogen, C₁-C₆ alkyl or C₂-C₆ alkenyl, or C₁-C₅ alkyl or C₂-C₅ alkenyl, or C₁-C₄ alkyl or C₂-C₄ alkenyl, or C₆ alkyl, or C₅ alkyl, or C₄ alkyl, or C₃ alkyl, or C₂ alkyl, or C₁ alkyl, or C₆ alkenyl, or C₅ alkenyl, or C₄ alkenyl, or C₃ alkenyl, or C₂ alkenyl; and all other remaining variables are as described for Formula (XV), Formula (XVI), Formula (XVII), Formula (XVIII) or any one of the preceding embodiments.

In an eighth embodiment, the ionizable lipid of the present disclosure is represented by Formula (XIX):

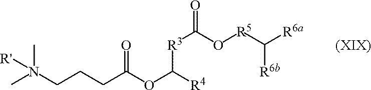

or a pharmaceutically acceptable salt thereof; and all other remaining variables are as described for Formula (XV), Formula (XVI), Formula (XVII), Formula (XVIII) or any one of the preceding embodiments.

In a ninth embodiment, in the ionizable lipid according to Formula (XV), Formula (XVI), Formula (XVII), Formula (XVIII), Formula (XIX) or any one of the preceding embodiments, or a pharmaceutically acceptable salt thereof, R³ is C₁-C₉ alkylene or C₂-C₉ alkenylene, C₁-C₇ alkylene or C₂-C₇ alkenylene, C₁-C₅ alkylene or C₂-C₅ alkenylene, or C₂-C₈ alkylene or C₂-C₈ alkenylene, or C₃-C₇ alkylene or C₃-C₇ alkenylene, or C₅-C₇ alkylene or C₅-C₇ alkenylene; or R³ is C₁₂ alkylene, C₁₁ alkylene, C₁₀ alkylene, C₉ alkylene, or C₈ alkylene, or C₇ alkylene, or C₆ alkylene, or C₅ alkylene, or C₄ alkylene, or C₃ alkylene, or C₂ alkylene, or C₁ alkylene, or C₁₂ alkenylene, C₁₁ alkenylene, C₁₀ alkenylene, C₉ alkenylene, or C₈ alkenylene, or C₇ alkenylene, or C₆ alkenylene, or C₅ alkenylene, or C₄ alkenylene, or C₃ alkenylene, or C₂ alkenylene; and all other remaining variables are as described for Formula (XV), Formula (XVI), Formula (XVII), Formula (XVIII), Formula (XIX) or any one of the preceding embodiments. Alternatively, as part of a ninth embodiment, in the cationic lipid according to Formula (XV), Formula (XVI), Formula (XVII), Formula (XVIII), Formula (XIX) or any one of the preceding embodiments, or a pharmaceutically acceptable salt thereof, R³ is C₁-C₉ alkylene or C₂-C₉ alkenylene, C₁-C₇ alkylene or C₂-C₇ alkenylene, C₁-C₆ alkylene or C₂-C₆ alkenylene, C₁-C₅ alkylene or C₂-C₅ alkenylene, or C₂-C₈ alkylene or C₂-C₈ alkenylene, or C₃-C₇ alkylene or C₃-C₇ alkenylene, or C₅-C₇ alkylene or C₅-C₇ alkenylene; or R³ is C₁₂ alkylene, C₁₁ alkylene, C₁₀ alkylene, C₉ alkylene, or C₈ alkylene, or C₇ alkylene, or C₆ alkylene, or C₅ alkylene, or C₄ alkylene, or C₃ alkylene, or C₂ alkylene, or C₁ alkylene, or C₁₂ alkenylene, C₁₁ alkenylene, C₁₀ alkenylene, C₉ alkenylene, or C₈ alkenylene, or C₇ alkenylene, or C₆ alkenylene, or C₅ alkenylene, or C₄ alkenylene, or C₃ alkenylene, or C₂ alkenylene; and all other remaining variables are as described for Formula (XV), Formula (XVI), Formula (XVII), Formula (XVIII), Formula (XIX) or any one of the preceding embodiments.

In a tenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇ alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₅ alkenylene, C₄ alkenylene, C₃ alkenylene, or C₂ alkenylene; and all other remaining variables are as described for Formula (XV), Formula (XVI), Formula (XVII), Formula (XVIII), Formula (XIX) or any one of the preceding embodiments.

In an eleventh embodiment, in the cationic lipid according to Formula (XV), Formula (XVI), Formula (XVII), Formula (XVIII), Formula (XIX) or any one of the preceding embodiments, or a pharmaceutically acceptable salt thereof, R⁴ is C₁-C₁₄ unbranched alkyl, C₂-C₁₄ unbranched alkenyl, or,

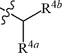

wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₇ unbranched alkyl or C₅-C₇ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is

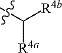

, wherein R^{4a} and R^{4b} are each independently C₂-C₁₀ unbranched alkyl or C₂-C₁₀ unbranched alkenyl; or R⁴ is

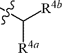

, wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; and all other remaining variables are as described for Formula (XV), Formula (XVI), Formula (XVII), Formula (XVIII), Formula (XIX) or any one of the preceding embodiments.

In a twelfth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} are each independently C₆-C₁₄ alkyl or C₆-C₁₄ alkenyl; or R^{6a} and R^{6b} are each independently C₈-C₁₂ alkyl or C₈-C₁₂ alkenyl; or R^{6a} and R^{6b} are each independently C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇ alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (XV), Formula (XVI), Formula (XVII), Formula (XVIII), Formula (XIX) or any one of the preceding embodiments.

In a thirteenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} contain an equal number of carbon atoms with each other; or R^{6a} and R^{6b} are the same; or R^{6a} and R^{6b} are both C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇ alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (XV), Formula (XVI), Formula (XVII), Formula (XVIII), Formula (XIX) or any one of the preceding embodiments.

In a fourteenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇ alkyl and R^{6a} is C₈ alkyl, R^{6a} is C₈ alkyl and R^{6a} is C₇ alkyl, R^{6a} is C₈ alkyl and R^{6a} is C₉ alkyl, R^{6a} is C₉ alkyl and R^{6a} is C₈ alkyl, R^{6a} is C₉ alkyl and R^{6a} is C₁₀ alkyl, R^{6a} is C₁₀ alkyl and R^{6a} is C₉ alkyl, R^{6a} is C₁₀ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₈ alkyl and R^{6a} is C₁₀ alkyl, R^{6a} is C₁₀ alkyl and R^{6a} is C₈ alkyl, R^{6a} is C₉ alkyl and R^{6a} is C₁₁ alkyl, R^{6a} is C₁₁ alkyl and R^{6a} is C₉ alkyl, R^{6a} is C₁₀ alkyl and R^{6a} is C₁₂ alkyl, R^{6a} is C₁₂ alkyl and R^{6a} is C₁₀ alkyl, R^{6a} is C₁₁ alkyl and R^{6a} is C₁₃ alkyl, or R^{6a} is C₁₃ alkyl and R^{6a} is C₁₁ alkyl, etc.; and all other remaining variables are as described for Formula 1, Formula II, Formula III, Formula IV, Formula V or any one of the preceding embodiments.

In one embodiment, the cationic lipid of the present disclosure or the cationic lipid of Formula (XV), Formula (XVI), Formula (XVII), Formula (XVIII), or Formula (XIX) is any one lipid selected from the lipids in Table 7 or a pharmaceutically acceptable salt thereof:

TABLE 7

Exemplary lipids of Formula (XV), Formula (XVI), Formula (XVII), Formula (XVIII), Formula (XIX)

| Lipid No. | Lipid Structure and Name |
|---|---|
| 77 | 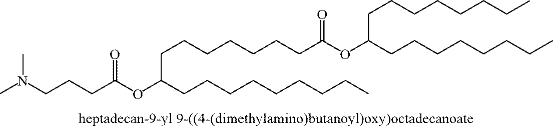 |
| 78 | 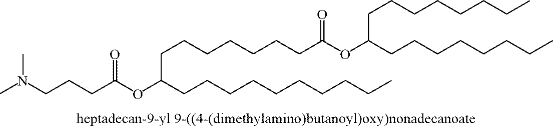 |
| 79 | 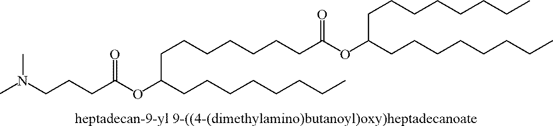 |
| 80 | 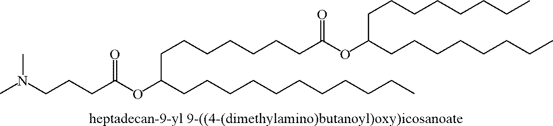 |
| 81 | 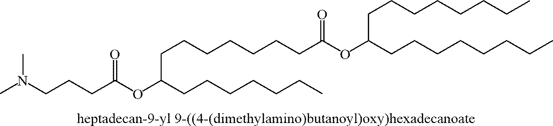 |
| 82 | 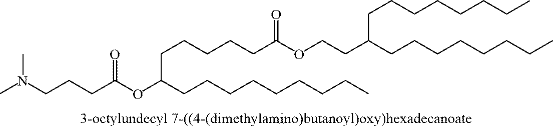 |
| 83 | 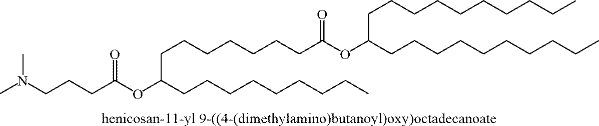 |
| 84 | 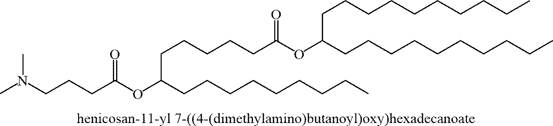 |
| 85 | 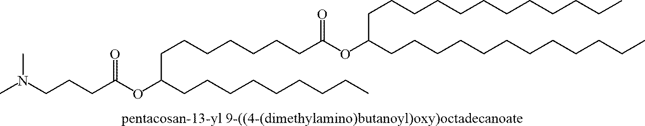 |
| 86 | 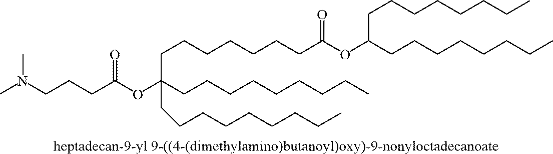 |
| 87 | 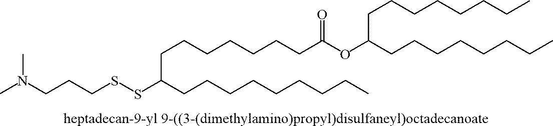 |
| 88 | 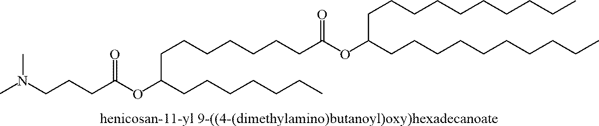 |
| 89 | 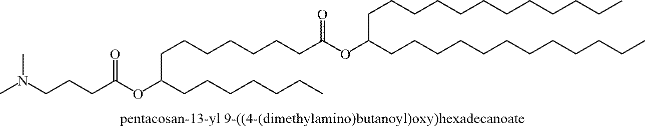 |
| 90 | 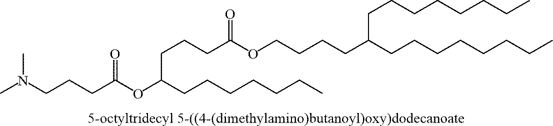 |
| 91 | 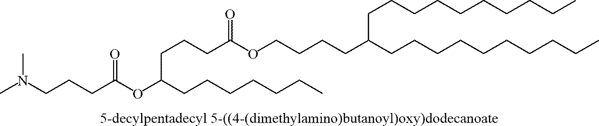 |
| 92 | 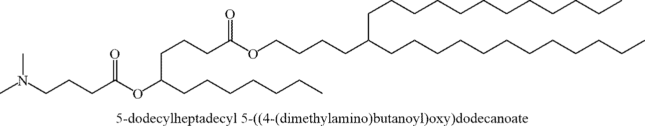 |
| 93 | 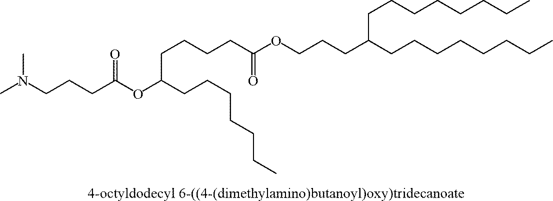 |
| 94 | 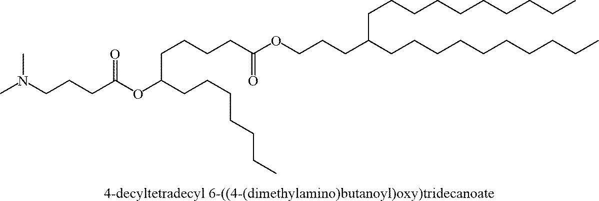 |
| 95 | 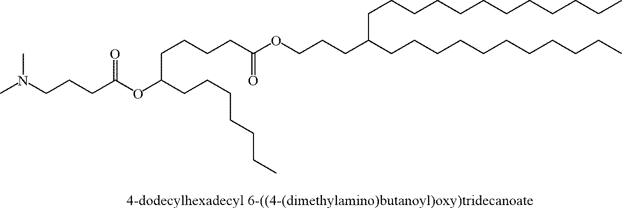 |
| 96 | 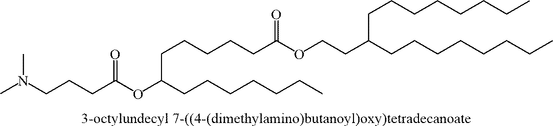 |
| 97 | 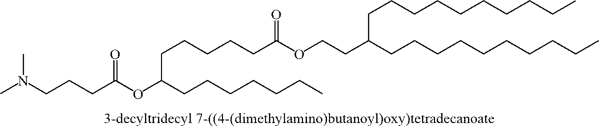 |
| 98 | 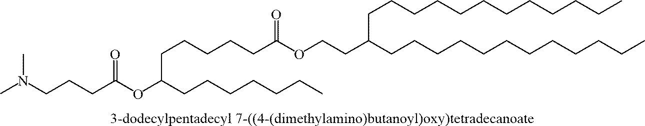 |
| 99 | 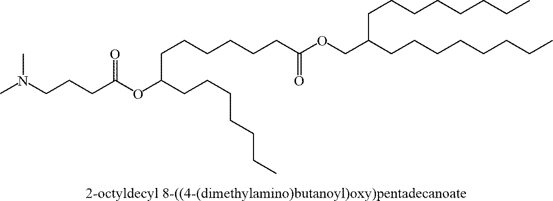 |
| 100 | 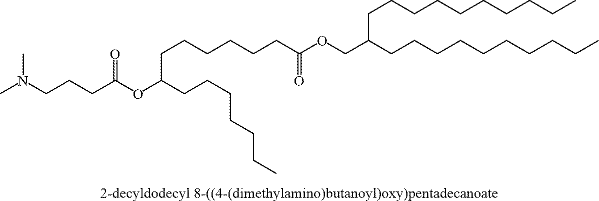 |
| 101 | 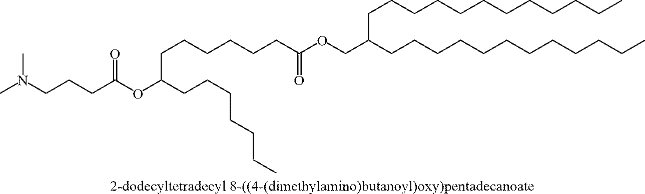 |

### Formula (XX)

In some aspects, the ionizable lipids are of the Formula (XX):

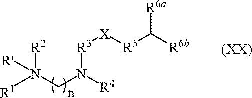

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆ unbranched alkenyl, or

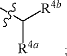

; wherein:
   R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment, in the ionizable lipid according to the first embodiment, or a pharmaceutically acceptable salt thereof, X is —OC(═O)—, —SC(═O)—, —OC(═S)—, —C(═O)O—, —C(═O)S—, or —S—S—; and all other remaining variables are as described for Formula (XX) or the first embodiment.

In a third embodiment, the ionizable lipid of the present disclosure is represented by Formula (XXI):

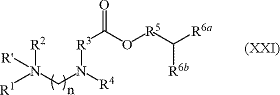

or a pharmaceutically acceptable salt thereof, wherein n is an integer selected from 1, 2, 3, and 4; and all other remaining variables are as described for Formula (XX) or any one of the preceding embodiments. In an alternative third embodiment, n is an integer selected from 1, 2, and 3; and all other remaining variables are as described for Formula (XX) or any one of the preceding embodiments.

In a fourth embodiment, the ionizable lipid of the present disclosure is represented by Formula (XXII):

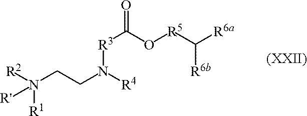

or a pharmaceutically acceptable salt thereof; and all other remaining variables are as described for Formula (XX), Formula (XXI) or any one of the preceding embodiments.

In a fifth embodiment, in the ionizable lipid according to the first embodiment, or a pharmaceutically acceptable salt thereof, R¹ and R² are each independently hydrogen or C₁-C₂ alkyl, or C₂-C₃ alkenyl; or R′, R¹, and R² are each independently hydrogen, C₁-C₂ alkyl; and all other remaining variables are as described for Formula (XX), Formula (XXI) or any one of the preceding embodiments.

In a sixth embodiment, the ionizable lipid of the present disclosure is represented by Formula (XXII):

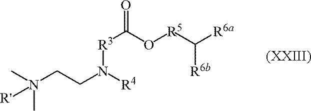

or a pharmaceutically acceptable salt thereof; and all other remaining variables are as described for Formula (XX), Formula (XXI), Formula (XXII) or any one of the preceding embodiments.

In a seventh embodiment, in the ionizable lipid according to Formula (XX), Formula (XXI), Formula (XXII), Formula (XXIII) or any one of the preceding embodiments, or a pharmaceutically acceptable salt thereof, R⁵ is absent or C₁-C₈ alkylene; or R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇ alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₅ alkenylene, C₄ alkenylene, C₃ alkenylene, or C₂ alkenylene; and all other remaining variables are as described for Formula (XX), Formula (XXI), Formula (XXII), Formula (XXIII) or any one of the preceding embodiments.

In an eighth embodiment, the ionizable lipid of the present disclosure is represented by Formula (XXIV):

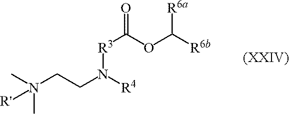

or a pharmaceutically acceptable salt thereof; and all other remaining variables are as described for Formula (XX), Formula (XXI), Formula (XXII), Formula (XXIII) or any one of the preceding embodiments.

In a ninth embodiment, in the ionizable lipid according to Formula (XX), Formula (XXI), Formula (XXII), Formula (XXIII), Formula (XXIV) or any one of the preceding embodiments, or a pharmaceutically acceptable salt thereof, R⁴ is C₁-C₁₄ unbranched alkyl, C₂-C₁₄ unbranched alkenyl, or

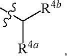

, wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₁₂ unbranched alkyl or C₅-C₁₂ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is,

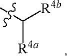

wherein R^{4a} and R^{4b} are each independently C₂-C₁₀ unbranched alkyl or C₂-C₁₀ unbranched alkenyl; or R⁴ is

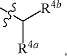

wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; and all other remaining variables are as described for Formula (XX), Formula (XXI), Formula (XXII), Formula (XXIII), Formula (XXIV) or any one of the preceding embodiments.

In a tenth embodiment, in the ionizable lipid according to Formula (XX), Formula (XXI), Formula (XXII), Formula (XXIII), Formula (XXIV) or any one of the preceding embodiments, or a pharmaceutically acceptable salt thereof, R³ is C₃-C₈ alkylene or C₃-C₈ alkenylene, C₃-C₇ alkylene or C₃-C₇ alkenylene, or C₃-C₅ alkylene or C₃-C₅ alkenylene,; or R³ is C₈ alkylene, or C₇ alkylene, or C₆ alkylene, or C₅ alkylene, or C₄ alkylene, or C₃ alkylene, or C₁ alkylene, or C₈ alkenylene, or C₇ alkenylene, or C₆ alkenylene, or C₅ alkenylene, or C₄ alkenylene, or C₃ alkenylene; and all other remaining variables are as described for Formula Formula (XX), Formula (XXI), Formula (XXII), Formula (XXIII), Formula (XXIV) or any one of the preceding embodiments.

In an eleventh embodiment, in the ionizable lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} are each independently C₇-C₁₂ alkyl or C₇-C₁₂ alkenyl; or R^{6a} and R^{6b} are each independently C₈-C₁₀ alkyl or C₈-C₁₀ alkenyl; or R^{6a} and R^{6b} are each independently C₁₂ alkyl, C₁₁ alkyl, C₁₀ alkyl, C₉ alkyl, C₈ alkyl, C₇ alkyl, C₁₂ alkenyl, C₁₁ alkenyl, C₁₀ alkenyl, C₉ alkenyl, C₈ alkenyl, or C₇ alkenyl; and all other remaining variables are as described for Formula (XX), Formula (XXI), Formula (XXII), Formula (XXIII), Formula (XXIV) or any one of the preceding embodiments.

In a twelfth embodiment, in the ionizable lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} contain an equal number of carbon atoms with each other; or R^{6a} and R^{6b} are the same; or R^{6a} and R^{6b} are both C₁₂ alkyl, C₁₁ alkyl, C₁₀ alkyl, C₉ alkyl, C₈ alkyl, C₇ alkyl, C₁₂ alkenyl, C₁₁ alkenyl, C₁₀ alkenyl, C₉ alkenyl, C₈ alkenyl, or C₇ alkenyl; and all other remaining variables are as described for Formula (XX), Formula (XXI), Formula (XXII), Formula (XXIII), Formula (XXIV) or any one of the preceding embodiments.

In a thirteenth embodiment, in the ionizable lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇ alkyl and R^{6a} is C₈ alkyl, R^{6a} is C₈ alkyl and R^{6a} is C₇ alkyl, R^{6a} is C₈ alkyl and R^{6a} is C₉ alkyl, R^{6a} is C₉ alkyl and R^{6a} is C₈ alkyl, R^{6a} is C₉ alkyl and R^{6a} is C₁₀ alkyl, R^{6a} is C₁₀ alkyl and R^{6a} is C₉ alkyl, R^{6a} is C₁₀ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₈ alkyl and R^{6a} is C₁₀ alkyl, R^{6a} is C₁₀ alkyl and R^{6a} is C₈ alkyl, R^{6a} is C₉ alkyl and R^{6a} is C₁₁ alkyl, R^{6a} is C₁₁ alkyl and R^{6a} is C₉ alkyl, R^{6a} is C₁₀ alkyl and R^{6a} is C₁₂ alkyl, R^{6a} is C₁₂ alkyl and R^{6a} is C₁₀ alkyl, etc.; and all other remaining variables are as described for Formula (XX), Formula (XXI), Formula (XXII), Formula (XXIII), Formula (XXIV)or any one of the preceding embodiments.

In a fourteenth embodiment, in the ionizable lipid according to Formula (XX), Formula (XXI), Formula (XXII), Formula (XXIII), Formula (XXIV)or any one of the preceding embodiments, or a pharmaceutically acceptable salt thereof, R′ is absent; and all other remaining variables are as described for Formula (XX), Formula (XXI), Formula (XXII), Formula (XXIII), Formula (XXIV)or any one of the preceding embodiments.

In one embodiment, the ionizable lipid of the present disclosure or the ionizable lipid of Formula (XX), Formula (XXI), Formula (XXII), Formula (XXIII), Formula (XXIV) is any one lipid selected from the lipids in Table 8 or a pharmaceutically acceptable salt thereof:

TABLE 8

Exemplary lipids of Formula (XX), Formula (XXI), Formula (XXII), Formula (XXIII), Formula (XXIV)

| Lipid No. | Lipid Structure and Name |
|---|---|
| 102 | 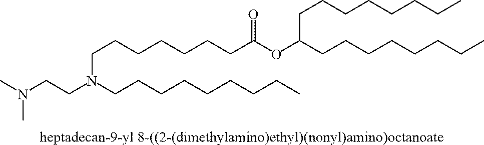 |
| 103 | 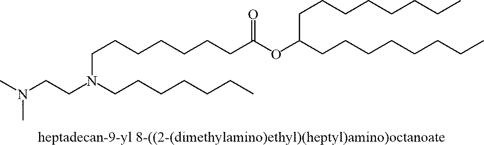 |
| 104 | 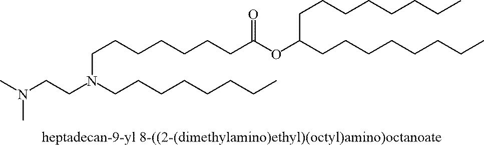 |
| 105 | 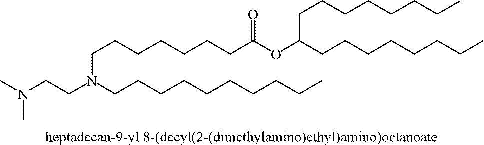 |
| 106 | 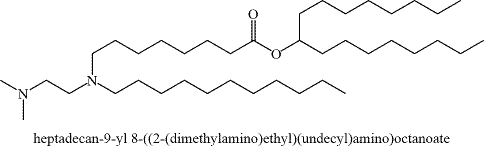 |
| 107 | 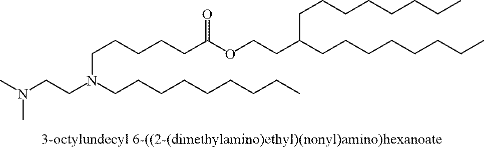 |
| 108 | 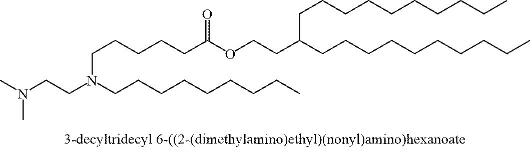 |
| 109 | 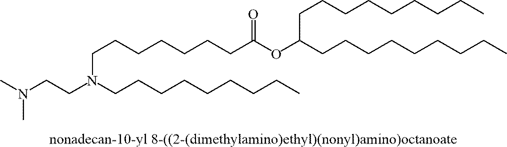 |
| 110 | 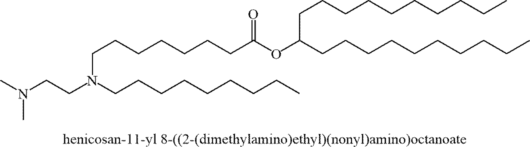 |
| 111 | 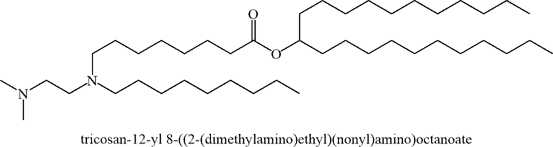 |
| 112 | 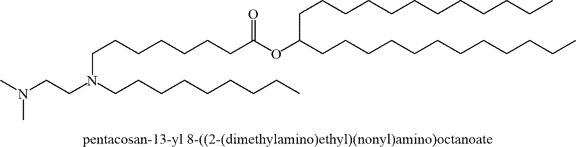 |

Specific examples are provided in the exemplification section below and are included as part of the ionizable lipids described herein. Pharmaceutically acceptable salts as well as neutral forms are also included.

### Cleavable Lipids

According to some embodiments, provided herein are pharmaceutical compositions comprising a cleavable lipid and a capsid free, non-viral vector (e.g., ceDNA) that can be used to deliver the capsid-free, non-viral DNA vector to a target site of interest (e.g., cell, tissue, organ, and the like). As used herein, the term “cleavable lipid” refers to a cationic lipid comprising a disulfide bond (“SS”) cleavable unit. In one embodiment, SS-cleavable lipids comprise a tertiary amine, which responds to an acidic compartment (e.g., an endosome or lysosome) for membrane destabilization and a disulfide bond that can cleave in a reductive environment (e.g., the cytoplasm). SS-cleavable lipids may include SS-cleavable and pH-activated lipid-like materials, such as ss-OP lipids, ssPalm lipids, ss-M lipids, ss-E lipids, ss-EC lipids, ss-LC lipids and ss-OC lipids, etc.

According to some embodiments, SS-cleavable lipids are described in International Patent Application Publication No. WO2019188867, incorporated by reference in its entirety herein.

As demonstrated herein, ceDNA lipid particles (e.g., lipid nanoparticles) comprising a cleavable lipid provide more efficient delivery of ceDNA to target cells (including, e.g., hepatic cells). The present disclosure provides a new formulation process and method that produce LNPs that are considerably smaller in size than previously described LNPs. According to some embodiments, the LNPs produced by the formulation process and methods described herein range in size from about 20 to about 70 nm in mean diameter, for example, a mean diameter of from about 20 nm to about 70 nm, about 25 nm to about 70 nm, from about 30 nm to about 70 nm, from about 35 nm to about 70 nm, from about 40 nm to about 70 nm, from about 45 nm to about 80 nm, from about 50 nm to about 70 nm, from about 60 nm to about 70 nm, from about 65 nm to about 70 nm, or about 20 nm, about 25 nm, about 30 nm, about 35 nm, about 40 nm, about 45 nm, about 50 nm, about 55 nm, about 60 nm, about 65 nm, about 70 nm. According to some embodiments, the mean diameter of the LNPs is about 50 nm to about 70 nm. which is significantly smaller and therefore advantageous in targeting and circumventing immune responses. Moreover, the LNPs described herein can encapsulate greater than about 60% to about 90% of rigid double stranded DNA, like ceDNA. According to some embodiments, the LNPs described herein can encapsulate greater than about 60% of rigid double stranded DNA, like ceDNA, greater than about 65% of rigid double stranded DNA, like ceDNA, greater than about 70% of rigid double stranded DNA, like ceDNA, greater than about 75% of rigid double stranded DNA, like ceDNA, greater than about 80% of rigid double stranded DNA, like ceDNA, greater than about 85% of rigid double stranded DNA, like ceDNA, or greater than about 90% of rigid double stranded DNA, like ceDNA.

The lipid particles (e.g., nanoparticles) (e.g., ceDNA lipid particles, mRNA lipid particles) described herein can advantageously be used to increase delivery of nucleic acids (e.g., ceDNA, mRNA) to target cells/tissues compared to LNPs produced by other processes, and compared to other lipids, e.g., ionizable cationic lipids. Thus, the lipid particles (e.g., nanoparticles) (e.g., ceDNA lipid particles, mRNA lipid particles) described herein provided maximum nucleic acid delivery compared to lipid particles prepared by processes and methods known in the art. Although the mechanism has not yet been determined, and without being bound by theory, it is thought that the lipid particles (e.g., nanoparticles) (e.g., ceDNA lipid particles, mRNA lipid particles) comprising a cleavable lipid prepared by the processes described herein provide improved delivery to hepatocytes escaping phagocytosis from and more efficient trafficking to the nucleus. Another advantage of the lipid particles (e.g., lipid nanoparticles) (e.g., ceDNA lipid particles, mRNA lipid particles) prepared by the processes described herein comprising a cleavable lipid described herein is better tolerability compared to other lipids, e.g., ionizable cationic lipids, e.g., MC3.

In one embodiment, a cleavable lipid may comprise three components: an amine head group, a linker group, and a hydrophobic tail(s). In one embodiment, the cleavable lipid comprises one or more phenyl ester bonds, one of more tertiary amino groups, and a disulfide bond. The tertiary amine groups provide pH responsiveness and induce endosomal escape, the phenyl ester bonds enhance the degradability of the structure (self- degradability) and the disulfide bond cleaves in a reductive environment.

In one embodiment, the cleavable lipid is an ss-OP lipid. In one embodiment, an ss-OP lipid comprises the structure shown in Formula A below:

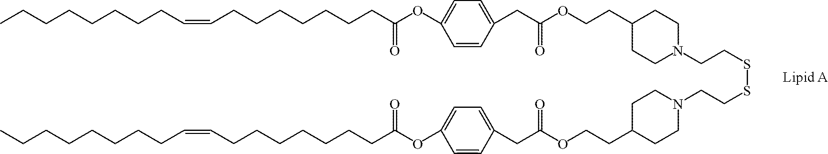 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In one embodiment, the SS-cleavable lipid is an SS-cleavable and pH-activated lipid-like material (ssPalm). ssPalm lipids are well known in the art. For example, see Togashi et al., Journal of Controlled Release, 279 (2018) 262-270, the entire contents of which are incorporated herein by reference. In one embodiment, the ssPalm is an ssPalmM lipid comprising the structure of Lipid B.

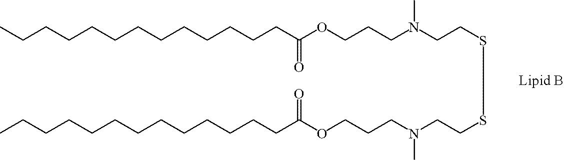 [SMILES: CCCCCCCCCCCCCC(=O)OCCCN(C)CCSSCCN(C)CCCOC(=O)CCCCCCCCCCCCC]

In one embodiment, the ssPalmE lipid is a ssPalmE—P4—C2 lipid, comprising the structure of Lipid C.

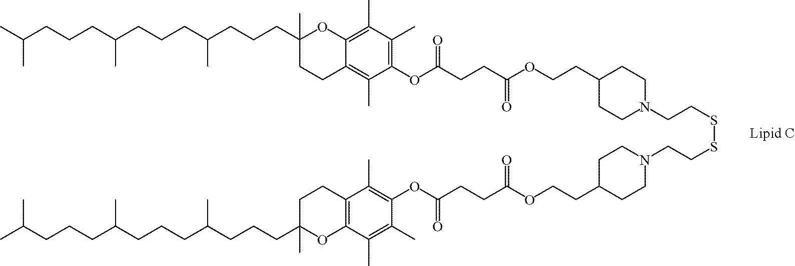 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the ssPalmE lipid is a ssPalmE—Paz4—C2 lipid, comprising the structure of Lipid D.

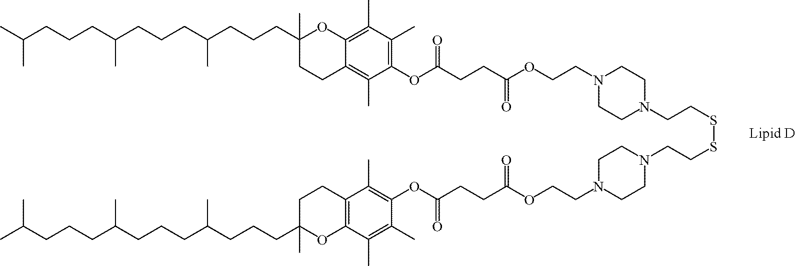 [SMILES: Cc1c(C)c2c(c(C)c1OC(=O)CCC(=O)OCCN1CCN(CCSSCCN3CCN(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-M lipid. In one embodiment, an ss-M lipid comprises the structure shown in Lipid E below:

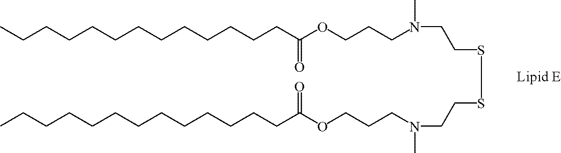 [SMILES: CCCCCCCCCCCCCC(=O)OCCCN(C)CCSSCCN(C)CCCOC(=O)CCCCCCCCCCCCC]

In one embodiment, the cleavable lipid is an ss-E lipid. In one embodiment, an ss-E lipid comprises the structure shown in Lipid F below:

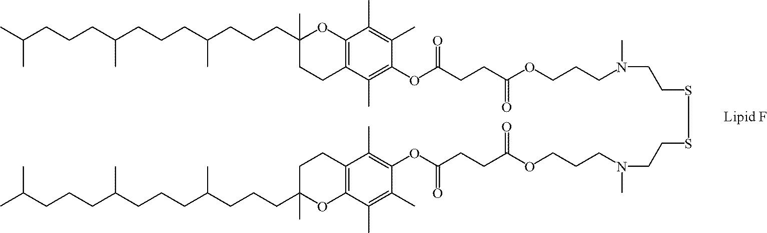 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

In one embodiment, the cleavable lipid is an ss-EC lipid. In one embodiment, an ss-EC lipid comprises the structure shown in Lipid G below:

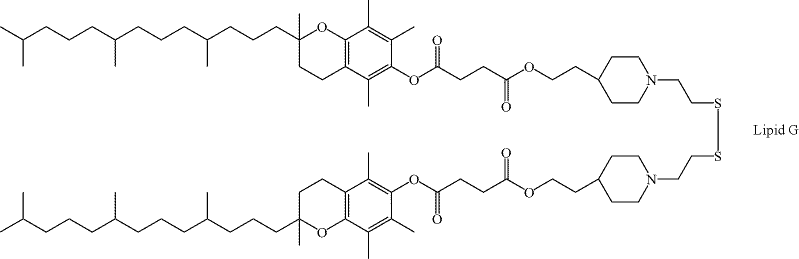 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-LC lipid. In one embodiment, an ss-LC lipid comprises the structure shown in Lipid H below:

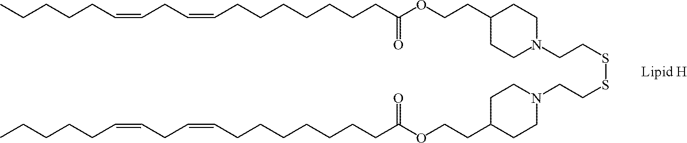 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

In one embodiment, the cleavable lipid is an ss-OC lipid. In one embodiment, an ss-OC lipid comprises the structure shown in Lipid J below:

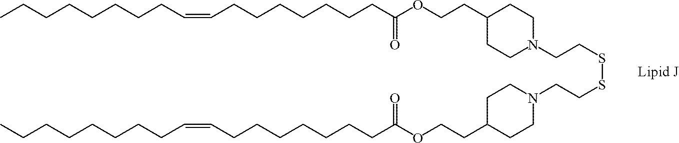 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

In one embodiment, a lipid particle (e.g., lipid nanoparticle) formulation is made and loaded with ceDNA obtained by the process as disclosed in International Patent Application No. PCT/US2018/050042, filed on Sep. 7, 2018, which is incorporated by reference in its entirety herein. This can be accomplished by high energy mixing of ethanolic lipids with aqueous ceDNA at low pH which protonates the lipid and provides favorable energetics for ceDNA/lipid association and nucleation of particles. The particles can be further stabilized through aqueous dilution and removal of the organic solvent. The particles can be concentrated to the desired level. In one embodiment, the disclosure provides a ceDNA lipid particle comprising a lipid of Formula I prepared by a process as described in Example 2.

Generally, the lipid particles (e.g., lipid nanoparticles) are prepared at a total lipid to ceDNA (mass or weight) ratio of from about 10:1 to 60:1. In some embodiments, the lipid to ceDNA ratio (mass/mass ratio; w/w ratio) can be in the range of from about 1:1 to about 60:1, from about 1:1 to about 55:1, from about 1:1 to about 50:1, from about 1:1 to about 45:1, from about 1:1 to about 40:1, from about 1:1 to about 35:1, from about 1:1 to about 30:1, from about 1:1 to about 25:1, from about 10:1 to about 14:1, from about 3:1 to about 15:1, from about 4:1 to about 10:1, from about 5:1 to about 9:1, about 6:1 to about 9:1; from about 30:1 to about 60:1. According to some embodiments, the lipid particles (e.g., lipid nanoparticles) are prepared at a ceDNA (mass or weight) to total lipid ratio of about 60:1. According to some embodiments, the lipid particles (e.g., lipid nanoparticles) are prepared at a ceDNA (mass or weight) to total lipid ratio of about 30:1. The amounts of lipids and ceDNA can be adjusted to provide a desired N/P ratio, for example, N/P ratio of 3, 4, 5, 6, 7, 8, 9, 10 or higher. Generally, the lipid particle formulation’s overall lipid content can range from about 5 mg/ml to about 30 mg/mL.

In some embodiments, the lipid nanoparticle comprises an agent for condensing and/or encapsulating nucleic acid cargo, such as ceDNA. Such an agent is also referred to as a condensing or encapsulating agent herein. Without limitations, any compound known in the art for condensing and/or encapsulating nucleic acids can be used as long as it is non-fusogenic. In other words, an agent capable of condensing and/or encapsulating the nucleic acid cargo, such as ceDNA, but having little or no fusogenic activity. Without wishing to be bound by theory, a condensing agent may have some fusogenic activity when not condensing/encapsulating a nucleic acid, such as ceDNA, but a nucleic acid encapsulating lipid nanoparticle formed with said condensing agent can be non-fusogenic. The formulation process described herein takes advantage of the finding that ceDNA compaction occurs in solvents with high ethanol content. When aqueous ceDNA (90% EtOH) is added to an ethanolic solution (e.g., 90% EtOH) of lipids in a ratio such that the resulting solution is 90-92% ethanol and 8-10% water, the ceDNA is observed to exist in a compacted state by dynamic light scattering. In such a solvent (90-92% ethanol, 8-10% water), both the lipids and ceDNA are solubilized with no detectable precipitation of either component.

According to some embodiments, formulation process and methods described by the present disclosure can encapsulate considerably more double stranded DNA (e.g., ceDNA) than has been previously reported. According to some embodiments, the LNPs describedherein can encapsulate greater than about 60% of rigid double stranded DNA, like ceDNA, greater than about 65% of rigid double stranded DNA, like ceDNA, greater than about 70% of rigid double stranded DNA, like ceDNA, greater than about 75% of rigid double stranded DNA, like ceDNA, greater than about 80% of rigid double stranded DNA, like ceDNA,n greater than about 85% of rigid double stranded DNA, like ceDNA, or greater than about 90% of rigid double stranded DNA, like ceDNA.

According to some embodiments, the solvent comprises about 80% ethanol and about 20% water. According to some embodiments, the solvent comprises about 81% ethanol and about 19% water. According to some embodiments, the solvent comprises about 82% ethanol and about 18% water. According to some embodiments, the solvent comprises about 83% ethanol and about 17% water. According to some embodiments, the solvent comprises about 84% ethanol and about 16% water. According to some embodiments, the solvent comprises about 85% ethanol and about 15% water. According to some embodiments, the solvent comprises about 86% ethanol and about 14% water. According to some embodiments, the solvent comprises about 87% ethanol and about 13% water. According to some embodiments, the solvent comprises about 88% ethanol and about 12% water. According to some embodiments, the solvent comprises about 89% ethanol and about 11% water. According to some embodiments, the solvent comprises about 90% ethanol and about 10% water. According to some embodiments, the solvent comprises about 91% ethanol and about 9% water. According to some embodiments, the solvent comprises about 92% ethanol and about 8% water. According to some embodiments, the solvent comprises about 93% ethanol and about 7% water. According to some embodiments, the solvent comprises about 94% ethanol and about 6% water. According to some embodiments, the solvent comprises about 95% ethanol and about 5% water.

The cationic lipid is typically employed to condense the nucleic acid cargo, e.g., ceDNA at low pH and to drive membrane association and fusogenicity. Generally, catonic lipids are lipids comprising at least one amino group that is positively charged or becomes protonated under acidic conditions, for example at pH of 6.5 or lower. Cationic lipids may also be ionizable lipids, e.g., ionizable cationic lipids. By a “non-fusogenic cationic lipid” is meant a cationic lipid that can condense and/or encapsulate the nucleic acid cargo, such as ceDNA, but does not have, or has very little, fusogenic activity.

In one embodiment, the cationic lipid can comprise 20-90% (mol) of the total lipid present in the lipid particles (e.g., lipid nanoparticles). For example, cationic lipid molar content can be 20-70% (mol), 30-60% (mol), 40-60% (mol), 40-55% (mol) or 45-55% (mol) of the total lipid present in the lipid particle (e.g., lipid nanoparticles). In some embodiments, cationic lipid comprises from about 50 mol % to about 90 mol % of the total lipid present in the lipid particles (e.g., lipid nanoparticles).

In one embodiment, the SS-cleavable lipid is not MC3 (6Z,9Z,28Z,3 1Z)-heptatriaconta-6,9,28,3 1-tetraen-19-yl-4-(dimethylamino)butanoate (DLin-MC3-DMA or MC3). DLin-MC3-DMA is described in Jayaraman et al., Angew. Chem. Int. Ed Engl. (2012), 51(34): 8529-8533, the contents of which is incorporated herein by reference in its entirety. The structure of D-Lin-MC3-DMA (MC3) is shown below as Lipid K:

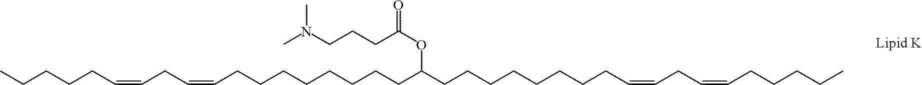 [SMILES: CCCCC/C=C\C/C=C\CCCCCCCCC(CCCCCCCC/C=C\C/C=C\CCCCC)OC(=O)CCCN(C)C]

In one embodiment, the cleavable lipid is not the lipid ATX-002. The lipid ATX-002 is described in WO2015/074085, the content of which is incorporated herein by reference in its entirety. In one embodiment, the cleavable lipid is not (13Z.16Z)-/V,/V-dimethyl-3-nonyldocosa- 13,16-dien-l-amine (Compound 32). Compound 32 is described in WO2012/040184, the contents of which is incorporated herein by reference in its entirety. In one embodiment, the cleavable lipid is not Compound 6 or Compound 22. Compounds 6 and 22 are described in WO2015/199952, the content of which is incorporated herein by reference in its entirety.

Non-limiting examples of cationic lipids include SS-cleavable and pH-activated lipid-like material-OP (ss-OP; Formula I), SS-cleavable and pH-activated lipid-like material-M (SS-M; Formula V), SS-cleavable and pH-activated lipid-like material-E (SS-E; Formula VI), SS-cleavable and pH-activated lipid-like material-EC (SS-EC; Formula VII), SS-cleavable and pH-activated lipid-like material-LC (SS-LC; Formula VIII), SS-cleavable and pH-activated lipid-like material—OC (SS—OC; Formula IX), polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N—[1-(2,3-dioleoloxy)-propyl]—N,N,N-trimethylammonium chloride (DOTMA), N—[1 - (2,3-dioleoloxy)-propyl]—N,N,N-trimethylammonium methylsulfate (DOTAP), 3b—[N—(N′,N′-dimethylaminoethane)carbamoyl]cholesterol (DC-Chol), 2,3,-dioleyloxy—N—[2(sperminecarboxamido)ethyl]—N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., ceDNA or CELiD) can also be complexed with, e.g., poly (L-lysine) or avidin and lipids can, or cannot, be included in this mixture, e.g., steryl-poly (L-lysine).

In one embodiment, the cationic lipid is ss-OP of Formula I. In another embodiment, the cationic lipid SS-PAZ of Formula II.

In one embodiment, a ceDNA vector as disclosed herein is delivered using a cationic lipid described in U.S. Pat. No. 8,158,601, or a polyamine compound or lipid as described in U.S. Pat. No. 8,034,376.

### Non-cationic Lipids

In one embodiment, the lipid particles (e.g., lipid nanoparticles) can further comprise a non-cationic lipid. The non-cationic lipid can serve to increase fusogenicity and also increase stability of the LNP during formation. Non-cationic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3 -pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof. It is to be understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Other examples of non-cationic lipids suitable for use in the lipid particles (e.g., lipid nanoparticles) include nonphosphorous lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In one embodiment, the non-cationic lipid is a phospholipid. In one embodiment, the non-cationic lipid is selected from the group consisting of DSPC, DPPC, DMPC, DOPC, POPC, DOPE, and SM. In some embodiments, the non-cationic lipid is DSPC. In other embodiments, the non-cationic lipid is DOPC. In other embodiments, the non-cationic lipid is DOPE.

In some embodiments, the non-cationic lipid can comprise 0-20% (mol) of the total lipid present in the lipid nanoparticle. In some embodiments, the non-cationic lipid content is 0.5-15% (mol) of the total lipid present in the lipid particle (e.g., lipid nanoparticle). In some embodiments, the non-cationic lipid content is 5-12% (mol) of the total lipid present in the lipid particle (e.g., lipid nanoparticle). In some embodiments, the non-cationic lipid content is 5-10% (mol) of the total lipid present in the lipid particle (e.g., lipid nanoparticle). In one embodiment, the non-cationic lipid content is about 6% (mol) of the total lipid present in the lipid particle (e.g., lipid nanoparticle). In one embodiment, the non-cationic lipid content is about 7.0% (mol) of the total lipid present in the lipid particle (e.g., lipid nanoparticle). In one embodiment, the non-cationic lipid content is about 7.5% (mol) of the total lipid present in the lipid particle (e.g., lipid nanoparticle). In one embodiment, the non-cationic lipid content is about 8.0% (mol) of the total lipid present in the lipid particle (e.g., lipid nanoparticle). In one embodiment, the non-cationic lipid content is about 9.0% (mol) of the total lipid present in the lipid particle (e.g., lipid nanoparticle). In some embodiments, the non-cationic lipid content is about 10% (mol) of the total lipid present in the lipid particle (e.g., lipid nanoparticle). In one embodiment, the non-cationic lipid content is about 11% (mol) of the total lipid present in the lipid particle (e.g., lipid nanoparticle).

Exemplary non-cationic lipids are described in International Patent Application Publication No. WO2017/099823 and US Patent Application Publication No. US2018/0028664, the contents of both of which are incorporated herein by reference in their entirety.

In one embodiment, the lipid particles (e.g., lipid nanoparticles) can further comprise a component, such as a sterol, to provide membrane integrity and stability of the lipid particle. In one embodiment, an exemplary sterol that can be used in the lipid particle is cholesterol, or a derivative thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholestryl hemisuccinate (CHEMS).

Exemplary cholesterol derivatives are described in International Patent Application Publication No. WO2009/127060 and U.S. Pat. Application Publication No. US2010/0130588, contents of both of which are incorporated herein by reference in their entirety.

In one embodiment, the component providing membrane integrity, such as a sterol, can comprise 0-50% (mol) of the total lipid present in the lipid particle (e.g., lipid nanoparticle). In some embodiments, such a component is 20-50% (mol) of the total lipid content of the lipid particle (e.g., lipid nanoparticle). In some embodiments, such a component is 30-40% (mol) of the total lipid content of the lipid particle (e.g., lipid nanoparticle). In some embodiments, such a component is 35-45% (mol) of the total lipid content of the lipid particle (e.g., lipid nanoparticle). In some embodiments, such a component is 38-42% (mol) of the total lipid content of the lipid particle (e.g., lipid nanoparticle).

In one embodiment, the lipid particle (e.g., lipid nanoparticle) can further comprise a polyethylene glycol (PEG) or a conjugated lipid molecule. Generally, these are used to inhibit aggregation of lipid particle (e.g., lipid nanoparticle) and/or provide steric stabilization. Exemplary conjugated lipids include, but are not limited to, PEG-lipid conjugates, polyoxazoline (POZ)-lipid conjugates, polyamide -lipid conjugates (such as ATTA-lipid conjugates), cationic-polymer lipid (CPL) conjugates, and mixtures thereof. In some embodiments, the conjugated lipid molecule is a PEGylated lipid, for example, a (methoxy polyethylene glycol)-conjugated lipid. In some other embodiments, the PEGylated lipid is PEG₂₀₀₀-DMG (dimyristoylglycerol).

Exemplary PEGylated lipids include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, or a mixture thereof. Additional exemplary PEG-lipid conjugates are described, for example, in US5,885,613, US6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In one embodiment, the PEG-DAA PEGylated lipid can be, for example, PEG-dilauryloxypropyl, PEG- dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine—N—[methoxy(polyethylene glycol)-2000]. In one embodiment, the PEG-lipid can be selected from the group consisting of PEG-DMG, 1,2-dimyristoyl-sn-glycero-3-phosphoethanolamine—N—[methoxy(polyethylene glycol)-2000],

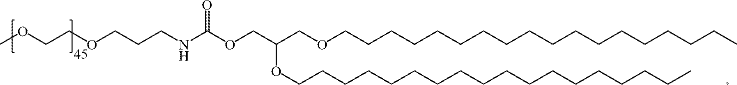

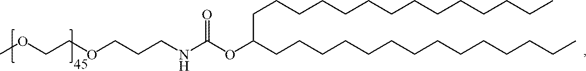

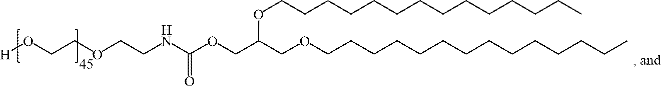

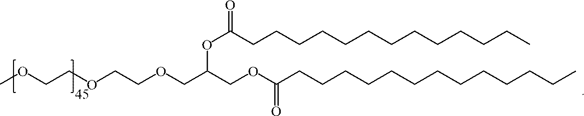

In some embodiments, the PEGylated lipid is selected from the group consisting N-(Carbonyl-methoxypolyethyleneglycoln)-1,2-dimyristoyl-sn-glycero-3 -phosphoethanolamine (DMPE-PEGₙ, where n is 350, 500, 750, 1000 or 2000), N-(Carbonyl-methoxypolyethyleneglycolₙ)-1,2-distearoyl-sn-glycero-3-phosphoethanolamine (DSPE-PEGₙ, where n is 350, 500, 750, 1000 or 2000), DSPE-polyglycelin-cyclohexyl-carboxylic acid, DSPE-polyglycelin-2-methylglutar-carboxylic acid, 1,2-Distearoyl-sn-Glycero-3-Phosphoethanolamine (DSPE) conjugated Polyethylene Glycol (DSPE-PEG-OH), polyethylene glycol-dimyristolglycerol (PEG-DMG), polyethylene glycol-distearoyl glycerol (PEG-DSG), or N-octanoyl-sphingosine-1-{succinyl[methoxy(polyethylene glycol)200011 (C8 PEG2000 Ceramide). In some examples of DMPE-PEGₙ, where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some examples of DSPE-PEGₙ. where n is 350, 500, 750, 1000 or 2000, the PEG-lipid is N-(Carbonyl-methoxypolyethyleneglycol 2000)-1,2-distearoyl-sn-glycero-3-phosphoethanolamine (DSPE-PEG 2,000). In some embodiments, the PEG-lipid is DSPE-PEG-OH. In some preferred embodiments, the PEG-lipid is PEG-DMG.

In some embodiments, the conjugated lipid, e.g., PEGylated lipid, includes a tissue-specific targeting ligand, e.g., first or second targeting ligand. For example, PEG-DMG conjugated with a GalNAc ligand.

In one embodiment, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic -polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International Patent Application Publication Nos. WO 1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, U.S. Pat. Application Publication Nos. US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. US5,885,613, US6,287,591, US6,320,017, and US6,586,559, the contents of all of which are incorporated herein by reference in their entireties.

In some embodiments, the PEGylated lipid can comprise 0-20% (mol) of the total lipid present in the lipid nanoparticle. In some embodiments, PEGylated lipid content is 0.5-10% (mol). In some embodiments, PEGylated lipid content is 1-5% (mol). In some embodiments, PEGylated lipid content is 2-4% (mol). In some embodiments, PEGylated lipid content is 2-3% (mol). In one embodiment, PEGylated lipid content is about 2% (mol). In one embodiment, PEGylated lipid content is about 2.5% (mol). In some embodiments, PEGylated lipid content is about 3% (mol). In one embodiment, PEGylated lipid content is about 3.5% (mol). In one embodiment, PEGylated lipid content is about 4% (mol).

It is understood that molar ratios of the cationic lipid, e.g., ionizable cationic lipid, with the non-cationic-lipid, sterol, and PEGylated lipid can be varied as needed. For example, the lipid particle (e.g., lipid nanoparticle) can comprise 30-70% cationic lipid by mole or by total weight of the composition, 0-60% cholesterol by mole or by total weight of the composition, 0-30% non-cationic lipid by mole or by total weight of the composition and 2-5% PEGylated lipid by mole or by total weight of the composition. In one embodiment, the composition comprises 40-60% cationic lipid by mole or by total weight of the composition, 30-50% cholesterol by mole or by total weight of the composition, 5-15% non-cationic lipid by mole or by total weight of the composition and 2-5% PEG or the conjugated lipid by mole or by total weight of the composition. In one embodiment, the composition is 40-60% cationic lipid by mole or by total weight of the composition, 30-40% cholesterol by mole or by total weight of the composition, and 5- 10% non-cationic lipid, by mole or by total weight of the composition and 2-5% PEGylated lipid by mole or by total weight of the composition. The composition may contain 60-70% cationic lipid by mole or by total weight of the composition, 25-35% cholesterol by mole or by total weight of the composition, 5-10% non-cationic-lipid by mole or by total weight of the composition and 2-5% PEGylated lipid by mole or by total weight of the composition. The composition may also contain up to 45-55% cationic lipid by mole or by total weight of the composition, 35-45% cholesterol by mole or by total weight of the composition, 2 to 15% non-cationic lipid by mole or by total weight of the composition, and 2-5% PEGylated lipid by mole or by total weight of the composition. The formulation may also be a lipid nanoparticle formulation, for example comprising 8-30% cationic lipid by mole or by total weight of the composition, 5-15% non-cationic lipid by mole or by total weight of the composition, and 0-40% cholesterol by mole or by total weight of the composition; 4-25% cationic lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% cationic lipid by mole or by total weight of the composition, 2-30% non-cationic lipid by mole or by total weight of the composition, 1 to 15% cholesterol by mole or by total weight of the composition, 2 to 35% PEGylated lipid by mole or by total weight of the composition, and 1-20% cholesterol by mole or by total weight of the composition; or even up to 90% cationic lipid by mole or by total weight of the composition and 2-10% non- cationic lipids by mole or by total weight of the composition, or even 100% cationic lipid by mole or by total weight of the composition. In some embodiments, the lipid particle formulation comprises cationic lipid, non-cationic phospholipid, cholesterol and a PEGylated lipid (conjugated lipid) in a molar ratio of about 50:9:38.5:2.5.

In one embodiment, the lipid particle (e.g., lipid nanoparticle) formulation comprises cationic lipid, non-cationic phospholipid, cholesterol and a PEGylated lipid (conjugated lipid) in a molar ratio of about 50:7:40:3.

In other aspects, the disclosure provides for a lipid nanoparticle formulation comprising phospholipids, lecithin, phosphatidylcholine and phosphatidylethanolamine.

In one embodiment, the lipid particle (e.g., lipid nanoparticle) comprises cationic lipid, non-cationic lipid (e.g. phospholipid), a sterol (e.g., cholesterol) and a PEGylated lipid (conjugated lipid), where the molar ratio of lipids ranges from 20 to 70 mole percent for the cationic lipid, with a target of 30-60, the mole percent of non-cationic lipid ranges from 0 to 30, with a target of 0 to 15, the mole percent of sterol ranges from 20 to 70, with a target of 30 to 50, and the mole percent of PEGylated lipid (conjugated lipid) ranges from 1 to 6, with a target of 2 to 5.

Lipid nanoparticles (LNPs) comprising ceDNA are disclosed in International Patent Application No. PCT/US2018/050042, filed on Sep. 7, 2018, which is incorporated herein in its entirety and envisioned for use in the methods and compositions as disclosed herein.

Lipid particle (e.g., lipid nanoparticle) size can be determined by quasi-elastic light scattering using a Malvern Zetasizer Nano ZS (Malvern, UK). According to some embodiments, LNP mean diameter as determined by light scattering is less than about 75 nm or less than about 70 nm. According to some embodiments, LNP mean diameter as determined by light scattering is between about 50 nm to about 75 nm or about 50 nm to about 70 nm.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al., Nature Biotechnology 28, 172-176 (20 1 0), both of which are incorporated by reference in their entireties). In one embodiment, the pKa of each cationic lipid is determined in lipid nanoparticles using an assay based on fluorescence of 2-(p- toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 mM stock solution in distilled water. Vesicles can be diluted to 24 mM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 mM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

In one embodiment, relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, a lipid particle (e.g., lipid nanoparticle) of the disclosure includes a lipid formulation that can be used to deliver a capsid-free, non-viral DNA vector to a target site of interest (e.g., cell, tissue, organ, and the like). Generally, the lipid particle (e.g., lipid nanoparticle) comprises capsid-free, non-viral DNA vector and a cationic lipid or a salt thereof.

In one embodiment, the lipid particle (e.g., lipid nanoparticle) comprises a cationic lipid / non-cationic-lipid / sterol / conjugated lipid at a molar ratio of 50:10:38.5:1.5.

In one embodiment, the disclosure provides for a lipid particle (e.g., lipid nanoparticle) formulation comprising phospholipids, lecithin, phosphatidylcholine and phosphatidylethanolamine.

### III. Rigid Therapeutic Nucleic Acid

Aspects of the present disclosure generally provide lipid particles (e.g., lipid nanoparticles) comprising a rigid therapeutic nucleic acid (TNA) like closed ended DNA (ceDNA)and a lipid.

### Closed-Ended DNA (ceDNA) Vectors

Embodiments of the disclosure are based on methods and compositions comprising closed-ended linear duplexed (ceDNA) vectors that can express a transgene (e.g. a therapeutic nucleic acid). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

ceDNA vectors preferably have a linear and continuous structure rather than a non-continuous structure. The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, it is likely to remain a single molecule. In some embodiments, ceDNA vectors can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

Provided herein are non-viral, capsid-free ceDNA molecules with covalently closed ends (ceDNA). These non-viral capsid free ceDNA molecules can be produced in permissive host cells from an expression construct (e.g., a ceDNA-plasmid, a ceDNA-bacmid, a ceDNA- baculovirus, or an integrated cell-line) containing a heterologous gene (e.g., a transgene, in particular a therapeutic transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g., self-complementary, over at least a portion of the molecule, such as the expression cassette (e.g. ceDNA is not a double stranded circular molecule). The ceDNA vector has covalently closed ends, and thus is resistant to exonuclease digestion (e.g. exonuclease I or exonuclease III), e.g. for over an hour at 37° C.

In one aspect, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR. In one embodiment, the first ITR (5′ ITR) and the second ITR (3′ ITR) are asymmetric with respect to each other - that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild- type ITR. In one embodiment, the first ITR and the second ITR are both modified but are different sequences, or have different modifications, or are not identical modified ITRs, and have different 3D spatial configurations. Stated differently, a ceDNA vector with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other.

In one embodiment, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other - that is, a ceDNA vector can comprise ITR sequences that have a symmetrical three-dimensional spatial organization such that their structure is the same shape in geometrical space, or have the same A, C—C′ and B—B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In one embodiment, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs can be are wild type (WT-ITRs) as described herein. That is, both ITRs have a wild type sequence, but do not necessarily have to be WT-ITRs from the same AAV serotype. In one embodiment, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The wild-type or mutated or otherwise modified ITR sequences provided herein represent DNA sequences included in the expression construct (e.g., ceDNA-plasmid, ceDNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In one embodiment, a ceDNA vector described herein comprising the expression cassette with a transgene which is a therapeutic nucleic acid sequence, can be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other, or symmetrical relative to each other.

In one embodiment, an expression cassette is located between two ITRs comprised in the following order with one or more of: a promoter operably linked to a transgene, a posttranscriptional regulatory element, and a polyadenylation and termination signal. In one embodiment, the promoter is regulatable - inducible or repressible. The promoter can be any sequence that facilitates the transcription of the transgene. In one embodiment the promoter is a CAG promoter, or variation thereof. The posttranscriptional regulatory element is a sequence that modulates expression of the transgene, as a non-limiting example, any sequence that creates a tertiary structure that enhances expression of the transgene which is a therapeutic nucleic acid sequence.

In one embodiment, the posttranscriptional regulatory element comprises WPRE. In one embodiment, the polyadenylation and termination signal comprise BGHpolyA. Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

In one embodiment, the expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene which is a therapeutic nucleic acid sequence in the range of 500 to 50,000 nucleotides in length. In one embodiment, the expression cassette can comprise a transgene which is a therapeutic nucleic acid sequence in the range of 500 to 75,000 nucleotides in length. In one embodiment, the expression cassette can comprise a transgene which is a therapeutic nucleic acid sequence in the range of 500 to 10,000 nucleotides in length. In one embodiment, the expression cassette can comprise a transgene which is a therapeutic nucleic acid sequence in the range of 1000 to 10,000 nucleotides in length. In one embodiment, the expression cassette can comprise a transgene which is a therapeutic nucleic acid sequence in the range of 500 to 5,000 nucleotides in length. The ceDNA vectors do not have the size limitations of encapsidated AAV vectors, and thus enable delivery of a large-size expression cassette to the host. In one embodiment, the ceDNA vector is devoid of prokaryote-specific methylation.

In one embodiment, the rigid therapeutic nucleic acid can be a plasmid.

In one embodiment, the ceDNA vectors disclosed herein are used for therapeutic purposes (e.g., for medical, diagnostic, or veterinary uses) or immunogenic polypeptides.

The expression cassette can comprise any transgene which is a therapeutic nucleic acid sequence. In certain embodiments, the ceDNA vector comprises any gene of interest in the subject, which includes one or more polypeptides, peptides, ribozymes, peptide nucleic acids, siRNAs, RNAis, antisense oligonucleotides, antisense polynucleotides, antibodies, antigen binding fragments, or any combination thereof.

In one embodiment, the ceDNA expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. In one embodiment, the exogenous sequence such as a donor sequence can encode a gene product that can function to correct the expression of a defective gene or transcript. In one embodiment, the expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). In one embodiment, expression cassettes can include an exogenous sequence that encodes a reporter protein to be used for experimental or diagnostic purposes, such as b-lactamase, b -galactosidase (LacZ), alkaline phosphatase, thymidine kinase, green fluorescent protein (GFP), chloramphenicol acetyltransferase (CAT), luciferase, and others well known in the art.

Accordingly, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease.

### Therapeutic Nucleic Acids

Illustrative therapeutic nucleic acids of the present disclosure can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, CELiD, linear covalently closed DNA (“ministring”), doggybone™, protelomere closed ended DNA, or dumbbell linear DNA), dicer-substrate dsRNA, small hairpin RNA (shRNA), asymmetrical interfering RNA (aiRNA), microRNA (miRNA), mRNA, tRNA, rRNA, and DNA viral vectors, viral RNA vector, and any combination thereof.

siRNA or miRNA that can downregulate the intracellular levels of specific proteins through a process called RNA interference (RNAi) are also contemplated by the present disclosure to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson - capable of binding to the mRNA by Crick base pairing. This binding prevents translation of a target mRNA, and / or triggers RNaseH degradation of the mRNA transcript. As a result, the antisense oligonucleotide has increased specificity of action (i.e., down-regulation of a specific disease-related protein).

In any of the methods provided herein, the therapeutic nucleic acid can be a therapeutic RNA. Said therapeutic RNA can be an inhibitor of mRNA translation, agent of RNA interference (RNAi), catalytically active RNA molecule (ribozyme), transfer RNA (tRNA) or an RNA that binds an mRNA transcript (ASO), protein or other molecular ligand (aptamer). In any of the methods provided herein, the agent of RNAi can be a double-stranded RNA, single-stranded RNA, micro RNA, short interfering RNA, short hairpin RNA, or a triplex-forming oligonucleotide.

According to some embodiments, formulation process and methods described by the present disclosure can encapsulate considerably more double stranded DNA (e.g., ceDNA) than has been previously reported. According to some embodiments, the LNPs describedherein can encapsulate greater than about 60% of rigid double stranded DNA, like ceDNA, greater than about 65% of rigid double stranded DNA, like ceDNA, greater than about 70% of rigid double stranded DNA, like ceDNA, greater than about 75% of rigid double stranded DNA, like ceDNA, greater than about 80% of rigid double stranded DNA, like ceDNA,n greater than about 85% of rigid double stranded DNA, like ceDNA, or greater than about 90% of rigid double stranded DNA, like ceDNA.

### IV. Denatured Therapeutic Nucleic Acid

Aspects of the present disclosure further provide pharmaceutical compositions comprising lipid particles (e.g., lipid nanoparticles) and a denatured therapeutic nucleic acid (TNA), where TNA is as defined above. In one embodiment, the denature TNA is a closed ended DNA (ceDNA). The term “denatured therapeutic nucleic acid” refers to a partially or fully TNA where the conformation has changed from the standard B-form structure. The conformational changes may include changes in the secondary structure (i.e., base pair interactions within a single nucleic acid molecule) and/or changes in the tertiary structure (i.e., double helix structure). Without being bound by theory, the inventors believed that TNA treated with an alcohol/water solution or pure alcohol solvent results in the denaturation of the nucleic acid to a conformation that enhances encapsulation efficiency by LNP and produces LNP formulations having a smaller diameter size (i.e., smaller than 75 nm, for example, the mean size of about 68 to 74 nm in diameter). All LNP mean diameter sizes and size ranges described herein apply to LNPs containing a denatured TNA.

When DNA is in an aqueous environment, it has a B-form structure with 10.4 base pairs in each complete helical turn. If this aqueous environment is gradually changed by adding a moderately less polar alcohol such as methanol, the twist of the helix relaxes, whereby the DNA changes smoothly into a form with only 10.2 base pairs per helical turn, as visualized by circular dichroism (CD) spectroscopy. In one embodiment, the denatured TNA in a pharmaceutical composition provided herein has a 10.2-form structure.

In contrast to this behavior, if the water is replaced with a slightly less polar alcohol such as ethanol, the same kind of conformational change will occur only until about 65% of the water is replaced with ethanol. At this point, the DNA abruptly changes to the A-form structure which has a more tightly-twisted helix containing 11 base pairs per helical turn, as visualized by CD. In one embodiment, the denatured TNA in a pharmaceutical composition provided herein has an A-form structure.

According to some embodiments, the denatured TNA in a pharmaceutical composition provided herein has a rod-like structure when visualized under transmission electron microscopy (TEM). According to some embodiments, the denatured TNA in a pharmaceutical composition provided herein has a circular-like structure when visualized under transmission electron microscopy (TEM). Comparatively, TNA that has not been denatured has a strand-like structure.

According to some embodiments, the denatured TNA in a pharmaceutical composition provided herein has a P-form structure that has little or no hydrogen bonding, devoide of base stacking, and has a condensed tertiary structure.

### V. Production of a ceDNA Vector

Embodiments of the disclosure are based on methods and compositions comprising closed-ended linear duplexed (ceDNA) vectors that can express a transgene (e.g. TNA). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

Methods for the production of a ceDNA vector as described herein comprising an asymmetrical ITR pair or symmetrical ITR pair as defined herein is described in section IV of PCT/US 18/49996 filed Sep. 7, 2018, which is incorporated herein in its entirety by reference. As described herein, the ceDNA vector can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell.

The following is provided as a non-limiting example.

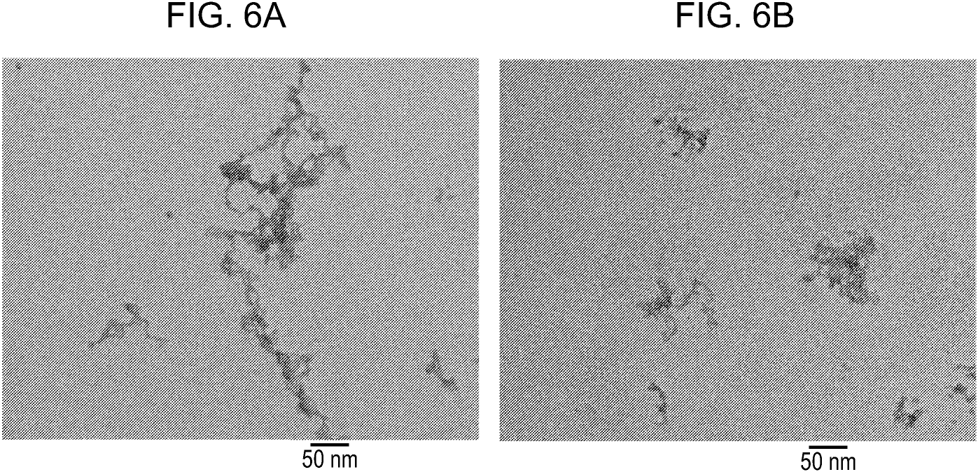

According to some embodiments, synthetic ceDNA is produced via excision from a double-stranded DNA molecule. Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIGS. 6 in International patent application PCT/US2018/064242, filed Dec. 6, 2018).

In some embodiments, a construct to make a ceDNA vector comprises additional components to regulate expression of the transgene, for example, regulatory switches, to regulate the expression of the transgene, or a kill switch, which can kill a cell comprising the vector.

A molecular regulatory switch is one which generates a measurable change in state in response to a signal. Such regulatory switches can be usefully combined with the ceDNA vectors described herein to control the output of expression of the transgene. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA vector in a controllable and regulatable fashion. In some embodiments, the switch can include a “kill switch” that can instruct the cell comprising the synthetic ceDNA vector to undergo cell programmed death once the switch is activated. Exemplary regulatory switches encompassed for use in a ceDNA vector can be used to regulate the expression of a transgene, and are more fully discussed in International application PCT/US18/49996, which is incorporated herein in its entirety by reference and described herein.

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122, incorporated by reference in its entirety herein, shows an exemplary method of ligating a 5′ ITR oligonucleotide and a 3′ ITR oligonucleotide to a double stranded polynucleotide comprising an expression cassette.

An exemplary method of producing a ceDNA vector using a synthetic method is provided in Example 4 of PCT/US19/14122, incorporated by reference in its entirety herein, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

In yet another aspect, the disclosure provides for host cell lines that have stably integrated the DNA vector polynucleotide expression template (ceDNA template) described herein, into their own genome for use in production of the non-viral DNA vector. Methods for producing such cell lines are described in Lee, L. et al. (2013) Plos One 8(8): e69879, which is herein incorporated by reference in its entirety. For example, the Rep protein is added to host cells at an MOI of 3. In one embodiment, the host cell line is an invertebrate cell line, preferably insect Sf9 cells. When the host cell line is a mammalian cell line, preferably 293 cells the cell lines can have polynucleotide vector template stably integrated, and a second vector, such as herpes virus can be used to introduce Rep protein into cells, allowing for the excision and amplification of ceDNA in the presence of Rep.

Any promoter can be operably linked to the heterologous nucleic acid (e.g. reporter nucleic acid or therapeutic transgene) of the vector polynucleotide. The expression cassette can contain a synthetic regulatory element, such as CAG promoter. The CAG promoter comprises (i) the cytomegalovirus (CMV) early enhancer element, (ii) the promoter, the first exon and the first intron of the chicken beta actin gene, and (ii) the splice acceptor of the rabbit beta globin gene. Alternatively, expression cassette can contain an Alpha-1-antitrypsin (AAT) promoter, a liver specific (LP1) promoter, or Human elongation factor-1 alpha (EF1-α) promoter. In some embodiments, the expression cassette includes one or more constitutive promoters, for example, the retroviral Rous sarcoma virus (RSV) LTR promoter (optionally with the RSV enhancer), cytomegalovirus (CMV) immediate early promoter (optionally with the CMV enhancer). Alternatively, an inducible or repressible promoter, a native promoter for a transgene, a tissue-specific promoter, or various promoters known in the art can be used. Suitable transgenes for gene therapy are well known to those of skill in the art.

The capsid-free ceDNA vectors can also be produced from vector polynucleotide expression constructs that further comprise cis-regulatory elements, or combination of cis regulatory elements, a non-limiting example include a woodchuck hepatitis virus posttranscriptional regulatory element (WPRE) and BGH polyA, or e.g. beta-globin polyA. Other posttranscriptional processing elements include, e.g. the thymidine kinase gene of herpes simplex virus, or hepatitis B virus (HBV). The expression cassettes can include any poly-adenylation sequence known in the art or a variation thereof, such as a naturally occurring isolated from bovine BGHpA or a virus SV40pA, or synthetic. Some expression cassettes can also include SV40 late polyA signal upstream enhancer (USE) sequence. The, USE can be used in combination with SV40pA or heterologous poly-A signal.

The time for harvesting and collecting DNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce DNA-vectors) but before thea majority of cells start to die because of the viral toxicity. The DNA-vectors can be isolated using plasmid purification kits such as Qiagen Endo-Free Plasmid kits. Other methods developed for plasmid isolation can be also adapted for DNA-vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

In one embodiment, the capsid free non-viral DNA vector comprises or is obtained from a plasmid comprising a polynucleotide template comprising in this order: a first adeno-associated virus (AAV) inverted terminal repeat (ITR), a nucleotide sequence of interest (for example an expression cassette of an exogenous DNA) and a modified AAV ITR, wherein said template nucleic acid molecule is devoid of AAV capsid protein coding. In a further embodiment, the nucleic acid template of the disclosure is devoid of viral capsid protein coding sequences (i.e. it is devoid of AAV capsid genes but also of capsid genes of other viruses). In addition, in a particular embodiment, the template nucleic acid molecule is also devoid of AAV Rep protein coding sequences. Accordingly, in a preferred embodiment, the nucleic acid molecule of the disclosure is devoid of both functional AAV cap and AAV rep genes.

In one embodiment, ceDNA can include an ITR structure that is mutated with respect to the wild type AAV2 ITR disclosed herein, but still retains an operable RBE, TRS and RBE′ portion.

### ceDNA Plasmid

A ceDNA-plasmid is a plasmid used for later production of a ceDNA vector. In one embodiment, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one embodiment, a ceDNA vector is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In one embodiment, the ceDNA-plasmid system is devoid of viral capsid protein coding sequences (i.e. it is devoid of AAV capsid genes but also of capsid genes of other viruses). In one embodiment, the ceDNA-plasmid is also devoid of AAV Rep protein coding sequences. In one embodiment, ceDNA-plasmid is devoid of functional AAV cap and AAV rep genes GG-3′ for AAV2) plus a variable palindromic sequence allowing for hairpin formation. In one embodiment, a ceDNA-plasmid of the present disclosure can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV 10, AAV 11, AAV 12, AAVrh8, AAVrhlO, AAV-DJ, and AAV-DJ8 genome, e.g., NCBI: NC 002077; NC 001401; NC001729; NC001829; NC006152; NC 006260; NC 006261; Kotin and Smith, The Springer Index of Viruses, available at the URL maintained by Springer. In one embodiment, the ceDNA-plasmid backbone is derived from the AAV2 genome. In one embodiment, the ceDNA-plasmid backbone is a synthetic backbone genetically engineered to include at its 5′ and 3′ ITRs derived from one of these AAV genomes.

In one embodiment, a ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S— resistance gene, kanamycin, geneticin, and the like.

### VI. Preparation of Lipid Particles

Lipid particles (e.g., lipid nanoparticles) can form spontaneously upon mixing of ceDNA and the lipid(s). Depending on the desired particle size distribution, the resultant nanoparticle mixture can be extruded through a membrane (e.g., 100 nrn cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. In one embodiment, the lipid nanoparticles are formed as described in Example 3 herein.

Generally, lipid particles (e.g., lipid nanoparticles) can be formed by any method known in the art. For example, the lipid particles (e.g., lipid nanoparticles) can be prepared by the methods described, for example, in US2013/0037977, US2010/0015218, US2013/0156845, US2013/0164400, US2012/0225129, and US2010/0130588, content of each of which is incorporated herein by reference in its entirety. In some embodiments, lipid particles (e.g., lipid nanoparticles) can be prepared using a continuous mixing method, a direct dilution process, or an in-line dilution process. The processes and apparatuses for apparatuses for preparing lipid nanoparticles using direct dilution and in-line dilution processes are described in US2007/0042031, the content of which is incorporated herein by reference in its entirety. The processes and apparatuses for preparing lipid nanoparticles using step-wise dilution processes are described in US2004/0142025, the content of which is incorporated herein by reference in its entirety.

According to some embodiments, the disclosure provides for an LNP comprising a rigid DNA vector, including a ceDNA vector as described herein and an ionizable lipid. For example, a lipid nanoparticle formulation that is made and loaded with rigid therapeutic nucleic acid like ceDNA obtained by the process as disclosed in International Patent Application No. PCT/US2018/050042, filed on Sep. 7, 2018, which is incorporated by reference in its entirety herein. The present disclosure involves a process of precompacting rigid therapeutic nucleic acid (TNA) like ceDNA in 80% to 100% a low molecular weight alcohol solution (e.g., ethanol, methanol, propanol, and isopropanol) prior to mixing the TNA with a lipid. Then, loading and encapsulating precompacted therapeutic nucleic acid can be accomplished by conventional high energy mixing by using a microfluidic device like Nanoassemblr™ of ethanolic lipids with the aqueous ceDNA (e.g., 80- 100% ethanol, methanol, propanol, isopropanol, or a mixture thereof) at low pH which protonates the ionizable lipid and provides favorable energetics for ceDNA/lipid association and nucleation of particles. The particles can be further stabilized through aqueous dilution and removal of the organic solvent. The particles can be concentrated to the desired level. Without wishing to be bound by theory, it is believed that the precompaction step of rigid DNA before mixing with lipids for encasultion of DNA provides beneficial effects on reducting the size of resultant LNPs by compacting the DNA molecule in low molecular weight alcohol solution prior to encapsulation.

According to some embodiments, the disclosure provides a method of producing a LNP formulation, wherein the LNP comprises a cationic lipid and TNA like a ceDNA, the method comprising: adding aqueous TNA (e.g., ceDNA) to a low molecular weight alcohol such as ethanol solution, wherein the concentration of the alcohol in the solution is between about 80% to about 95% and the concentration of water in the solution is between about 20% to about 5%; mixing the TNA (e.g., ceDNA) with lipid solution (e.g., 80% to 100% EtOH); and an acidic aqueous buffer (e.g., malic acid); and optionally buffer exchanging with a neutral-pH aqueous buffer, thereby producing an LNP formulation.

According to some embodiments, the concentration of a low molecular weight alcohol (e.g., ethanol, methanol, propanol, or isopropanol) in the solution is between about 80% to about 95%, between about 80% to about 90%, between about 80% to about 85%, between about 85% to about 95%, between about 85% to about 90%, between about 90% to about 95%, or about 80%, about 81%, about 82%, about 83%, about 84%, about 85%, about 86%, about 87%, about 88%, about 89%, about 90%, about 91%, about 92%, about 93%, about 94% or about 95%.

According to some embodiments, the concentration of water in the solution is between about 20% to about 5%, between about 15% to about 5%, between about 10% to about 5%, between about 20% to about 10%, between about 20% to about 15%, between about 15% to about 5%, between about 15% to about 10%, or about 20%, about 19%, about 18%, about 17%, about 164%, about 15%, about 14%, about 13%, about 12%, about 11%, about 10%, about 9%, about 8%, about 7%, about 6% or about 5%.

According to some embodiments, the low molecular weight alcohol is selected from the group consisting of ethanol, methanol, propanol, and isopropanol. In some embodiments, aqueous TNA (e.g., ceDNA) is in a solution comprising a mixture of two or three low molecular weight alcohol. In one embodiment, the low molecular weight alcoholic solution is a mixture of ethanol and methanol. In another embodiment, the low molecular weight alcoholic solution is a mixture of any combination of ethanol, methanol, propanol, and isopropanol. In another embodiment, the low molecular weight alcoholic solution is a mixture of ethanol and propanol. In another embodiment, the low molecular weight alcoholic solution comprises 45% ethanol, 45% methanol and 10% wather.

According to some embodiments, the method further comprises a step of diluting the mixed ceDNA/lipid solution with an acidic aqueous buffer. According to some emboidments, the acid aqueous buffer is selected from malic acid/sodium malate or acetic acid/sodium acetate. According to some emboidments, the acidic aqueous buffer is at a concentration of between about 10 to 40 millimolar (mM), between about 10 mM to about 35 mM, between about 10 mM to about 30 mM, between about 10 mM to about 25 mM, between about 10 mM to about 20 mM, between about 10 mM to about 15 mM, between about 15 to 40 mM, between about 15 mM to about 35 mM, between about 15 mM to about 30 mM, between about 15 mM to about 25 mM, between about 15 mM to about 20 mM, between about 20 to 40 mM, between about 20 mM to about 35 mM, between about 210 mM to about 30 mM, between about 20 mM to about 25 mM, between about 25 to 40 mM, between about 25 mM to about 35 mM, between about 25 mM to about 30 mM, between about 310 to 40 mM, between about 30 mM to about 35 mM, between about 35 mM to about 40 mM, or about, between about 10 mM to about 25 mM, between about 10 mM to about 20 mM, between about 10 mM to about 15 mM, or about 10 mM, about 12 mM, about 14 mM, about 16 mM, about 18 mM, about 20 mM, about 22 mM, about 24 mM, about 26 mM, about 28 mM, about 30 mM, about 32 mM, about 34 mM, about 36 mM, about 38 mM, or about 40 mM.

According to some embodiments, the acidic aqueous buffer is at a pH of between about 3 to about 5, between about 3 to about 4.5, between about 3 to about 4, between about 3 to about 3.5, between about 3.5 to about 5, between about 3.5 to about 4.5, between about 3.5 to about 4, between about 4 to about 5, between about 4 to about 4.5, between about 4.5 to about 5, or about 3, about 3.25, about 3.5, about 3.75, about 4, about 4.25, about 4.5, about 4.75, or about 5.

According to some embodiments, the neutral-pH aqueous buffer is Dulbecco’s phosphate buffered saline, pH 7.4.

According to some embodiments, the process of preparing LNPs that takes advantage of the discovery that rigid TNA like ceDNA compaction occurs in solvents with high alcohol (ethanol, methanol, propanol and/or isopropanol) content (>80%). According to some embodiments, the formulation process described herein produces LNPs that range in size from about 50 to about 70 nm. According to some embodiments, the lipid particles of the disclosure typically have a mean diameter of from about 20 nm to about 70 nm, about 25 nm to about 70 nm, from about 30 nm to about 70 nm, from about 35 nm to about 70 nm, from about 40 nm to about 70 nm, from about 45 nm to about 80 nm, from about 50 nm to about 70 nm, from about 60 nm to about 70 nm, from about 65 nm to about 70 nm, or about 20 nm, about 25 nm, about 30 nm, about 35 nm, about 40 nm, about 45 nm, about 50 nm, about 55 nm, about 60 nm, about 65 nm, about 70 nm. According to some embodiments, the formulation process described herein produces LNPs that encapsulate greater than about 80% of rigid TNA like double stranded ceDNA. According to some embodiments, the LNPs described herein can encapsulate greater than about 60% of rigid TNA like double stranded ceDNA, greater than about 65% of rigid TNA like double stranded ceDNA, greater than about 70% of rigid TNA like double stranded ceDNA,, greater than about 75% of rigid TNA like double stranded ceDNA, greater than about 80% of rigid TNA like double stranded ceDNA, greater than about 85% of rigid TNA like double stranded ceDNA, or greater than about 90% of rigid TNA like double stranded ceDNA.

According to some embodiments, when TNA like ceDNA in a compacted state in a low molecular weight alcohol is mixed with anethanolic solution of lipids (80%-100% EtOH) in a ratio such that the resulting solution is 85-95% ethanol and 15-5% water, the TNA like ceDNA is observed to exist in a compacted state by dynamic light scattering. In such a solvent, both the lipids and ceDNA are solubilized with no detectable precipitation of either component. The formulation of LNPs that results in encapsulation of the compacted TNA leads to much smaller size in diameter.

According to some embodiments, when aqueous TNA like ceDNA is mixed with an ethanolic solution of lipids in a ratio such that the resulting solution is 90-92% ethanol and 8-10% water in an acidic condition (malic acid), the TNA lik eceDNA is observed to exist in a compacted state by dynamic light scattering and resulting encapsulation leads to LNPs having much smaller size in diameter.

LNP formation is then driven by mixing of the ethanolic solution of ceDNA/lipids solution with acidic aqueous buffer using microfluidic mixing. According to some embodiments, the flow rate ratio between the acidic aqueous buffer and the ethanolic mixture of ceDNA/lipids can be 2:1, 3:2, 3:1, 4:1, 5:1, 6:1, 7:1, 8:1, 9:1, 10:1, 15:1 or 20:1. According to some embodiments, after exiting the mixer, the final solution is diluted with acid aqueous buffer such that the final ethanol content is about 4% to about 15%. According to some embodiments, after exiting the mixer, the final solution is diluted with acid aqueous buffer such that the final ethanol content is about 4%, about 5%, about 6%, about 7%, about 8%, about 9%, about 10%, about 11%, about 12%, about 13%, about 14% or about 15%. According to some embodiments, the final ethanol content is 4%. According to some embodiments, the final ethanol content is 12%. This solution containing the LNPs is then buffer-exchanged with neutral-pH aqueous buffer.

In one embodiment, the lipid particles (e.g., lipid nanoparticles) can be prepared by an impinging jet process. Generally, the particles are formed by mixing lipids dissolved in alcohol (e.g., ethanol) with ceDNA dissolved in a buffer, e.g, a citrate buffer, a sodium acetate buffer, a sodium acetate and magnesium chloride buffer, a malic acid buffer, a malic acid and sodium chloride buffer, or a sodium citrate and sodium chloride buffer. The mixing ratio of lipids to ceDNA can be about 45-55% lipid and about 65-45% ceDNA.

The lipid solution can contain a cationic lipid (e.g. an ionizable cationic lipid), a non-cationic lipid (e.g., a phospholipid, such as DSPC, DOPE, and DOPC), PEG or PEG conjugated molecule (e.g., PEG-lipid), and a sterol (e.g., cholesterol) at a total lipid concentration of 5-30 mg/mL, more likely 5-15 mg/mL, most likely 9-12 mg/mL in an alcohol, e.g., in ethanol. In the lipid solution, mol ratio of the lipids can range from about 25-98% for the cationic lipid, preferably about 35-65%; about 0-15% for the non-ionic lipid, preferably about 0-12%; about 0-15% for the PEG or PEG conjugated lipid molecule, preferably about 1-6%; and about 0-75% for the sterol, preferably about 30-50%.

The ceDNA solution can comprise the ceDNA at a concentration range from 0.3 to 1.0 mg/mL, preferably 0.3-0.9 mg/mL in buffered solution, with pH in the range of 3.5-5.

For forming the LNPs, in one exemplary but nonlimiting embodiment, the two liquids are heated to a temperature in the range of about 15-40° C., preferably about 30-40° C., and then mixed, for example, in an impinging jet mixer, instantly forming the LNP. The mixing flow rate can range from 10-600 mL/min. The tube ID can have a range from 0.25 to 1.0 mm and a total flow rate from 10-600 mL/min. The combination of flow rate and tubing ID can have the effect of controlling the particle size of the LNPs between 30 and 200 nm. The solution can then be mixed with a buffered solution at a higher pH with a mixing ratio in the range of 1:1 to 1:3 vol:vol, preferably about 1:2 vol:vol. If needed this buffered solution can be at a temperature in the range of 15-40° C. or 30-40° C. The mixed LNPs can then undergo an anion exchange filtration step. Prior to the anion exchange, the mixed LNPs can be incubated for a period of time, for example 30mins to 2 hours. The temperature during incubating can be in the range of 15-40° C. or 30-40° C. After incubating the solution is filtered through a filter, such as a 0.8 µm filter, containing an anion exchange separation step. This process can use tubing IDs ranging from 1 mm ID to 5 mm ID and a flow rate from 10 to 2000 mL/min.

After formation, the LNPs can be concentrated and diafiltered via an ultrafiltration process where the alcohol is removed and the buffer is exchanged for the final buffer solution, for example, phosphate buffered saline (PBS) at about pH 7, e.g., about pH 6.9, about pH 7.0, about pH 7.1, about pH 7.2, about pH 7.3, or about pH 7.4.

The ultrafiltration process can use a tangential flow filtration format (TFF) using a membrane nominal molecular weight cutoff range from 30-500 kD. The membrane format is hollow fiber or flat sheet cassette. The TFF processes with the proper molecular weight cutoff can retain the LNP in the retentate and the filtrate or permeate contains the alcohol; citrate buffer and final buffer wastes. The TFF process is a multiple step process with an initial concentration to a ceDNA concentration of 1-3 mg/mL. Following concentration, the LNPs solution is diafiltered against the final buffer for 10-20 volumes to remove the alcohol and perform buffer exchange. The material can then be concentrated an additional 1-3-fold. The concentrated LNP solution can be sterile filtered.

### VII. Pharmaceutical Compositions and Formulations

Also provided herein is a pharmaceutical composition comprising the TNA like ceDNA lipid particle and a pharmaceutically acceptable carrier or excipient.

In one embodiment, the TNA (e.g, ceDNA) lipid particles (e.g., lipid nanoparticles) are provided with full encapsulation, partial encapsulation of the therapeutic nucleic acid. In one embodiment, the nucleic acid therapeutics is fully encapsulated in the lipid particles (e.g., lipid nanoparticles) to form a nucleic acid containing lipid particle. In one embodiment, the nucleic acid may be encapsulated within the lipid portion of the particle, thereby protecting it from enzymatic degradation.

Depending on the intended use of the lipid particles (e.g., lipid nanoparticles), the proportions of the components can be varied and the delivery efficiency of a particular formulation can be measured using, for example, an endosomal release parameter (ERP) assay.

In one embodiment, the lipid particles (e.g., lipid nanoparticles) may be conjugated with other moieties to prevent aggregation. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

In one embodiment, the TNA (e.g., ceDNA) can be complexed with the lipid portion of the particle or encapsulated in the lipid position of the lipid particle (e.g., lipid nanoparticle). In one embodiment, the TNA can be fully encapsulated in the lipid position of the lipid particle (e.g., lipid nanoparticle), thereby protecting it from degradation by a nuclease, e.g., in an aqueous solution. In one embodiment, the TNA in the lipid particle (e.g., lipid nanoparticle) is not substantially degraded after exposure of the lipid particle (e.g., lipid nanoparticle) to a nuclease at 37° C. for at least about 20, 30, 45, or 60 minutes. In some embodiments, the TNA in the lipid particle (e.g., lipid nanoparticle) is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In one embodiment, the lipid particles (e.g., lipid nanoparticles) are substantially non-toxic to a subject, e.g., to a mammal such as a human.

In one embodiment, a pharmaceutical composition comprising a therapeutic nucleic acid of the present disclosure may be formulated in lipid particles (e.g., lipid nanoparticles). In some embodiments, the lipid particle comprising a therapeutic nucleic acid can be formed from a cationic lipid. In some other embodiments, the lipid particle comprising a therapeutic nucleic acid can be formed from non-cationic lipid. In a preferred embodiment, the lipid particle of the disclosure is a nucleic acid containing lipid particle, which is formed from a cationic lipid comprising a therapeutic nucleic acid selected from the group consisting of mRNA, antisense RNA and oligonucleotide, ribozymes, aptamer, interfering RNAs (RNAi), Dicer-substrate dsRNA, small hairpin RNA (shRNA), asymmetrical interfering RNA (aiRNA), microRNA (miRNA), minicircle DNA, minigene, viral DNA (e.g., Lentiviral or AAV genome) or non-viral synthetic DNA vectors, closed-ended linear duplex DNA (ceDNA / CELiD), plasmids, bacmids, doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

In another preferred embodiment, the lipid particle of the disclosure is a nucleic acid containing lipid particle, which is formed from a non-cationic lipid, and optionally a conjugated lipid that prevents aggregation of the particle.

In one embodiment, the lipid particle formulation is an aqueous solution. In one embodiment, the lipid particle (e.g., lipid nanoparticle) formulation is a lyophilized powder.

According to some aspects, the disclosure provides for a lipid particle formulation further comprising one or more pharmaceutical excipients. In one embodiment, the lipid particle (e.g., lipid nanoparticle) formulation further comprises sucrose, tris, trehalose and/or glycine.

In one embodiment, the lipid particles (e.g., lipid nanoparticles) disclosed herein can be incorporated into pharmaceutical compositions suitable for administration to a subject for in vivo delivery to cells, tissues, or organs of the subject. Typically, the pharmaceutical composition comprises the TNA (e.g., ceDNA) lipid particles (e.g., lipid nanoparticles) disclosed herein and a pharmaceutically acceptable carrier. In one embodiment, the TNA (e.g., ceDNA) lipid particles (e.g., lipid nanoparticles) of the disclosure can be incorporated into a pharmaceutical composition suitable for a desired route of therapeutic administration (e.g., parenteral administration). Passive tissue transduction via high pressure intravenous or intraarterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable for high TNA (e.g., ceDNA) vector concentration. Sterile injectable solutions can be prepared by incorporating the TNA (e.g., ceDNA) vector compound in the required amount in an appropriate buffer with one or a combination of ingredients enumerated above, as required, followed by filtered sterilization.

A lipid particle as disclosed herein can be incorporated into a pharmaceutical composition suitable for topical, systemic, intra-amniotic, intrathecal, intracranial, intraarterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutically active compositions comprising TNA (e.g., ceDNA) lipid particles (e.g., lipid nanoparticles) can be formulated to deliver a transgene in the nucleic acid to the cells of a recipient, resulting in the therapeutic expression of the transgene therein. The composition can also include a pharmaceutically acceptable carrier.

Pharmaceutical compositions for therapeutic purposes typically must be sterile and stable under the conditions of manufacture and storage. The composition can be formulated as a solution, microemulsion, dispersion, liposomes, or other ordered structure suitable to high TNA (e.g., ceDNA) vector concentration. Sterile injectable solutions can be prepared by incorporating the ceDNA vector compound in the required amount in an appropriate buffer with one or a combination of ingredients enumerated above, as required, followed by filtered sterilization.

In one embodiment, lipid particles (e.g., lipid nanoparticles) are solid core particles that possess at least one lipid bilayer. In one embodiment, the lipid particles (e.g., lipid nanoparticles) have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the lipid particles (e.g., lipid nanoparticles) can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid particles (lamellar vs. non-lamellar) can readily be assessed and characterized using, e.g., Cryo-TEM analysis as described in US2010/0130588, the content of which is incorporated herein by reference in its entirety.

In one embodiment, the lipid particles (e.g., lipid nanoparticles) having a non-lamellar morphology are electron dense.

In one embodiment, the disclosure provides for a lipid particle (e.g., lipid nanoparticle) that is either unilamellar or multilamellar in structure. In some aspects, the disclosure provides for a lipid particle (e.g., lipid nanoparticle) formulation that comprises multi-vesicular particles and/or foam-based particles. By controlling the composition and concentration of the lipid components, one can control the rate at which the lipid conjugate exchanges out of the lipid particle and, in turn, the rate at which the lipid particle (e.g., lipid nanoparticle) becomes fusogenic. In addition, other variables including, for example, pH, temperature, or ionic strength, can be used to vary and/or control the rate at which the lipid particle (e.g., lipid nanoparticle) becomes fusogenic. Other methods which can be used to control the rate at which the lipid particle (e.g., lipid nanoparticle) becomes fusogenic will be apparent to those of ordinary skill in the art based on this disclosure. It will also be apparent that by controlling the composition and concentration of the lipid conjugate, one can control the lipid particle size.

In one embodiment, the pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which are incorporated by reference in their entireties). In one embodiment, the preferred range of pKa is ~5 to ~ 7. In one embodiment, the pKa of the cationic lipid can be determined in lipid particles (e.g., lipid nanoparticles) using an assay based on fluorescence of 2- (p-toluidino)-6-napthalene sulfonic acid (TNS).

In one embodiment, encapsulation of TNA (e.g., ceDNA) in lipid particles (e.g., lipid nanoparticles) can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated TNA (e.g., ceDNA), allowing it to interact with the membrane-impermeable dye. Encapsulation of ceDNA can be calculated as E═ (Io — I)/Io, where I and Io refer to the fluorescence intensities before and after the addition of detergent.

### Unit Dosage

In one embodiment, the pharmaceutical compositions can be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### VIII. Methods of Treatment

The TNA (e.g., ceDNA vector lipid particles) as described herein can be used to introduce a nucleic acid sequence (e.g., a therapeutic nucleic acid sequence) in a host cell. In one embodiment, introduction of a nucleic acid sequence in a host cell using the TNA (e.g., ceDNA vectors) lipid particles can be monitored with appropriate biomarkers from treated patients to assess gene expression.

The pharmaceutical compositions provided herein can be used to deliver a transgene (a nucleic acid sequence) for various purposes. In one embodiment, the ceDNA vectors (e.g., ceDNA vector lipid nanoparticles) can be used in a variety of ways, including, for example, ex situ, in vitro and in vivo applications, methodologies, diagnostic procedures, and/or gene therapy regimens.

Provided herein are methods of treating a disease or disorder in a subject comprising introducing into a target cell in need thereof (for example, a muscle cell or tissue, or other affected cell type) of the subject a therapeutically effective amount of a TNA (e.g., ceDNA) lipid nanoparticles as described herein, optionally with a pharmaceutically acceptable carrier. While the TNA lipid nanoparticles can be introduced in the presence of a carrier, such a carrier is not required. The TNA (e.g., ceDNA) lipid nanoparticlesimplemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The TNA (e.g., ceDNA) lipid nanoparticles can be administered via any suitable route as described herein and known in the art. In one embodiment, the target cells are in a human subject.

Provided herein are methods for providing a subject in need thereof with a diagnostically- or therapeutically-effective amount of a ceDNA vector (e.g., ceDNA vector lipid particles (e.g., lipid nanoparticles) as described herein), the method comprising providing to a cell, tissue or organ of a subject in need thereof, an amount of the ceDNA vector (e.g., ceDNA vector lipid particles (e.g., lipid nanoparticles) as described herein); and for a time effective to enable expression of the transgene from the ceDNA vector thereby providing the subject with a diagnostically- or a therapeutically- effective amount of the protein, peptide, nucleic acid expressed by the ceDNA vector (e.g., ceDNA vector lipid particles (e.g., lipid nanoparticles) as described herein). In one embodiment, the subject is human.

Provided herein are methods for diagnosing, preventing, treating, or ameliorating at least one or more symptoms of a disease, a disorder, a dysfunction, an injury, an abnormal condition, or trauma in a subject. Generally, the method includes at least the step of administering to a subject in need thereof one or more TNA (e.g., ceDNA) lipid nanoparticles as described herein, in an amount and for a time sufficient to diagnose, prevent, treat or ameliorate the one or more symptoms of the disease, disorder, dysfunction, injury, abnormal condition, or trauma in the subject. In one embodiment, the subject is human.

Provided herein are methods comprising using of the TNA (e.g., ceDNA) lipid nanoparticle as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, TNA such as ceDNA lipid nanoparticles can be used to deliver transgenes to bring a normal gene into affected tissues for replacement therapy, as well, in some embodiments, to create animal models for the disease using antisense mutations. For unbalanced disease states, TNA (e.g., ceDNA) lipid nanoparticle can be used to create a disease state in a model system, which could then be used in efforts to counteract the disease state. Thus, the TNA (e.g., ceDNA) lipid nanoparticles and methods disclosed herein permit the treatment of genetic diseases. As used herein, a disease state is treated by partially or wholly remedying the deficiency or imbalance that causes the disease or makes it more severe.

In general, the TNA such as ceDNA lipid nanoparticles as described herein can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer’s disease, Parkinson’s disease, Huntington’s disease, amyotrophic lateral sclerosis, epilepsy, and other neurological disorders, cancer, diabetes mellitus, muscular dystrophies (e.g., Duchenne, Becker), Hurler’s disease, adenosine deaminase deficiency, metabolic defects, retinal degenerative diseases (and other diseases of the eye), mitochondriopathies (e.g., Leber’s hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the ceDNA vectors as disclosed herein can be advantageously used in the treatment of individuals with metabolic disorders (e.g., ornithine transcarbamylase deficiency).

In one embodiment, the TNA such as ceDNA lipid nanoparticle described herein can be used to treat, ameliorate, and/or prevent a disease or disorder caused by mutation in a gene or gene product. Exemplary diseases or disorders that can be treated with ceDNA vectors (e.g., ceDNA vector lipid particles (e.g., lipid nanoparticles) as described herein include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

In one embodiment, TNA such as ceDNA lipid nanoparticles as described herein may be employed to deliver a heterologous nucleotide sequence in situations in which it is desirable to regulate the level of transgene expression (e.g., transgenes encoding hormones or growth factors, as described herein).

In one embodiment, the TNA such as ceDNA lipid nanoparticles can be used to correct an abnormal level and/or function of a gene product (e.g., an absence of, or a defect in, a protein) that results in the disease or disorder. The ceDNA vectors in lipid nanoparticles as described herein can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MFD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In one embodiment, the TNA (e.g., ceDNA) lipid nanoparticles as described herein can be used to provide an RNA-based therapeutic to a cell in vitro or in vivo. Examples of RNA-based therapeutics include, but are not limited to, mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). For example, in one embodiment, the ceDNA vectors (e.g., ceDNA vector lipid particles (e.g., lipid nanoparticles) as described herein) can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In one embodiment, the TNA lipid nanoparticles as described herein can be used to provide a DNA-based therapeutic to a cell in vitro or in vivo. Examples of DNA-based therapeutics include, but are not limited to, minicircle DNA, minigene, viral DNA (e.g., Lentiviral or AAV genome) or non-viral synthetic DNA vectors, closed-ended linear duplex DNA (ceDNA / CELiD), plasmids, bacmids, doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”). For example, in one embodiment, the ceDNA vectors (e.g., ceDNA vector lipid particles (e.g., lipid nanoparticles) as described herein) can be used to provide minicircle to a cell in vitro or in vivo. For example, where the transgene is a minicircle DNA, expression of the minicircle DNA in the target cell diminishes expression of a particular protein by the cell. Accordingly, transgenes which are minicircle DNAs may be administered to decrease expression of a particular protein in a subject in need thereof. Minicircle DNAs may also be administered to cells in vitro to regulate cell physiology, e.g., to optimize cell or tissue culture systems.

In one embodiment, exemplary transgenes encoded by the TNA such as ceDNA vector include, but are not limited to: lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, b-interferon, interferon-g, interleukin-2, interleukin-4, interleukin 12, granulocyte- macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-a and -b, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

### Administration

In one embodiment, TNA lipid nanoparticle of the present disclosure can be administered to an organism for transduction of cells in vivo. In one embodiment, the TNA can be administered to an organism for transduction of cells ex vivo.

Generally, administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Exemplary modes of administration of the TNA such as ceDNA vectors (e.g., ceDNA lipid nanoparticles) as described herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the TNA lipid particle as described herein can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. In one embodiment, administration of the TNA lipid nanoparticles as described herein can also be to a tumor (e.g., in or near a tumor or a lymph node). The most suitable route in any given case will depend on the nature and severity of the condition being treated, ameliorated, and/or prevented and on the nature of the particular ceDNA (e.g., ceDNA lipid nanoparticles) as described herein that is being used. Additionally, ceDNA permits one to administer more than one transgene in a single vector, or multiple ceDNA vectors (e.g., a ceDNA cocktail).

In one embodiment, administration of the ceDNA vectors (e.g., ceDNA vector lipid particles (e.g., lipid nanoparticles) as described herein) to skeletal muscle includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA vectors (e.g., ceDNA vector lipid particles (e.g., lipid nanoparticles) as described herein) can be delivered to skeletal muscle by intravenous administration, intra-arterial administration, intraperitoneal administration, limb perfusion, (optionally, isolated limb perfusion of a leg and/or arm; see, e.g., Arruda et al., (2005) Blood 105: 3458-3464), and/or direct intramuscular injection. In particular embodiments, the ceDNA vector (e.g., a ceDNA vector lipid particle as described herein) is administered to a limb (arm and/or leg) of a subject (e.g., a subject with muscular dystrophy such as DMD) by limb perfusion, optionally isolated limb perfusion (e.g., by intravenous or intra-articular administration. In one embodiment, the ceDNA vector (e.g., a ceDNA vector lipid particle as described herein) can be administered without employing “hydrodynamic” techniques.

Administration of the ceDNA vectors (e.g., a ceDNA vector lipid particles (e.g., lipid nanoparticles) as described herein) to cardiac muscle includes administration to the left atrium, right atrium, left ventricle, right ventricle and/or septum. The ceDNA vectors (e.g., ceDNA vector lipid particles (e.g., lipid nanoparticles) as described herein) can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In one embodiment, ceDNA vectors (e.g., ceDNA vector lipid particles (e.g., lipid nanoparticles) as described herein) are administered to skeletal muscle, diaphragm muscle and/or cardiac muscle (e.g., to treat, ameliorate, and/or prevent muscular dystrophy or heart disease (e.g., PAD or congestive heart failure).

ceDNA vectors (e.g., ceDNA vector lipid particles (e.g., lipid nanoparticles) as described herein) can be administered to the CNS (e.g., to the brain or to the eye). The ceDNA vectors (e.g., ceDNA vector lipid particles (e.g., lipid nanoparticles) as described herein) may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vectors (e.g., ceDNA vector lipid particles (e.g., lipid nanoparticles) as described herein) may also be administered to different regions of the eye such as the retina, cornea and/or optic nerve. The ceDNA vectors (e.g., ceDNA vector lipid particles (e.g., lipid nanoparticles) as described herein) may be delivered into the cerebrospinal fluid (e.g., by lumbar puncture). The ceDNA vectors (e.g., ceDNA vector lipid particles (e.g., lipid nanoparticles) as described herein) may further be administered intravascularly to the CNS in situations in which the blood-brain barrier has been perturbed (e.g., brain tumor or cerebral infarct).

In one embodiment, the ceDNA vectors (e.g., ceDNA vector lipid particles (e.g., lipid nanoparticles) as described herein) can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon’s region) delivery as well as intramuscular delivery with retrograde delivery to motor neurons.

According to some embodiment, the ceDNA vectors (e.g., ceDNA vector lipid particles (e.g., lipid nanoparticles) as described herein) is administered in a liquid formulation by direct injection (e.g., stereotactic injection) to the desired region or compartment in the CNS. According to other embodiments, the ceDNA vectors (e.g., ceDNA vector lipid particles (e.g., lipid nanoparticles) as described herein) can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898, incorporated by reference in its entirety herein). In one embodiment, the ceDNA vectors (e.g., ceDNA vector lipid particles (e.g., lipid nanoparticles) as described herein) can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vectors (e.g., ceDNA vector lipid particles (e.g., lipid nanoparticles) as described herein) can be delivered to muscle tissue from which it can migrate into neurons.

In one embodiment, repeat administrations of the therapeutic product can be made until the appropriate level of expression has been achieved. Thus, in one embodiment, a therapeutic nucleic acid can be administered and re-dosed multiple times. For example, the therapeutic nucleic acid can be administered on day 0. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the therapeutic nucleic acid.

In one embodiment, one or more additional compounds can also be included. Those compounds can be administered separately or the additional compounds can be included in the lipid particles (e.g., lipid nanoparticles) of the disclosure. In other words, the lipid particles (e.g., lipid nanoparticles) can contain other compounds in addition to the ceDNA or at least a second ceDNA, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In one embodiment, the one or more additional compound can be a therapeutic agent. The therapeutic agent can be selected from any class suitable for the therapeutic objective. Accordingly, the therapeutic agent can be selected from any class suitable for the therapeutic objective. The therapeutic agent can be selected according to the treatment objective and biological action desired. For example, in one embodiment, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody- drug conjugate). In one embodiment, if the LNP containing the ceDNA is useful for treating an infection, the additional compound can be an antimicrobial agent (e.g., an antibiotic or antiviral compound). In one embodiment, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In one embodiment, different cocktails of different lipid particles containing different compounds, such as a ceDNA encoding a different protein or a different compound, such as a therapeutic may be used in the compositions and methods of the disclosure. In one embodiment, the additional compound is an immune modulating agent. For example, the additional compound is an immunosuppressant. In some embodiments, the additional compound is immunostimulatory.

### EXAMPLES

The following examples are provided by way of illustration not limitation.

### Example 1: Synthesis of Ionizable Lipids of Formula I of Formula I′

Ionizable lipids of Formula (I) or Formula (I′) can be designed and synthesized using general synthesis methods described below. While the methods are exemplified with ionizable lipids, they are applicable to synthesis of cleavable lipids contemplated under Formula (I) or Formula (I′) .

### General Synthesis (e.g., R⁴ = -C)

Synthesis of the ionizable lipids of Formula (I) or (I′) described herein, as illustrated in Scheme 1, can start from a lipid acid (a) and coupling to N,O-dimethyl hydroxylamine gives a Weinreb amide (b). Grignard addition generates ketone (c). Titanium mediated reductive amination gives products of type (d), which are reacted with a disulfide of the general structure (e), with both terminal alcohols having leaving groups i.e. methanesulfonyl groups, to yield final products of the general structure (f). Specific synthesis procedures for Lipids 1-51 are as described below or as described in International Patent Application No. PCT/US2020/061801, filed Nov. 23, 2020, which is incorporated herein by reference in its entirety.

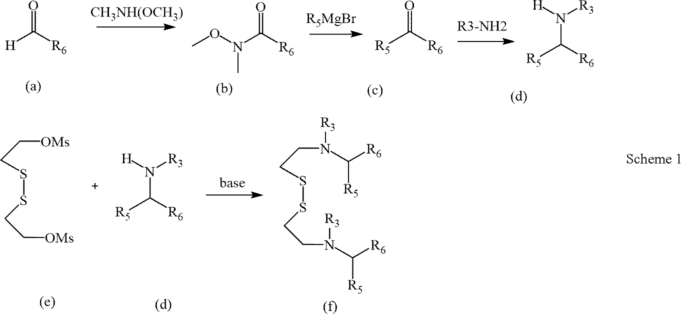

### Synthesis for Lipid 1

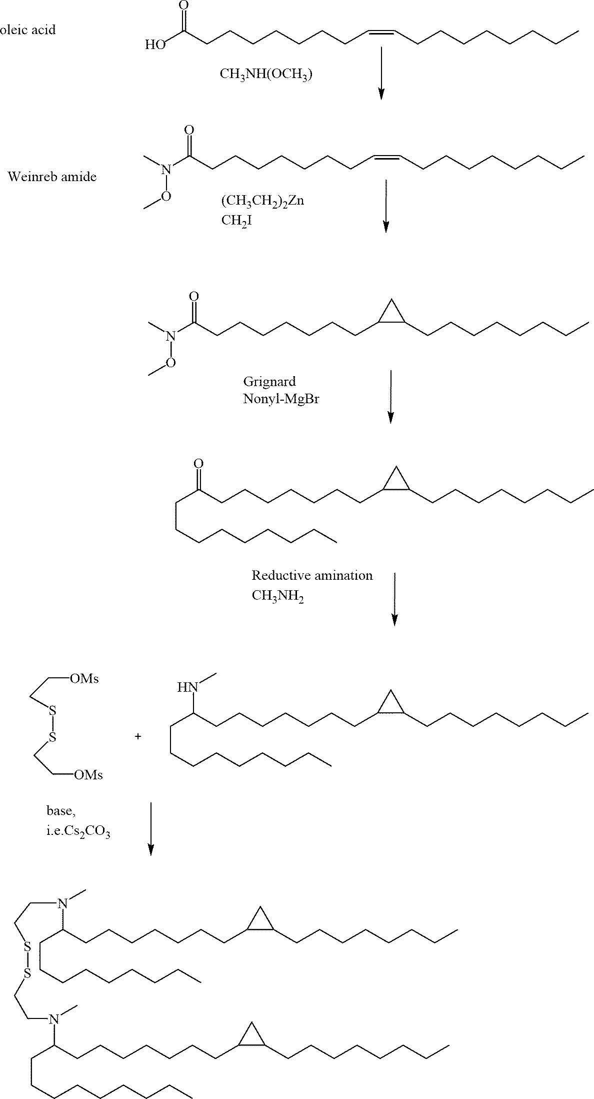

### Individual Synthesis Steps With Short Procedures

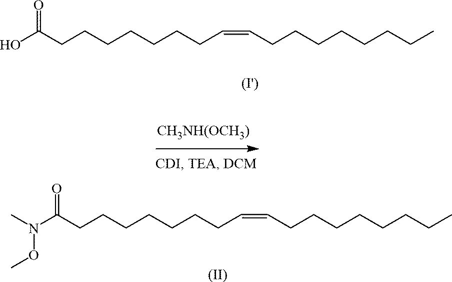

To a solution of oleic acid (I) in dichloromethane (DCM) cooled to 0° C. was added CDI. The reaction was warmed to ambient temperature for 30 minutes before cooling to 0° C. and treating first with triethylamine and then dimethyl hydroxylamine hydrochloride. After 1 hour, the reaction was partitioned between water and heptane. The organics were dried over magnesium sulfate, filtered and evaporate in vacuo to give crude Weinreb amide (II) which was carried directly into next reaction.

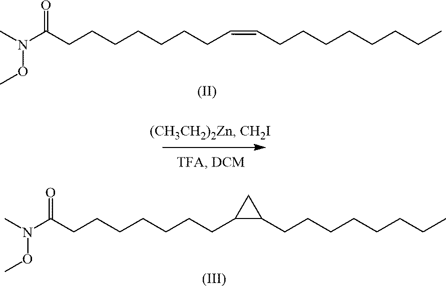

A 1 M solution of diethylzinc in dichloromethane was cooled to -1° C. and treated dropwise with TFA. After 30 minutes, diiodomethane was added and this was aged for 30 minutes in the ice bath. To this solution was added Weinreb amide (II). The reaction was warmed to ambient temperature and stirred for 1 hour. The reaction was quenched with ammonium chloride solution and the organic layer partitioned off, washed with 10% sodium thiosulfate, dried over magnesium sulfate, filtered and evaporated in vacuo. Purification was accomplished by flash chromatography to yield (III).

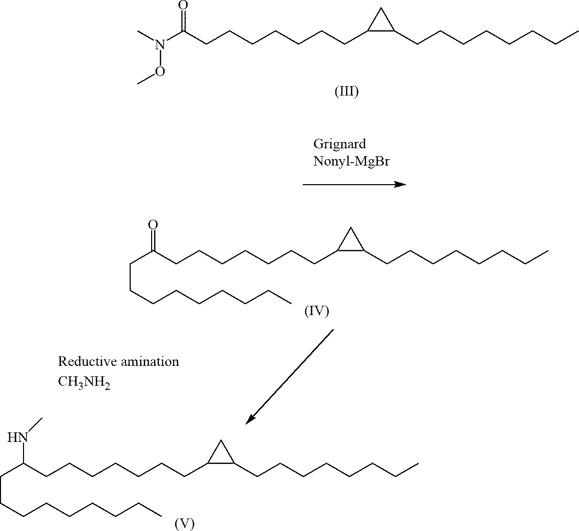

Compound (III) was dissolved in dry THF, then 1 M nonylmagnesium bromide was added under nitrogen at ambient temperature. After 10 min, the reaction was slowly quenched with excess sat.aq NH₄Cl. The reaction was washed into a separatory funnel with hexane and water, shaken, the lower aqueous layer discarded, the upper layer dried with sodium sulfate, filtered, and evaporated to give crude ketone. To the above crude ketone (IV) was added dimethylamine (2 M in THF) followed by T₁(O-₁-Pr)₄ and let stir overnight. The next day, ethanol was added followed by NaBH₄. After 5 min of stirring, the entire reaction was directly injected onto a silica column for purification yielding compound (IV).

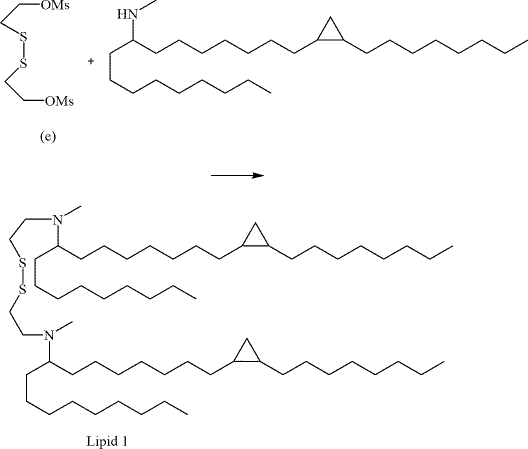

Disulfide (e) and 4 molar equivalents amine (V) were dissolved in acetonitrile, and heated for about 48 h in the presence of Cs₂CO₃. The crude reaction mixture was loaded onto silica for flash chromatography to yield the final target Lipid 1.

### Example 2: Synthesis of Ionizable Lipids of Formula II

### General Synthesis

Ionizable lipids of Formula (II) were designed and synthesized using similar synthesis methods described in the general procedure below in Scheme 2. Specific synthesis procedures for Lipids 52-71 are as described below or as described in International Patent Application No. PCT/US2021/024413, filed Mar. 26, 2021, which is incorporated herein by reference in its entirety.

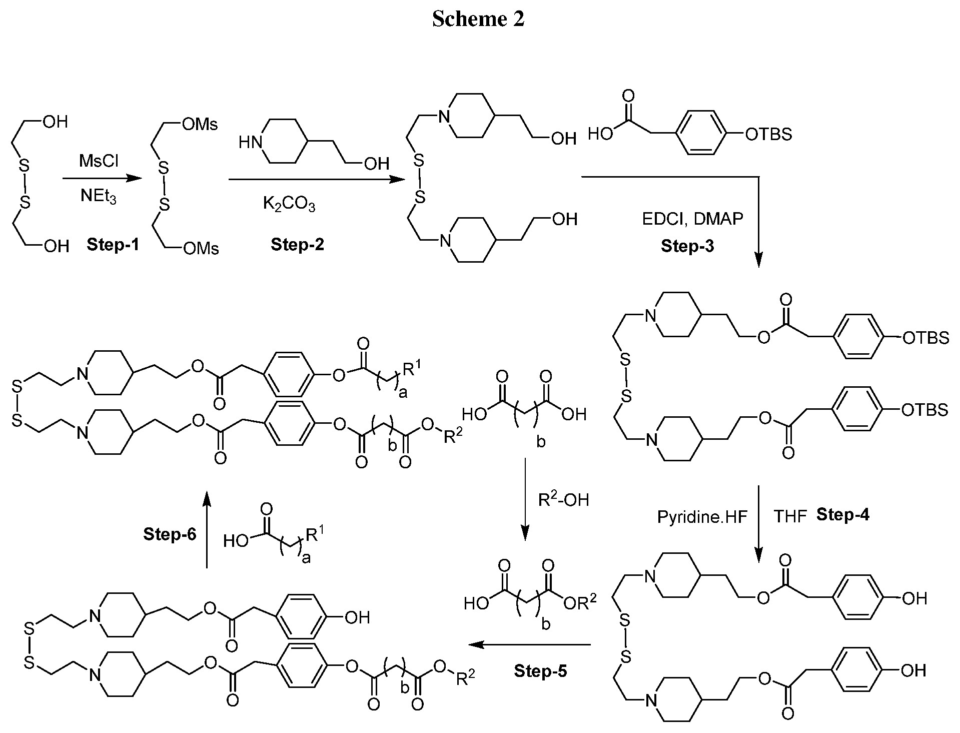

### Synthesis of 1-(Heptadecan-9-yl) 9-(4-(2-(2-(1-(2-((2-(4-(2-(2-(4-(Oleoyloxy)Phenyl)Acetoxy)Ethyl)Piperidin-1-yl)Ethyl)Disulfaneyl)Ethyl)Piperidin-4-yl)Ethoxy)-2-Oxoethyl)phenyl) Nonanedioate (Lipid 52)

### Synthesis of Cleavable, Ionizable Head Group ((Disulfanediylbis(Ethane-2,1-Diyl))Bis(Piperidine-1,4-Diyl))Bis(Ethane-2,1-Diyl) Bis(2-(4-Hydroxyphenyl)Acetate) (7)

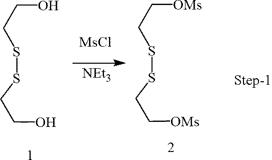

Synthesis of disulfanediylbis(ethane-2,1-diyl) dimethanesulfonate (2). Commercially available 2,2′-disulfanediylbis(ethan-1-ol) (1) (15 g, 97.2 mmol) was dissolved in acetonitrile (143 ml) followed by the addition of triethylamine (NEt₃) (33.3 g, 328 mmol). To the reaction mixture was added methanesulfonyl chloride (MsCl) (34.5 g, 300 mmol) dropwise at 0° C. The resulting reaction mixture was stirred at room temperature for 3 h. To the reaction mixture was added ethanol (EtOH) (39 ml) to quench the reaction and the insoluble materials were removed through filtration. The filtrate was partitioned between dichloromethane (DCM) (150 ml) and 10% sodium bicarbonate / water (150 ml). The organic layer was washed with 100 ml water four times, dried over magnesium sulfate (MgSO₄), and evaporated to give 2 as a brown oil (25 g, 81%), which solidified upon standing. ¹H-NMR (300 MHz, d-chloroform): δ 4.43-4.48 (t, 4H), 3.00-3.10 (m, 10H).

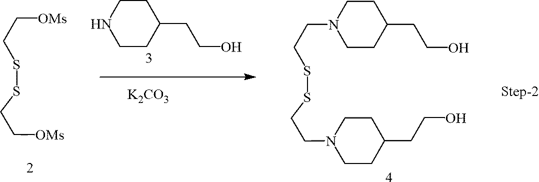

Synthesis of 2,2′-((disulfanediylbis(ethane-2,1-diyl))bis(piperidine-1,4-diyl))bis(ethan-1-ol) (4). To a solution of 2 (12 g, 38.7 mmol) in acetonitrile (310 ml) was added potassium carbonate (K₂CO₃) (13.4 g, 96.6 mmol) followed by 2-(piperidin-4-yl)ethan-1-ol (3) (20 g, 155 mmol). The resulting mixture was stirred at room temperature overnight before the insoluble material was removed through filtration. The filtrate was evaporated to dryness to afford the crude product, which was dissolved in DCM (100 ml), washed with water twice (50 ml), dried over MgSO₄, and evaporated give 4 as a yellow oil (11.8 g, 79%). ¹H-NMR (300 MHz, d-chloroform): δ 3.63-3.68 (t, 4H), 2.78-2.90 (m, 8H), 2.62-2.65 (t, 4H), 1.94-2.02 (t, 4H), 1.70 (s, 2H), 1.65-1.70 (d, 4H), 1.27-1.48 (t, 4H), 1.40-1.50 (m, 2H), 1.23-1.27 (m, 4H).

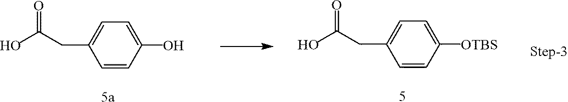

Synthesis of 2-(4-((tert-butyldimethylsilyl)oxy)phenyl)acetic acid (5). To a stirred solution of 4-hydroxyphenylacetic acid (5a) (10 g, 65 mmol) in dimethylformamide (DMF) (40 ml) at 0° C. was added NEt₃ (10 g, 100 mmol) followed by tert-butyldimethylsilylchloride (TBSC1) (15 g, 100 mmol). The resulting reaction mixture was stirred at room temperature overnight, then treated with water (200 ml) and DCM (150 ml). The organic phase was separated. The aqueous phase was extracted with DCM (100 ml). The combined organic phase was washed with a saturated solution of sodium bicarbonate, brine and dried over sodium sulfate (Na₂SO₄). Solvent was removed under reduced pressure and the residue was purified by silica gel column chromatography using 0-10% methanol (MeOH) in DCM as eluent. The fractions containing the desired compound were pooled and evaporated to afford 5 (4.8 g, 27%) and the di-tert-butyldimethylsilyl ether (di-TBS) by-product (10.5 g, 42%). ¹H-NMR of 5 (300 MHz, d-chloroform): δ 7.12 (d, 2H), 6.78 (d, 2H), 3.56 (s, 2H), 0.97 (s, 9H), 0.18 (s, 6H).

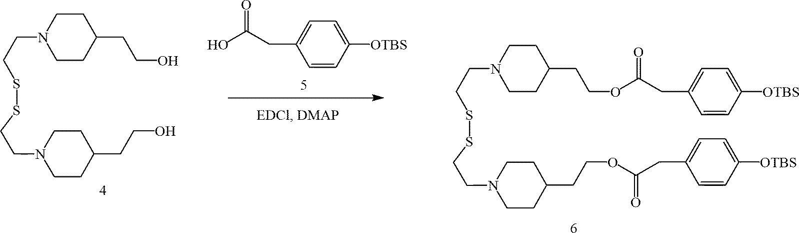

Synthesis of ((disulfanediylbis(ethane-2,1-diyl))bis(piperidine-1,4-diyl))bis(ethane-2,1-diyl) bis(2-(4-((tert-butyldimethylsilyl)oxy)phenyl)acetate) (6). To a stirred solution of the disulfide 4 yielded from Step-2 (1.92 g, 5 mmol) and phenylacetic acid 5 (3.4 g, 12.8 mmol) in DCM (100 ml) was added 4-dimethylaminopyridine (DMAP) (1.5 g, 12.5 mmol) followed by 1-ethyl-3-(3-dimethylaminopropyl)carbodiimide (EDCI) (2.4 g, 12.5 mmol). The resulting mixture was stirred at room temperature overnight, then washed with a saturated solution of sodium bicarbonate (200 ml), brine (150 ml) and dried over Na₂SO₄. Solvent was removed under reduced pressure and the residue was purified by silica gel column chromatography using 0-10% MeOH in DCM as eluent. The fractions containing the desired compound were evaporated to afford 6 (4.1 g, 92%). ¹H-NMR of 6 (300 MHz, d-chloroform): δ 7.12 (d, 4H), 6.75 (d, 4H), 4.1 (t, 4H), 3.5 (s, 4H), 2.82 (m, 8H), 2.62 (m, 4H), 1.93 (t, 4H), 1.61-1.45 (m, 8H), 1.26 (m, 6H), 0.97 (s, 18H), 0.17 (s, 4H).

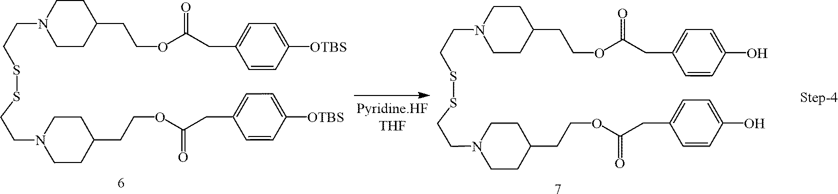

Synthesis of ((disulfanediylbis(ethane-2,1-diyl))bis(piperidine-1,4-diyl))bis(ethane-2,1-diyl) bis(2-(4-hydroxyphenyl)acetate) (7). To a stirred solution of disulfide 6 (3.1 g, 3.6 mmol) in tetrahydrofuran (THF) (40 ml) was added hydrogen fluoride pyridine (1 ml, 3.8 mmol) at 0° C. The resulting mixture was stirred at 0° C. for 2 h, then room temperature for another 2 h. The reaction mixture was treated with a saturated solution of sodium bicarbonate (200 ml) and extracted with ethyl acetate (2 ×150 ml). The combined organic phase was washed with brine (100 ml), dried over Na₂SO₄ and concentrated. The residue was purified by silica gel column chromatography using 0-10% MeOH in DCM as eluent providing the desired product 7 (1.92 g, 82%). ¹H-NMR (300 MHz, d-chloroform): δ 7.13 (d, 4H), 6.70 (d, 4H), 4.1 (t, 4H), 3.5 (s, 4H), 2.89 (m, 8H), 2.70 (m, 4H), 1.95 (t, 4H), 1.48 (m, 8H), 1.17 (m, 6H).

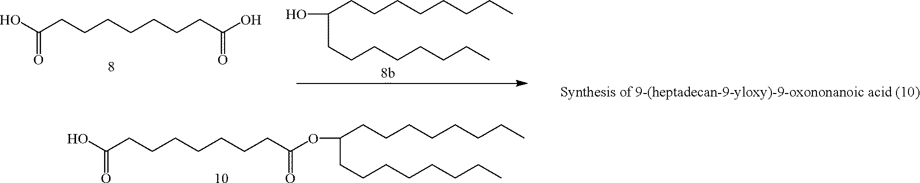

Synthesis of 9-(heptadecan-9-yloxy)-9-oxononanoic acid (10). To a stirred solution of nonanedioic acid (8) (7.34 g, 39 mmol) and heptadecan-9-ol (8b) (5 g, 19 mmol) in dichloromethane (1000 ml) was added DMAP (2.37 g, 19 mmol) followed by EDCI (3 g, 19 mmol). The resulting mixture was stirred at room temperature overnight, then washed with 250 ml 1 N HCl and 250 ml water. The organic layer was dried over MgSO₄, evaporated to dryness and purified by silica gel column chromatography using 0-10% MeOH in DCM as eluent. The fractions containing the desired compound were pooled and evaporated to afford 10 (6.2 g, 75%) as a white solid. ¹H-NMR (300 MHz, d-chloroform): δ 4.80-4.90 (m, 1H), 2.25-2.34 (m, 4H), 1.55-1.70 (m, 4H), 1.40-1.50 (m, 4H), 1.20-1.40 (m, 30H), 0.84-0.90 (t, 3H).

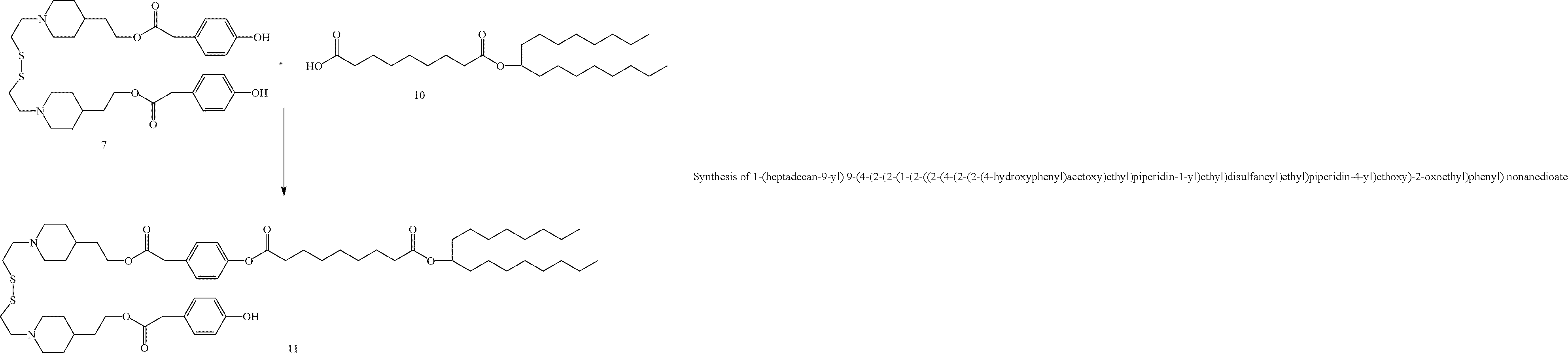

Synthesis of 1-(heptadecan-9-yl) 9-(4-(2-(2-(1-(2-((2-(4-(2-(2-(4-hydroxyphenyl)acetoxy)ethyl)piperidin-1-yl)ethyl)disulfaneyl)ethyl)piperidin-4-yl)ethoxy)-2-oxoethyl)phenyl) nonanedioate (11). To a stirred solution of the disulfide 7 produced in Step-4 (580 mg, 0.9 mmol) and acid 10 (422 mg, 0.99 mmol) in DMF (20 ml) was added DMAP (165 mg, 1.35 mmol) followed by EDCI (258 mg, 1.35 mmol). The resulting mixture was stirred at room temperature overnight, then a saturated sodium bicarbonate solution (50 ml) was added. The reaction mixture was extracted with dichloromethane (2 × 50 ml). The combined organic phase was washed with brine (30 ml), dried over Na₂SO₄ and concentrated. The residue was purified by silica gel column chromatography using 0-10% MeOH in DCM as eluent to give the desired product 11 (427 mg, 45%). ¹H-NMR (300 MHz, d-chloroform): δ 7.27 (d, 2H), 7.11 (d, 2H), 7.03 (d, 2H), 6.69 (d, 2H), 4.85 (m, 1H), 4.1 (m, 4H), 3.56 (s, 2H), 3.48 (s, 2H), 2.92 (d, 2H), 2.85-2.69 (m, 12H), 2.71 (t, 2H), 2.28 (t, 2H), 1.95 (t, 2H), 1.52-1.01 (m, 53H), 0.85 (m, 6H).

### Synthesis of Lipid 52

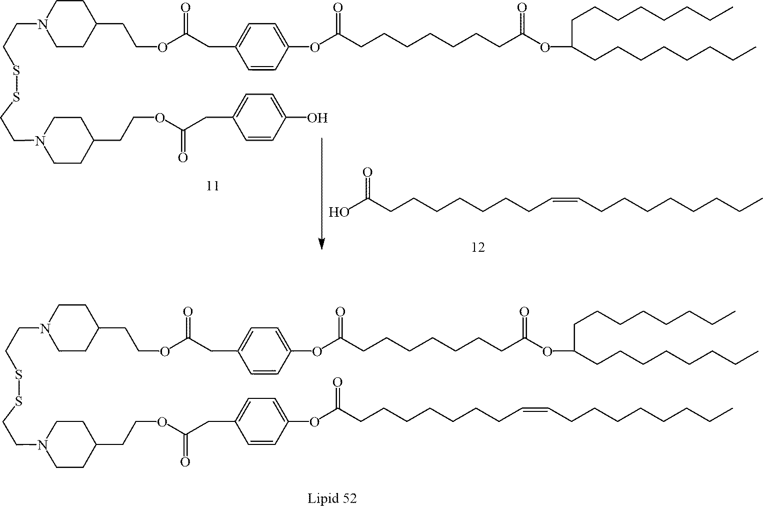

Synthesis of 1-(heptadecan-9-yl) 9-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) nonanedioate (Lipid 52). To a stirred solution of disulfide 11 (151 mg, 0.14 mmol) and oleic acid 12 (61 mg, 0.22 mmol) in dichloromethane (10 ml) was added DMAP (28 mg, 0.22 mmol) followed by EDCI (42 mg, 0.22 mmol). The resulting mixture was stirred at room temperature overnight, then washed with saturated sodium bicarbonate solution (20 ml), brine (20 ml) and dried over Na₂SO₄. Solvent was removed under reduced pressure and the residue was purified by silica gel column chromatography using 0-10% MeOH in DCM as eluent. The fractions containing the desired compound was evaporated to afford Lipid 52 (126 mg, 68%). ¹H-NMR of Lipid 52 (300 MHz, d-chloroform): δ 7.25 (d, 4H), 7.01 (d, 4H), 5.34 (m, 2H), 4.86 (m, 1H), 4.11 (t, 4H), 3.58 (s, 4H), 2.91-2.70 (m, 8H), 2.62 (m, 4H), 2.53 (t, 4H), 2.28 (t, 2H), 2.05-1.87 (m, 8H), 1.78-1.46 (m, 22H), 1.48-1.23 (m, 54H), 0.86 (t, 9H). MS [M+H]⁺ 1318.

### Example 3: Synthesis of Ionizable Lipids of Formula V

Ionizable lipids of Formula (V) were designed and synthesized using similar synthesis methods described in the general procedure below in Scheme 3. Specific synthesis procedures for Lipids 72-76 are also described below. The variables R¹, R^{1′}, R², R^{2′}, R³, R^{3′}, R⁴, R^{4′}, R⁵, and R^{5′} are as defined in Formula (V). R^{x} is shorter than R⁴ by 2 carbon atoms and similarly, R^{x′} is shorter than R^{4′} by 2 carbon atoms.

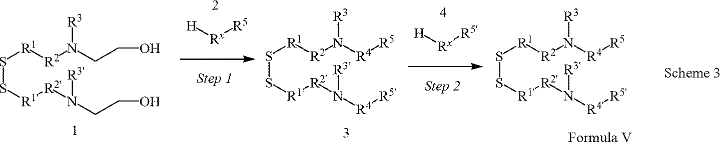

At Step 1, to a stirred solution of disulfide 1 and acid 2 in dichloromethane (DCM) was added 4-dimethylaminopyridine (DMAP) followed by 1-ethyl-3-(3-dimethylaminopropyl)carbodiimide (EDCI). The resulting mixture was stirred at room temperature for 2 days, then a saturated sodium bicarbonate solution was added. The reaction mixture was extracted with DCM. The combined organic phase was washed with brine, dried over sodium sulfate (Na₂SO₄) and concentrated. The residue was purified by silica gel column chromatography using 0-5% methanol (MeOH) in DCM as eluent to afford 3. Step 2 reagents and conditions were mostly identical to those in Step 1, which yielded a lipid of Formula (V) as a final product.

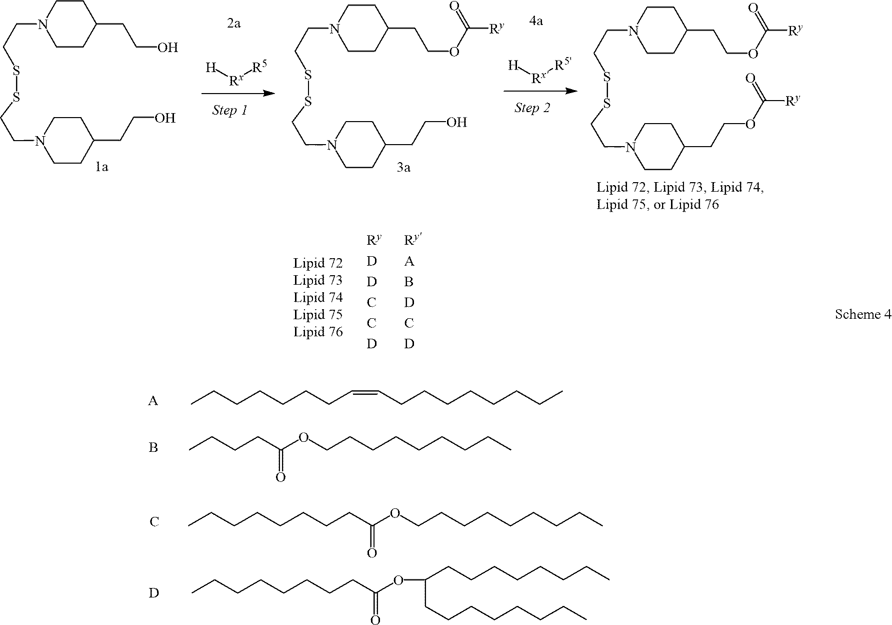

### Synthesis of O′1,O1-(((Disulfanediylbis(Ethane-2,1-Diyl))Bis(Piperidine-1,4-Diyl))Bis(Ethane-2,1-Diyl)) 9,9′-di(Heptadecan-9-yl) di(Nonanedioate) (Lipid 76) and 1-(Heptadecan-9-yl) 9-(2-(1-(2-((2-(4-(2-(Oleoyloxy)Ethyl)Piperidin-1-yl)Ethyl)Disulfaneyl)Ethyl)Piperidin-4-yl)Ethyl)Nonanedioate (Lipid 72)

Referring to Scheme 4, to a stirred a solution of disulfide 1a (synthesis thereof described in Example 2) (1.17 g, 3.1 mmol) and 9-(heptadecan-9-yloxy)-9-oxononanoic acid (2.0 g, 4.6 mmol) in DCM (50 ml) was added DMAP (565 mg, 4.6 mmol) followed by EDCI (878 mg, 4.6 mmol). The resulting mixture was stirred at room temperature for 2 days, then washed with saturated sodium bicarbonate solution (60 ml), brine (20 ml) and dried over Na₂SO₄. Solvent was removed under reduced pressure and the residue was purified twice by silica gel column chromatography using 0-10% MeOH in DCM as eluent. The fractions containing the desired compounds were evaporated to afford Lipid 76 (620 mg, 23%) and 1-(heptadecan-9-yl) 9-(2-(1-(2-((2-(4-(2-hydroxyethyl)piperidin-1-yl)ethyl)disulfaneyl)ethyl)piperidin-4-yl)ethyl) nonanedioate or compound 3a-D (i.e., Compound 3a in Scheme 4 where R^{y} = D) (389 mg, 22%).

¹H-NMR of Lipid 76 (300 MHz, d-chloroform): δ 4.85 (m, 2H), 4.09 (t, 4H), 2.91-2.74 (m, 8H), 2.63-2.67 (m, 4H), 2.27-2.22 (m, 8H), 1.97 (t, 4H), 1.75-1.43 (m, 24H), 1.45-1.16 (m, 66H), 0.86 (t, 12H). MS [M+H]⁺ 1194.

¹H-NMR of 3a-D (300 MHz, d-chloroform): δ 4.83 (m, 1H), 4.06 (t, 2H), 3.63 (t, 2H), 2.97-2.69 (m, 9H), 2.66 (m, 4H), 2.25 (t, 4H), 1.93 (t, 4H), 1.76-1.43 (m,16H), 1.39-1.22 (m, 36H), 0.86 (t, 6H).

Next, to a stirred solution of disulfide 3a-D (185 mg, 0.23 mmol) and oleic acid (131 mg, 0.46 mmol) in dichloromethane (10 ml) was added DMAP (55 mg, 0.46 mmol) followed by EDCI (87 mg, 0.46 mmol). The resulting mixture was stirred at room temperature overnight, then washed with saturated sodium bicarbonate solution (20 ml), brine (20 ml) and dried over Na₂SO₄. Solvent was removed under reduced pressure and the residue was purified by silica gel column chromatography using 0-10% MeOH in DCM as eluent. The fraction containing the desired compound was evaporated to afford Lipid 72 (165 mg, 68%).

¹H-NMR of Lipid 72 (300 MHz, d-chloroform): δ 5.32 (m, 2H), 4.85 (m, 1H), 4.09 (t, 4H), 2.96-2.77 (m, 8H), 2.67-2.53 (m, 4H), 2.28-2.20 (m, 6H), 2.16-1.92 (t, 8H), 1.75-1.47 (m, 14H), 1.41-1.13 (m, 60H), 0.86 (t, 9H). MS [M+H]⁺ 1049.

### Synthesis of O′1,O1-(((Disulfanediylbis(Ethane-2,1-Diyl))Bis(Piperidine-1,4-Diyl))Bis(Ethane-2,1-Diyl)) 9,9′-Dinonyl di(Nonanedioate) (Lipid 75)

Referring to Scheme 4, to a stirred solution of disulfide 1a (376 mg, 1 mmol) and 9-(octyloxy)-9-oxononanoic acid (629 mg, 2 mmol) in DCM (25 ml) was added DMAP (244 mg, 2 mmol) followed by EDCI (310 mg, 2 mmol). The resulting mixture was stirred at room temperature overnight, then a saturated sodium bicarbonate solution (20 ml) was added. The reaction mixture was extracted with DCM (2 × 50 ml). The combined organic phase was washed with brine (30 ml), dried over Na₂SO₄ and concentrated. The residue was purified by silica gel column chromatography using 0-5% MeOH in DCM as eluent to afford Lipid 75 (240 mg, 25%) as a light yellow solid. ¹H-NMR (300 MHz, d-chloroform): δ 4.04-4.09 (m, 8 H), 2.5-3.0 (m, 10 H), 2.25-2.30 (t, 8 H), 2.0 (t, 4 H), 1.58-1.90 (m, 24 H), 1.20-1.40 (m, 42 H), 0.87 (t, 6 H).

### Synthesis of 1-(Heptadecan-9-yl) 9-(2-(1-(2-((2-(4-(2-((9-(Nonyloxy)-9-Oxononanoyl)Oxy)Ethyl)Piperidin-1-yl)Ethyl)Disulfaneyl)Ethyl)Piperidin-4-yl)Ethyl)Nonanedioate (Lipid 74)

Referring to Scheme 4, to a stirred solution of disulfide 1a (376 mg, 1 mmol) and and 9-(octyloxy)-9-oxononanoic acid (629 mg, 2 mmol) in DCM (25 ml) was added DMAP (244 mg, 2 mmol) followed by EDCI (310 mg, 2 mmol). The resulting mixture was stirred at room temperature overnight, then a saturated sodium bicarbonate solution (20 ml) was added. The reaction mixture was extracted with DCM (2 × 50 ml). The combined organic phase was washed with brine (30 ml), dried over Na₂SO₄ and concentrated. The residue was purified by silica gel column chromatography using 0-5% MeOH in dichloromethane as eluent to afford 1-(2-(1-(2-((2-(4-(2-hydroxyethyl)piperidin-1-yl)ethyl)disulfaneyl)ethyl)piperidin-4-yl)ethyl) 9-nonyl nonanedioate or compound 3a-C (i.e., Compound 3a in Scheme 4 where R^{y} = C) (250 mg, 26%), which was used directly for next conversion without characterization.

Next, to a stirred solution of disulfide 3a-C (650 mg, 0.97 mmol) and 9-(heptadecan-9-yloxy)-9-oxononanoic acid (411 mg, 0.96 mmol) in DCM (50 ml) was added DMAP (117 mg, 0.96 mmol) followed by EDCI (149 mg, 0.96 mmol). The resulting mixture was stirred at room temperature for 2 days, then washed with water and dried over Na₂SO₄. Solvent was removed under reduced pressure and the residue was purified by silica gel column chromatography using 0-10% MeOH in DCM as eluent. The fraction containing the desired compound was evaporated to afford Lipid 74 (420 mg, 40%). ¹H-NMR (300 MHz, d-chloroform): δ 4.9 (m, 1 H), 4.05-4.09 (m, 6 H), 2.80-3.0 (m, 8 H), 2.60-2.70 (m, 4 H), 2.25-2.27 (m, 8 H), 1.92-2.01 (t, 4 H), 1.48-1.62 (m, 25 H), 1.24-1.40 (m, 52 H), 0.87 (t, 9 H).

### Synthesis of 1-(Heptadecan-9-yl) 9-(2-(1-(2-((2-(4-(2-((5-(Nonyloxy)-5-Oxopentanoyl)Oxy)Ethyl)Piperidin-1-yl)Ethyl)Disulfaneyl)Ethyl)Piperidin-4-yl)Ethyl) Nonanedioate (Lipid 73)

To a stirred solution of disulfide 4 (3.76 g, 10 mmol) and 9-(heptadecan-9-yloxy)-9-oxononanoic acid (2.13 g, 5 mmol) in DCM (100 ml) was added DMAP (776 mg, 5 mmol) followed by EDCI (610 mg, 5 mmol). The resulting mixture was stirred at room temperature for 2 days, then a saturated sodium bicarbonate solution (40 ml) was added. The reaction mixture was extracted with DCM (2 × 100 ml). The combined organic phase was washed with brine (60 ml), dried over Na₂SO₄ and concentrated. The residue was purified by silica gel column chromatography using 0-5% MeOH in DCM as eluent to afford 1-(heptadecan-9-yl) 9-(2-(1-(2-((2-(4-(2-hydroxyethyl)piperidin-1-yl)ethyl)disulfaneyl)ethyl)piperidin-4-yl)ethyl) nonanedioate or compound 3a-D (i.e., Compound 3a in Scheme 4 where R^{y} = D) (1.4 g, 36%). ¹H-NMR (300 MHz, d-chloroform): δ 4.90 (m, 1 H), 4.09-4.10 (m, 3 H), 3.68 (t, 2 H), 2.79-2.99 (m, 8 H), 2.66 (m, 4 H), 2.30 (m, 4 H), 2.03 (t, 4H), 1.22-1.78 (m, 55 H), 0.86 (s, 6 H).

Next, to a stirred solution of disulfide 3a-D (300 mg, 0.38 mmol) and 5-(nonyloxy)-5-oxopentanoic acid (115 mg, 0.45 mmol) in DCM (20 ml) was added DMAP (49 mg, 0.4 mmol) followed by EDCI (62 mg, 0.4 mmol). The resulting mixture was stirred at room temperature overnight, then washed with saturated sodium bicarbonate solution (20 ml), brine (20 ml) and dried over Na₂SO₄. Solvent was removed under reduced pressure and the residue was purified by silica gel column chromatography using 0-5% MeOH in DCM as eluent. The fraction containing the desired compound was evaporated to afford Lipid 73 (165 mg, 42%). ¹H-NMR (300 MHz, d-chloroform): δ 5.85 (m, 1 H), 4.05-4.10 (m, 6 H), 2.79-2.88 (m, 8 H), 2.63-2.66 (m, 4 H), 2.33-2.36 (t, 4 H), 2.26-2.33 (t, 4 H), 1.94-1.98 (m, 6 H), 1.55-1.59 (m, 22 H), 1.24-1.40 (m, 48 H), 0.84-0.89 (t, 9 H).

### Example 4: Synthesis of Ionizable Lipids of Formula XV

Lipids of Formula (XV) were designed and synthesized using similar synthesis methods depicted in Scheme 5 (R⁵ is absent) and Scheme 6 (R⁵ is C₁-C₈ alkylene or C₂-C₈ alkenylene) below. All other variables in the compounds shown in Schemes 5-6, i.e., R¹, R², R³, R⁴, R^{6a}, R^{6b}, X¹, X², and n, are as defined in Formula (XV). X^{1′} is X¹ as defined but with an additional protecting group, such as benzyl or pyridine. Additional synthesis procedures and specific synthesis procedures for Lipids 77-87 are as described in U.S. Pat. Application No. 63/176,943, filed Apr. 20, 2021, which is incorporated herein by reference in its entirety.

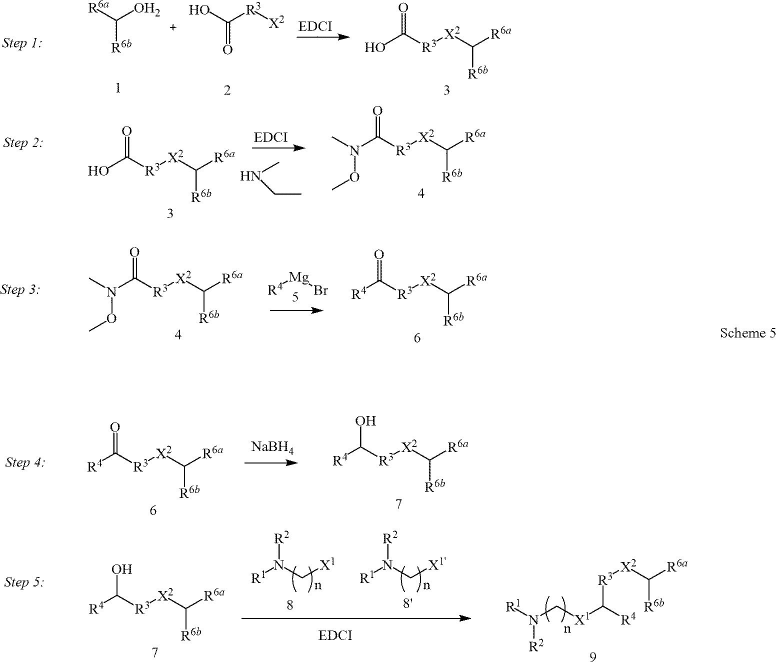

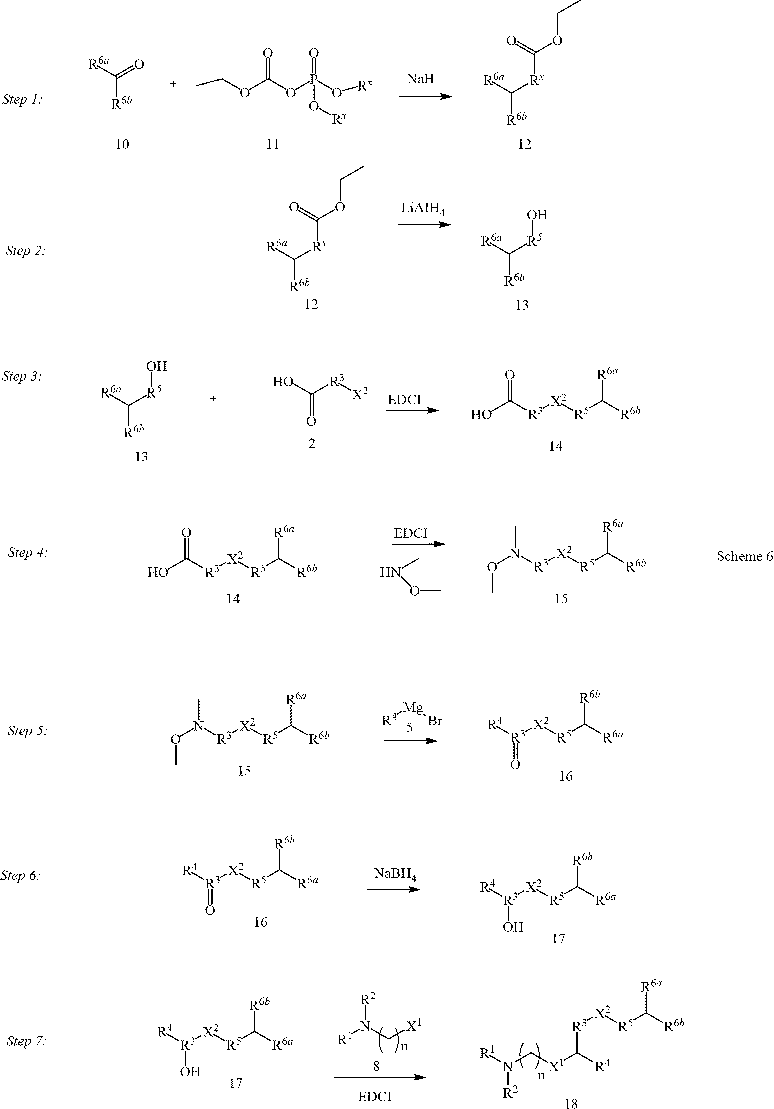

R^{x} is alkylene or alkenylene having one less carbon atom than R⁵.

Diester lipids of Formula (XVII) were designed and synthesized using similar synthesis methods depicted in Scheme 7 (R⁵ is absent) and Scheme 8 (R⁵ is C₁-C₈ alkylene or C₂-C₈ alkenylene) below. All other variables in the compounds shown in Scheme 7 and Scheme 8, i.e., R¹, R², R³, R⁴, R^{6a}, R^{6b}, and n, are as defined in Formula (XVII).

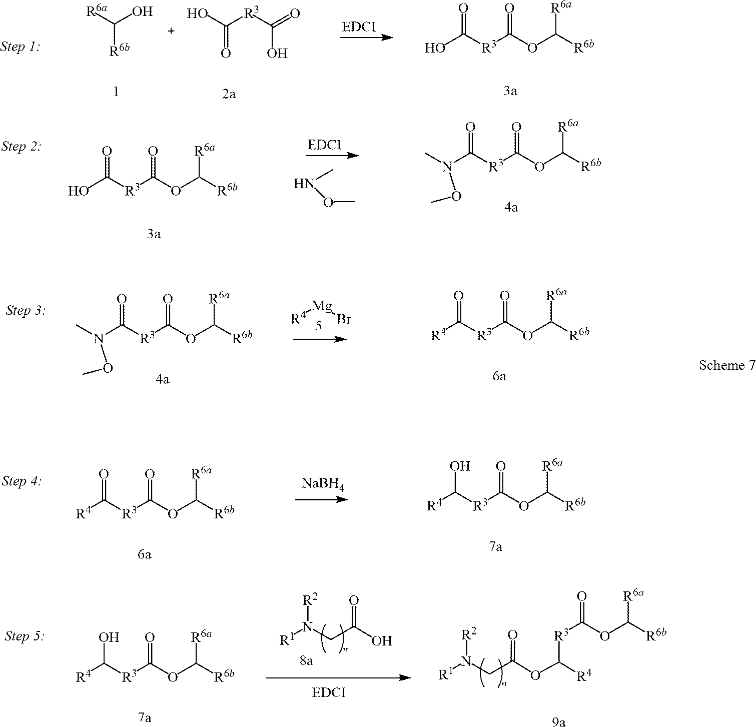

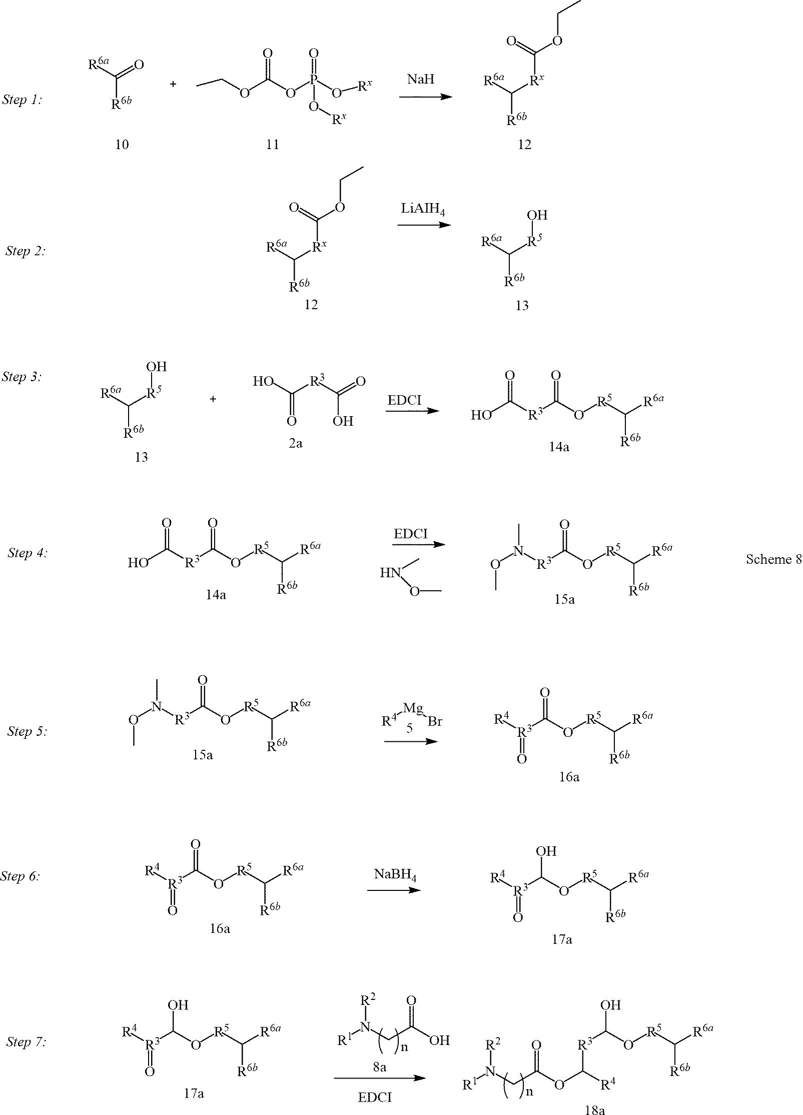

R^{x} is alkylene or alkenylene having one less carbon atom than R⁵.

### Scheme 5 and Scheme 6

Referring to Scheme 5 and Scheme 6, at Step 1, to a stirred solution of the acid 1 and alcohol 2 (or 2a) in dichloromethane (DCM), was added 4-dimethylaminopyridine (DMAP) followed by 1-ethyl-3-(3-dimethylaminopropyl)carbodiimide (EDCI). The resulting mixture was stirred at room temperature overnight, then washed with hydrochloric acid (HCl) and water. The organic layer was dried over magnesium sulfate (MgSO₄), evaporated to dryness, and purified by silica gel column chromatography using 0-10% methanol (MeOH) in DCM as eluent. The fractions containing the desired compound were pooled and evaporated to afford acid 3 as a white solid.

At Step 2, to a solution of acid 3 (or 3a) in DCM, EDCI and triethylamine (TEA) were added, and the mixture was stirred for 15 min at room temperature. Then, N,O-dimethylhydroxylamine hydrochloride and DMAP were added and the mixture was stirred overnight at room temperature. The next day, the reaction was quenched with an ammonium chloride aqueous solution (NH₄Cl (aq)) and diluted with DCM. The organic layer was washed with NH₄Cl and brine and dried over anhydrous sodium sulfate (Na₂SO₄). Solvent was evaporated under vacuo. The product 4 (or 4a) was used in next step without further purification.

At Step 3, the compound 4 (or 4a) was dissolved in anhydrous tetrahydrofuran (THF). Then 5, a magnesium bromide solution in diethyl ether (Et₂O) was added dropwise at 0° C. The resulted mixture was stirred at room temperature for 16 h under nitrogen gas (N₂). The reaction was quenched with saturated NH₄Cl solution and extracted with ether. The organic layer was washed with brine and dried over anhydrous Na₂SO₄. Solvent was evaporated under vacuo and purified by column chromatography using 0-10% ethyl acetate (EtOAc) in hexane as eluent to afford 6 (or 6a).

At Step 4, to a solution of 6 (or 6a) in anhydrous THF was added sodium borohydride (NaBH₄) at 0° C. and the mixture was stirred overnight under N₂ atmosphere. The reaction was quenched with saturated NH₄Cl solution and extracted with EtOAc. The organic phase was washed with brine and dried over anhydrous Na₂SO₄. Solvent was evaporated under vacuo and purified by column chromatography using 0-10% EtOAc in hexane as eluent to afford 7.

At Step 5, to a solution of compound 7 (or 7a) and compound 8 (or 8a) in DCM, N,N-diisopropylethylamine (DIPEA) was added. Then EDCI and DMAP (0.012 g, 0.1 mmol) were added, and the mixture was stirred overnight at room temperature under N₂ atmosphere. Next day reaction was diluted with DCM. The organic layer was washed with sodium bicarbonate aqueous solution (NaHCO₃ (aq)) and dried over anhydrous Na₂SO₄. Solvent was evaporated under vacuo and purified by column chromatography using 0-5% MeOH in DCM as eluent to afford the final product 9 (or diester 9a).

### Scheme 7 and Scheme 8

Referring to Scheme 7 and Scheme 8, at Step 1, to an ice-cold solution of 3 g (11.8 mmol) of ketone 10 in THF, the phosphoric anhydride solution 11 was added dropwise. The reaction was stirred for 30 min and then sodium hydride (NaH) was added. The reaction gave 12.

At Step 2, compound 2 in THF was reacted with lithium aluminum hydride solution (LiAlH₄). After 48 h, the crude was quenched with water and extracted with ether to give the alcohol 13.

The subsequent Step 3 through Step 7 of Scheme 6 and Scheme 7 were carried out similar procedures as described in Step 1 through Step 5 of Scheme 4 and Scheme 5, with the alcohol 13 as the appropriate starting material and with other modifications that would be within the knowledge of the person having ordinary skill in the art.

### Synthesis of Lipid 77, Lipid 78, Lipid 79, Lipid 80, and Lipid 81

Procedures for synthesizing Lipid 77, Lipid 78, Lipid 79, Lipid 80, and Lipid 81 are described below with reference to Scheme 9, also provided below.

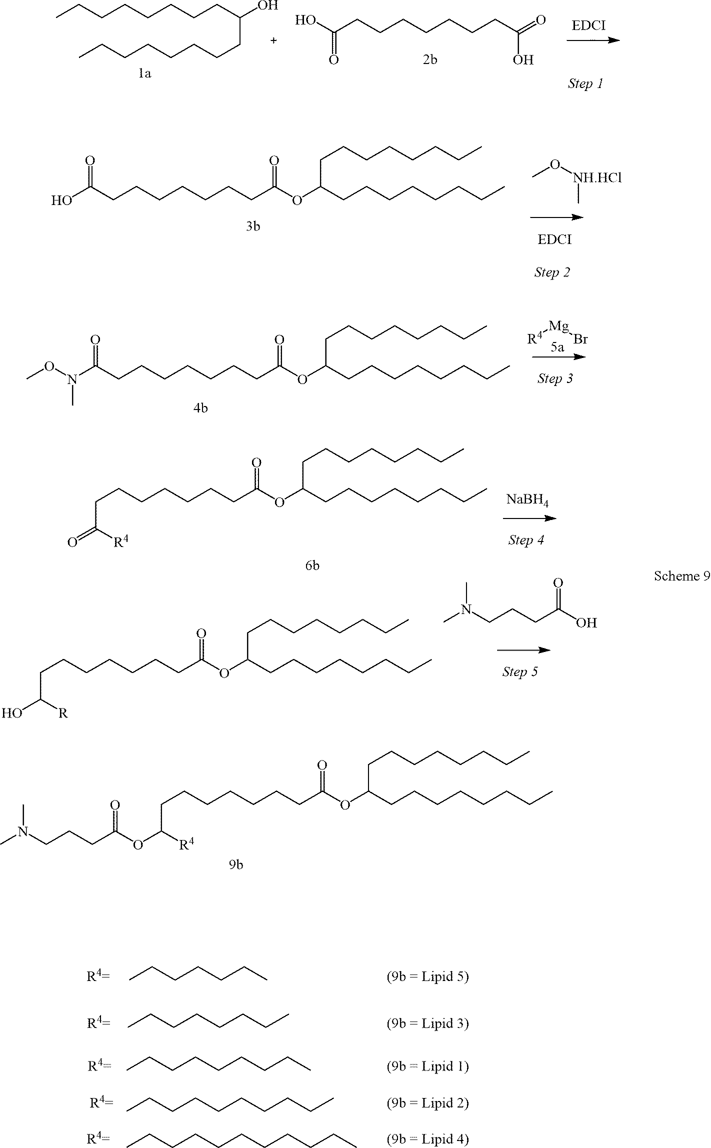

### Step 1: Synthesis of 9-(Heptadecan-9-Yloxy)-9-Oxononanoic Acid (3b)

To a stirred solution of nonanedioic acid (2b, also called azelaic acid) (7.34 g, 39 mmol) and heptadecan-9-ol (1a) (5 g, 19 mmol) in DCM (1000 ml) was added DMAP (2.37 g, 19 mmol) followed by EDCI (3 g, 19 mmol). The resulting mixture was stirred at room temperature overnight, then washed with 250 ml 1 N HCl and 250 ml water. The organic layer was dried over MgSO₄, evaporated to dryness, and purified by silica gel column chromatography using 0-10% methanol in DCM as eluent. The fractions containing the desired compound were pooled and evaporated to afford 3b (6.2 g, 75%) as a white solid. ¹H-NMR (300 MHz, d-chloroform): δ 4.80-4.90 (m, 1H), 2.25-2.34 (m, 4H), 1.55-1.70 (m, 4H), 1.40-1.50 (m, 4H), 1.20-1.40 (m, 30H), 0.84-0.90 (t, 3H).

### Step 2: Synthesis of Heptadecan-9-yl 9-(Methoxy(Methyl)Amino)-9-Oxononanoate (4b)

To a solution of compound 3 (5.4 g, 12.7 mmol) in DCM (60 mL), EDCI (3.6 g, 19.7 mmol), and TEA (3.5 mL, 25.4 mmol) were added, and the mixture was stirred for 15 min at room temperature. Then N,O-dimethylhydroxylamine hydrochloride (1.36 g, 13.97 mmol) and DMAP (0.15 g, 1.27 mmol) were added and stirred overnight at room temperature. Next day, the reaction was quenched with NH₄Cl (aq) and diluted with DCM. The organic layer was washed with NH₄Cl and brine and dried over anhydrous Na₂SO₄. Solvent was evaporated under vacuo. The product 4b was used in next step without further purification. ¹H NMR (300 MHz, d-chloroform) δ 4.85 (t, J = 6.2 Hz, 1H), 3.67 (s, 3H), 3.58 (s, 2H), 3.17 (s, 3H), 2.40 (t, J= 7.6 Hz, 2H), 2.27 (t, J= 7.5 Hz, 2H), 1.63 (dd, J = 14.8, 5.5 Hz, 6H), 1.49 (d, J = 5.4 Hz, 4H), 1.37 - 1.19 (m, 32H), 0.86 (d, J = 6.8 Hz, 6H).

### Step 3: Synthesis of Heptadecan-9-yl 9-Oxohexadecanoate (6b Where R⁴ is C₇ alkyl), Heptadecan-9-yl 9-Oxoheptadecanoate (6b Where R⁴ is C₈ Alkyl), Heptadecan-9-yl 9-Oxooctadecanoate (6b Where R⁴ is C₉ Alkyl),Heptadecan-9-yl 9-Oxononadecanoate (6b Where R⁴ is C₁₀ Alkyl), or Heptadecan-9-yl 9-Oxoicosanoate (6b Where R⁴ is C₁₁ Alkyl)

### Heptadecan-9-yl 9-Oxohexadecanoate (6b Where R⁴ is C₇ Alkyl)

Compound 4b (1.0 g, 2.13 mmol) was dissolved in 10 ml of anhydrous THF. Then, 1 M heptyl magnesium bromide solution (Compound 5a where R⁴ is C₇ alkyl) in Et₂O (3.2 ml, 3.2 mmol) was added dropwise at 0° C. The resulted mixture was stirred at room temperature for 16 h under N₂. The reaction was quenched with saturated NH₄Cl solution and extracted with ether. The organic layer was washed with brine and dried over anhydrous Na₂SO₄. Solvent was evaporated under vacuo and purified by column chromatography using 0-10% EtOAc in hexane as eluent to afford 6b where R⁴ is C₇ alkyl (0.3 g, 30%). ¹H NMR (300 MHz, d-chloroform) δ 4.85 (t, J = 6.2 Hz, 1H), 2.37 (t, J = 7.4 Hz, 4H), 2.27 (t, J = 7.5 Hz, 2H), 1.64-1.43 (m, 12H), 1.27 (s, 36), 0.87 (t, J = 6.7 Hz, 9H).

### Heptadecan-9-yl 9-Oxoheptadecanoate (6b Where R⁴ is C₈ Alkyl)

Compound 4b (1.0 g, 2.13 mmol) was dissolved in 10 ml of anhydrous THF. Then 1 M octyl magnesium bromide solution (Compound 5 where R⁴ is C₈ alkyl) in Et₂O (1.6 ml, 3.2 mmol) was added dropwise at 0° C. The resulted mixture was stirred at room temperature for 16 h under N₂. The reaction was quenched with saturated NH₄Cl solution and extracted with ether. The organic layer was washed with brine and dried over anhydrous Na₂SO₄. Solvent was evaporated under vacuo and purified by column chromatography using 0-10% EtOAc in hexane as eluent to afford 6b where R⁴ is C₈ alkyl (0.41 g, 40%). ¹H NMR (300 MHz, d-chloroform) δ 4.85 (t, J = 6.2 Hz, 1H), 2.37 (t, J = 7.4 Hz, 4H), 2.26 (t, J= 7.5 Hz, 2H), 1.65 - 1.38 (m, 8H), 1.33 - 1.18 (m, 42H), 0.87 (t, J = 6.5 Hz, 9H).

### Heptadecan-9-yl 9-Oxooctadecanoate (6b Where R⁴ is C₉ Alkyl)

Compound 4b (1.1 g, 2.3 mmol) was dissolved in 20 ml of anhydrous THF. Then 1 M nonyl magnesium bromide solution (Compound 5 where R⁴ is C₉ alkyl) in Et₂O (6.13 ml, 3.2 mmol) was added dropwise at 0° C. The resulted mixture was allowed to reach room temperature over 2 h. The reaction was quenched with saturated NH₄Cl solution and extracted with ether. The organic layer was washed with brine and dried over anhydrous Na₂SO₄. Solvent was evaporated under vacuo and purified by column chromatography using 0-30% EtOAc in hexane as eluent to afford 6b where R⁴ is C₉ alkyl (1.2 g, 96%). ¹H NMR (300 MHz, d-chloroform) δ 4.85 (t, J = 6.2 Hz, 1H), 2.37 (t, J = 7.4 Hz, 4H), 2.26 (t, J= 7.5 Hz, 2H), 1.65 - 1.38 (m, 8H), 1.33 - 1.18 (m, 44H), 0.87 (t, J = 6.5 Hz, 9H).

### Heptadecan-9-yl 9-Oxononadecanoate (6b Where R⁴ is C₁₀ Alkyl)

Compound 4b (0.3 g, 0.64 mmol) was dissolved in 2 ml of anhydrous THF. Then 1 M decyl magnesium bromide solution (Compound 5 where R⁴ is C₁₀ alkyl) in Et₂O (1.28 ml, 0.77 mmol) was added dropwise at 0° C. The resulted mixture was allowed to reach room temperature over 2 h. The reaction was quenched with saturated NH₄Cl solution and extracted with hexane. The organic layer was washed with brine and dried over anhydrous Na₂SO₄. Solvent was evaporated under vacuo and purified by column chromatography using 0-10% EtOAc in hexane as eluent to afford 6b where R⁴ is C₁₀ alkyl (0.2 g, 47%).

### Heptadecan-9-yl 9-Oxoicosanoate (6b Where R⁴ is C₁₁ Alkyl)

To a solution of 1-bromoundecane (0.47 g, 2 mmol) and in 2 mL of anhydrous ether, was added Mg (0.072 g, 3 mmol) and 1 drop of 1,2-dibromoethane. The resulting mixture was stirred for 1 h and filtered and dried. The product undecylmagnesium bromide (Compound 5 where R⁴ is C₁₁ alkyl) was used in next step without further purification.

Compound 4b (0.47 g, 1 mmol) was dissolved in 3 ml of anhydrous THF. Then undecylmagnesium bromide solution in THF (1.1 ml, 1 mmol) was added dropwise at 0° C. The resulted mixture was allowed to reach room temperature over 2 h. The reaction was quenched with saturated NH₄Cl solution and extracted with hexane. The organic layer was washed with brine and dried over anhydrous Na₂SO₄. Solvent was evaporated under vacuo and purified by column chromatography using 0-10% EtOAc in hexane as eluent to afford (Compound 5 where R⁴ is C₁₁ alkyl (0.27 g, 48%). ¹H NMR (300 MHz, d-chloroform) δ 4.86 (t, J = 6.2 Hz, 1H), 2.37 (t, J = 7.4 Hz, 4H), 2.27 (t, J = 7.5 Hz, 2H), 1.70 - 1.45 (m, 8H), 1.29-1.25 (m, 48H), 0.87 (t, J = 6.6 Hz, 9H).

### Step 4: Synthesis of Heptadecan-9-yl 9-Hydroxyhexadecanoate (7b Where R⁴ is C₇ Alkyl), Heptadecan-9-yl 9-Hydroxyheptadecanoate (7b Where R⁴ is C₈ Alkyl),Heptadecan-9-yl 9-Hydroxyoctadecanoate (7b Where R⁴ is C₉ Alkyl),Heptadecan-9-yl 9-Hydroxynonadecanoate (7b Where R⁴ is C₁₀ Alkyl), or Heptadecan-9-yl 9-Hydroxyicosanoate (7b Where R⁴ is C₁₁ Alkyl)

### Heptadecan-9-yl 9-Hydroxyhexadecanoate (7b Where R⁴ is C₇ Alkyl)

To a solution of heptadecan-9-yl 9-oxohexadecanoate (6b where R⁴ is C₇ alkyl) (0.3 g, 0.6 mmol) in 10 mL of anhydrous THF was added NaBH₄ (0.09 g, 2.4 mmol) at 0° C. and stirred overnight under N₂ atmosphere. The reaction was quenched with saturated NH₄Cl solution and extracted with EtOAc. The organic phase was washed with brine and dried over anhydrous Na₂SO₄. Solvent was evaporated under vacuo and purified by column chromatography using 0-10% EtOAc in hexane as eluent to afford 7b where R⁴ is C₇ alkyl (0.25 g, 82 %). ¹H NMR (300 MHz, d-chloroform) δ 4.92 - 4.78 (m, 1H), 3.57 (m, 1H), 2.27 (t, J = 7.5 Hz, 2H), 1.66 - 1.36 (m, 12H), 1.31-1.25 (m, 40H), 0.87 (t, J = 6.1 Hz, 9H).

### Heptadecan-9-yl 9-Hydroxyheptadecanoate (7b Where R⁴ Is C₈ Alkyl)

To a solution of heptadecan-9-yl 9-oxoheptadecanoate (6b where R⁴ is C₈ alkyl) (0.4 g, 0.77 mmol) in 10 mL of anhydrous THF was added NaBH₄ (0.04 g, 1.15 mmol) at 0° C. and stirred overnight under N₂ atmosphere. The reaction was quenched with saturated NH₄Cl solution and extracted with EtOAc. The organic phase was washed with brine and dried over anhydrous Na₂SO₄. Solvent was evaporated under vacuo and purified by column chromatography using 0-10% EtOAc in hexane as eluent to afford 7b where R⁴ is C₈ alkyl (0.21 g, 52 %). ¹H NMR (300 MHz, d-chloroform) δ 4.92 - 4.80 (m, 1H), 3.57 (m, 1H), 2.27 (t, J = 7.5 Hz, 2H), 1.64 - 1.40 (m, 12H), 1.36 - 1.18 (m, 42H), 0.87 (t, J = 6.5 Hz, 9H).

### Heptadecan-9-yl 9-Hydroxyoctadecanoate (7b Where R⁴ is C₉ Alkyl)

To a solution of heptadecan-9-yl 9-oxooctadecanoate (6b where R⁴ is C₉ alkyl) (1.1 g, 2.05 mmol) in 40 mL of DCM:MeOH (1:1) mixture was added NaBH₄ (0.3 g, 8 mmol) at 0° C. and stirred for 2 h under N₂ atmosphere. The reaction was quenched with 1 M HCl (aq) solution and extracted with DCM. The organic phase was washed with brine and dried over anhydrous Na₂SO₄. Solvent was evaporated under vacuo and purified by column chromatography using 5-40% EtOAc in hexane as eluent to afford 7b where R⁴ is C₉ alkyl (0.9 g, 83 %). ¹H NMR (300 MHz, d-chloroform) δ 4.88 -4.83 (m, 1H), 3.57 (m, 1H), 2.27 (t, J= 7.5 Hz, 2H), 1.61 (t, J= 7.5 Hz, 2H), 1.48 - 1.41 (m, 8H), 1.36 - 1.18 (m, 44H), 0.87 (t, J = 6.5 Hz, 9H).

### Heptadecan-9-yl 9-Hydroxynonadecanoate (7b Where R⁴ is C₁₀ Alkyl)

To a solution of heptadecan-9-yl 9-oxononadecanoate (6b where R⁴ is C₁₀ alkyl) (0.2 g, 0.36 mmol) in 3 mL of THF:DCM:MeOH (1:1:1) mixture was added NaBH₄ (0.03 g, 0.8 mmol) at 0° C. and stirred for 3 h under N₂ atmosphere. The reaction was quenched with 0.5 mL of H₂O and extracted with DCM. The organic phase was washed with brine and dried over anhydrous MgSO₄. Solvent was evaporated under vacuo and purified by column chromatography using 5-40% EtOAc in hexane as eluent to afford 7b where R⁴ is C₁₀ alkyl (0.16 g, 80 %). ¹H NMR (300 MHz, d-chloroform) δ 4.86 (t, J = 6.2 Hz, 1H), 3.58 (m, 1H), 2.27 (t, J = 7.5 Hz, 2H), 1.61-1.37 (m, 12H), 1.32 - 1.18 (m, 46H), 0.87 (t, J = 6.6 Hz, 9H).

### Heptadecan-9-yl 9-Hydroxyicosanoate (7b Where R⁴ is C₁₁ Alkyl)

To a solution of heptadecan-9-yl 9-oxoicosanoate (6b where R⁴ is C₁₁ alkyl) (0.27 g, 0.48 mmol) in 3 mL of THF:DCM:MeOH (1:1:1) mixture was added NaBH₄ (0.05 g, 1.35 mmol) at 0° C. and stirred for 3 h under N₂ atmosphere. The reaction was quenched with 0.5 mL of H₂O and extracted with DCM. The organic phase was washed with brine and dried over anhydrous MgSO₄. Solvent was evaporated under vacuo and purified by column chromatography using 5-40% EtOAc in hexane as eluent to afford 7b where R⁴ is C₁₁ alkyl (0.25 g, 92 %). ¹H NMR (301 MHz, d-chloroform) δ 4.86 (t, J = 6.2 Hz, 1H), 3.57 (s, 1H), 2.27 (t, J = 7.5 Hz, 2H), 1.69 - 1.37 (m, 12H), 1.29-1.17 (m, 48H), 0.87 (t, J = 6.5 Hz, 9H).

### Step 5: Synthesis of Heptadecan-9-yl 9-((4-(Dimethylamino)butanoyl)Oxy)Hexadecanoate (Lipid 81), Heptadecan-9-yl 9-((4-(Dimethylamino)butanoyl)Oxy)Heptadecanoate (Lipid 79), Heptadecan-9-yl 9-((4-(Dimethylamino)Butanoyl)Oxy)Octadecenoate (Lipid 77), Heptadecan-9-yl 9-((4-(Dimethylamino)Butanoyl)Oxy)Nonadecanoate (Lipid 78),or Heptadecan-9-yl 9-((4-(Dimethylamino)Butanoyl)Oxy)Icosanoate (Lipid 80)

### Heptadecan-9-yl 9-((4-(Dimethylamino)Butanoyl)Oxy)Hexadecanoate (Lipid 81)

To a solution of heptadecan-9-yl 9-hydroxyhexadecanoate (7b where R⁴ is C₇ alkyl) (0.25 g, 0.49 mmol) and 4-(dimethylamino)butanoic acid (0.125 g, 0.75 mmol) in DCM (5 mL), 0.27 mL of DIPEA was added. Then EDCI (0.143 g, 0.75 mmol), and DMAP (0.012 g, 0.1 mmol) were added, and the mixture was stirred overnight at room temperature under N₂ atmosphere. Next day, the reaction was diluted with DCM. The organic layer was washed with NaHCO₃ (aq) and dried over anhydrous Na₂SO₄. Solvent was evaporated under vacuo and purified by column chromatography using 0-5% MeOH in DCM as eluent to afford Lipid 81(0.14 g, 45 %). ¹H NMR (300 MHz, d-chloroform) δ 4.93 - 4.77 (m, 2H), 2.37 - 2.23 (m, 5H), 2.21 (s, 6H), 1.83-1.73 (m, 2H), 1.70 - 1.40 (m, 10H), 1.25 (s, 43H), 0.87 (t, J = 6.6 Hz, 9H). MS found 624.5 [M+H]⁺, calcd 623.59 for [C₃₉H₇₇NO₄].

### Heptadecan-9-yl 9-((4-(Dimethylamino)Butanoyl)Oxy)Heptadecanoate (Lipid 79)

To a solution of compound heptadecan-9-yl 9-hydroxyheptadecanoate (7b where R⁴ is C₈ alkyl) (0.21 g, 0.4 mmol) and 4-(dimethylamino)butanoic acid (0.08 g, 0.45 mmol) in DCM (3 mL), 0.16 mL of DIPEA was added. Then EDCI (0.09 g, 0.45 mmol), and DMAP (0.008 g, 0.06 mmol) were added, and the mixture was stirred overnight at room temperature under N₂ atmosphere. Next day, the reaction was diluted with DCM. The organic layer was washed with NaHCO₃ (aq) and dried over anhydrous Na₂SO₄. The solvent was evaporated under vacuo and purified by column chromatography using 0-5% MeOH in DCM as eluent to afford Lipid 79 (0.112 g, 44 %). ¹H NMR (300 MHz, d-chloroform) δ 4.86 (m, 2H), 2.34-2.24 (m, 5H), 2.21 (s, 6H), 1.78 (p, J = 7.6 Hz, 2H), 1.68 - 1.56 (m, 2H), 1.54 - 1.40 (m, 8H), 1.25 (s, 45H), 0.87 (t, J = 6.7 Hz, 9H). MS found 638.5 [M+H]⁺, calcd 637.60 for [C₄₀H₇₉NO₄].

### Heptadecan-9-yl 9-((4-(Dimethylamino)Butanoyl)Oxy)Octadecenoate (Lipid 77)

To a solution of heptadecan-9-yl 9-hydroxyoctadecanoate (7b where R⁴ is C₉ alkyl) (0.3 g, 0.56 mmol) in DCM (25 mL) and, EDCI (0.21 g, 1.12 mmol) and DMAP (0.07 g, 0.56 mmol) were added and stirred for 15 min under N₂ atmosphere. Then, 4-(dimethylamino)butanoic acid (0.25 g, 1.5 mmol) was added to the reaction mixture and stirred overnight. Next day, the solvent was evaporated and redissolved in EtOAc (300 mL). The organic layer was washed with H₂O (300 mL), NaHCO₃ (aq) (200 mL) and brine (200 mL) and dried over anhydrous Na₂SO₄. The solvent was evaporated under vacuo and purified by column chromatography using 5-40% EtOAc in hexane as eluent to afford Lipid 77 (0.124 g, 34 %). ¹H NMR (300 MHz, d-chloroform) δ 4.86 (m, 2H), 2.38 - 2.23 (m, 6H), 2.21 (s, 6H), 1.85 - 1.71 (m, 2H), 1.67 - 1.55 (m, 2H), 1.50-1.44 (m, 8H), 1.24 (s, 46H), 0.86 (t, J = 6.5 Hz, 9H). MS found 652.7 [M+H]⁺, calcd 651.62 for [C₄₁H₈₁NO₄].

### Heptadecan-9-yl 9-((4-(Dimethylamino)Butanoyl)Oxy)Nonadecanoate (Lipid 78)

To a solution of heptadecan-9-yl 9-hydroxynonadecanoate (7b where R⁴ is C₁₀ alkyl) (0.16 g, 0.29 mmol) in 1 mL DCM, EDCI (0.052 g, 0.27 mmol), and DMAP (0.04 g, 0.0.33 mmol) were added and stirred for 15 min under N₂ atmosphere. Then, 4-(dimethylamino)butanoic acid (0.056 g, 0.33 mmol) was added to the reaction mixture and stirred overnight. Next day, the reaction was diluted with DCM. The organic layer was washed with NaHCO₃ (aq) and dried over anhydrous MgSO₄. The solvent was evaporated under vacuo and purified by column chromatography using 0-5% MeOH in DCM as eluent to afford Lipid 78 (0.07 g, 36 %). ¹H NMR (300 MHz, d-chloroform) δ 4.93 - 4.81 (m, 2H), 2.34-2.24 (m, 5H), 2.22 (s, 6H), 1.85 - 1.67 (m, 4H), 1.63-1.57 (m, 2H), 1.48 (s, 7H), 1.24 (s, 47H), 0.87 (t, J = 6.6 Hz, 9H). MS found 665.63 [M+H]⁺, calcd 666.5 for [C₄₂H₈₃NO₄].

### Heptadecan-9-yl 9-((4-(Dimethylamino)Butanoyl)Oxy)Icosanoate (Lipid 80)

To a solution of compound heptadecan-9-yl 9-hydroxyicosanoate (7b where R⁴ is C₁₁ alkyl) (0.25 g, 0.44 mmol) in DCM (1 mL) and, EDCI (0.068 g, 0.36 mmol), and DMAP (0.054 g, 0.0.44 mmol) were added and stirred for 15 min under N₂ atmosphere. Then 4-(dimethylamino)butanoic acid (0.074 g, 0.44 mmol) was added to the reaction mixture and stirred overnight. Next day, the reaction was diluted with DCM. The organic layer was washed with NaHCO₃ (aq) and dried over anhydrous MgSO₄. The solvent was evaporated under vacuo and purified by column chromatography using 0-5% MeOH in DCM as eluent to afford Lipid 80 (0.134 g, 45 %). ¹H NMR (300 MHz, d-chloroform) δ 4.87-4.81 (m, 2H), 2.34-2.24 (m, 5H), 2.23 (d, J = 7.2 Hz, 6H), 1.87 - 1.76 (m, 2H), 1.74-1.70 (m, 2H), 1.65-1.57 (m, 2H), 1.48 (s, 7H), 1.24 (s, 50H), 0.87 (t, J = 6.6 Hz, 9H). MS found 680.6 [M+H]⁺, calcd 679.65 for [C₄₃H₈₅NO₄].

### Example 5: Synthesis of Ionizable Lipids of Formula XX

Lipids of Formula (XX) were designed and synthesized using similar synthesis methods depicted in Scheme 9 below. All variables in the compounds shown in Scheme 9, i.e., R¹, R², R³, R⁴, R^{6a}, R^{6b}, X, and n, are as defined in Formula (XX). R^{x} is R⁴ as defined but with one less carbon atom in the aliphatic chain.

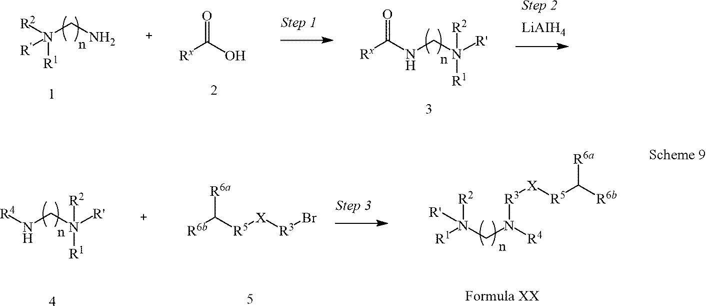

Monoester lipids of the present disclosure, i.e., Formula (XX) wherein X is —C(═O))—, were designed and synthesized using similar synthesis methods depicted in Scheme 10 below. All variables in the compounds shown in Scheme 9, i.e., R¹, R², R³, R⁴, R^{6a}, R^{6b}, X, and n, are as defined in Formula (XX). R^{x} is R⁴ as defined but with one less carbon atom in the aliphatic chain.

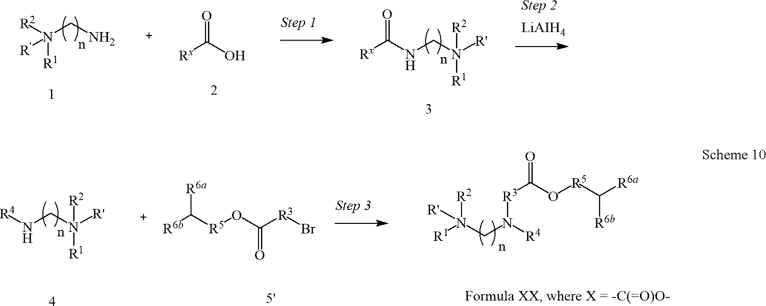

### Scheme 9 and Scheme 10

Referring to Scheme 9 and Scheme 10, at Step 1, to a stirred solution of the acid 2 in dichloromethane (DCM), was added 4-dimethylaminopyridine (DMAP) followed by 1-ethyl-3-(3-dimethylaminopropyl)carbodiimide (EDCI). The resulting mixture was stirred at room temperature for 15 min under nitrogen (N₂) atmosphere. Then, compound 1 was added dropwise and the mixture was stirred overnight. Next day, the reaction was diluted with DCM and washed with water and brine. The organic layer was dried over anhydrous sodium sulfate (Na₂SO₄) and, evaporated to dryness. The crude was purified by silica gel column chromatography using 0-10% methanol in DCM as eluent. The fractions containing the desired compound were pooled and evaporated to afford compound 3 (0.78 g, 54%).

At Step 2, to a solution of 3 in tetrahydrofuran (THF) was added lithium aluminum hydride (LiAlH₄). The reaction mixture was heated at 50° C. overnight. Next day, the reaction was cooled to 0° C. and water was added dropwise to quench. Subsequently, the reaction was filtered through Celite to get the crude product 4. The product was used in next step without further purification.

At Step 3, Compound 5 or 5′ (synthesized in accordance with the procedures described in International Patent Application Publication No. WO2017/49245, incorporated herein by reference in its entirety) was dissolved in of dimethylformamide/methanol mixture DMF:MeOH (1:1) and 4 was added. The reaction was stirred overnight at room temperature. The product was extracted with ethyl acetate (EtOAc) and the organic layer was washed with saturated sodium bicarbonate aqueous solution (NaHCO₃(aq)) and brine and dried over anhydrous Na₂SO₄. Solvent was evaporated under vacuo. and purified by column chromatography using 0-10% methanol in DCM as eluent to afford an ionizable lipid of Formula (XX) (where X is —C(═O)O)— is 5′ is used as reactant at Step 3).

### Synthesis of Lipid 102

Procedures for synthesizing Lipid 102 are described below with reference to Scheme 11, also provided below.

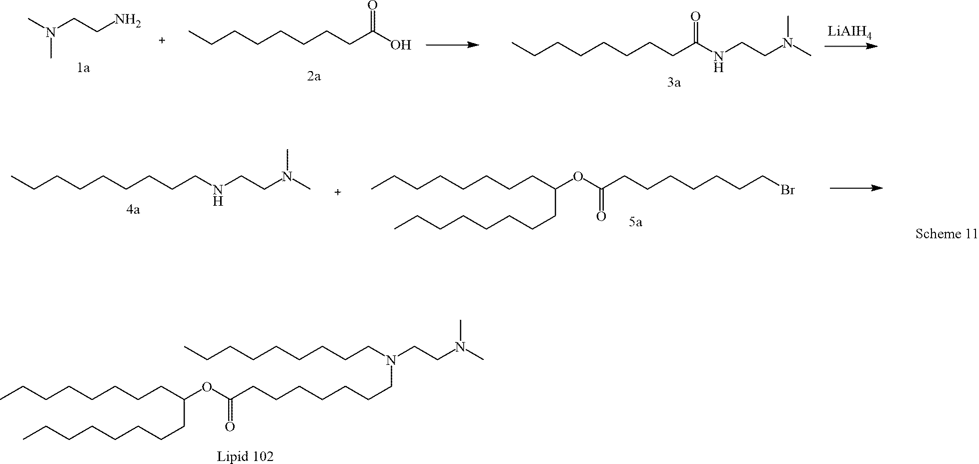

### Step 1: Synthesis of N-(2-(dimethylamino)ethyl)nonanamide (3a)

To a stirred solution of nonanoic acid (2a) (1.0 g, 6.3 mmol) in 60 mL of DCM, was added DMAP (0.91 g, 7.5 mmol) followed by EDCI (1.44 g, 7.5 mmol). The resulting mixture was stirred at room temperature for 15 min under N₂ atmosphere. Then, N¹,N¹-dimethylethane-1,2-diamine (1a) (0.66 g, 7.5 mmol) was added dropwise and the mixture was stirred overnight. Next day, the reaction was diluted with DCM and washed with H₂O and brine. The organic layer was dried over anhydrous Na₂SO₄ and, evaporated to dryness. The crude was purified by silica gel column chromatography using 0-10% methanol in DCM as eluent. The fractions containing the desired compound were pooled and evaporated to afford 3a (0.78 g, 54%).

### Step 2: Synthesis of N¹,N¹-Dimethyl—N2—Nonylethane-1,2-Diamine (4a)

To a solution of 3a (0.78 g, 3.4 mmol) in THF was added LiAlH₄. The reaction mixture was heated at 50° C. overnight. Next day, the reaction was cooled to 0° C. and water was added dropwise to quench. Subsequently, the reaction was filtered through Celite to get the crude product 4a (0.6 g, 82 %). The product was used in next step without further purification.

### Step 3: Synthesis of Lipid 102

Compound 5a (synthesized in accordance with the procedures described in International Patent Application Publication No. WO2017/49245, incorporated herein by reference in its entirety) (0.6 g, 1.3 mmol) was dissolved in 20 mL of DMF:MeOH (1:1) and 4a (0.35 g, 1.5 mmol) was added. The reaction was stirred overnight at room temperature. The product was extracted with EtOAc (200 mL) and the organic layer was washed with saturated NaHCO₃(aq) and brine and dried over anhydrous Na₂SO₄. Solvent was evaporated under vacuo. and purified by column chromatography using 0-10% methanol in DCM as eluent to afford Lipid 102 (0.062 g, 10 %). ¹H NMR (300 MHz, chloroform-d) δ 4.85 (quint, J = 6.2 Hz, 1H), 2.57 - 2.48 (m, 2H), 2.43 - 2.32 (m, 6H), 2.31 - 2.25 (m, J = 7.5 Hz, 2H), 2.23 (s, 6H), 1.66 - 1.34 (m, 8H), 1.24 (s, 47H), 0.86 (t, J = 6.6 Hz, 9H).

### Example 6: Preparation of Lipid Nanoparticle Formulations

### Preparation of Lipid Ethanol Stocks

ceDNA lipid nanoparticle (LNP) formulations (0.25 mg ceDNA-luciferase) comprising exemplary ionizable lipids described herein (e.g., Lipid A; Lipid 35, Lipid 37, and Lipid 39 that are encompassed by Formula (I) or Formula (I′); Lipid 57, Lipid 58, Lipid 61, and Lipid 62 that are encompassed by Formula (II)) were prepared as follows.

Ten G2 dialysis filters were soaked in 30% ethanol for 1-2 hours, then emptied, washed, and soaked in deionized H₂O until formulations were ready (> 3 hours). Individual lipid ethanol stocks were prepared the prior week and stored at -20° C. The concentrations of individual lipid ethanol stocks are shown in the table below. Each stock was prepared at 5x the desired concentration of the final mixture. Thus, to prepare the base lipid mixture, equal volumes of each stock were mixed together.

TABLE 9

Preparation of individual lipid ethanol stocks

| Lipid Name | Lipid Mass (mg) | Ethanol Volume (mL) | Ethanol mass (g) | Molecular Weight (Da) | Concentration (mM) | Mol % in LNP mixture |
|---|---|---|---|---|---|---|
| Ionizable Lipid | 150.1 | 16.8 | 13.2552 | 1173.8 | 7.611623623 | 0.507810797 |
| DOPC | 15.2 | 17.7 | 13.9653 | 786.1 | 1.092427251 | 0.072881474 |
| Chol | 38.0 | 17.0 | 13.4 | 386.7 | 5.780434749 | 0.385642712 |
| DMG-PEG₂₀₀₀ | 14.9 | 13.9 | 10.9671 | 2474 | 0.433283122 | 0.028906559 |
| DSPE-PEG₂₀₀₀ | 5.9 | 18.8 | 14.8332 | 4400 | 0.071324951 | 0.004758456 |
| TOTAL | - | - | - | - | 14.98909369 | 1 |

Ionizable lipid = any ionizable lipid described herein (e.g., Lipid A, Lipid 35, Lipid 37, Lipid 39, Lipid 57, Lipid 58, Lipid 61, Lipid 62, etc.); DOPC = dioleoylphosphatidylcholine; Chol = cholesterol; DMG-PEG₂₀₀₀ = 1,2-dimyristoyl-rac-glycero-3-methoxypolyethylene glycol-2000; DSPE-PEG₂₀₀₀ = 1,2-distearoyl-sn-glycero-3-phosphoethanolamine—N—[amino(polyethylene glycol)-2000].

### Preparation of LNP-ceDNA-Luciferase Formulations

Briefly and generally, to make 2.5 mL of an LNP lipid mixture containing any of the ionizable lipids described herein, 0.5 mL of each of the five different lipid stocks was added and mixed together. The molar percentage of each lipid component in the LNP lipid mixture is shown in Table 8.

The objective of Study A was to compare the effects of the standard aqueous process and the ethanol-based process of preparing Lipid A LNP formulations on particle size and encapsulation efficiency. A further objective of Study A was to evaluate whether the improvements attributed by the ethanol-based process, if any, would be observed if more components were added upon the base LNP formulations. To this end, 0.25 mL of ceDNA-luciferase (1.05 mg/mL) was added dropwise while the solution was gently swirled by hand until the solution was clear. This formed the lipid/ceDNA base formulation (with Lipid A) that was prepared using the alcohol-based process described herein, which is LNP 3 as shown in Table 9, with an intensity-based mean hydrodynamic diameter (Zave) of 64.2 nm.

To 2.5 mL of an equivolume mixture of the above stocks (0.5 mL of each lipid) was added first 34 uL of 10 mg/mL solution mPEG-C18 in EtOH, then 0.25 mL of ceDNA-luciferase (1.05 mg/mL) was added dropwise while the solution was gently swirled by hand until the solution was clear. This formed the lipid/ceDNA formulation (with Lipid A) + 2% mPEG-C18, which is LNP 4 as shown in Table 9, with a mean diameter of 55.2 nm.

To 2.5 mL of an equivolume mixture of the above stocks (0.5 mL of each lipid) was added was added first 69 uL of 10 mg/mL solution mPEG-C18 in EtOH, then 0.25 mL of ceDNA-luciferase (1.05 mg/mL) dropwise while the solution was gently swirled by hand until the solution was clear. This formed the lipid/ceDNA mixture formulation (with Lipid A) + 4% mPEG-C18, which is LNP 5 as shown in Table 9, with a mean diameter of 62.2 nm.

1.20 mg of b-sito was weighed in a small vial and added to a 20 mL vial. A solution of DOPE in chloroform (17 uL of a 25 mg/mL solution) was added to the vial and the chloroform was evaporated under a focused stream of N₂ gas (pipette). The vial was then stored in the vacuum desiccator for 2-3 hours. The dried lipids were dissolved in 1.0 mL of ethanol, then 0.5 mL of the ssOP lipid stock, 0.5 mL of the DMG-PEG₂₀₀₀ stock, and 0.5 mL of the GalNAc4 stock were added. 0.25 mL of ceDNA-luciferase (1.05 mg/mL) was then added to the solution dropwise while the solution was gently swirled by hand. This formed the lipid/ceDNA formulation (with Lipid A) + DOPE/b-sito, which is LNP 6 as shown in Table 9, with a mean diameter of 78.7 nm.

46 uL of a chloroform solution of mono-GalNAc (2.5 mg/mL) was added to a 20 mL vial. The chloroform was evaporated under a focused stream of N₂ gas (pipette). The vial was then stored in the vacuum desiccator for 2-3 hours. It was then dissolved in 0.5 mL of ethanol. 0.5 mL of each the SSOP, DOPC, Chol, and DMG-PEG₂₀₀₀ stocks were then added to the solution. 0.25 mL of ceDNA-luciferase (1.05 mg/mL) was then added to the solution. This formed the lipid/ceDNA formulation (with Lipid A) + 0.25% mono-GalNAc, which is LNP 7 in Table 10.

TABLE 10

Preparation of LNP-ceDNA-luciferase formulations for Study A

| LNP | LNP Descriptor | Process | Zave (nm) | PDI | Encapsulation Efficiency (%) |
|---|---|---|---|---|---|
| 1 | ceDNA-luciferase + Reference Lipid Z + DOPC + Chol + DMG-PEG₂₀₀₀ + DSPE-PEG₂₀₀₀ (control) | Standard (aqueous) | 90.7 | 0.092 | 79.4 |
| 2 | ceDNA-luciferase + Lipid A + DOPC + Chol + DMG-PEG₂₀₀₀ + DSPE-PEG₂₀₀₀ | Standard (aqueous) | 93.3 | 0.111 | 62.9 |
| 3 | ceDNA-luciferase + Lipid A + DOPC + Chol + DMG-PEG₂₀₀₀ + DSPE-PEG₂₀₀₀ | EtOH | 64.2 | 0.160 | 88.0 |
| 4 | ceDNA-luciferase + Lipid A + DOPC + Chol + DMG-PEG₂₀₀₀ + DSPE-PEG₂₀₀₀ + 2% mPEG-C18 | EtOH | 55.2 | 0.140 | 80.5 |
| 5 | ceDNA-luciferase + Lipid A + DOPC + Chol + DMG-PEG₂₀₀₀ + DSPE-PEG₂₀₀₀ + 4% mPEG-C18 | EtOH | 62.2 | 0.115 | 73.7 |
| 6 | ceDNA-luciferase + Lipid A + DOPC + Chol + DMG-PEG₂₀₀₀ + DSPE-PEG₂₀₀₀ + DOPE/b-sito | EtOH | 78.7 | 0.141 | 67.7 |
| 7 | ceDNA-luciferase + Lipid A + DOPC + Chol + DMG-PEG₂₀₀₀ + DSPE-PEG₂₀₀₀ + 0.25% mono-GalNAc | EtOH | 69.3 | 0.180 | 95.7 |

DOPC = dioleoylphosphatidylcholine; Chol = cholesterol; DMG-PEG₂₀₀₀= 1,2-dimyristoyl-rac-glycero-3-methoxypolyethylene glycol-2000; DSPE-PEG₂₀₀₀= 1,2-distearoyl-sn-glycero-3-phosphoethanolamine—N—[amino(polyethylene glycol)-2000]; mPEG-C18 = polyethylene glycol (mono)octadecyl; b-sito = betasitosterol; GalNAc═N—Acetylgalactosamine; mono-GalNAc = mono-antennary GalNAc.

As can be seen in Table 9, the control LNP 2 formulation containing Lipid A as the ionizable lipid and other lipid components, prepared using the standard aqueous process, had an intensity-based mean hydrodynamic diameter (Zave) of 93.3 nm and an encapsulation efficiency of 62.9%. In LNP 3 where the lipid and ceDNA compositions of the formulation were identical to those of LNP 2 but an ethanol-based process was used to prepare the formulation, the mean diameter of the particles was reduced to 64.2 nm and the encapsulation efficiency was increased to 88.0%. When additional components were added to the other LNP formulations also prepared using the ethanol-based process, such as mPEG-C18 in LNP 4 and LNP 5, DOPE/b-sito in LNP 6, and mono-GalNAc in LNP 7, smaller mean diameter measurements were also observed, thereby providing corroborating evidence to the hypothesis that the use of alcohol in the preparation of the LNP formulations had a compacting effect on the ceDNA, thereby resulting in LNPs having a smaller mean diameter. Further, increased encapsulation efficiencies were observed in LNP 4, LNP5, and LNP 7 that were prepared using the alcohol-based process. For the polydispersity index (PDI), in general PDI values that were about 0.15 or lower would be considered as satisfactory.

LNP-ceDNA-luciferase formulations containing all other ionizable lipids described herein, including the formulations used in Studies B-E described in Examples 6-9, were prepared using similar procedures as described above for the formulations containing Lipid A as the ionizable lipid. A GalNAc ligand such as mono-antennary GalNAc (mono-GalNAc), tri-antennary GalNAc (GalNAc3) or tetra-antennary GalNAc (GalNAc4) can be synthesized as known in the art (see, WO2017/084987 and WO2013/166121) and chemically conjugated to lipid or PEG as well-known in the art (see, Resen et al., J. Biol. Chem. (2001) “Determination of the Upper Size Limit for Uptake and Processing of Ligands by the Asialoglycoprotein Receptor on Hepatocytes in Vitro and in Vivo” 276:375577-37584).

### Example 7: Characterization of New Process of Precompaction

### Nanoassembling

Stock solutions of lipid in ethanol were made at the concentrations described in Table 8. The ionizable lipid in the ionizable lipid ethanol stock was Lipid A.

3.15 mL of each lipid ethanol stock was combined for the base lipid mixture (15.75 mL total). Each stock was 5x the desired concentration of the lipid in the final lipid mix.

0.3 mL of 1 mg/mL ceDNA-luciferase (1.05 mg/mL) was added to 3 mL of the lipid mixture. slowly with gentle swirling by hand. Final mixture was clear.

This mixture was then mixed with pH=4 malic acid buffer (no NaCl) on the NanoAssemblr at various flow rate ratios (FRR) as shown below:
FRR = 3:2, total volume 1 mL, into an 8 mL reservoir, malic acid buffer
FRR = 3:1, total volume 1.6 mL, into a 7.4 mL reservoir, malic acid buffer
FRR = 5:1, total volume 2.4 mL, into a 6.6 mL reservoir, malic acid buffer
FRR = 10:1, total volume 4.4 mL into a 4.6 mL reservoir, malic acid buffer

Specifically, each lipid/ceDNA mix was then mixed with 20 mM pH=4 malic acid (no NaCl) using the NanoAssemblr. A 3:1 flow rate ratio of malic acid buffer to lipid/ceDNA was utilized. A 3 mL syringe was used for the lipid/ceDNA mixture, a 10 mL syringe was used for the malic buffer. The NanoAssemblr outlet was collected in an empty 15 mL falcon tube, then immediately after the run added to a 50 mL falcon tube containing 10 mL of malic acid acid. The final ethanol content of each solution was ~12.5%, the final volume ~ 20 mL. The collection method described herein deviates from earlier trials, which were diluted to 4% ethanol and the outlet was dispensed directly into the diluent, which was not convenient for larger scale. Prior to nano assembling, a 40 ug trial with base lipid mixture was carried out with this modified collection procedure and found to also produce particles < 70 nm in a pre-dialysis DLS measurement.

Each sample was then split into two 10 mL G2 dialysis filters and dialyzed overnight into 1 xDPBS (5 L). The next day, the dialysis medium was changed two additional times.

The final ethanol content of each solution was ~ 4%. Dialysis was performed overnight, followed by standard process/ characterization. Analytics are shown below in Table 11. It can be seen from this table that as determined by DLS and at different flow rate ratios (FRR), the particle diameters were all smaller than 70 nm and the encapsulation efficiencies were higher than 85%.

TABLE 11

Dynamic light scattering (DLS) analytics of pre-compacted LNP formulations

| FRR | DLS Particle Diameter (nm) | Dispersity Index | Encapsulation Efficiency (%) |
|---|---|---|---|
| 3:2 | 67.4 | 0.199 | 87.5 |
| 3:1 | 65.9 | 0.191 | 87.3 |
| 5:1 | 66.5 | 0.164 | 86.4 |
| 10:1 | 65.9 | 0.155 | 83.7 |

### Analysis of Lipid Particle Formulations

Particle size was determined by dynamic light scattering (DLS).

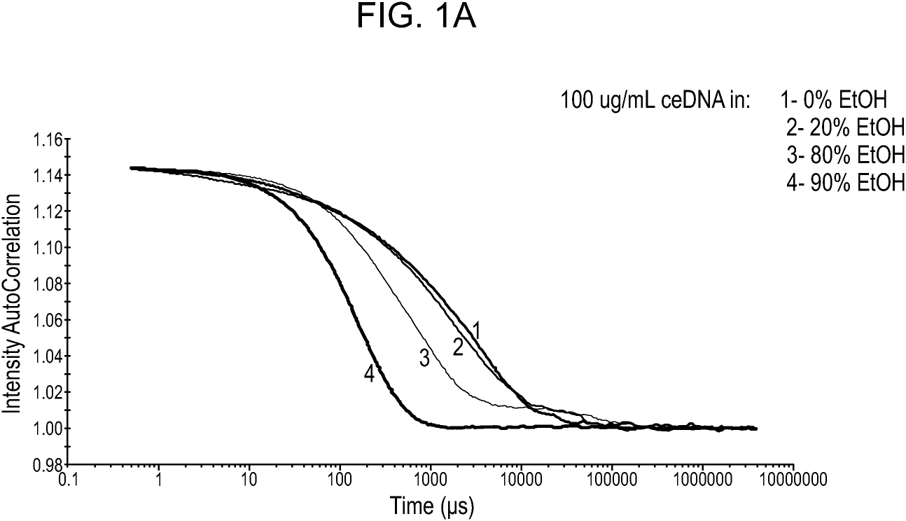

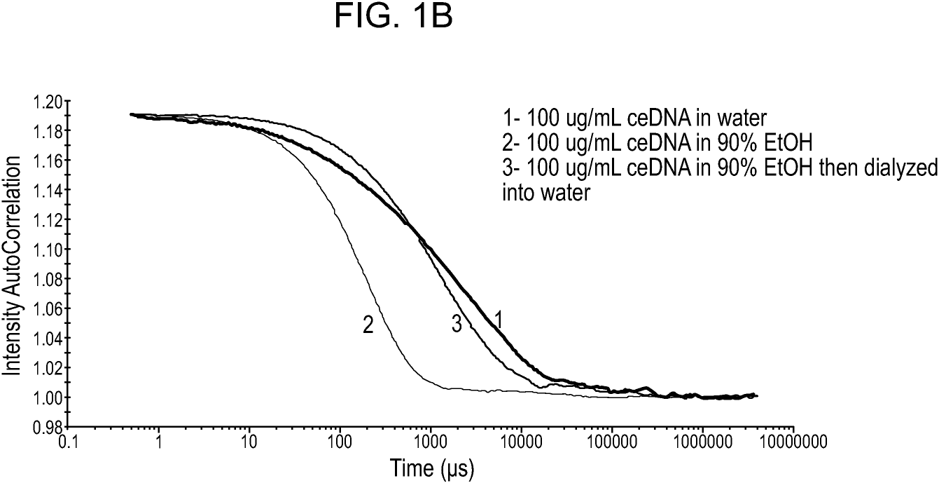

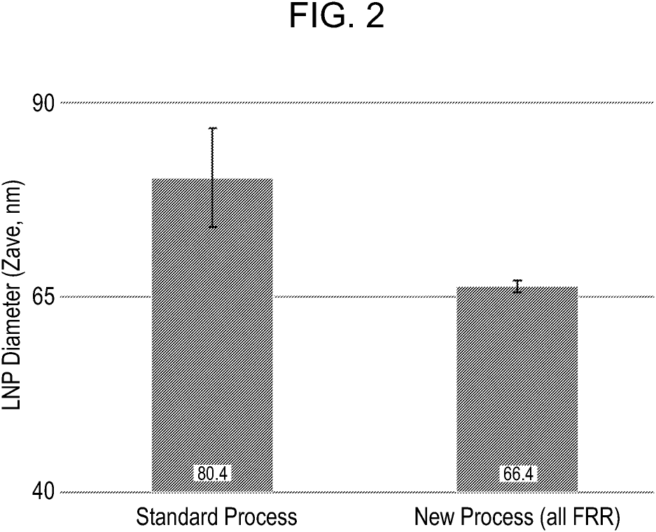

LNPs produced using the described method encapsulate > 80% of ceDNA that is 5.4 kbp (kilo-basepairs) long and possess a mean diameter of 66 nm. Statistical comparison of the LNP diameter produced using the new method (n = 4) and that of the old method (n = 28) leads to a high degree of confidence (P = 1.7 E -15) that this new process successfully reduces the LNP diameter, while maintaining similar or better ceDNA encapsulation efficiency relative to the standard process (FIG. 2). FIG. 1A is a graph that shows condensation of ceDNA as determined by dynamic light scattering. Dynamic light scattering correlation functions show condensation of ceDNA as ethanol content increases. FIG. 1B is a graph that shows compaction is reversible upon rehydration. No significant effect of the flow rate ratio on either the LNP diameter or the encapsulation efficiency of ceDNA was observed. Without wishing to be bound by theory, the improvements seen with the new process is likely due to compaction of ceDNA in 90-92% ethanol solvent prior to formation of LNPs. When the LNP formation is then initiated by mixing with the acidic aqueous buffer solution, the lipids are able to nucleate around a much smaller ceDNA core as opposed to the ‘standard’ process, resulting in significantly smaller particles.

Encapsulation of ceDNA in lipid particles was determined by Oligreen^{®} (Invitrogen Corporation; Carlsbad, Calif.) or PicoGreen^{®} (Thermo Scientific) kit. Oligreen® or PicoGreen^{®} is an ultra-sensitive fluorescent nucleic acid stain for quantitating oligonucleotides and single-stranded DNA or RNA in solution. Briefly, encapsulation was determined by performing a membrane-impermeable fluorescent dye exclusion assay. The dye was added to the lipid particle formulation. Fluorescence intensity was measured and compared to the fluorescence observed upon addition of a small amount of nonionic detergent. Detergent-mediated disruption of the lipid bilayer releases the encapsulated ceDNA, allowing it to interact with the membrane-impermeable dye. Encapsulation of ceDNA was calculated as E═ (I₀ — I)/I₀, where I₀ refers to the fluorescence intensities with the addition of detergent and I₀ refers to the fluorescence intensities without the addition of detergent.

Release of ceDNA from LNPs was determined. Endosome mimicking anionic liposome was prepared by mixing DOPS:DOPC:DOPE (mol ratio 1:1:2) in chloroform, followed by solvent evaporation at vacuum. The dried lipid film was resuspended in DPBS with brief sonication, followed by filtration through 0.45 µm syringe filer to form anionic liposome. Serum was added to LNP solution at 1:1 (vol/vol) and incubated at 37° C. for 20 min. The mixture was then incubated with anionic liposome at desired anionic/cationic lipid mole ratio in DPBS at either pH 7.4 or 6.0 at 37° C. for another 15 min. Free ceDNA at pH 7.4 or pH 6.0 was calculated by determining unencapsulated ceDNA content by measuring the fluorescence upon the addition of PicoGreen (Thermo Scientific) to the LNP slurry (C_{free}) and comparing this value to the total ceDNA content that was obtained upon lysis of the LNPs by 1% Triton X-100 (Cₜₒₜₐₗ), where % free = C_{free}/ Cₜₒₜₐₗ × 100. The % ceDNA released after incubation with anionic liposome is calculated based on the equation below:

$$\begin{matrix}\% \text{ceDNA released =} \\ {\text{\% free ceDNA}}_{\text{mixed with anionic liposome}} {\text{- \% free ceDNA}}_{\text{mixed with DPBS}}\end{matrix}$$

The pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which were incorporated by reference in their entirety). The preferred range of pKa is ~5 to ~ 7. The pKa of each cationic lipid was determined in lipid nanoparticles using an assay based on fluorescence of 2-(p-toluidino)-6-napthalene sulfonic acid (TNS). Lipid nanoparticles comprising cationic lipid/DOPC/cholesterol/PEG-lipid (51/7.5/38.5/3 mol %) in DPBS at a concentration of 0.4 mM total lipid were prepared using the in-line process as described herein and elsewhere. TNS was prepared as a 100 µM stock solution in distilled water. Vesicles were diluted to 24 µM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution was added to give a final concentration of 1 µM and following vortex mixing fluorescence intensity was measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

Binding of the lipid nanoparticles to ApoE will be determined as follows. LNP (10 µg/mL of ceDNA) is incubated at 37° C. for 20 min with equal volume of recombinant ApoE3 (500 µg/mL) in DPBS. After incubation, LNP samples are diluted 10-fold using DPBS and will be analyzed by heparin sepharose chromatography on AKTA pure 150 (GE Healthcare).

### Example 8: Study B - Varying GalNAc Amounts in Lipid A LNP Formulations

The objective of Study B was to evaluate the effects of varying tetra-antennary GalNAc (GalNAc4) amounts in Lipid A LNP formulations (prepared using the ethanol-based process) on particle size and encapsulation efficiency. Table 12 shows the compositions and mol ratios of the LNP formulations studied and their mean diameter (Zave), polydispersity index (PDI), and encapsulation efficiency (EE).

TABLE 12

LNP formulations in Study B

| LNP | Components of LNP (mol ratio) and Process | Zave (nm) | PDI | EE (%) |
|---|---|---|---|---|
| 8 | Lipid A: DOPC : Chol : DMG-PEG₂₀₀₀ : DSPE-PEG₂₀₀₀-GalNAc4 50.7 : 7.2 : 38.6 : 2.9 : 0.48 (standard aqueous process, control) | 95.8 | 0.085 | 73.6 |
| 9 | Lipid A: DOPC : Chol : DMG-PEG₂₀₀₀ : DSPE-PEG₂₀₀₀-GalNAc4 50.7 : 7.2 : 38.6 : 2.9 : 0.48 (EtOH process) | 67.9 | 0.067 | 87.1 |
| 10 | Lipid A: DOPC : Chol : DMG-PEG₂₀₀₀ : DSPE-PEG₂₀₀₀-GalNAc4 50.8 : 7.3 : 38.7 : 2.9 : 0.24 (EtOH process) | 73.4 | 0.050 | 88.6 |
| 11 | Lipid A: DOPC : Chol : DMG-PEG₂₀₀₀ : DSPE-PEG₂₀₀₀-GalNAc4 50.9 : 7.3 : 38.8 : 2.9 : 0.10 (EtOH process) | 77.7 | 0.034 | 89.8 |
| 12 | Lipid A: DOPC : Chol : DMG-PEG₂₀₀₀ : DSPE-PEG₂₀₀₀-GalNAc4 50.9 : 7.2 : 38.8 : 2.9 : 0.05 (EtOH process) | 76.8 | 0.026 | 90.6 |

DOPC = dioleoylphosphatidylcholine; Chol = cholesterol; DMG-PEG₂₀₀₀ = 1,2-dimyristoyl-rac-glycero-3-methoxypolyethylene glycol-2000; DSPE-PEG₂₀₀₀ = 1,2-distearoyl-sn-glycero-3-phosphoethanolamine—N—[amino(polyethylene glycol)-2000]; GalNAc = N-Acetylgalactosamine; GalNAc4 = tetra-antennary GalNAc. Note: LNP 9, LNP 10, LNP 11, and LNP 12 each have an N/P ratio of ~9.3.

The results in Table 12 indicate that when the ethanol-based process was used to prepare LNP 9 having 0.48% DSPE-PEG₂₀₀₀-GalNAc4 instead of the aqueous process (i.e., LNP 8), the mean diameter was reduced from 95.8 nm to 67.9 nm while the encapsulation efficiency was increased from 73.6% to 87.1%. The reduction in mean diameter size and increase in encapsulation efficiency were consistently observed in LNP 10, LNP 11, and LNP 12 that respectively contain 0.24%, 0.10%, and 0.05% DSPE-PEG₂₀₀₀-GalNAc4.

### Example 9: Study C - Formula (I) or Formula (I′) LNP Formulations Prepared Using the Ethanol-Based Process

The objective of Study C was to compare the physical properties of representative Formula (I) or Formula (I′) LNP formulations prepared using the standard aqueous process or the ethanol-based process (EtOH 92%) as described in Example 6. Table 13 shows the compositions and mol ratios of the LNP formulations studied and their mean diameter (Zave), polydispersity index (PDI), and encapsulation efficiency (EE).

TABLE 13

LNP formulations in Study C

| LNP | Components of LNP (mol ratio) and Process | Zave (nm) | PDI | EE (%) |
|---|---|---|---|---|
| 13 | Lipid A: DOPC : Chol : DMG-PEG₂₀₀₀ : DSPE-PEG₂₀₀₀-GalNAc4 50.7 : 7.3 : 38.6 : 2.9 : 0.48 (standard aqueous process, control) | 84.1 | 0.089 | 69.3 |
| 14 | Lipid 35 : DOPC : Chol : DMG-PEG₂₀₀₀ : DSPE-PEG₂₀₀₀-GalNAc4 50.7 : 7.3 : 38.6 : 2.9 : 0.48 (standard aqueous process) | 73.6 | 0.135 | 32.1 |
| 15 | Lipid 35 : DOPC : Chol : DMG-PEG₂₀₀₀ : DSPE-PEG₂₀₀₀-GalNAc4 50.7 : 7.3 : 38.6 : 2.9 : 0.48 (EtOH process) | 72.2 | 0.198 | 82.2 |
| 16 | Lipid 37 : DOPC : Chol : DMG-PEG₂₀₀₀ : DSPE-PEG₂₀₀₀-GalNAc4 50.7 : 7.3 : 38.6 : 2.9 : 0.48 (standard aqueous process) | 71.4 | 0.105 | 45.4 |
| 17 | Lipid 37 : DOPC : Chol : DMG-PEG₂₀₀₀ : DSPE-PEG₂₀₀₀-GalNAc4 50.7 : 7.3 : 38.6 : 2.9 : 0.48 (EtOH process) | 69.8 | 0.193 | 80.7 |
| 18 | Lipid 39 : DOPC : Chol : DMG-PEG₂₀₀₀ : DSPE-PEG₂₀₀₀-GalNAc4 50.7 : 7.3 : 38.6 : 2.9 : 0.48 (standard aqueous process) | 79.2 | 0.105 | 51.6 |
| 19 | Lipid 39 : DOPC : Chol : DMG-PEG₂₀₀₀ : DSPE-PEG₂₀₀₀-GalNAc4 50.7 : 7.3 : 38.6 : 2.9 : 0.48 (EtOH process) | 68.7 | 0.231 | 34.8 |

DOPC = dioleoylphosphatidylcholine; Chol = cholesterol; DMG-PEG₂₀₀₀ = 1,2-dimyristoyl-rac-glycero-3-methoxypolyethylene glycol-2000; DSPE-PEG₂₀₀₀ = 1,2-distearoyl-sn-glycero-3-phosphoethanolamine—N—[amino(polyethylene glycol)-2000]; GalNAc = N-Acetylgalactosamine; GalNAc4 = tetra-antennary GalNAc.

The results in Table 13 indicate that when the ethanol-based process was used to prepare LNP formulations having Lipid 35 (i.e., LNP 15), Lipid 37 (i.e., LNP 17), or Lipid 39 (i.e., LNP 19), reduced diameter sizes were observed consistently in all formulations, as compared to their corresponding formulations prepared using the aqueous process (i.e., LNP 14, LNP 16, and LNP 18, respectively). Of note, the mean diameter sizes of LNP 15, LNP 17, and LNP 19 were all smaller than 75 nm. Additionally, for Lipid 35 and Lipid 37, the encapsulation efficiency was significantly improved in LNP formulations prepared using the ethanol-based process.

### Example 10: Study D - Formula (II) LNP Formulations Prepared Using the Ethanol-Based Process

The objective of Study D was to compare the physical properties of representative Formula (II) LNP formulations prepared using the standard aqueous process or the ethanol-based process (EtOH 92%) as described in Example 6. Table 14 and Table 15 show the compositions and mol ratios of the LNP formulations studied and their mean diameter (Zave), polydispersity index (PDI), and encapsulation efficiency (EE).

TABLE 14

LNP formulations in Study D (Part I)

| LNP | Components of LNP (mol ratio) and Process | Zave (nm) | PDI | EE (%) |
|---|---|---|---|---|
| 20 | Lipid A: DOPC : Chol : DMG-PEG₂₀₀₀ : DSPE-PEG₂₀₀₀-GalNAc4 50.7 : 7.3 : 38.6 : 2.9 : 0.50 (standard aqueous process, control) | 84.2 | 0.056 | 72.2 |
| 21 | Lipid 57 : DOPC : Chol : DMG-PEG₂₀₀₀ : DSPE-PEG₂₀₀₀ 50.7 : 7.3 : 38.6 : 2.9 : 0.48 (standard aqueous process) | 83.8 | 0.079 | 62.0 |
| 22 | Lipid 57 : DOPC : Chol : DMG-PEG₂₀₀₀ : DSPE-PEG₂₀₀₀ 50.7 : 7.3 : 38.6 : 2.9 : 0.48 (EtOH process) | 70.0 | 0.113 | 72.8 |
| 23 | Lipid 58 : DOPC : Chol : DMG-PEG₂₀₀₀ : DSPE-PEG₂₀₀₀ 50.7 : 7.3 : 38.6 : 2.9 : 0.48 (standard aqueous process) | 82.8 | 0.085 | 60.1 |
| 24 | Lipid 58 : DOPC : Chol : DMG-PEG₂₀₀₀ : DSPE-PEG₂₀₀₀ 50.7 : 7.3 : 38.6 : 2.9 : 0.48 (EtOH process) | 73.9 | 0.101 | 78.2 |

DOPC = dioleoylphosphatidylcholine; Chol = cholesterol; DMG-PEG₂₀₀₀ = 1,2-dimyristoyl-rac-glycero-3-methoxypolyethylene glycol-2000; DSPE-PEG₂₀₀₀ = 1,2-distearoyl-sn-glycero-3-phosphoethanolamine—N—[amino(polyethylene glycol)-2000]; GalNAc = N-Acetylgalactosamine; GalNAc4 = tetra-antennary GalNAc

TABLE 15

LNP formulations in Study D (Part II)

| | | | | |
|---|---|---|---|---|
| LNP | Components of LNP (mol ratio) and Process | Zave (nm) | PDI | EE (%) |
| 25 | Lipid A : DOPC : Chol : DMG-PEG₂₀₀₀ : DSPE-PEG₂₀₀₀-GalNAc4 50.7 : 7.3 : 38.6 : 2.9 : 0.48 (standard aqueous process, control) | 85.8 | 0.065 | 64.8 |
| 26 | Lipid 61 : DOPC : Chol : DMG-PEG₂₀₀₀ : DSPE-PEG₂₀₀₀ 50.7 : 7.3 : 38.6 : 2.9 : 0.48 (standard aqueous process) | 94.3 | 0.080 | 65.2 |
| 27 | Lipid 61 : DOPC : Chol : DMG-PEG₂₀₀₀ : DSPE-PEG₂₀₀₀ 50.7 : 7.3 : 38.6 : 2.9 : 0.48 (EtOH process) | 77.7 | 0.141 | 72.0 |
| 28 | Lipid 62 : DOPC : Chol : DMG-PEG₂₀₀₀ : DSPE-PEG₂₀₀₀ 50.7 : 7.3 : 38.6 : 2.9 : 0.48 (standard aqueous process) | 84.5 | 0.069 | 68.6 |
| 29 | Lipid 62 : DOPC : Chol : DMG-PEG₂₀₀₀ : DSPE-PEG₂₀₀₀ | 77.5 | 0.126 | 72.9 |
| | 50.7 : 7.3 : 38.6 : 2.9 : 0.48 (EtOH process) | | | |

DOPC = dioleoylphosphatidylcholine; Chol = cholesterol; DMG-PEG₂₀₀₀ = 1,2-dimyristoyl-rac-glycero-3-methoxypolyethylene glycol-2000; DSPE-PEG₂₀₀₀ = 1,2-distearoyl-sn-glycero-3-phosphoethanolamine—N—[amino(polyethylene glycol)-2000]; GalNAc = N-Acetylgalactosamine; GalNAc4 = tetra-antennary GalNAc

The results in Tables 14 and 15 indicate that when the ethanol-based process was used to prepare LNP formulations having Lipid 57 (i.e., LNP 22), Lipid 58 (i.e., LNP 24), Lipid 61 (i.e., LNP 27), or Lipid 62 (i.e., LNP 29), reduced diameter sizes and increased encapsulation efficiencies were observed consistently in all formulations, as compared to their corresponding formulations prepared using the aqueous process (i.e., LNP 21, LNP 23, LNP 26, and LNP 28, respectively). Of note, the mean diameter sizes of LNP 22 and LNP 24 were smaller than 75 nm.

### Example 11: Study E - Formula (XV) LNP Formulations Prepared Using the LMW Alcohol-Based Process

The objective of Study E was to compare the physical properties of representative Formula (XV) LNP formulations prepared using the standard aqueous process or the LMW alcohol-based process (EtOH—MeOH; 1:1 ratio in total concentration of 95%) similar to the description in Example 6. Briefly, in the standard aqueous process, lipids in EtOH—MeOH solution was mixed with aqueous buffer containing ceDNA in NanoAssemblr to form LNP (one channel introducing lipids and the other channel introducing ceDNA in aqueous buffer). In the LMW alcohol-based process, ceDNA and lipids were pre-mixed in a solution having the final concentration of 95% LMW alcohol (EtOH—MeOH (1:1)) similar to the mixture formation described in Example 6 and the resultant 95% LMW alcohol mixture containing ceDNA and lipids was introduced to NanoAssemblr through one channel and the aqueous buffer (20 mM pH=4 malic acid (no NaCl)) was introduced to NanoAssemblr via another channel to create LNPs encapsulating ceDNA. Table 16 shows the compositions and mol ratios of the LNP formulations studied and their mean diameter (Zave), polydispersity index (PDI), and encapsulation efficiency (EE).

TABLE 16

LNP formulations in Study E

| LNP | Components of LNP (mol ratio) and Process | Zave (nm) | PDI | EE (%) |
|---|---|---|---|---|
| 30 | Reference Lipid Z : DSPC : Chol : DMG-PEG₂₀₀₀ 47.5 : 10.0 : 40.7 : 1.8 (standard aqueous process, control) | 78.6 | 0.09 | 95.9 |
| 31 | Lipid 77 : DSPC : Chol : DMG-PEG₂₀₀₀ 47.5 : 10.0 : 40.7 : 1.8 (standard aqueous process) | 75.5 | 0.132 | 98.8 |
| 32 | Lipid 77 : DSPC : Chol : DMG-PEG₂₀₀₀ 47.5 : 10.0 : 40.7 : 1.8 (EtOH—MeOH process) | 71.8 | 0.019 | 98.0 |
| 33 | Lipid 77 : DSPC : Chol : DMG-PEG₂₀₀₀ 47.5 : 10.0 : 39.5 : 3.0 | 75.4 | 0.179 | 94.5 |
| | (standard aqueous process) | | | |
| 34 | Lipid 77 : DSPC : Chol : DMG-PEG₂₀₀₀ 47.5 : 10.0 : 39.5 : 3.0 (EtOH—MeOH process) | 58.9 | 0.096 | 97.8 |
| 35 | Lipid 77 : DSPC : Chol : DMG-PEG₂₀₀₀ 47.5 : 10.0 : 40.2 : 2.3 (EtOH process) | 62.9 | 0.096 | 98.4 |

DSPC = distearoylphosphatidylcholine; Chol = cholesterol; DMG-PEG₂₀₀₀ = 1,2-dimyristoyl-rac-glycero-3-methoxypolyethylene glycol-2000.

The results in Table 16 indicate that when the LWM alcohol-based process (EtOH—MeOH; 1:1) %) was used to prepare an LNP formulation having Lipid 77 (i.e., LNP 32 and LNP 34), reduced diameter sizes and increased encapsulation efficiencies were observed in LNP 32 and LNP 34, as compared to its corresponding formulations prepared using the aqueous process (i.e., LNP 31 and LNP 33). Consistently, the LNP formulation having Lipid 77 and 2.3% of DMG-PEG₂₀₀₀ and prepared using the ethanol-based process had a mean diameter size of less than 75 nm.

### Example 12: In Vitro Phagocytosis Assay for Functional Assessment of Formulations

An in vitro phagocytosis assay will be performed using the ceDNA lipid nanoparticle (LNP) formulations comprising MC3, MC3-5% DSG-PEG2000 (1,2-Distearoyl-rac-glycero-3-methylpolyoxyethylene) (abbreviated as “5DSG”) produced using the processes described herein, with ss-OP4 as the cationic lipid component and optionally GalNac, a liver specific ligand.

A phagocytosis assay will be carried out for ceDNA LNPs treated with 0.1% DiD (DiIC18(5); 1,1′-dioctadecyl-3,3,3′,3′-tetramethylindodicarbocyanine, 4-chlorobenzenesulfonate salt) lipophilic carbocyanine dye. Various concentrations of ceDNA will be used in the LNPs, in the presence or absence of 10% human serum (+ serum), which will then be introduced to macrophage differentiated from THP-1 cells.

Phagocytic cells that internalize ceDNA will appear in red fluorescence. It is expected that the ss-OP4 LNPs comprising ceDNA will be highly associated with the lowest number of fluorescent phagocytotic cells. Thus, without being bound by theory, it is thought that the ss-OP4 LNPs will be better able to avoid phagocytosis by immune cells as compared to the MC3-5DSG and MC3 LNPs. Phagocytosis will be quantified by red object count/ % confluence.

It is expected that the ceDNA-LNPs comprising a mean diameter of 60 nm to 75 nm will exhibit greater hepatocyte targeting compared to ceDNA-LNP having a mean diameter greater than 75 nm.

### Example 13: Pre-Clinical In Vivo Studies of LNP Formulations

Pre-clinical studies were also carried out in each of Studies A-E in order to evaluate the in vivo expression of ceDNA-luciferase and the tolerability of and the LNP formulations in mice. The study design and procedures involved in these pre-clinical studies are as described below.

### Materials and Methods

TABLE 17

Blood Collection

| Group | Sample Collection Times |
|---|---|
| | Whole Blood (Tail, saphenous or orbital) |
| | SERUM^{a} |
| | Day 0 about 5 - 6 hours post Test Material dose (no less than 5.0 hours, no more than 6.5 hours) |
| Volume / Portion | about 150 µL whole blood |
| Processing / Storage | 1 aliquot frozen at nominally -70° C. |

^{a}Whole blood was collected into serum separator tubes, with clot activator Species (number, sex, age): CD-1 mice (N = 65 and 5 spare, male, about 4 weeks of age at arrival).

Cage Side Observations: Cage side observations were performed daily.

Clinical Observations: Clinical observations were performed about 1, about 5 to about 6 and about 24 hours post the Day 0 Test Material dose. Additional observations were made per exception. Body weights for all animals, as applicable, were recorded on Days 0, 1, 2, 3, 4 & 7 (prior to euthanasia). Additional body weights were recorded as needed.

Dose Administration: Test articles (LNPs: ceDNA-Luc) were dosed at 5 mL/kg on Day 0 by intravenous administration to lateral tail vein.

In-life Imaging: On Day 4, all animals in were dosed with luciferin at 150 mg/kg (60 mg/mL) via intraperitoneal (IP) injection at 2.5 mL/kg. ≤15 minutes post each luciferin administration; all animals had an IVIS imaging session according to in vivo imaging protocol described below.

Anesthesia Recovery: Animals were monitored continuously while under anesthesia, during recovery and until mobile.

Interim Blood Collection: All animals had interim blood collected on Day 0; 5-6 hours post-test (no less than 5.0 hours, no more than 6.5 hours).

After collection animals received 0.5 - 1.0 mL lactated Ringer’s; subcutaneously.

Whole blood for serum were collected by tail-vein nick, saphenous vein or orbital sinus puncture (under inhalant isoflurane). Whole blood was collected into a serum separator with clot activator tube and processed into one (1) aliquot of serum.

### In Vivo Imaging Protocol

Luciferin stock powder was stored at nominally -20° C.
Stored formulated luciferin in 1 mL aliquots at 2 - 8° C. protect from light.
Formulated luciferin was stable for up to 3 weeks at 2 - 8° C., protected from light and stable for about 12 h at room temperature (RT).
Dissolved luciferin in PBS to a target concentration of 60 mg/mL at a sufficient volume and adjusted to pH=7.4 with 5-M NaOH (about 0.5 µl/mg luciferin) and HCl (about 0.5µL/mg luciferin) as needed.
Prepared the appropriate amount according to protocol including at least a about 50% overage.

### Injection and Imaging

Shaved animal’s hair coat (as needed).
Per protocol, injected 150 mg/kg of luciferin in PBS at 60 mg/mL via IP.
Imaging was performed immediately or up to 15 minutes post dose.
Set isoflurane vaporizer to 1 - 3 % (usually 2.5%) to anesthetize the animals during imaging sessions.
Isoflurane anesthesia for imaging session:
   Placed the animals into the isoflurane chamber and wait for the isoflurane to take effect, about 2-3 min.
   Ensured that the anesthesia level on the side of the IVIS machine was positioned to the “on” position.
   Placed animal(s) into the IVIS machine

Performed desired Acquisition Protocol with settings for highest sensitivity.

### Results

All LNP formulations prepared using the ethanol-based (92% EtOH) or LMW alcohol-based process (95% EtOH—MeOH (1:1) for ceDNA and lipids as a premix prior to LNP formation in Nanoassemblr) and used in Studies A-E and as described in Examples 4 and 6-9 exhibited satisfactory or equivalent luciferase expression (IVIS measured at Day 4 after administration) compared to their corresponding formulations that were prepared using the standard aqueous process. In terms of tolerability, all LNP formulations prepared using the ethanol-based process and used in Studies A-E and as described in Examples 6 and 8-11 exhibited excellent tolerability with no significant changes in the mice body weight as measured at Day 1 after treatment.

### Example 14: Transmission Electron Microscopy (TEM) of ceDNA and Plasmid DNA

Transmission electron microscopy (TEM) was used to explore the morphology of ceDNA and plasmid DNA (pDNA) stored in different conditions (e.g., deionized (DI) water, 91% 1:1 EtOH:MeOH in DI; 100 mM NaOH; 100% 50:50 EtOH—MeOH). Without being bound by theory, the inventors hypothesized that ceDNA and pDNA treated with an alcohol/water solution or pure alcohol solvent results in the denaturation of the nucleic acid to a conformation that enhances encapsulation efficiency by LNP and produces LNP formulations having a smaller diameter size (i.e., smaller than 75 nm ± 3 nm). Briefly, each sample was applied onto a grid, washed with a buffer and then the sample was stained with 0.06% uranyl acetate in methanol. The grid was then placed directly into the grid box, allowed to try before observation under the microscope.

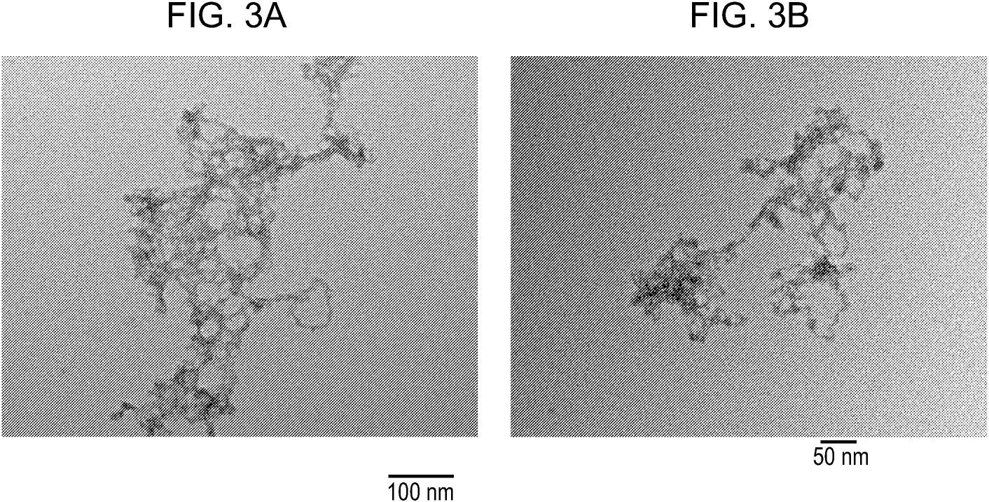

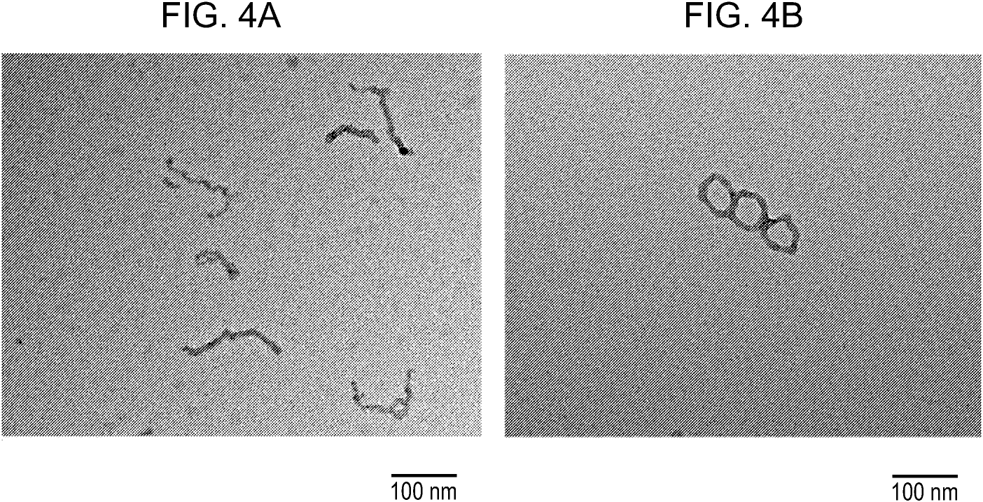

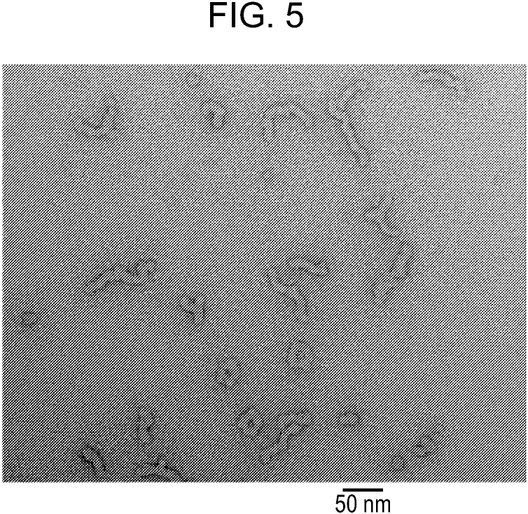

The TEM images shown in FIGS. 3A and 3B show that both ceDNA and pDNA (plasmid) exhibited mostly aggregated or self-entangled shape with some strand-like structures when the nucleic acid samples were stored in deionized water. When stored in a low molecular weight alcohol/water solution of 90.9% 1:1 ethanol:methanol in deionized water, the ceDNA sample formed a distinct rod-like structure (see FIG. 4A) and the pDNA formed a circular structure (see FIG. 4B). A ceDNA sample stored in 100% low molecular weight alcohol (i.e., 1:1 ethanol:methanol with no water) was also visualized by TEM and the ceDNA was seen to exhibited slightly thicker rods (see FIG. 5) than the sample stored in the alcohol/water solution described above. Furthermore, ceDNA and pDNA samples stored in 100 nM NaOH were also examined under the microscope. FIGS. 6A and 6B indicate that both nucleic acid samples remained mostly unchanged in a basic condition as compared to storage in deionized water.

### REFERENCES

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

What is claimed is:

1. A pharmaceutical composition comprising lipid nanoparticle (LNP), wherein the LNP comprises a lipid and a rigid therapeutic nucleic acid (rTNA), wherein the mean diameter of the LNP is between about 20 nm and about 75 nm.

2. The pharmaceutical composition of claim 1, wherein the rigid therapeutic nucleic acid is a closed-ended DNA (ceDNA).

3. The pharmaceutical composition of claim 1, wherein the rigid therapeutic nucleic acid is a double stranded nucleic acid.

4. The pharmaceutical composition of any one of claims 1-3, wherein the lipid is selected from an ionizable lipid, a non-cationic lipid, a sterol or a derivative thereof, a PEGylated lipid, or any combination thereof.

5. The pharmaceutical composition of claim 4, wherein the ionizable lipid is a cationic lipid.

6. The pharmaceutical composition of claim 4, wherein the cationic lipid is an SS-cleavable lipid.

7. The pharmaceutical composition of claim 5 or claim 6, wherein the cationic lipid is represented by Formula (I): 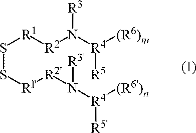
or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl;
or alternatively, when R²is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or —[C(R^{a})₂]₂CR^{a};
R^{a}, for each occurrence, is independently H or C₁₋₃ alkyl;
or alternatively, when R⁴is —C(R^{a})₂CR^{a}, or —[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or —[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{5′} are each independently C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

8. The pharmaceutical composition of claim 5 or claim 6, wherein the cationic lipid is represented by Formula (II): 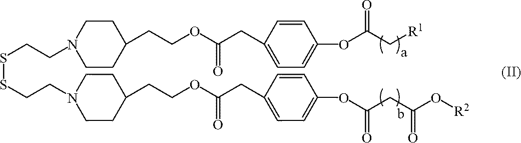
or a pharmaceutically acceptable salt thereof, wherein:
a is an integer ranging from 1 to 20;
b is an integer ranging from 2 to 10;
R¹ is absent or is selected from (C₂-C₂₀)alkenyl, -C(O)O(C₂-C₂₀)alkyl, and cyclopropyl substituted with (C₂-C₂₀)alkyl; and
R² is (C₂-C₂₀)alkyl.

9. The pharmaceutical composition of claim 5 or claim 6, wherein the lipid is represented by the Formula (V): 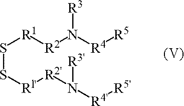
or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5’} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

10. The pharmaceutical composition of claim 5, wherein the cationic lipid is represented by Formula (XV): 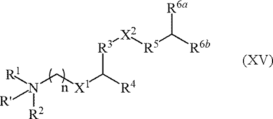
or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C1₆ unbranched alkyl, C₂-C₁₆ unbranched alkenyl, or 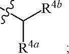
wherein:
R^{4a} and R^{4b} are each independently C₁-C₁₆ unbranched alkyl or C₂-C₁₆ unbranched alkenyl;
R⁵ is absent, C₁-C₈ alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6.

11. The pharmaceutical composition of claim 5, wherein the cationic lipid is represented by Formula (XX): 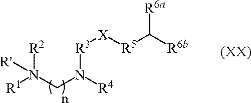
or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆ unbranched alkenyl, or 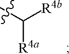
wherein:
R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6.

12. The pharmaceutical composition of claim 5 or claim 6, wherein the cationic lipid is selected from any lipid in Table 2, Table 5, Table 6, Table 7, or Table 8.

13. The pharmaceutical composition of claim 5 or claim 6, wherein the cationic lipid is a lipid having the structure: 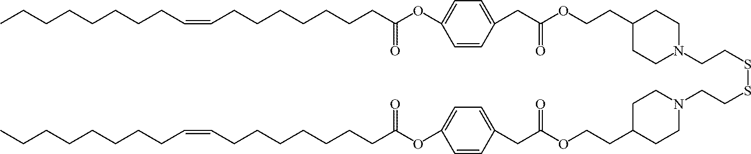
or a pharmaceutically acceptable salt thereof.

14. The pharmaceutical composition of claim 5 or claim 6, wherein the cationic lipid is MC3 (6Z,9Z,28Z,31Z)-heptatriaconta-6,9,28,31-tetraen-19-yl-4-(dimethylamino) butanoate (DLin-MC3-DMA or MC3) having the following structure: 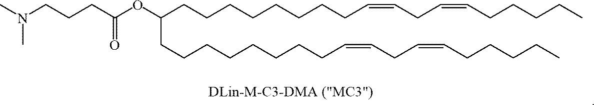
.

15. The pharmaceutical composition of any one of claims 1-14, wherein the LNP further comprises a sterol.

16. The pharmaceutical composition of claim 14, wherein the sterol is cholesterol.

17. The pharmaceutical composition of claim 14, wherein the sterol is b-sitosterol.

18. The pharmaceutical composition of any one of claims 1-17, wherein the LNP further comprises a PEGylated lipid.

19. The pharmaceutical composition of claim 18, wherein the PEGylated lipid is selected from 1-(monomethoxy-polyethyleneglycol)-2,3-dimyristoylglycerol (PEG-DMG), 1,2-distearoyl-sn-glycero-3-phosphoethanolamine—N—[amino(polyethylene glycol) (PEG-DSPE), or both.

20. The pharmaceutical composition of any one of claims 1-19, wherein the LNP further comprises a non-cationic lipid.

21. The pharmaceutical composition of claim 20, wherein the non-cationic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3 -pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof.

22. The pharmaceutical composition of claim 21, wherein the non-cationic lipid is selected from the group consisting of dioleoylphosphatidylcholine (DOPC), distearoylphosphatidylcholine (DSPC), and dioleoyl-phosphatidylethanolamine (DOPE).

23. The pharmaceutical composition of any one of claims 1-22, wherein the LNP further comprises a tissue-specific targeting ligand.

24. The pharmaceutical composition of claim 23, wherein the tissue-specific targeting ligand is selected from mono-antennary GalNAc, tri-antennary GalNAc, and tetra-antennary GalNAc.

25. The pharmaceutical composition of claim 23 or claim 24, wherein the tissue-specific targeting ligand is conjugated to the PEGylated lipid.

26. The pharmaceutical composition of claim 25, wherein the PEGylated lipid is 1,2-distearoyl-sn-glycero-3-phosphoethanolamine—N—[amino(polyethylene glycol) (PEG-DSPE).

27. The pharmaceutical composition of claim 25 or claim 26, wherein the PEGylated lipid conjugated to the tissue-specific targeting ligand is present at a molar percentage of about 0.5%.

28. The pharmaceutical composition of any one of claims 18-27, wherein the PEGylated lipid is present at a molar percentage of about 1.5% to about 3%.

29. The pharmaceutical composition of any one of claims 15-28, wherein the sterol is present at a molar percentage of about 20% to about 40%, and wherein the cationic lipid is present at a molar percentage of about 80% to about 60%.

30. The pharmaceutical composition of claim 29, wherein the sterol is present at a molar percentage of about 40%, and wherein the cationic lipid is present at a molar percentage of about 50%.

31. The pharmaceutical composition of any one of claims 1-30, wherein the composition further comprises a cholesterol, a PEGylated lipid, and a non-cationic lipid.

32. The pharmaceutical composition of claim 25, wherein the PEGylated lipid is present at a molar percentage of about 1.5% to about 3%.

33. The pharmaceutical composition of claim 31 or claim 32, wherein the cholesterol is present at a molar percentage of about 30% to about 50%.

34. The pharmaceutical composition of any one of claims 31-33, wherein the lipid is present at a molar percentage of about 42.5% to about 62.5%.

35. The pharmaceutical composition of any one of claims 31-34, wherein the non-cationic lipid is present at a molar percentage of about 2.5% to about 12.5%.

36. The pharmaceutical composition of any one of claims 31-35, wherein the cholesterol is present at a molar percentage of about 40%, the lipid is present at a molar percentage of about 52.5%, the non-cationic lipid is present at a molar percentage of about 7.5%, and wherein the PEG is present at about 3%.

37. The pharmaceutical composition of any one of claims 31-36, wherein the LNP further comprises a tissue-specific targeting ligand.

38. The pharmaceutical composition of 37, wherein the tissue-specific targeting ligand is selected from mono-antennary GalNAc, tri-antennary GalNAc, and tetra-antennary GalNAc.

39. The pharmaceutical composition of claim 37 or claim 38, wherein the tissue-specific targeting ligand is conjugated to the PEGylated lipid.

40. The pharmaceutical composition of claim 39, wherein the PEGylated lipid conjugated to the tissue-specific targeting ligand is present at a molar percentage of about 0.5%.

41. The pharmaceutical composition of any of claims 1-40, wherein the composition further comprises dexamethasone palmitate.

42. The pharmaceutical composition of any one of claims 1-41, wherein the LNP has a mean diameter of less than about 75 nm.

43. The pharmaceutical composition of any one of claims 1-42, wherein the LNP has a mean diameter of less than about 70 nm.

44. The pharmaceutical composition of any one of claims 1-43, wherein the composition has a total lipid to rigid therapeutic nucleic acid (rTNA) ratio of about 15:1.

45. The pharmaceutical composition of any one of claims 1-43, wherein the composition has a total lipid to rigid therapeutic nucleic acid (rTNA) ratio of about 30:1.

46. The pharmaceutical composition of any one of claims 1-43, wherein the composition has a total lipid to rigid therapeutic nucleic acid (rTNA) ratio of about 40:1.

47. The pharmaceutical composition of any one of claims 1-46, wherein the composition has a total lipid to rigid therapeutic nucleic acid (rTNA) ratio of about 50:1.

48. The pharmaceutical composition of any one of claims 1-47, wherein the rTNA is selected from the group consisting of minigenes, plasmids, minicircles, small interfering RNA (siRNA), microRNA (miRNA), antisense oligonucleotides (ASO), ribozymes, ceDNA, ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof.

49. The pharmaceutical composition of any one of claims 1-48, wherein the rigid therapeutic nucleic acid (rTNA) comprises an expression cassette comprising a promoter sequence and a transgene.

50. The pharmaceutical composition of any one of claims 1-49, wherein the rigid therapeutic nucleic acid (rTNA) comprises an expression cassette comprising a polyadenylation sequence.

51. The pharmaceutical composition of claim 49 or claim 50, wherein the rigid therapeutic nucleic acid (rTNA) comprises at least one inverted terminal repeat (ITR) flanking either a 5′ end or a 3′ end of said expression cassette.

52. The pharmaceutical composition of claim 51, wherein said expression cassette is flanked by two ITRs, wherein the two ITRs comprise one 5′ ITR and one 3′ ITR.

53. The pharmaceutical composition of claim 52, wherein the expression cassette is connected to an ITR at the 3′ end (3′ ITR).

54. The pharmaceutical composition of claim 52, wherein the expression cassette is connected to an ITR at the 5′ end (5′ ITR).

55. The pharmaceutical composition of claim 52, wherein at least one of the 5′ ITR or the 3′ ITR is a wild-type AAV ITR.

56. The pharmaceutical composition of claim 52, wherein at least one of the 5′ ITR and the 3′ ITR is a modified ITR.

57. The pharmaceutical composition of claim 52, wherein the rigid therapeutic nucleic acid (rTNA) further comprises a spacer sequence between the 5′ ITR and the expression cassette.

58. The pharmaceutical composition of claim 52, wherein the rigid therapeutic nucleic acid (rTNA) further comprises a spacer sequence between the 3′ ITR and the expression cassette.

59. The pharmaceutical composition of claim 57 or claim 58, wherein the spacer sequence is at least 5 base pairs long.

60. The pharmaceutical composition of claim 59, wherein the spacer sequence is between about 5 to about 100 base pairs long.

61. The pharmaceutical composition of claim 59, wherein the spacer sequence is between about 5 to about 500 base pairs long.

62. The pharmaceutical composition of any one of claims 1-61, wherein the rigid therapeutic nucleic acid (rTNA) comprises a nick or a gap.

63. The pharmaceutical composition of claim 52, wherein the ITRs are selected from an ITR derived from an AAV serotype, an ITR derived from an ITR of goose virus, an ITR derived from a B19 virus ITR, or a wild-type ITR from a parvovirus.

64. The pharmaceutical composition of claim 63, wherein said AAV serotype is selected from the group consisting of: AAV1, AAV2, AAV3, AAV4, AAV5, AAV6, AAV7, AAV8, AAV9, AAV10, AAV11 and AAV12.

65. The pharmaceutical composition of claim 51, wherein the rTNA comprises a first and a second ITR, wherein the first ITR is a mutant ITR, and the second ITR is different from the first ITR.

66. The pharmaceutical composition of claim 51, wherein the rTNA comprises two mutant ITRs in both 5′ and 3′ ends of the expression cassette, optionally wherein the two mutant ITRs are symmetric mutants with respect to each other.

67. The pharmaceutical composition of claim 1, wherein the rTNA is ceDNA.

68. The pharmaceutical composition of claim 67, wherein the ceDNA is selected from the group consisting of a CELiD, DNA-based minicircle, a MIDGE, a ministering DNA, a dumbbell shaped linear duplex closed-ended DNA (ceDNA) comprising two hairpin structures of ITRs in the 5′ and 3′ ends of an expression cassette, or a doggybone™ DNA.

69. The pharmaceutical composition of claim 1, where the rigid therapeutic nucleic acid is a plasmid.

70. A pharmaceutical composition comprising lipid nanoparticle (LNP), wherein the LNP comprises a lipid and a denatured therapeutic nucleic acid (TNA), wherein the mean diameter of the LNP is between about 20 nm and about 75 nm.

71. The pharmaceutical composition of claim 70, wherein the denatured TNA has a P-form structure.

72. The pharmaceutical composition of claim 70 or claim 71, wherein the denatured TNA is prepared by contacting the denatured TNA in a low molecular weight alcohol/aqueous solution or a non-aqueous solvent system comprising one or more low molecular weight alcohols.

73. The pharmaceutical composition of claim 72, wherein the low molecular weight alcohol/aqueous solution or the non-aqueous solvent system comprises one or more alcohols selected from the group consisting of: ethanol, methanol, and isopropanol.

74. The pharmaceutical composition of any one of claims 70-73, wherein the LNP has a mean diameter of less than about 75 nm.

75. The pharmaceutical composition of any one of claims 70-74, wherein the LNP has a mean diameter of less than about 70 nm.

76. The pharmaceutical composition of any one of claims 70-75, wherein the denatured nucleic acid therapeutic is a double stranded nucleic acid.

77. The pharmaceutical composition of any one of claims 70-76, wherein the denatured nucleic acid therapeutic is a closed-ended DNA (ceDNA).

78. A pharmaceutical composition comprising a lipid nanoparticle (LNP), wherein the LNP comprises a lipid and denatured therapeutic nucleic acid (TNA), wherein the LNP is prepared by a method comprising:
adding aqueous TNA to one or more low molecular weight alcohol solution comprising cationic or ionizable lipids to form a TNA/lipid solution, wherein the final concentration of the low molecular weight alcohol in the solution is between about 80% to about 98%;
mixing the TNA/lipid solution with an acidic aqueous buffer; and
buffer exchanging with a neutral-pH aqueous buffer, thereby producing the LNP formulation.

79. The pharmaceutical composition of claim 78, wherein the final concentration of the low molecular weight alcohol in the solution is between about 87% to about 97%.

80. The pharmaceutical composition of claim 79, wherein the final concentration of the low molecular weight alcohol in the solution is between about 90% to about 95%.

81. The pharmaceutical composition of claim 79, wherein the final concentration of the low molecular weight alcohol in the solution is between about 92% to about 95%.

82. The pharmaceutical composition of claim 79, wherein the mean diameter of the LNP is between about 20 nm and about 75 nm.

83. The pharmaceutical composition of any one of claims 78-82, wherein the LNP has a mean diameter of less than about 75 nm.

84. The pharmaceutical composition of any one of claims 78-83, wherein the LNP has a mean diameter of less than about 70 nm.

85. The pharmaceutical composition of any one of claims 78-84, wherein the rigid or denatured nucleic acid therapeutic is a double stranded nucleic acid.

86. The pharmaceutical composition of any one of claims 78-85, wherein the rigid or denatured nucleic acid therapeutic is a closed-ended DNA (ceDNA).

87. The pharmaceutical composition of any one of claims 1-86, further comprising a pharmaceutically acceptable excipient.

88. A method of producing a lipid nanoparticle (LNP) formulation, wherein the LNP comprises an ionizable lipid and a closed-ended DNA (ceDNA), the method comprising:
adding aqueous ceDNA to one or more low molecular weight alcohols solution comprising cationic or ionizable lipids, wherein the final concentration of alcohol in the solution is between about 80% to about 98% form a ceDNA/lipid solution;
mixing the ceDNA/lipid solution with an acidic aqueous buffer; and
buffer exchanging with a neutral-pH aqueous buffer, thereby producing an LNP formulation.

89. A method of producing a lipid nanoparticle (LNP) formulation comprising an ionizable lipid and a closed-ended DNA (ceDNA), the method comprising:
adding ceDNA to one or more low molecular weight alcohol solution, wherein the alcohol content of the resulting solution is greater than 80%,
adding said ceDNA in >80% alcohol content to cationic or ionizable lipids in low molecular weight alcohol to form a ceDNA/lipid solution, wherein the final concentration of the low molecular weight alcohol in the ceDNA-lipid solution is between about 80% to about 95%;
mixing the ceDNA/lipid solution with an acidic aqueous buffer; and
buffer exchanging with a neutral-pH aqueous buffer, thereby producing the LNP formulation.

90. The pharmaceutical composition of claim 89, wherein the final concentration of the low molecular weight alcohol in the solution is between about 87% to about 97%.

91. The pharmaceutical composition of claim 90, wherein the final concentration of the low molecular weight alcohol in the solution is between about 90% to about 95%.

92. The pharmaceutical composition of claim 90, wherein the final concentration of the low molecular weight alcohol in the solution is between about 92% to about 95%.

93. The method of claim 89, further comprising a step of diluting the mixed ceDNA/lipid solution with an acidic aqueous buffer.

94. The method of any one of claims 89-93, wherein the one or more low molecular weight alcohol is selected from the group consisting of methanol, ethanol, propanol and/or isopropanol.

95. The method of claim 94, wherein the one or more low molecular weight alcohol is ethanol.

96. The method of claim 94, wherein the one or more low molecular weight alcohol is propanol.

97. The method of claim 94, wherein the one or more low molecular weight alcohol is methanol.

98. The method of claim 94, wherein the one or more low molecular weight alcohol is a mixture of ethanol and methanol.

99. The method of any one of claims 88-98, wherein the acid aqueous buffer is selected from malic acid/sodium malate or acetic acid/sodium acetate.

100. The method of any one of claims 88-99, wherein the acidic aqueous buffer is at a concentration of between about 10 to 40 millimolar (mM).

101. The method of any one of claims 88-100, wherein the acidic aqueous buffer is at a pH of between about 3 to 5.

102. The method of any one of claims 88-101, wherein the neutral-pH aqueous buffer is Dulbecco’s phosphate buffered saline, pH 7.4.

103. The method of claim 88, wherein the ceDNA/lipid solution is mixed with the acidic aqueous buffer using microfluidic mixing.

104. The method of claim 88, wherein the final alcohol content following the diluting step is between about 4% to about 15%.

105. The method of claim 88, wherein the flow rate ratio between the acidic aqueous buffer and the ceDNA/lipid solution is 2:1, 3:2, 3:1, 5:1, 6:1, 7:1, 8:1, 9:1, 10:1, 15:1 or 20:1.

106. The method of any one of claims 88-105, wherein the LNP has a mean diameter of between about 20 nm and about 75 nm.

107. The method of any one of claims 88-106, wherein the cationic lipid is MC3 (6Z,9Z,28Z,31Z)-heptatriaconta-6,9,28,31-tetraen-19-yl-4-(dimethylamino) butanoate (DLin-MC3-DMA or MC3) having the following structure: 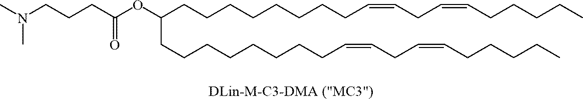 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]
.

108. The method of any one of claims 88-107, wherein the ionizable lipid is a SS-cleavable lipid compring a disulfide bond and a tertiary amine.

109. The method of claim 108, wherein the SS-cleavable lipid is a lipid having the structure: 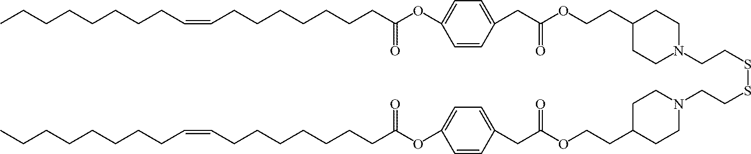 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]
or a pharmaceutically acceptable salt thereof.

110. An LNP formulation produced by the method of any one of claims 88-109.

111. A method of treating a genetic disorder in a subject, the method comprising administering to the subject an effective amount of the pharmaceutical composition according to any one of claims 1-110.

112. The method of claim 111, wherein the subject is a human.

113. The method of claim 111 or claim 112, wherein the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson’s disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi’s anemia, retinitis pigmentosa, ataxia telangiectasia, Bloom’s syndrome, retinoblastoma, mucopolysaccharide storage diseases (e.g., Hurler syndrome (MPS Type I), Scheie syndrome (MPS Type IS), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson’s disease, Alzheimer’s disease, Huntington’s disease, spinocerebellar ataxia, spinal muscular atrophy, Friedreich’s ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency.

114. The method of claim 113, wherein the genetic disorder is Leber congenital amaurosis (LCA) 10.

115. The method of claim 113, wherein the genetic disorder is Stargardt macular dystrophy.

116. The method of claim 113, wherein the genetic disorder is hemophilia A (Factor VIII deficiency).

117. The method of claim 113, wherein the genetic disorder is hemophilia B (Factor IX deficiency).

118. The method of claim 113, wherein the genetic disorder is Wilson’s disease.

119. The method of claim 113, wherein the genetic disorder is Gaucher disease.

120. The method of claim 113, wherein the genetic disorder is phenylketonuria (PKU).

121. The method of claim 113, wherein the genetic disorder is hyaluronidase deficiency.

122. The method of any one of claims 111-121, further comprising administering an immunosuppressant.

123. The method of claim 122, wherein the immunosuppressant is dexamethasone.

124. The method of any one of claims 111-123, wherein the subject exhibits a diminished immune response level against the pharmaceutical composition, as compared to an immune response level observed with an LNP comprising MC3 as a main cationic lipid, wherein the immune response level against the pharmaceutical composition is at least 50% lower than the level observed with the LNP comprising MC3.

125. The method of claim 124, wherein the immune response is measured by detecting the levels of a pro-inflammatory cytokine or chemokine.

126. The method of claim 125, wherein the pro-inflammatory cytokine or chemokine is selected from the group consisting of IL-6, IFNα, IFNy, IL-18, TNFα, IP-10, MCP-1, MIP1α, MIP1β, and RANTES.

127. The method of claim 125, wherein at least one of the pro-inflammatory cytokines is under a detectable level in serum of the subject at 6 hours after the administration of the pharmaceutical composition.

128. The method of any one of claims 111-127, wherein the LNP comprising a SS-cleavable lipid and the closed-ended DNA (ceDNA) is not phagocytosed; or exhibits diminished phagocytic levels by at least 50% as compared to phagocytic levels of LNPs comprising MC3 as a main cationic lipid administered at a similar condition.

129. The method of claim 128, wherein the SS-cleavable lipid is a lipid having the structure: 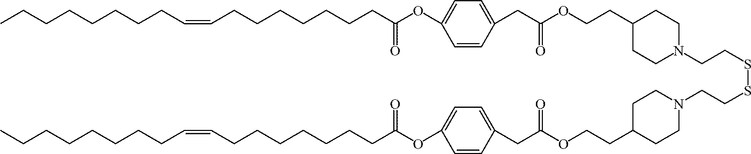
or a pharmaceutically acceptable salt thereof.

130. The method of claim 129, wherein the LNP further comprises cholesterol and a PEGylated lipid.

131. The method of claim 130, wherein the LNP further comprises a noncationic lipid.

132. The method of claim 131, wherein the noncationic lipid is selected from the group consisting of dioleoylphosphatidylcholine (DOPC), distearoylphosphatidylcholine (DSPC), and dioleoyl-phosphatidylethanolamine (DOPE).

133. The method of any of claims 130-132, wherein the LNP further comprises N-Acetylgalactosamine (GalNAc).

134. The method of claim 133, wherein the GalNAc is conjugated to the PEGylated lipid and the PEGylated lipid conjugated to the GalNAc is present in the LNP at a molar percentage of 0.5%.

135. A method of increasing therapeutic nucleic acid targeting to the liver of a subject in need of treatment, the method comprising administering to the subject an effective amount of the pharmaceutical composition according to any one of claims 1-110, wherein the LNP comprises a rigid therapeutic nucleic acid (rTNA), an ss-cleavable lipid, a sterol, and a polyethylene glycol (PEG) and N-Acetylgalactosamine (GalNAc).

136. The method of claim 135, wherein the PEG is 1-(monomethoxy-polyethyleneglycol)-2,3-dimyristoylglycerol (PEG-DMG).

137. The method of claim 135, wherein the LNP further comprises a non-cationic lipid.

138. The method of claim 137, wherein the non-cationic lipid is selected from the group consisting of dioleoylphosphatidylcholine (DOPC), distearoylphosphatidylcholine (DSPC), and dioleoyl-phosphatidylethanolamine (DOPE).

139. The method of claim 135, wherein the GalNAc is conjugated to the PEGylated lipid and the PEGylated lipid conjugated to the GalNAc is present in the LNP at a molar percentage of 0.5%.

140. The method of claim 135, wherein the subject is suffering from a genetic disorder.

141. The method of claim 140, wherein the genetic disorder is hemophilia A (Factor VIII deficiency).

142. The method of claim 140, wherein the genetic disorder is hemophilia B (Factor IX deficiency).

143. The method of claim 140, wherein the genetic disorder is phenylketonuria (PKU).

144. The method of claim 135, wherein the rigid therapeutic nucleic acid is selected from the group consisting of minigenes, plasmids, minicircles, small interfering RNA (siRNA), microRNA (miRNA), antisense oligonucleotides (ASO), ribozymes, ceDNA, ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof.

145. The method of claim 135, wherein the rigid therapeutic nucleic acid is a ceDNA.

146. The method of claim 145, wherein the ceDNA comprises an expression cassette comprising a promoter sequence and a transgene.

147. The method of claim 146, wherein the ceDNA comprises at least one inverted terminal repeat (ITR) flanking either the 5′ or the 3′ end of said expression cassette.

148. The method of claim 135, wherein the ceDNA is selected from the group consisting of a CELiD, a MIDGE, a ministering DNA, a dumbbell shaped linear duplex closed-ended DNA comprising two hairpin structures of ITRs in the 5′ and 3′ ends of an expression cassette, or a doggybone™ DNA, wherein the ceDNA is capsid free and linear duplex DNA.

149. A method of mitigating a complement response in a subject in need of treatment with a rigid therapeutic nucleic acid (rTNA), the method comprising administering to the subject an effective amount of the pharmaceutical composition according to any of the previous claims, wherein the LNP comprises the rTNA, a cationic lipid, a sterol, and a PEGylated lipid.

150. The method of claim 149, wherein the subject is suffering from a genetic disorder.

151. The method of claim 150, wherein the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson’s disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi’s anemia, retinitis pigmentosa, ataxia telangiectasia, Bloom’s syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson’s disease, Alzheimer’s disease, Huntington’s disease, spinocerebellar ataxia, spinal muscular atrophy, Friedreich’s ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency.

152. The method of any one of claims 149-151, wherein the rigid therapeutic nucleic acid is selected from the group consisting of minigenes, plasmids, minicircles, small interfering RNA (siRNA), microRNA (miRNA), antisense oligonucleotides (ASO), ribozymes, ceDNA, ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof.

153. The method of claim 152, wherein the rigid therapeutic nucleic acid is a ceDNA, wherein the ceDNA is selected from the group consisting of a CELiD, a MIDGE, a ministering DNA, a dumbbell shaped linear duplex closed-ended DNA comprising two hairpin structures of ITRs in the 5′ and 3′ ends of an expression cassette, or a doggybone™ DNA, wherein the ceDNA is capsid free and linear duplex DNA.

154. The method of any one of claims 149-153, wherein the PEGylated lipid is 1-(monomethoxy-polyethyleneglycol)-2,3-dimyristoylglycerol (PEG-DMG).

155. The method of claim 154, wherein the PEG is present in the LNP at a molar percentage of about 2 to 4%.

156. The method of claim 155, wherein the PEG is present in the LNP at a molar percentage of about 3%.

157. The method of any one of claims 149-156, wherein the LNP further comprises a non-cationic lipid.

158. The method of claim 157, wherein the non-cationic lipid is selected from the group consisting of dioleoylphosphatidylcholine (DOPC), distearoylphosphatidylcholine (DSPC), and dioleoyl-phosphatidylethanolamine (DOPE).
